SEQUENCE LISTING (47 sequences)

SEQ ID NO: 1 - RNA, 141 nt, Homo sapiens
  source 1..141: mol_type = other RNA
agctttgcgcagtggcagtatcgtagccaatgaggtttatccgaggcgcgattattgctaattgaaaacttttcccaataccccgccatgacgacttgaaatatagtcggcattggcaatttttgacagtctctacggaga

SEQ ID NO: 2 - RNA, 164 nt, Homo sapiens
  source 1..164: mol_type = other RNA
atacttacctggcaggggagataccatgatcacgaaggtggttttcccagggcgaggcttatccattgcactccggatgtgctgacccctgcgatttccccaaatgtgggaaactcgactgcataatttgtggtagtgggggactgcgtccgcgctttcccctg

SEQ ID NO: 3 - RNA, 164 nt, Homo sapiens
  source 1..164: mol_type = other RNA
atacttacctggcaggagagataccctggtcacgaaggtggtttttccagagcgagtcttatccattgcactccggatctgctgacccctgcagtttccccaaatgtgggaaacttgactgtataatttgtggcagtggtagactgcattcgcgctttcccctg

SEQ ID NO: 4 - RNA, 164 nt, Homo sapiens
  source 1..164: mol_type = other RNA
atacttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg

SEQ ID NO: 5 - RNA, 164 nt, Homo sapiens; identical to SEQ ID NO: 4
  source 1..164: mol_type = other RNA

SEQ ID NO: 6 - RNA, 164 nt, Homo sapiens; identical to SEQ ID NO: 4
  source 1..164: mol_type = other RNA

SEQ ID NO: 7 - RNA, 162 nt, Homo sapiens
  source 1..162: mol_type = other RNA
atacttatgtaacaggagaaaatacggccatgaagttggtgtttttcgggggcggtttttccattgtactcagtatgtgctgactgactcctgttacttccacatgtggggaaactggactgtaatttgtggtggtggggaattgcgttcgcgctttcttct

SEQ ID NO: 8 - RNA, 146 nt, Homo sapiens
  source 1..146: mol_type = other RNA
ccacttacctggcaggaaagagaccgtggtcacgaagggggttctcccagagtgaagcttcttcatcgcactctagagttgctgatctctgtgatttcttcaatgtgggaaacggtgtttgtgctagaagagggctgcgctctcta

SEQ ID NO: 9 - RNA, 162 nt, Homo sapiens
  source 1..162: mol_type = other RNA
atacttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttcttct

SEQ ID NO: 10 - RNA, 165 nt, Homo sapiens
  source 1..165: mol_type = other RNA
atatttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgcttttccctg

SEQ ID NO: 11 - RNA, 164 nt, Homo sapiens
  source 1..164: mol_type = other RNA
atacttacctggcaggggagataccatgatcacgaatgtggttttcccagggcgaggcttatccattgcactccggatgtgctgacccctgcgatttccccaaatgtgggaaactcgactgcataatttgtggtagtgggggactgcgttcgcgctttcccctg

SEQ ID NO: 12 - RNA, 167 nt, Homo sapiens
  source 1..167: mol_type = other RNA
ccccttatgtggcgggggacatacagtggtcaggaagggggttctccccggaggaggctagtccaccacacttcggctccgctgacccctgcgatttctccacatgcggggccctcgtccgcggtggtgtttgcgctatccggcggctgggttcgcgcactcactct

SEQ ID NO: 13 - RNA, 164 nt, Homo sapiens
  source 1..164: mol_type = other RNA
atacttacctggcaggggagataccatgatcacgaaggtggttttccctgggcgaggcttatccattgcactccggatgtgctgacccctgcgatttccccaaatgtgggaaacttgactgcataatttgtggtagtgggggactgcgttcgcgctttcccctg

SEQ ID NO: 14 - RNA, 160 nt, Homo sapiens
  source 1..160: mol_type = other RNA
atacttacgtaacaggagaaaatacggccatgaagttggtgtttttcgggggcgatttctccattgtactcagtatgtgctgactgactcctgttacttccacatgtggggaaactggactgtaatttgtggtggtggggaattgcgttcgctttcttct

SEQ ID NO: 15 - RNA, 165 nt, Homo sapiens
  source 1..165: mol_type = other RNA
atacttacctggcaggggagataccatgatcatgaaggcgcttttctcatggtcgcggcttatccattgcgttccagatgtgctgacctctgcgatttccccaaatgtgggaaactcgactgcataatttgtgatagtggggggctgcgttcgtgctttcccctg

SEQ ID NO: 16 - RNA, 164 nt, Homo sapiens
  source 1..164: mol_type = other RNA
atgtttatctgacagaagaaatgttatgatcacgaaggtggtttttccagagcaaggcttagcaattttgctgtggatgtgctgacccctgtgatttccccgaatgtgggaatctccattacataacttgtggcagtggggaactgtgtctgtgttttcccctg

SEQ ID NO: 17 - RNA, 117 nt, Homo sapiens
  source 1..117: mol_type = other RNA
agttttgtgcagtggcagtatcatagccaatgaggtttgtccaatgtacaattattgctaattgaaaacgtttcaggccaagcacagtggctcacgcctataatcccagcactctgg

SEQ ID NO: 18 - RNA, 106 nt, Homo sapiens
  source 1..106: mol_type = other RNA
agctttgtacagtgacaggattatagccaatgaggcatgattattgctaattgaaaacttttcccaataccctggcattggcaatttttgacaatctctatggaga

SEQ ID NO: 19 - RNA, 62 nt, Homo sapiens
  source 1..62: mol_type = other RNA
gagtgttacagcccttttagaatttgtctagcaagtttagaaaatgattcaaagagactgac

SEQ ID NO: 20 - RNA, 123 nt, Homo sapiens
  source 1..123: mol_type = other RNA
agctttgcacagtggcagtatcgtagccaatgagctttacccgaggcgcgattattgctagttaaatatttataaaaacctttcgagcagcgcctgaacaagaataggttcagaggagactcc

SEQ ID NO: 21 - RNA, 63 nt, Homo sapiens
  source 1..63: mol_type = other RNA
cagtgttacagatcttttagaatttgtctagtacattttccagttttcaccaggaatctgcct

SEQ ID NO: 22 - RNA, 82 nt, Homo sapiens
  source 1..82: mol_type = other RNA
agctttgtgcagtggcagtatcatagccaatgaggtttatccgaggcgtgattattgctaattgaaaaattgaggaatagtt

SEQ ID NO: 23 - RNA, 87 nt, Homo sapiens
  source 1..87: mol_type = other RNA
agctttgtgtactggcaatatcataaccaatgaggttcatccaagacatgataattgctaatggcaaacagcataaaaccaactgcc

SEQ ID NO: 24 - RNA, 127 nt, Homo sapiens
  source 1..127: mol_type = other RNA
agctttgcgtgtggcagtatcatagccaatgaggtttatccgaggcgtgattattgctaattgaaaacaaaatggaagacaggtgacttttcccctcccttgaataaacacttcttccacacatgga

SEQ ID NO: 25 - RNA, 85 nt, Homo sapiens
  source 1..85: mol_type = other RNA
agctttgtgcagtggcagtatcgtagccagtgaggtttatcggaggcgtgatcactgctaattgaaaacttttctataaaggaac

SEQ ID NO: 26 - RNA, 63 nt, Homo sapiens
  source 1..63: mol_type = other RNA
gagtgttgcagctcttacagaatttgtctagcaggttttccagtctttaccagaaatgccccc

SEQ ID NO: 27 - RNA, 63 nt, Homo sapiens
  source 1..63: mol_type = other RNA
cagtgttacagctcttttagaatttgttaggctgggcgtactggctcacgcctataatcccaa

SEQ ID NO: 28 - RNA, 64 nt, Homo sapiens
  source 1..64: mol_type = other RNA
gagtgttacagctctattagaatttgtctagcaggttttctggtcttcactggaaagcaccccc

SEQ ID NO: 29 - RNA, 176 nt, Homo sapiens
  source 1..176: mol_type = other RNA
atacttacctggcagggcagataccatgatcttaaaggcagttttcccagggcaaggcttatccattccactctggatccatcatagggatatgctgatccctggaattgccccaaatgtgggaagctctactgcaaaatttttggtagtgagcgatggcattatgcattcatgta

SEQ ID NO: 30 - RNA, 60 nt, Homo sapiens
  source 1..60: mol_type = other RNA
cagtgttacagctcttttagaatttttctagcaggttttccagttttcactggaacctct

SEQ ID NO: 31 - RNA, 61 nt, Homo sapiens
  source 1..61: mol_type = other RNA
cagtgttacagctcttttggaattagtctagcagatttctcagttttcactggaaaccctt

SEQ ID NO: 32 - RNA, 116 nt, Homo sapiens
  source 1..116: mol_type = other RNA
atactctggtttctcttcagatcgtataaatctttcgccttttactaaagatttccgtggagaggaacaactctgagtcttaagctaattttttgaggccttgttccgacaaggct

SEQ ID NO: 33 - RNA, 150 nt, Homo sapiens
  source 1..150: mol_type = other RNA
atgccttaaacttatgagtaaggaaaataacgattcggggtgacgcccgaatcctcactgctaatgtgagacgaatttttgagcgggtaaaggtcgccctcaaggtgacccgcctactttgcgggatgcctgggagttgcgatctgcccg

SEQ ID NO: 34 - RNA, 134 nt, Homo sapiens
  source 1..134: mol_type = other RNA
aaaaagggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 35 - RNA, 120 nt, Homo sapiens
  source 1..120: mol_type = other RNA
atactctggtttctcttcaaatcgtataaatctttcgccttttactaaagatttccgtggagagaaacgagtgtgagtctgaaaccaattttttgaggccttgcgtttcttagcagggct

SEQ ID NO: 36 - RNA, 107 nt, Homo sapiens
  source 1..107: mol_type = other RNA
gtgctcgcttcggcagcacatatactaaaattggaacgatacagagaagattagcatggcccctgcgcaaggatgacacgcaaattcgtgaagcgttccatattttt

SEQ ID NO: 37 - RNA, 116 nt, Homo sapiens
  source 1..116: mol_type = other RNA
atgctctggtttctcttcaaatcgtataaatctttcgccttttactaaagatttccgtggagagaaaccgttttgagtttcaagcaaattttttgaagccctatgttggtggggtt

SEQ ID NO: 38 - RNA, 116 nt, Homo sapiens
  source 1..116: mol_type = other RNA
atactctggtttctcttcagatcgcataaatctttcgccttttactaaagatttccgtggagaggaacaactctgagtcttaacccaattttttgaggccttgctttggcaaggct

SEQ ID NO: 39 - RNA, 165 nt, Homo sapiens
  source 1..165: mol_type = other RNA
atacttacttggcgggggagataccatgatcacgaaggtggttttctcagggcgaggcttatccgttatgttccgggtgtactgacccctgccattttcccccaatgtgaggaactcgactgcataacttgtgatagtaggggactgcgttcgcgctttcccctg

SEQ ID NO: 40 - RNA, 164 nt, Homo sapiens
  source 1..164: mol_type = other RNA
atacttacctggcaggggagatacgatgatcacgaaggtggttttcccagggagaggcttatccattgcactccggatgtgctgacccctgccgtttccccaaatgtgggaaactcgactgcataatttgtggtagtgggggactgcgttcgcgctgtcctctg

SEQ ID NO: 41 - RNA, 126 nt, Homo sapiens
  source 1..126: mol_type = other RNA
gtgttgtatgaaaggagagaaggttagcactccccttgacaaggatggaagaggccctcgggcctgacaacacgcatacggttaaggcattgccacctacttcgtggcatctaaccatcgtttttt

SEQ ID NO: 42 - RNA, 167 nt, Homo sapiens
  source 1..167: mol_type = other RNA
atacttacctggcaggggagatactattatcaaacgaaggtggtttttctcagggcgagggttatccattgtgttccggatgtgctgacctctgcgatttccccaaacgtgggaaactcgactgcataatttgtggtagtgggggactgcgttcgcgctttcctctg

SEQ ID NO: 43 - RNA, 141 nt, Homo sapiens
  source 1..141: mol_type = other RNA
agctttgcgcagtggcagtatcgtagccaatgaggtctatccgaggcgcgattattgctaattgaaaacttttcccaataccccgccgtgacgacttgcaatatagtcggcactggcaatttttgacagtctctacggaga

SEQ ID NO: 44 - RNA, 117 nt, Homo sapiens
  source 1..117: mol_type = other RNA
gatctctggtttctcttcataacgaataaatctttcgccttttactaaagatttccgtggagaaaaacaactatgagtttatggttaaattttttgaagtcttgcctaggcaaggct

SEQ ID NO: 45 - RNA, 127 nt, Homo sapiens
  source 1..127: mol_type = other RNA
accatccttttcttggggttgcgctactgtccaatgagcgcatagtgagggcagtactgctaacgcctgaacaacacacccgcatcaactagagcttttgctttattttggtgcaatttttggaaaa

SEQ ID NO: 46 - RNA, 107 nt, Homo sapiens; identical to SEQ ID NO: 36
  source 1..107: mol_type = other RNA

SEQ ID NO: 47 - RNA, 107 nt, Homo sapiens; identical to SEQ ID NO: 36
  source 1..107: mol_type = other RNA

(19) United States
(12) Patent Application Publication
(10) Pub. No.: US 2026/0226557 A1
(43) Pub. Date: Aug. 6, 2026

(54) CANCER SUBTYPE IDENTIFIER AND METHOD FOR CLASSIFYING TUMORS

(71) Applicant: Indivumed GmbH, Hamburg (DE)

(72) Inventors: Aslihan Ambeskovic, Rochester, NY (US); Karl Hartmut Land, Rochester, NY (US); Jonathan Woodsmith, Hamburg (DE)

(21) Appl. No.: 19/423,247

(22) Filed: Dec. 17, 2025

Related U.S. Application Data
(63) Continuation-in-part of application No. 18/989,420, filed on Dec. 20, 2024, now abandoned.

(51) Int. Cl. C12Q 1/6886

(52) U.S. Cl. CPC C12Q 1/6886; C12Q 2600/106; C12Q 2600/112; C12Q 2600/118; C12Q 2600/158

(57) ABSTRACT

The present disclosure relates to cancer subtype identifiers, which classify tumors and methods based thereon. Further it relates to methods and kits for predicting the prognosis for a subject suffering from cancer. Moreover, it relates to methods for treating a subject suffering from cancer. Further, the disclosure relates to the use of cancer subtype identifiers.

### CROSS REFERENCE TO RELATED APPLICATIONS

This application depends from and claims priority to U.S. application Ser. No. 18/989,420 filed Dec. 20, 2025, the entire contents of which are incorporated herein by reference.

### SEQUENCE LISTING

The instant application contains a Sequence Listing which has been submitted in .xml format via Patent Center and is hereby incorporated by reference in its entirety. Said .xml copy, created on Dec. 15, 2025, is named Sequence_Listing_IDIV0004IA.xml and is 47,192 bytes in size.

### FIELD

The present disclosure relates to a cancer subtype identifiers, which classify tumors and a methods, kits for predicting the prognosis for a subject suffering from cancer based thereon, methods for treating a subject suffering from cancer, and the use of the cancer subtype identifiers.

### BACKGROUND

Cancer is a group of diseases involving abnormal cell growth with the potential to invade or spread to other parts of the body. Each year a great number of humans are newly diagnosed with cancer and more than 100 different types of cancer are known. Two of the most common cancer diseases are colorectal cancer and lung cancer (e.g., non-small lung cancer). There is a great demand for methods and diagnostics that allow predicting disease progression and outcome for a cancer patient in specially in view of a certain treatment.

Clinical experience and cancer research have led to the understanding that cancer is not one disease but occurs as several different subtypes. These subtypes are distinguished by distinct patterns of gene expression that influence or determine tumor biology.

A colorectal cancer staging system based on TNM (T—primary tumor, N—regional lymph node status, M—distant metastasis) is used as the main prognostic predictor post-surgery and governs the rationale for subsequent adjuvant therapy. Despite the prognostic utility of tumor staging, the clinical outcome can vary significantly across patients within the same stage. J. Taieb et al., Cancers, 2020; 12, 2679 and S. M. Qaderi et al., Int J Colorectal Dis. 2021; 36:2399-2410 describe that fewer than 30% of patients in stage III CRC will benefit from chemotherapy and more than 30% will go on to develop distant metastases. Thus, there is an unmet need for biomarkers to address heterogeneity among existing risk strata to enable more accurate diagnoses and tailored treatment options.

To resolve the heterogeneity among the CRC patients, “Omic” approaches identified various molecular features of tumors and classified CRCs into consensus molecular subtypes (CMS) (J. Guinney et al., Nat Med. 2015; 21 (11), 1350-1356). The study by I. Joanito I, et al., Nat. Genet. 2022, 54, 963-975, refines CMS by identifying 2 intrinsic epithelial subtypes. However, these intrinsic subtypes are significantly associated with conventional CMS and do not refine tumor staging. Furthermore, studies have shown that CMS stratification does not outperform TNM staging prognostically (R. V. Purcell et al., BMC Cancer, 2019, 19, 1155).

E. Domingo et al. J Clin Oncol. 2024, 42 (18), 2207-2218 discusses in situ immune cell infiltrate, which also has been associated with tumor progression. The prognostic value of a scoring method (Immunoscore) based on quantification of host CD3⁺ and CD8⁺ T-cell densities in the tumor and its invasive margin in stage III CRC patients has been effectively demonstrated. However, the prognostic predictive power of Immunoscore varies across different subgroups of patients. For example, Immunoscore is not effective in patients with DNA mismatch repair deficient cases thus limiting its applicability. Furthermore, Immunoscore requires standardized testing and interpretation of immune cell densities which may make Immunoscore difficult to incorporate into routine clinical practice.

Studies from R. Salazar et al. J Clin Oncol. 2011, 29 (1), 17-24 and A. Sveen et al. American Association for Cancer Research, 2012, 18 (21), 6001-6010 identified gene expression signatures that can refine stage II and stage III CRC patients respectively denoted as ColoPrint. From these studies, Salazar et al. cannot address the utility of ColoPrint in stage Ill patients as the study utilized a relatively small sample size (n=62). An inherent limitation of most gene-expression-based subtyping methods lies in their dependence on older microarray technology, which lacks the dynamic range, precision in quantification, and the unbiased detection capabilities offered by RNA sequencing. This limitation has been confirmed by a meta-analysis study, which demonstrated that signatures from various studies, including ColoGuidePro, failed to reliably predict prognosis (Z. Sztupinszki, Sci Rep., 2016, 6, 37169).

WO2024/218161 and Ambeskovic A. et al., Gastroenterology, 2024, 167, 1358-1370, disclose a colorectal cancer subtype identifier based on percent spliced-in (PSI) values from exon skipping. It was found that a colorectal cancer subtype identifier utilizing percent spliced-in (PSI) values derived from alternative splicing events is capable of classifying consensus molecular subtypes (CMS). It has to be mentioned that the consensus molecular subtype model for predicting the prognosis of a cancer patient is not equivalent to the cancer subtype identifier based on the classification of MXE1 and MXE2 subtype.

US2023/0021094 relates to a biomarker composition for predicting the prognosis of a patient diagnosed with rectal cancer based on the molecular subtype model (CMS). In particular, the expression of small nuclear RNAs (e.g., RNU5A-1, RNU5B-1, RNU5E-1, RNU4-1, and RNU6ATAC) has been reported to be associated with better prognosis in general; however, in MXE2 tumors, these same RNAs are associated with worse prognosis

US2021/0388449 relates to method for determining an integrated, pan-cancer subtype (clustering of cluster assignments (COCA) subtype of cancer) for predicting the prognosis of a patient diagnosed with cancer. The method includes detecting the expression level of genes to classify biomarkers for categorization of tumors into one of 21 of cluster assignments (COCA) subtypes. Again, such model is not equivalent to the cancer subtype identifier based on the classification of MXE1 and MXE2 subtype.

US20140066319 relates to a method for assessing metastatic potential of breast cancer. The method is based on Epithelial-to-Mesenchymal Transition (EMT)-associated splicing pattern. The document does not show that the splicing events are associated with prognosis of patients. The subtypes MXE1 and MXE2 according to the invention are independent of CMS (consensus molecular subtypes), which includes CMS4 defined by EMT. Thus, MXE subtypes are independent of EMT.

S. Zeeshan et al., Oncogene 2025, 44, 958-967, relates to alternative splicing events associated with the subtypes of lung cancer, i.e., adenocarcinoma (LUAD) and squamous cell carcinoma (LUSC). This document does not mention any evidence for prognostic values of these events.

H. Chen et al., BMC cancer 2020, 20, 904, 1-11 relates to a study of identification and validation of alternative splicing signature based on survival associated alternative splicing events as an independent factor for colon cancer and the alternative splicing signature does not include MXE events.

Z. Zhang et al., Genomic, 2020, 112 (6), 4032-4040 relates to a study of alternative splicing (AS) with relapse in CRC. A prognostic signature predicting relapse in CRS in stages I-III based on alternative splicing is also constructed. It has to be mentioned that stage I-III in CRC is not equivalent to the cancer subtype identifier based on the classification of MXE1 and MXE2 subtype.

There are several disadvantages in conventional/known subtype determination for various cancer tumors that can be addressed. Consequently, to determine new subtypes and the associated prognosis of tumors, there is an urgent need for a time and cost-effective clinical test.

Furthermore, there is considerable variability in patient outcomes within stage III colorectal cancer (CRC). This variability highlights the potential risk of overtreating some stage III patients who may not benefit from chemotherapy regimens. Therefore, there is a need for reliable biomarkers that can enable effective stratification of patients within a given stage, thus allowing matching of the most appropriate treatment option for each patient.

Therefore, it is an object of the invention to provide a method for classifying new subtypes for patients having various types of cancer, optionally colorectal cancer. It is further an object of the invention to provide a method for predicting the prognosis for a subject suffering from a cancer.

Surprisingly, it was found that a cancer subtype identifier utilizing percent spliced-in (PSI) values derived from mutually exclusive exon event, expression of small nuclear RNAs or expression of protein-coding genes is capable of classifying new subtypes for cancer diseases.

### SUMMARY

The invention relates to a cancer subtype identifier, which classifies tumors by at least one method selected from
percent spliced-in (PSI) values based on the occurrence of at least one mutually exclusive exon usage event,
expression of small nuclear RNAs and
expression of predictive protein-coding genes
and combinations thereof.

The invention relates further to a cancer subtype identifier, which classifies tumors by at least one method selected from
percent spliced-in (PSI) values based on the occurrence of at least one mutually exclusive exon usage event,
expression of small nuclear RNAs and
expression of predictive protein-coding genes
and combinations thereof,
   wherein the tumor is classified as MXE1 subtype and MXE2 subtype.

The invention further relates to a cancer subtype identifier, which classifies tumors based on at least one of
PSI (percent spliced-in) values based on the occurrence of at least one mutually exclusive exon usage event,
expression of small nuclear RNAs,
expression of predictive protein-coding genes,
a combination thereof,
   wherein the tumor is classified as MXE1 subtype and MXE2 subtype, wherein the MXE1 subtype is characterized by
i) a distinct combination of PSI values from mutually exclusive exon usage events,
ii) a distinct combination of expressions of small nuclear RNAs
iii) a distinct combination of protein coding genes,
a combination of i) and ii), i) and iii), ii) and iii) and i), ii) and iii),
wherein MXE1 subtype is associated with a favorable prognosis,
and/or
the MXE2 subtype is characterized by
iv) a distinct combination of PSI values from mutually exclusive exon usage events,
v) a distinct combination of expressions of small nuclear RNAs
vi) a distinct combination of protein coding genes,
a combination of iv) and v), iv) and vi), v) and vi) and iv), v) and vi),
wherein the MXE2 subtype is associated with an unfavorable prognosis compared to the MXE1 subtype.
wherein i) is different from iv), ii) is different from v), iii) is different from vi).

In a special embodiment, the invention relates to a cancer subtype identifier, which classifies tumors by determining
the percent spliced-in (PSI) values based on the occurrence of at least one mutually exclusive usage exon event,
the expression of small nuclear RNAs and
the expression of protein-coding genes.

The invention further relates to method for classifying tumors using a cancer subtype identifier based on at least one
PSI values based on the occurrence of at least one mutually exclusive exon usage event,
expression of small nuclear RNAs
expression of predictive protein-coding genes,
a combination thereof.

The invention relates further to method for classifying tumors using a cancer subtype identifier based on at least one
percent spliced-in (PSI) values based on the occurrence of at least one mutually exclusive exon usage event,
expression of small nuclear RNAs and
expression of predictive protein-coding genes
and combinations thereof,
   wherein the tumor is classified as MXE1 subtype and MXE2 subtype.

The invention further relates to method for classifying tumors using a cancer subtype identifier based on at least one
PSI (percent spliced-in) values based on the occurrence of at least one mutually exclusive exon usage event,
expression of small nuclear RNAS,
expression of predictive protein-coding genes,
a combination thereof,
   wherein the tumor is classified as MXE1 subtype and MXE2 subtype, wherein
   the MXE1 subtype is characterized by
   i) a distinct combination of PSI values from mutually exclusive exon usage events,
   ii) a distinct combination of expressions of small nuclear RNAs
   iii) a distinct combination of protein coding genes,
   a combination of i) and ii), i) and iii), ii) and iii) and i), ii) and iii),
   wherein MXE1 subtype is associated with a good prognosis compared to the MXE2 subtype, and/or the MXE2 subtype is characterized by
   iv) a distinct combination of PSI values from mutually exclusive exon usage events,
   v) a distinct combination of expressions of small nuclear RNAs
   vi) a distinct combination of protein coding genes,
   a combination of iv) and v), iv) and vi), v) and vi) and iv), v) and vi),
   wherein the MXE2 subtype is associated with a poor prognosis compared to the MXE1 subtype.
   wherein i) is different from iv), ii) is different from v), iii) is different from vi).

The invention further relates to method for predicting the prognosis for a subject suffering from cancer, the method comprising using a cancer subtype identifier based on PSI values based on an occurrence of at least one mutually exclusive exon usage event, and/or expression of small nuclear RNAs and/or expression of predictive protein-coding genes comprising the steps:
a) collecting a sample from a tumor and/or body fluid and/or body excretion of a subject;
b) determining at least one of
   the PSI values based on the occurrence of at least one mutually exclusive exon usage event,
   deregulation of the expression of small nuclear RNAs,
   expression of subtype predictive protein-coding genes,
   combinations thereof; and
c) conversion of values of step b) into a rating scale to indicate the disease prognosis.

In particular, the invention relates to method for predicting the prognosis for a subject suffering from cancer, the method comprising using a cancer subtype identifier based on PSI values based on an occurrence of at least one mutually exclusive exon usage event, and/or expression of small nuclear RNAs and/or expression of predictive protein-coding genes comprising the steps:
a) collecting a sample from a tumor and/or body fluid and/or body excretion of a subject in a non-therapeutic manner;
b) determining at least one of
   the PSI values based on the occurrence of at least one mutually exclusive exon usage event,
   deregulation of the expression of small nuclear RNAs,
   expression of subtype predictive protein-coding genes,
   combinations thereof; and
c) conversion of values of step b) into a rating scale to indicate the disease prognosis, the conversion of values of step b) to indicate the disease prognosis classifies the tumors as MXE1 subtype or MXE2 subtype, preferably wherein in step c) the conversion of the values of step b) to indicate the disease prognosis is used to decide whether the subject is subjected to a treatment by anti-tumor therapy and/or surveillance of progress of the disease.

In a special embodiment the rating scale classifies tumors as MXE1 subtype or MXE2 subtype.

In particular, the MXE1 subtype is associated with a good (favorable) prognosis compared to the MXE2 subtype. The MXE2 subtype is associated with a poor (unfavorable) prognosis compared to the MXE1 subtype.

The invention further relates to a method for treating a subject suffering from cancer the method comprising using a cancer subtype identifier based on PSI values based on an occurrence of at least one mutually exclusive exon event, and/or the deregulation of expression of small nuclear RNAs and/or deregulation of expression of subtype predictive protein-coding genes comprising the steps:
a′) collecting a sample from a tumor and/or body fluid and/or body excretion of a subject;
b′) determining at least one of
   the PSI values based on the occurrence of at least one mutually exclusive exon usage event,
   deregulation of the expression of small nuclear RNAs,
   expression of subtype predictive protein-coding genes, combinations thereof;
c′) conversion of the values of step b) into a rating scale to indicate the disease prognosis by classifying the tumors as MXE1 subtype or MXE2 subtype; and
d′) subjecting the subject to an anti-tumor treatment if the rating scale indicates that the subject is likely to experience increased progression of the disease.

The invention further relates to a composition (e.g., biomarker) for predicting the prognosis for a subject suffering from cancer, in particular colorectal cancer, selected from the group consisting of U1 small nuclear 1 RNA (RNU1-1); U12 small nuclear RNA (RNU12); U4 small nuclear 2 RNA (RNU4-2); U5A small nuclear 1 RNA (RNU5A-1); U5B small nuclear 1 RNA (RNU5B-1); U5D small nuclear 1 RNA (RNU5D-1); U5E small nuclear 1 RNA (RNU5E-1); U6 small nuclear 9 RNA (RNU6-9); U6atac small nuclear RNA (RNU6ATAC); variant U1 small nuclear 1 RNA (RNVU1-1); variant U1 small nuclear 15 RNA (RNVU1-15); variant U1 small nuclear 18 RNA (RNVU1-18); variant U1 small nuclear 19 RNA (RNVU1-19); variant U1 small nuclear 6 RNA (RNVU1-6); variant U1 small nuclear 7 RNA (RNVU1-7); U11 small nuclear RNA (RNU11); U1 small nuclear 4 RNA (RNU1-4); U4 small nuclear 1 RNA (RNU4-1); U4atac small nuclear RNA (RNU4ATAC); U5F small nuclear 1 RNA (RNU5F-1); U6 small nuclear 1 RNA (RNU6-1); U6 small nuclear 2 RNA (RNU6-2); U1 small nuclear RNA (RF00003); U4 small nuclear RNA (RF00015) and U7 small nuclear RNA (RF00066).

In a special embodiment RNU11, RNU12, RNU4-1, RNU4ATAC, RNU5A-1, RNU5B-1, RNU5D-1, RNUSE-1, RNU5F-1, RNU6-1, RNU6-2, RNU6-9, RNU6ATAC, RNVU1-1, RNVU1-15, RNVU1-19 are upregulated in particular, in MXE2 subtype compared to MXE1 subtype and RNU1-1, RNU1-4, RNU4-2, RNVU1-18, RNVU1-6, RNVU1-7, RF00015, RF00066 and RF00003 are downregulated, in particular in MXE2 subtype compared to MXE1 subtype.

In particular, RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC, RNVU1-1, RNVU1-15, RNVU1-19 are upregulated in MXE2 subtype compared to MXE1 subtype and RNU1-1, RNU1-4, RNU4-2, RNVU1-18, RNVU1-6, RNVU1-7, RF00015, RF00066 and RF00003 are downregulated in MXE2 subtype compared to MXE1 subtype, which is associated with poor prognosis.

The invention further relates to a composition (e.g., biomarker) for predicting the prognosis for a subject suffering from cancer, in particular non-small cell lung cancer (lung adenocarcinoma), selected from RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC, RF00015 and RF00066.

In a special embodiment RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC are upregulated and RF00015 and RF00066 are downregulated.

In particular, RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC are upregulated in MXE2 subtype compared to MXE1 subtype and RF00015 and RF00066 are downregulated in in MXE2 subtype compared to MXE1 subtype, which is associated with poor prognosis.

The invention further relates to a kit for predicting the prognosis for a subject suffering from cancer using at least one of PSI values based on an occurrence of at least one mutually exclusive exon usage event, deregulation of the expression of small nuclear RNAs expression of subtype predictive protein-coding genes, combinations thereof, comprising a composition for determining at least one of the PSI value, the expression of small nuclear RNAs and the expression protein-coding genes.

In particular, the invention relates to a kit for predicting the prognosis for a subject suffering from cancer using the cancer subtype identifier as defined herein, comprising a composition for determining the PSI value, the expression of small nuclear RNAs and the expression protein-coding genes.

The invention further relates to the use at least one of the PSI values based on the occurrence of at least one mutually exclusive exon usage event, the expression of small nuclear RNAs the expression of protein-coding genes, combinations thereof as a marker of the prognosis for a subject suffering from cancer, in particular colorectal cancer and non-small lung cancer (lung adenocarcinoma).

In particular, the invention relates to the use of the cancer subtype identifier as defined herein comprising a composition for determining the PSI value, the expression of small nuclear RNAs and the expression protein-coding genes.

### BRIEF DESCRIPTION OF THE DRAWINGS

FIG. 1: (A) Cophenetic correlation coefficients for NMF clusters show the stability of groups. (B) Heatmap of z-scores for PSI values associated with 2 emerging groups.

FIG. 2: Estimated progression-free survival associated with MXE-based groups as shown by Kaplan-Meier analysis. Group-2 is associated with increased progression of the disease (P<0.0001)

FIG. 3: Estimated progression-free survival associated with MXE-based groups stratified further by tumor-staging as shown by Kaplan-Meier analysis. MXE-based groups can refine prognosis for patients with stage III tumors. Univariate Cox proportional hazards regression was performed and the resulting hazard ratios (HRs) along with their 95% confidence intervals (CIs) and P-values are shown.

FIG. 4: Estimated progression-free survival associated with predicted MXE-based groups as shown by Kaplan-Meier analysis. A logistic-regression model based on stable MXE events distinguishing the prognostic subtypes was validated on the internal validation cohort (n=246, samples with ambiguous CMS).

FIG. 5: Estimated progression-free survival associated with predicted MXE-based groups stratified further by tumor-staging in the validation cohort (n=246), as shown by Kaplan-Meier analysis. Univariate Cox proportional hazards regression was performed and the resulting hazard ratios (HRs) along with its 95% confidence intervals (CIs) and P-values are shown.

FIG. 6: Portion of events distinguishing MXE subtypes that correlate with the indicated snRNAs, splicing and RNA binding factors.

FIG. 7: The snRNAs deregulated between MXE-based groups, as shown by Volcano plot.

FIG. 8: Estimated progression-free survival associated with predicted MXE-based groups, as shown by Kaplan-Meier analysis. A logistic-regression model based on expression of snRNAs was validated on the internal validation cohort (n=246, samples with ambiguous CMS).

FIG. 9: Estimated progression-free survival associated with snRNA based prognostic groups stratified further by tumor-staging in the validation cohort (n=246), as shown by Kaplan-Meier analysis. Univariate Cox proportional hazards regression was performed and the resulting hazard ratios (HRs) along with its 95% confidence intervals (CIs) and P-values are shown.

FIG. 10: Estimated progression-free survival associated with predicted MXE-based groups, as shown by Kaplan-Meier analysis. A logistic-regression model based on stable protein-coding genes distinguishing the prognostic subtypes was validated on the internal (n=246, samples with ambiguous CMS) and external (n=266, TCGA) validation cohorts.

FIG. 11: Estimated progression-free survival associated with protein-coding gene based prognostic groups stratified further by tumor-staging in the (A) internal (n=246, samples with ambiguous CMS) and (B) external (n=266, TCGA) validation cohorts, as shown by Kaplan-Meier analysis.

FIG. 12: (A) Univariate Cox proportional hazards regression was performed for internal (n=246, samples with ambiguous CMS) and (B) external (n=266, TCGA) validation cohorts and the resulting hazard ratios (HRs) for stages stratified by prognostic groups along with their 95% confidence intervals (CIs) and P-values are shown. Proportion of progression events (defined as metastasis, local relapse or tumor associated death) for each tumor stage stratified by predicted MXE-based subtype are shown for internal (n=246, samples with ambiguous CMS) and (B) external (n=266, TCGA) validation cohorts.

FIG. 13: (A) Visium HD spatial transcriptomic profiling of MXE1 (n=3) and MXE2 (n=4) samples revealed distinct clusters. Heatmaps display differentially expressed genes between MXE1 and MXE2 within each spatial cluster. (B) Cox regression analysis of spatial signatures derived from individual clusters in bulk RNA-seq data (n=429, validation n=246). Patient stratification based on median MCP-counter enrichment scores for MXE2 up-regulated genes (enriched vs. depleted).

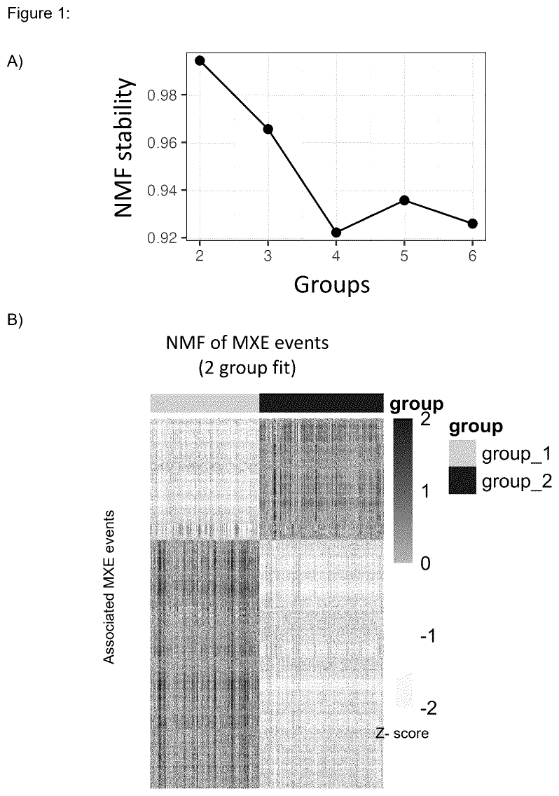

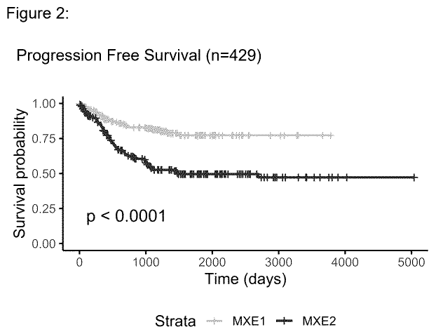

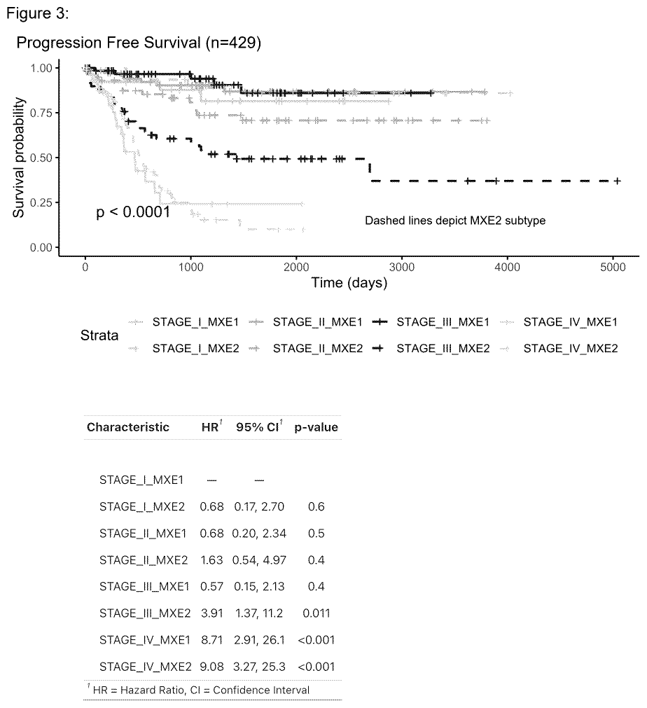

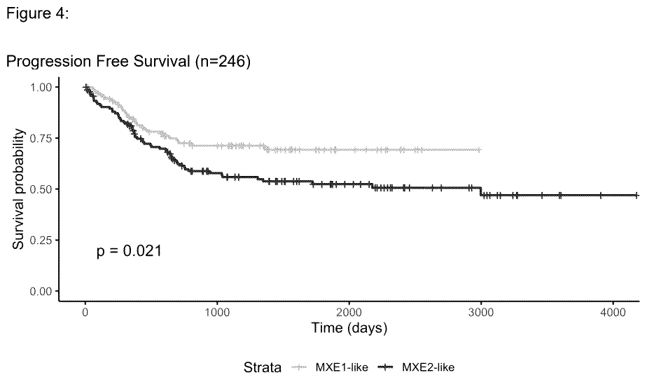

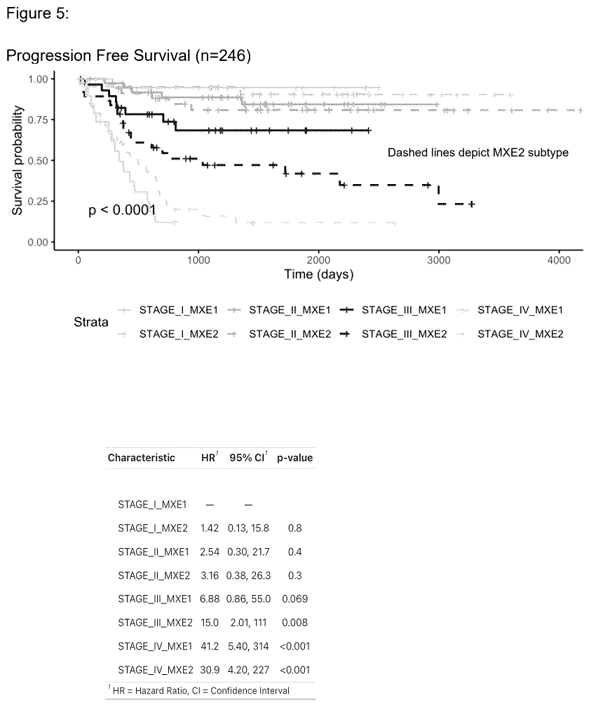

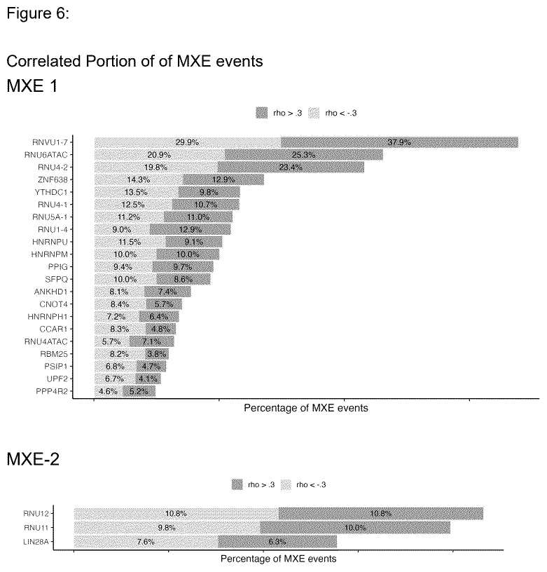

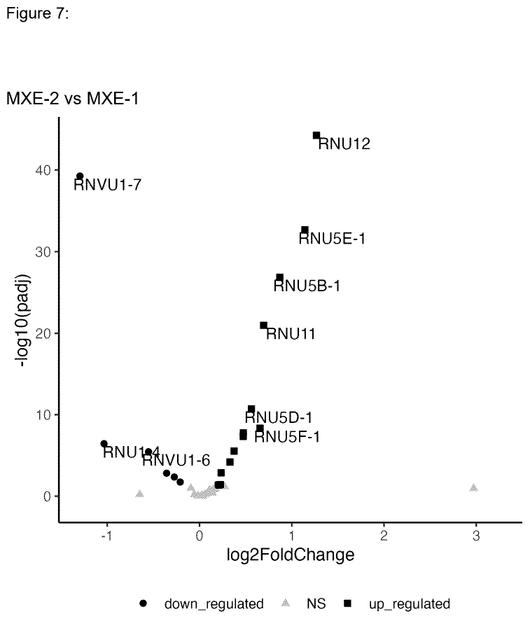

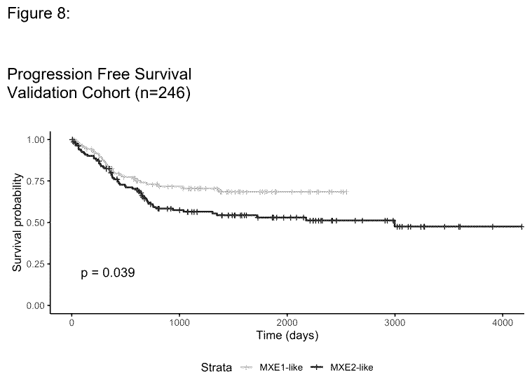

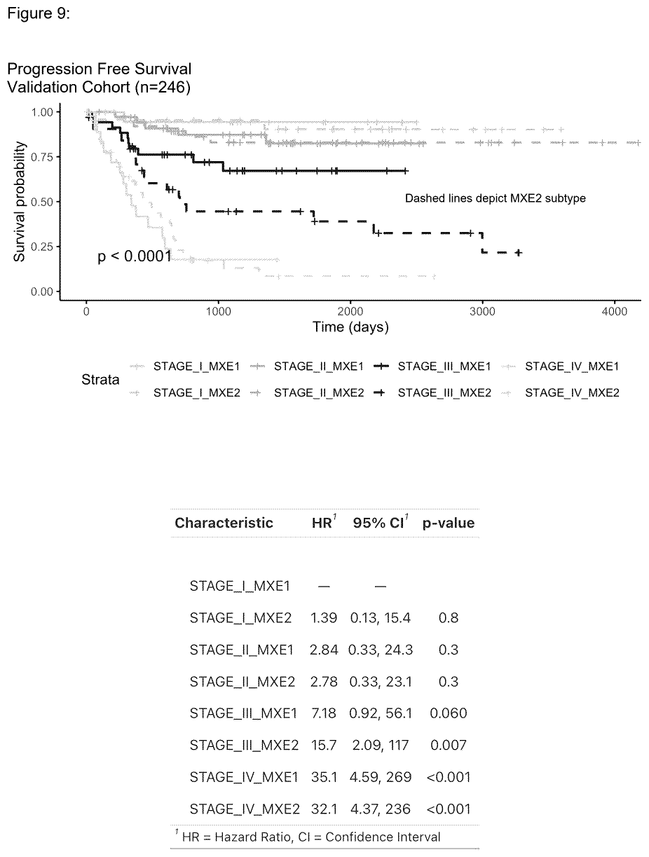

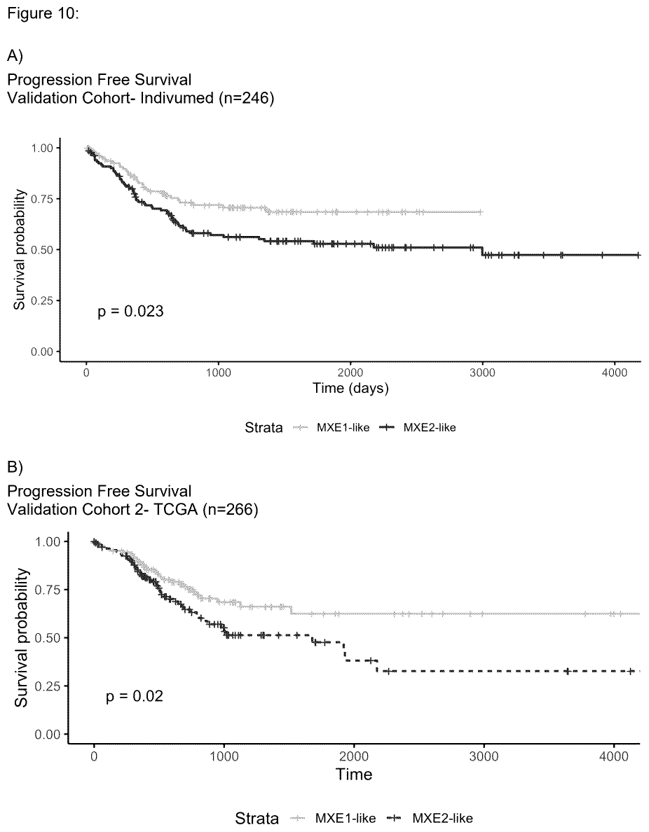

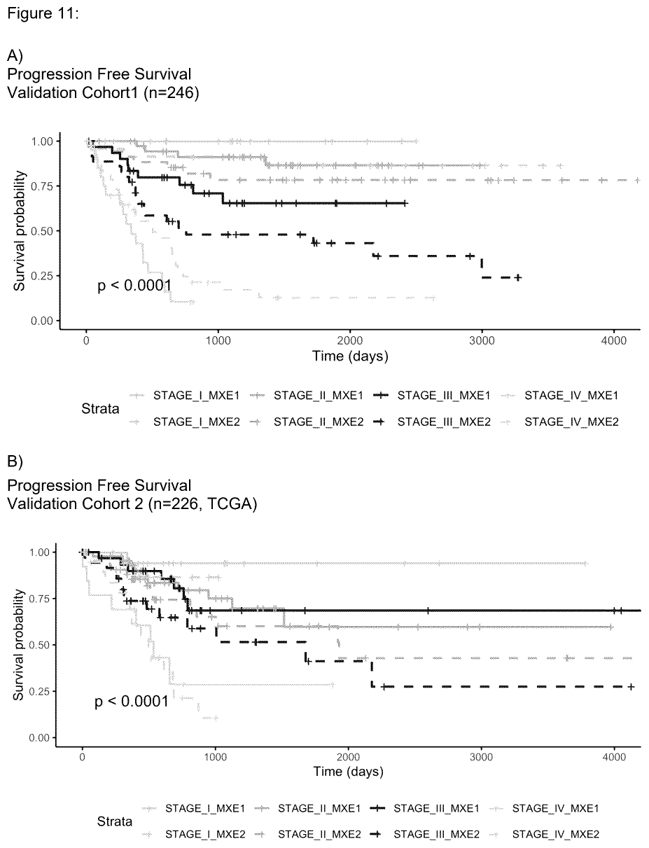

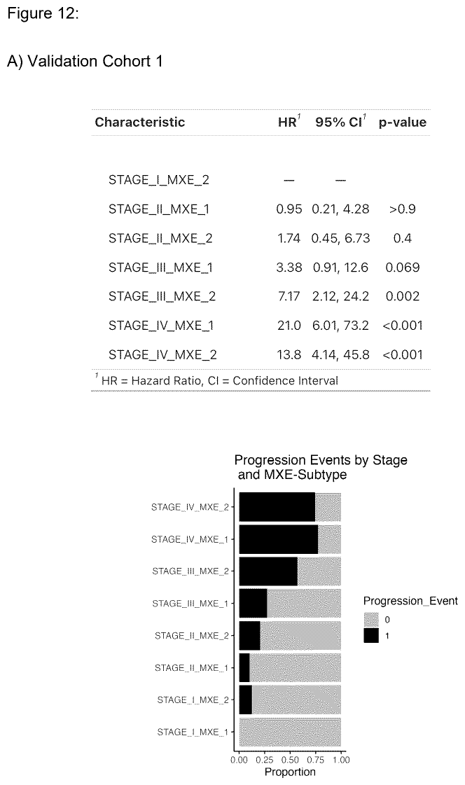

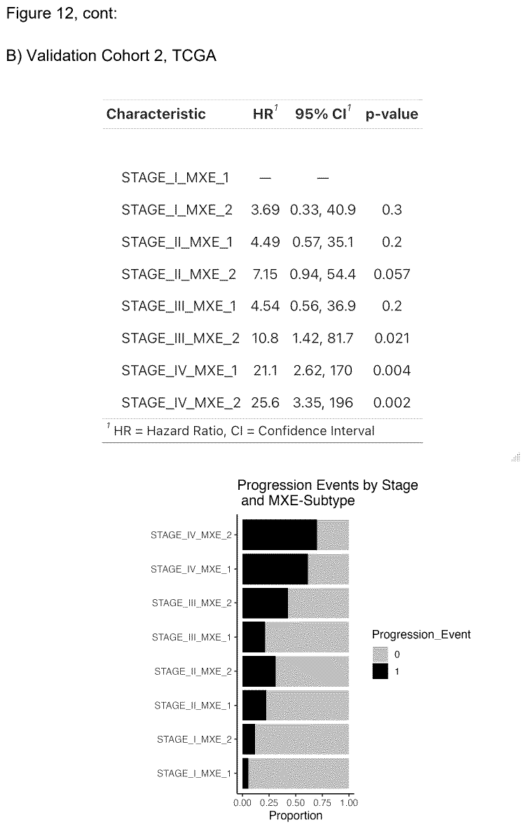

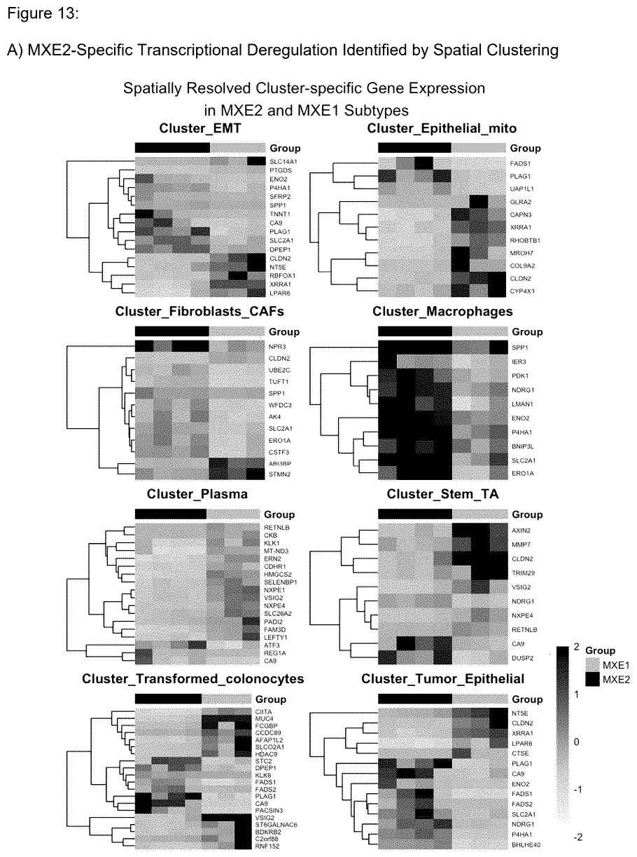

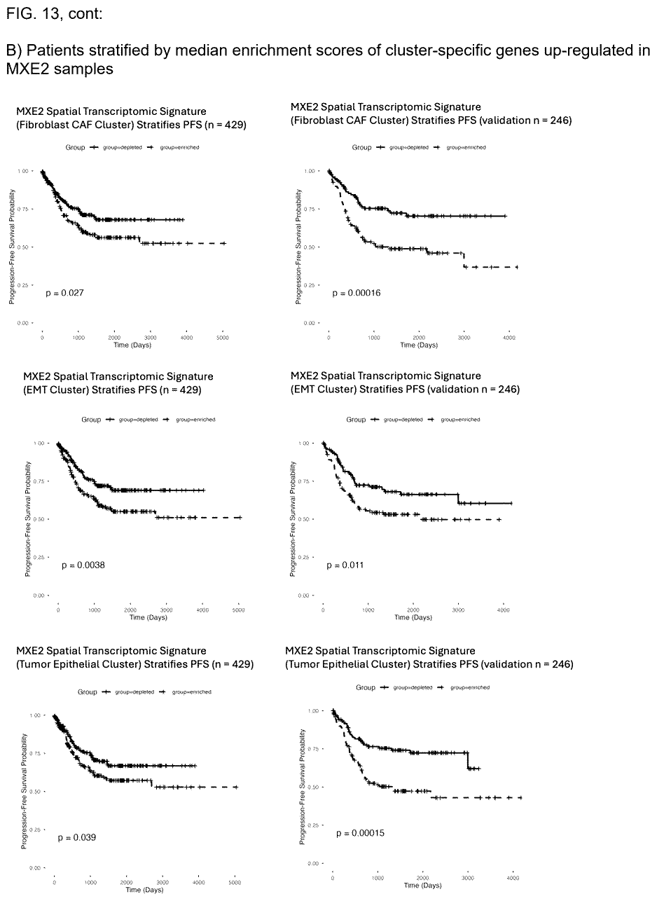

### DETAILED DESCRIPTION

The cancer subtype identifier as well as the method according to the invention has the following advantages:
A novel diagnostic method and a cancer identifier is presented, which utilize the patterns of PSI values based on the occurrence of at least one mutually exclusive exon usage event, the expression of small nuclear RNAs and/or the expression of protein-coding genes to predict prognosis of a cancer patient.
These distinct prognostic groups are independent of the molecular subtype model (CMS), MSI status, Epithelial-to-Mesenchymal Transition (EMT)-associated splicing pattern, clustering of cluster assignments (COCA) subtype and immune infiltrate. Additionally, these prognostic groups can be combined with conventional classification approaches and tumor staging to refine patient prognosis for stage III CRC patients.
The classification system generates new prognostic groups that are independent from traditional staging and are more predictive of patient outcomes. Furthermore, the underlying biology between these two groups is closely associated with changes in splicing and expression of spliceosomal small nuclear RNAs.
A reliable colorectal cancer identifier is presented that can enable effective stratification of patients within a given stage, thus allowing matching of the most appropriate treatment option for each patient.

The designation of the reference genome is GRCh38, annotation is Gencode v28 (primary assembly).

In the sense of the invention the term “subject” refers to any human or animal. A (non-human) animal includes all vertebrates, e.g., mammals and non-mammals, including cows, sheep, pigs, goats, horses, poultry, dogs, cats, non-human primates, rodents etc. In one embodiment, the subject is a human subject.

The terms “marker” and “biomarker” as used herein refer to
PSI value, based on the occurrence of at least one mutually exclusive usage exon event, and/or
expression of small nuclear RNAs and/or
expression of protein-coding genes
   for determining cancer subtypes, in particular the MXE1 subtype and/or MXE2 subtype.

In the sense of the invention the term “outcome” is the result of a treatment or series of treatments of a certain disease.

The outcome is determined at a point in time during or after the treatment on the basis of one criterion or a set of several criteria. Consideration of outcome helps to determine the effectiveness and appropriateness of medical interventions and to evaluate them in relation to alternatives, particularly nonintervention.

There are a number of definitions of the outcome of a disease, defined e.g., by using different endpoints. One way of determining the outcome is the “long-term” survival that refers to survival after diagnosis and/or initial treatment for a particular time period, e.g., for at least 3 years. An alternative way is the “Progression-Free Survival” (PFS) that refers to survival for a time period (usually in years) from diagnosis and/or initial treatment to cancer recurrence, metastasis or death due to recurrence of cancer. Another way is the “Recurrence-Free Survival” (RFS) that refers to survival for a time period (usually in years) from diagnosis and/or initial treatment to cancer recurrence or death due to recurrence of cancer. Another way of determining is the “Overall Survival” (OS) that refers to the time (in years) from diagnosis and/or initial treatment to death from any cause. A further way is “Disease-Free Survival” (DFS) that refers to survival for a time period (usually in years) from diagnosis and/or initial treatment to first cancer recurrence or death from any cause.

The term “prognosis prediction” used here refers to act of predicting the course of a tumor disease and the outcome of death or survival.

“Outcome analyses” often focus on changes in quality of life; the respective preventive or therapeutic measures are thus to be evaluated in a more meaningful way for future subjects than is possible with the so-called surrogate markers, parameters or endpoints (these are measurable variables that are not directly relevant for the person concerned, such as measurement and laboratory values, tumor diameter).

The term decreased progression (“good outcome”, “good prognosis”) refers to a prospect of recovery from a disease or disorder according to the invention that is associated with an extended likelihood of a positive outcome. Further, it refers to an increased subject (patient) survival and/or a delayed disease progression and/or a decreased disease recurrence and/or a decreased metastasis formation. Further, the subject (patient) is more likely to respond to therapy.

The term “increased progression” (“poor outcome”, “poor prognosis”) refers to a prospect of recovery from a disease or disorder according to the invention that is associated with a reduced survival rate, reduced survival time, higher likelihood of disease progression. Further, it refers to a decreased subject (patient) survival and/or an early disease progression and/or an increased disease recurrence and/or an increased metastasis formation. Further, the subject (patient) is less likely to respond to therapy.

Instead of surrogate parameters or an intuitive description of the case (“cured”/“not cured” or similar), a description of the overall situation should be as precisely defined as possible.

Advantageously, the cancer subtype identifier classifies tumor as MXE1 subtype and MXE2 subtype. The MXE1 subtype is associated with a good (favorable prognosis) prognosis compared to the MXE2 subtype. The MXE2 subtype is associated with a poor (unfavorable) prognosis compared to the MXE1 subtype.

The term “MXE-subtypes” relates to two groups of patients, each being characterized by a concrete mutually exclusive event, denoted as MXE, selected from mutually exclusive exon usage events (mutually exclusive splicing events) and/or expression of small nuclear RNAs and/or expression of predictive protein-coding genes.

The MXE1 subtype is characterized by
i) a distinct combination of PSI values from mutually exclusive exon usage events,
ii) a distinct combination of expressions of small nuclear RNAs,
iii) a distinct combination of protein coding genes,
a combination of i) and ii), i) and iii), ii) and iii) and i), ii) and iii),
wherein MXE1 subtype is associated with a good prognosis compared to the MXE2 subtype.

The MXE2 subtype is characterized by
iv) a distinct combination of PSI values from mutually exclusive exon usage events,
v) a distinct combination of expressions of small nuclear RNAs,
vi) a distinct combination of protein coding genes,
a combination of iv) and v), iv) and vi), v) and vi) and iv), v) and vi),
wherein the MXE2 subtype is associated with a poor prognosis compared to the MXE1 subtype.

With the proviso that i) is different from iv), ii) is different from v), iii) is different from vi).

According to the invention the cancer is preferably selected from the group consisting of lung cancer, breast cancer, gastric cancer, esophageal cancer, skin cancer, ovarian cancer, cervical cancer, liver cancer, bladder cancer, kidney cancer, pancreatic cancer, prostate cancer, thyroid cancer, head cancer, neck cancer, oral cancer, laryngeal cancer, rectal cancer and colon cancer. In particular, the cancer is selected from colorectal cancer and non-small cell lung cancer (lung adenocarcinoma).

In a special embodiment non-small cell lung cancer is stratified using small nuclear RNA (snRNA) expression patterns only.

### Determination of the PSI Value

In a first embodiment the invention is directed to a cancer subtype identifier, which classifies tumors, based on PSI values based on the occurrence of at least one mutually exclusive exon usage event. In other words, the PSI values based on the occurrence of at least one mutually exclusive exon usage event is determined.

The term “PSI”, the abbreviation of “percent spliced-in”, refers to the exon-inclusion ratio. It is a known statistic value for measuring alternative splicing events. Alternative splicing allows a gene to be transcribed into multiple isoforms (or mRNA transcripts). PSI is defined as the ratio of the relative abundance of all isoforms containing a certain exon over the relative abundance of all isoforms of the gene containing the exon. In other words, the PSI value defines how often the exon occurs in all the isoforms of the gene that contains the exon. The PSI value of an alternative splicing event reflects the intensity/frequency of such events and has been widely used in detection of differentially spliced exons.

Alternative splicing (AS) events contribute to the complexity of gene expression patterns and can be categorized into five distinct types: (1) exon-skipping (ES), (2) intron retention (IR), (3) alternative 5′ splice (A5SS), (4) alternative 3′ splice (A3SS) and (5) mutually exclusive exon usage (MXE) events. These events are modulated in a tissue and cell type-specific manner and introduce qualitative changes into the existing RNA pool, adding an additional layer of biological information.

According to the invention, the alternative splicing event relates to mutually exclusive exon usage (MXE) events. The term “mutually exclusive exon” (also refers to “MXE”) is characterized by splicing of exons in a coordinated manner such that two or more splicing events are not independent. Exactly one out of two exons (or one group of two exon groups) is retained, while the other one is spliced out (retention of only one of the two exons). A sample has a mixture of mRNAs where some retain exon1 (excluding exon 2) and vice versa. In MXE events, if the PSI value of exon1 is lower than the PSI value of exon2, this indicates that the sample has more mRNA molecules where exon2 is retained. In other words, exon2 has higher inclusion than exon1. In MXE events, if the PSI value of exon2 is lower than the PSI value of exon1, this indicates that the sample has more mRNA molecules where exon1 is retained. In other words, exon1 has higher inclusion than exon2.

Preferably, for identification of MXE1 subtype at least one primary mutually exclusive exon usage event is present. This primary mutually exclusive exon usage event exhibits the highest predictive importance for MXE subtype classification, characterized by differential splicing of exons that are incorporated into different transcripts, as exon1 and exon2. Therein the mutually exclusive splicing events are preferably selected from exon1 (with genomic coordinates chr21: 45480466-45480520) of gene collagen type XVIII alpha 1 chain (COL18A1) (ENSG00000182871.14), compared to exon2 (with genomic coordinates chr21: 45480699-45480858); exon1 (with genomic coordinates chr19: 33012646-33012724) of gene rhophilin Rho GTPase binding protein 2 (RHPN2) (ENSG00000131941.7), compared to exon2 (with genomic coordinates chr19: 33021570-33021646); exon1 (with genomic coordinates chr19: 10986188-10986593) of gene SWI/SNF related, matrix associated, actin dependent regulator of chromatin, subfamily a, member 4 (SMARCA4) (ENSG00000127616.18), compared to exon2 (with genomic coordinates chr19: 10986904-10987003); exon1 (with genomic coordinates chr17: 69304668-69304810) of gene ATP binding cassette subfamily A member 5 (ABCA5) (ENSG00000154265.15), compared to exon2 (with genomic coordinates chr17: 69306724-69306954); exon1 (with genomic coordinates chr11: 66564087-66564146) of gene cathepsin F (CTSF) (ENSG00000174080.10), compared to exon2 (with genomic coordinates chr11: 66564557-66564648); exon1 (with genomic coordinates chr1: 200844781-200844869) of gene calmodulin regulated spectrin associated protein family member 2 (CAMSAP2) (ENSG00000118200.14), compared to exon2 (with genomic coordinates chr1: 200848031-200850234); exon1 (with genomic coordinates chr2: 188988080-188988134) of gene collagen type III alpha 1 chain (COL3A1) (ENSG00000168542.14), compared to exon2 (with genomic coordinates chr2: 188989395-188989449); exon1 (with genomic coordinates chr1: 54205294-54205410) of gene mitochondrial ribosomal protein L37 (MRPL37) (ENSG00000116221.15), compared to exon2 (with genomic coordinates chr1: 54209945-54210131); exon1 (with genomic coordinates chr6: 149836084-149836297) of gene LDL receptor related protein 11 (LRP11) (ENSG00000120256.9), compared to exon2 (with genomic coordinates chr6: 149837337-149837463); exon1 (with genomic coordinates chrX: 41140655-41140771) of gene ubiquitin specific peptidase 9 X-linked (USP9X) (ENSG00000124486.12), compared to exon2 (with genomic coordinates chrX: 41140965-41141217); exon1 (with genomic coordinates chr6: 31642828-31643115) of gene BAG cochaperone 6 (BAG6) (ENSG00000204463.12), compared to exon2 (with genomic coordinates chr6: 31644081-31644194); exon1 (with genomic coordinates chr6: 33303954-33303989) of gene TAP binding protein (TAPBP) (ENSG00000231925.11), compared to exon2 (with genomic coordinates chr6: 33304127-33304217); exon1 (with genomic coordinates chr7: 34966893-34966971) of gene dpy-19 like C-mannosyltransferase 1 (DPY19L1) (ENSG00000173852.14), compared to exon2 (with genomic coordinates chr7: 34969432-34969532); exon1 (with genomic coordinates chr2: 24299908-24300171) of gene intersectin 2 (ITSN2) (ENSG00000198399.14), compared to exon2 (with genomic coordinates chr2: 24301153-24301239); exon1 (with genomic coordinates chr10: 112438335-112438389) of gene zinc finger DHHC-type palmitoyltransferase 6 (ZDHHC6) (ENSG00000023041.11), compared to exon2 (with genomic coordinates chr10: 112440533-112440695); exon1 (with genomic coordinates chr10: 112438335-112438389) of gene zinc finger DHHC-type palmitoyltransferase 6 (ZDHHC6) (ENSG00000023041.11), compared to exon2 (with genomic coordinates chr10: 112442191-112442351).

Preferably, additionally in the identification of MXE1 subtype at least one secondary mutually exclusive exon usage event is present. This secondary mutually exclusive exon usage event exhibits complementary predictive importance for MXE subtype classification, characterized by differential splicing of exons that are incorporated into different transcripts, as exon1 and exon2. Therein the secondary mutually exclusive exon usage events are preferably selected from exon1 (with genomic coordinates chr19: 49862043-49862105) of gene polynucleotide kinase 3′-phosphatase (PNKP) (ENSG00000039650.11), compared to exon2 (with genomic coordinates chr19: 49862184-49862281); exon1 (with genomic coordinates chr19: 47372729-47372923) of gene DExH-box helicase 34 (DHX34) (ENSG00000134815.18), compared to exon2 (with genomic coordinates chr19: 47373598-47373700); exon1 (with genomic coordinates chr15: 65701050-65701192) of gene DENN domain containing 4A (DENND4A) (ENSG00000174485.15), compared to exon2 (with genomic coordinates chr15: 65701761-65701890); exon1 (with genomic coordinates chr17: 67145742-67145890) of gene helicase with zinc finger (HELZ) (ENSG00000198265.11), compared to exon2 (with genomic coordinates chr17: 67149866-67149985); exon1 (with genomic coordinates chr11: 2527927-2528018) of gene potassium voltage-gated channel subfamily Q member 1 (KCNQ1) (ENSG00000053918.16), compared to exon2 (with genomic coordinates chr11: 2587569-2587692); exon1 (with genomic coordinates chr5: 9050409-9050457) of gene semaphorin 5A (SEMA5A) (ENSG00000112902.11), compared to exon2 (with genomic coordinates chr5: 9051872-9052028); exon1 (with genomic coordinates chr20: 10641112-10641244) of gene jagged canonical Notch ligand 1 (JAG1) (ENSG00000101384.12), compared to exon2 (with genomic coordinates chr20: 10641459-10641693).

The ranking as primary and secondary events is performed via stability scores (max 1, min 0.9) indicating consistent and robust predictive value for identification of MXE subtypes. Primary events have stability scores >=0.95 and secondary events have stability scores <0.95.

Also preferred for identification of MXE2 subtype at least one primary mutually exclusive exon usage event is present. This primary mutually exclusive exon usage event exhibits the highest predictive importance for MXE subtype classification, characterized by differential splicing of exons that are incorporated into different transcripts, as exon1 and exon 2 Therein the mutually exclusive splicing events are preferably selected from exon1 (with genomic coordinates chr20: 48988490-48988662) of gene ADP ribosylation factor guanine nucleotide exchange factor 2 (ARFGEF2) (ENSG00000124198.8), compared to exon2 (with genomic coordinates chr20: 48989284-48989436); exon1 (with genomic coordinates chr18: 62266700-62266749) of gene RAB11 Binding and LisH domain (KIAA1468) (ENSG00000134444.14), compared to exon2 (with genomic coordinates chr18: 62268868-62268948); exon1 (with genomic coordinates chr16: 30081258-30081310) of gene protein phosphatase 4 catalytic subunit (PPP4C) (ENSG00000149923.13), compared to exon2 (with genomic coordinates chr16: 30082483-30082534); exon1 (with genomic coordinates chr15: 34255261-34255392) of gene solute carrier family 12 member 6 (SLC12A6) (ENSG00000140199.11), compared to exon2 (with genomic coordinates chr15: 34258812-34258944); exon1 (with genomic coordinates chr14: 22905605-22905659) of gene RNA binding motif protein 23 (RBM23) (ENSG00000100461.17), compared to exon2 (with genomic coordinates chr14: 22908332-22908380); exon1 (with genomic coordinates chr12: 7142085-7142139) of gene calsyntenin 3 (CLSTN3) (ENSG00000139182.13), compared to exon2 (with genomic coordinates chr12: 7142868-7143026); exon1 (with genomic coordinates chr20: 13534021-13534141) of gene taspase 1 (TASP1) (ENSG00000089123.15), compared to exon2 (with genomic coordinates chr20: 13559007-13559114); exon1 (with genomic coordinates chr8: 52630469-52630528) of gene RB1 inducible coiled-coil 1 (RB1CC1) (ENSG00000023287.12), compared to exon2 (with genomic coordinates chr8: 52634920-52634968); exon1 (with genomic coordinates chr1: 150156065-150156206) of gene pleckstrin homology domain containing 01 (PLEKHO1) (ENSG00000023902.13), compared to exon2 (with genomic coordinates chr1: 150157384-150157486); exon1 (with genomic coordinates chr2: 32607454-32607643) of gene baculoviral IAP repeat containing 6 (BIRC6) (ENSG00000115760.13), compared to exon2 (with genomic coordinates chr2: 32611447-32611582); exon1 (with genomic coordinates chr11: 9430874-9431003) of gene importin 7 (IPO7) (ENSG00000205339.9), compared to exon2 (with genomic coordinates chr11: 9433569-9433636); exon1 (with genomic coordinates chr17: 82081163-82081352) of gene fatty acid synthase (FASN) (ENSG00000169710.8), compared to exon2 (with genomic coordinates chr17: 82081600-82081843); exon1 (with genomic coordinates chr6: 157873101-157873176) of gene sorting nexin 9 (SNX9) (ENSG00000130340.15), compared to exon2 (with genomic coordinates chr6: 157875050-157875176); exon1 (with genomic coordinates chr7: 5390484-5390628) of gene trinucleotide repeat containing 18 (TNRC18) (ENSG00000182095.14), compared to exon2 (with genomic coordinates chr7: 5394439-5394595); exon1 (with genomic coordinates chr17: 50195922-50195976) of gene collagen type I alpha 1 chain (COL1A1) (ENSG00000108821.13), compared to exon2 (with genomic coordinates chr17: 50196154-50196199).

Preferably, additionally the identification of MXE2 subtype at least one secondary mutually exclusive exon usage event is present. This secondary mutually exclusive exon usage event exhibits complementary predictive importance for MXE subtype classification, characterized by differential splicing of exons that are incorporated into different transcripts, as exon1 and exon2. Therein the mutually exclusive exon events are preferably selected from exon1 (with genomic coordinates chr19: 55091649-55091700) of gene protein phosphatase 1 regulatory subunit 12C (PPP1R12C) (ENSG00000125503.12), compared to exon2 (with genomic coordinates chr19: 55091858-55091909); exon1 (with genomic coordinates chr17: 69301138-69301286) of gene ATP binding cassette subfamily A member 5 (ABCA5) (ENSG00000154265.15), compared to exon2 (with genomic coordinates chr17: 69302717-69302906); exon1 (with genomic coordinates chr4: 148152468-148152613) of gene nuclear receptor subfamily 3 group C member 2 (NR3C2) (ENSG00000151623.14), compared to exon2 (with genomic coordinates chr4: 148154550-148154901); exon1 (with genomic coordinates chr9: 120453455-120453873) of gene CDK5 regulatory subunit associated protein 2 (CDK5RAP2) (ENSG00000136861.17), compared to exon2 (with genomic coordinates chr9: 120460571-120460667).

The ranking as primary and secondary events is performed via stability scores (max 1, min 0.9) indicating consistent and robust predictive value for identification of MXE subtypes. Primary events have stability scores >=0.95 and secondary events have stability scores <0.95.

If two events are mutually exclusive, then each MXE should result in two subtypes. The PSI value for each MXE event is calculated by dividing the number of reads aligning to the splice junction of exon1 (out of the two neighboring exons) by the total number of reads aligning to the junctions of both exon1 and exon2. For the MXE events that distinguish the subtypes, when comparing two neighboring exons that are incorporated into different transcripts, if exon1 is more spliced than exon2 in MXE1, subtype exon2 is either less spliced or shows no difference in splicing compared to exon1, respectively. Then in MXE2 subtype, exon2 is more spliced than exon1 in MXE2, subtype exon1 is either less spliced or shows no difference in splicing compared to exon2, respectively.

In other words, the cancer subtype identifier according to the invention, wherein for distinction of MXE1 from MXE2, the PSI value of exon1 is higher than exon2 in MXE1 subtype indicating that exon1 is preferentially retained over exon2. The cancer subtype identifier according to the invention, wherein for distinction of MXE1 from MXE2, the PSI value of exon1 is higher than exon2 in MXE2 subtype for the exons indicating exon1 is preferentially retained over exon2.

In particular, the term “exon1” in the following denotes “PSI value of exon1”. The term “exon2” in the following denotes “PSI value of exon2”.

Preferably, in MXE1 subtype, primarily exon1 (with genomic coordinates chr21: 45480466-45480520) of gene collagen type XVIII alpha 1 chain (COL18A1) (ENSG00000182871.14), is 18.3%+/−0.9 (SE) more spliced-in (the PSI value is higher) compared to exon2 (with genomic coordinates chr21: 45480699-45480858); exon1 (with genomic coordinates chr19: 33012646-33012724) of gene rhophilin Rho GTPase binding protein 2 (RHPN2) (ENSG00000131941.7), is 12.2%+/−0.6 (SE) more spliced-in (the PSI value is higher) compared to exon2 (with genomic coordinates chr19: 33021570-33021646); exon1 (with genomic coordinates chr19: 10986188-10986593) of gene SWI/SNF related, matrix associated, actin dependent regulator of chromatin, subfamily a, member 4 (SMARCA4) (ENSG00000127616.18), is 16.8%+/−0.9 (SE) more spliced-in (the PSI value is higher) compared to exon2 (with genomic coordinates chr19: 10986904-10987003); exon1 (with genomic coordinates chr17: 69304668-69304810) of gene ATP binding cassette subfamily A member 5 (ABCA5) (ENSG00000154265.15), is 23%+/−1.7 (SE) more spliced-in (the PSI value is higher) compared to exon2 (with genomic coordinates chr17: 69306724-69306954); exon1 (with genomic coordinates chr11: 66564087-66564146) of gene cathepsin F (CTSF) (ENSG00000174080.10), is 20.7%+/−1.1 (SE) more spliced-in (the PSI value is higher) compared to exon2 (with genomic coordinates chr11: 66564557-66564648); exon1 (with genomic coordinates chr1: 200844781-200844869) of gene calmodulin regulated spectrin associated protein family member 2 (CAMSAP2) (ENSG00000118200.14), is 16.7%+/−1.2 (SE) more spliced-in (the PSI value is higher) compared to exon2 (with genomic coordinates chr1: 200848031-200850234); exon1 (with genomic coordinates chr2: 188988080-188988134) of gene collagen type III alpha 1 chain (COL3A1) (ENSG00000168542.14), is 15.8%+/−0.6 (SE) more spliced-in (the PSI value is higher) compared to exon2 (with genomic coordinates chr2: 188989395-188989449); exon1 (with genomic coordinates chr1: 54205294-54205410) of gene mitochondrial ribosomal protein L37 (MRPL37) (ENSG00000116221.15), is 13.7%+/−0.5 (SE) more spliced-in (the PSI value is higher) compared to exon2 (with genomic coordinates chr1: 54209945-54210131); exon1 (with genomic coordinates chr6: 149836084-149836297) of gene LDL receptor related protein 11 (LRP11) (ENSG00000120256.9), is 13.6%+/−0.6 (SE) more spliced-in (the PSI value is higher) compared to exon2 (with genomic coordinates chr6: 149837337-149837463); exon1 (with genomic coordinates chrX: 41140655-41140771) of gene ubiquitin specific peptidase 9 X-linked (USP9X) (ENSG00000124486.12), is 16.4%+/−0.7 (SE) more spliced-in (the PSI value is higher) compared to exon2 (with genomic coordinates chrX: 41140965-41141217); exon1 (with genomic coordinates chr6: 31642828-31643115) of gene BAG cochaperone 6 (BAG6) (ENSG00000204463.12), is 14.8%+/−0.9 (SE) more spliced-in (the PSI value is higher) compared to exon2 (with genomic coordinates chr6: 31644081-31644194); exon1 (with genomic coordinates chr6: 33303954-33303989) of gene TAP binding protein (TAPBP) (ENSG00000231925.11), is 13.4%+/−0.6 (SE) more spliced-in (the PSI value is higher) compared to exon2 (with genomic coordinates chr6: 33304127-33304217); exon1 (with genomic coordinates chr7: 34966893-34966971) of gene dpy-19 like C-mannosyltransferase 1 (DPY19L1) (ENSG00000173852.14), is 18.3%+/−1.3 (SE) more spliced-in (the PSI value is higher) compared to exon2 (with genomic coordinates chr7: 34969432-34969532); exon1 (with genomic coordinates chr2: 24299908-24300171) of gene intersectin 2 (ITSN2) (ENSG00000198399.14), is 14.9%+/−0.7 (SE) more spliced-in (the PSI value is higher) compared to exon2 (with genomic coordinates chr2: 24301153-24301239); exon1 (with genomic coordinates chr10: 112438335-112438389) of gene zinc finger DHHC-type palmitoyltransferase 6 (ZDHHC6) (ENSG00000023041.11), is 16.4%+/−0.8 (SE) more spliced-in (the PSI value is higher) compared to exon2 (with genomic coordinates chr10: 112440533-112440695); exon1 (with genomic coordinates chr10: 112438335-112438389) of gene zinc finger DHHC-type palmitoyltransferase 6 (ZDHHC6) (ENSG00000023041.11), is 15.9%+/−0.8 (SE) more spliced-in (the PSI value is higher) compared to exon2 (with genomic coordinates chr10: 112442191-112442351).

Preferably, in MXE1 subtype, secondarily exon1 (with genomic coordinates chr19: 49862043-49862105) of gene polynucleotide kinase 3′-phosphatase (PNKP) (ENSG00000039650.11), is 14.9%+/−0.9 (SE) more spliced-in (the PSI value is higher) compared to exon2 (with genomic coordinates chr19: 49862184-49862281); exon1 (with genomic coordinates chr19: 47372729-47372923) of gene DExH-box helicase 34 (DHX34) (ENSG00000134815.18), is 15%+/−1 (SE) more spliced-in (the PSI value is higher) compared to exon2 (with genomic coordinates chr19: 47373598-47373700); exon1 (with genomic coordinates chr15: 65701050-65701192) of gene DENN domain containing 4A (DENND4A) (ENSG00000174485.15), is 15%+/−1.1 (SE) more spliced-in (the PSI value is higher) compared to exon2 (with genomic coordinates chr15: 65701761-65701890); exon1 (with genomic coordinates chr17: 67145742-67145890) of gene helicase with zinc finger (HELZ) (ENSG00000198265.11), is 14.2%+/−0.8 (SE) more spliced-in (the PSI value is higher) compared to exon2 (with genomic coordinates chr17: 67149866-67149985); exon1 (with genomic coordinates chr11: 2527927-2528018) of gene potassium voltage-gated channel subfamily Q member 1 (KCNQ1) (ENSG00000053918.16), is 12.3%+/−0.8 (SE) more spliced-in (the PSI value is higher) compared to exon2 (with genomic coordinates chr11: 2587569-2587692); exon1 (with genomic coordinates chr5: 9050409-9050457) of gene semaphorin 5A (SEMA5A) (ENSG00000112902.11), is 12.6%+/−1.2 (SE) more spliced-in (the PSI value is higher) compared to exon2 (with genomic coordinates chr5: 9051872-9052028); exon1 (with genomic coordinates chr20: 10641112-10641244) of gene jagged canonical Notch ligand 1 (JAG1) (ENSG00000101384.12), is 13.3%+/−0.6 (SE) more spliced-in (the PSI value is higher) compared to exon2 (with genomic coordinates chr20: 10641459-10641693).

Also preferred in MXE2 subtype primarily, exon1 (with genomic coordinates chr20: 48988490-48988662) of gene ADP ribosylation factor guanine nucleotide exchange factor 2 (ARFGEF2) (ENSG00000124198.8), is 15%+/−0.6 (SE) more spliced-in (the PSI value is higher) compared to exon2 (with genomic coordinates chr20: 48989284-48989436); exon1 (with genomic coordinates chr18: 62266700-62266749) of gene RAB11 binding and LisH domain (KIAA1468) (ENSG00000134444.14), is 15.8%+/−1 (SE) more spliced-in (the PSI value is higher) compared to exon2 (with genomic coordinates chr18: 62268868-62268948); exon1 (with genomic coordinates chr16: 30081258-30081310) of gene protein phosphatase 4 catalytic subunit (PPP4C) (ENSG00000149923.13), is 16.1%+/−0.8 (SE) more spliced-in (the PSI value is higher) compared to exon2 (with genomic coordinates chr16: 30082483-30082534); exon1 (with genomic coordinates chr15: 34255261-34255392) of gene solute carrier family 12 member 6 (SLC12A6) (ENSG00000140199.11), is 14.1%+/−1 (SE) more spliced-in (the PSI value is higher) compared to exon2 (with genomic coordinates chr15: 34258812-34258944); exon1 (with genomic coordinates chr14: 22905605-22905659) of gene RNA binding motif protein 23 (RBM23) (ENSG00000100461.17), is 19.2%+/−1.2 (SE) more spliced-in (the PSI value is higher) compared to exon2 (with genomic coordinates chr14: 22908332-22908380); exon1 (with genomic coordinates chr12: 7142085-7142139) of gene calsyntenin 3 (CLSTN3) (ENSG00000139182.13), is 17.2%+/−1 (SE) more spliced-in (the PSI value is higher) compared to exon2 (with genomic coordinates chr12: 7142868-7143026); exon1 (with genomic coordinates chr20: 13534021-13534141) of gene taspase 1 (TASP1) (ENSG00000089123.15), is 14.1%+/−0.8 (SE) more spliced-in (the PSI value is higher) compared to exon2 (with genomic coordinates chr20: 13559007-13559114); exon1 (with genomic coordinates chr8: 52630469-52630528) of gene RB1 inducible coiled-coil 1 (RB1CC1) (ENSG00000023287.12), is 14.1%+/−0.8 (SE) more spliced-in (the PSI value is higher) compared to exon2 (with genomic coordinates chr8: 52634920-52634968); exon1 (with genomic coordinates chr1: 150156065-150156206) of gene pleckstrin homology domain containing 01 (PLEKHO1) (ENSG00000023902.13), is 12.8%+/−0.7 (SE) more spliced-in (the PSI value is higher) compared to exon2 (with genomic coordinates chr1: 150157384-150157486); exon1 (with genomic coordinates chr2: 32607454-32607643) of gene baculoviral IAP repeat containing 6 (BIRC6) (ENSG00000115760.13), is 13.4%+/−0.6 (SE) more spliced-in (the PSI value is higher) compared to exon2 (with genomic coordinates chr2: 32611447-32611582); exon1 (with genomic coordinates chr11: 9430874-9431003) of gene importin 7 (IPO7) (ENSG00000205339.9), is 14.4%+/−0.6 (SE) more spliced-in (the PSI value is higher) compared to exon2 (with genomic coordinates chr11: 9433569-9433636); exon1 (with genomic coordinates chr17: 82081163-82081352) of gene fatty acid synthase (FASN) (ENSG00000169710.8), is 13.5%+/−0.7 (SE) more spliced-in (the PSI value is higher) compared to exon2 (with genomic coordinates chr17: 82081600-82081843); exon1 (with genomic coordinates chr6: 157873101-157873176) of gene sorting nexin 9 (SNX9) (ENSG00000130340.15), is 15.5%+/−0.6 (SE) more spliced-in (the PSI value is higher) compared to exon2 (with genomic coordinates chr6: 157875050-157875176); exon1 (with genomic coordinates chr7: 5390484-5390628) of gene trinucleotide repeat containing 18 (TNRC18) (ENSG00000182095.14), is 21.6%+/−1 (SE) more spliced-in (the PSI value is higher) compared to exon2 (with genomic coordinates chr7: 5394439-5394595); exon1 (with genomic coordinates chr17: 50195922-50195976) of gene collagen type I alpha 1 chain (COL1A1) (ENSG00000108821.13), is 13%+/−0.4 (SE) more spliced-in (the PSI value is higher) compared to exon2 (with genomic coordinates chr17: 50196154-50196199).

Preferably, in MXE2 subtype, secondarily exon1 (with genomic coordinates chr19: 55091649-55091700) of gene protein phosphatase 1 regulatory subunit 12C (PPP1R12C) (ENSG00000125503.12), is 15.3%+/−1 (SE) more spliced-in (the PSI value is higher) compared to exon2 (with genomic coordinates chr19: 55091858-55091909); exon1 (with genomic coordinates chr17: 69301138-69301286) of gene ATP binding cassette subfamily A member 5 (ABCA5) (ENSG00000154265.15), is 17.6%+/−1.5 (SE) more spliced-in (the PSI value is higher) compared to exon2 (with genomic coordinates chr17: 69302717-69302906); exon1 (with genomic coordinates chr4: 148152468-148152613) of gene nuclear receptor subfamily 3 group C member 2 (NR3C2) (ENSG00000151623.14), is 12.2%+/−1.5 (SE) more spliced-in (the PSI value is higher) compared to exon2 (with genomic coordinates chr4: 148154550-148154901); exon1 (with genomic coordinates chr9: 120453455-120453873) of gene CDK5 regulatory subunit associated protein 2 (CDK5RAP2) (ENSG00000136861.17), is 15%+/−0.9 (SE) more spliced-in (the PSI value is higher) compared to exon2 (with genomic coordinates chr9: 120460571-120460667).

The ranking as primary and secondary events is performed via stability scores (max 1, min 0.9) indicating consistent and robust predictive value for identification of MXE subtypes. Primary events have stability scores >=0.95 and secondary events have stability scores <0.95.

### Expression of Small Nuclear RNAs

In a second preferred embodiment the invention is directed to a cancer subtype identifier, which classifies tumors, based on the expression of small nuclear RNAs. Preferably, the expression of the small nuclear RNA correlates with PSI values of at least one mutually exclusive exon usage event or differs between the MXE subtypes significantly. The statistical significance level is of adjusted P<0.05. In particular the small nuclear RNA is derived from stranded total RNA-sequencing of cancer tumors.

Preferred for the identification of the MXE1 subtype the small nuclear RNA expression is characterized by their up-regulation in the MXE1 subtype as compared to the MXE2 subtype.

Preferred for the identification of the MXE1 subtype, the small nuclear RNA expression shows up-regulation compared to the MXE2 subtype for primary small nuclear RNA exhibiting the highest predictive importance for MXE subtype classification, selected from:

RNU4-2 (ENSG00000202538.1) SEQ ID NO: 1:
AGCTTTGCGCAGTGGCAGTATCGTAGCCAATGAGGTTTATCCGAGGCGCGATTATTGCTAATTG
AAAACTTTTCCCAATACCCCGCCATGACGACTTGAAATATAGTCGGCATTGGCAATTTTTGACAG
TCTCTACGGAGA
RNVU1-7 (ENSG00000206585.1) SEQ ID NO: 2:
ATACTTACCTGGCAGGGGAGATACCATGATCACGAAGGTGGTTTTCCCAGGGCGAGGCTTATC
CATTGCACTCCGGATGTGCTGACCCCTGCGATTTCCCCAAATGTGGGAAACTCGACTGCATAAT
TTGTGGTAGTGGGGGACTGCGTCCGCGCTTTCCCCTG
RNVU1-6 (ENSG00000201558.1) SEQ ID NO: 3:
ATACTTACCTGGCAGGAGAGATACCCTGGTCACGAAGGTGGTTTTTCCAGAGCGAGTCTTATCC
ATTGCACTCCGGATCTGCTGACCCCTGCAGTTTCCCCAAATGTGGGAAACTTGACTGTATAATTT
GTGGCAGTGGTAGACTGCATTCGCGCTTTCCCCTG.

Preferably, additionally in the identification of the MXE1 subtype, the small nuclear RNA expression shows up-regulation compared to the MXE2 subtype for secondary small nuclear RNAs exhibiting complementary predictive importance for MXE subtype classification, selected from:

RNU1-4 (ENSG00000207389.1) SEQ ID NO: 4:
ATACTTACCTGGCAGGGGAGATACCATGATCACGAAGGTGGTTTTCCCAGGGCGAGGCTTATC
CATTGCACTCCGGATGTGCTGACCCCTGCGATTTCCCCAAATGTGGGAAACTCGACTGCATAAT
TTGTGGTAGTGGGGGACTGCGTTCGCGCTTTCCCCTG
RNVU1-18 (ENSG00000206737.1) SEQ ID NO: 5:
ATACTTACCTGGCAGGGGAGATACCATGATCACGAAGGTGGTTTTCCCAGGGCGAGGCTTATC
CATTGCACTCCGGATGTGCTGACCCCTGCGATTTCCCCAAATGTGGGAAACTCGACTGCATAAT
TTGTGGTAGTGGGGGACTGCGTTCGCGCTTTCCCCTG
RNU1-1(ENSG00000206652.1) SEQ ID NO: 6:
ATACTTACCTGGCAGGGGAGATACCATGATCACGAAGGTGGTTTTCCCAGGGCGAGGCTTATC
CATTGCACTCCGGATGTGCTGACCCCTGCGATTTCCCCAAATGTGGGAAACTCGACTGCATAAT
TTGTGGTAGTGGGGGACTGCGTTCGCGCTTTCCCCTG
RF00003 (U1 family) (ENSG00000273727.1) SEQ ID NO: 7:
ATACTTATGTAACAGGAGAAAATACGGCCATGAAGTTGGTGTTTTTCGGGGGGGGTTTTTCCATT
GTACTCAGTATGTGCTGACTGACTCCTGTTACTTCCACATGTGGGGAAACTGGACTGTAATTTGT
GGTGGTGGGGAATTGCGTTCGCGCTTTCTTCT
RF00003 (U1 family) (ENSG00000273516.1) SEQ ID NO: 8:
CCACTTACCTGGCAGGAAAGAGACCGTGGTCACGAAGGGGGTTCTCCCAGAGTGAAGCTTCTT
CATCGCACTCTAGAGTTGCTGATCTCTGTGATTTCTTCAATGTGGGAAACGGTGTTTGTGCTAGA
AGAGGGCTGCGCTCTCTA
RF00003 (U1 family) (ENSG00000275291.1) SEQ ID NO: 9 :
ATACTTACGTAACAGGAGAAAATACGGCCATGAAGTTGGTGTTTCTCGGGGGCGATTTCTCCAT
TGTACTCAGTATGTGCTGACTGACTCCTGTTACTTCCACATGTGGGGAAACTGGACTGTAATTTG
TGGTGGTGGGGAATTGCGTTCGCGCTTTCTTCT
RF00003 (U1 family) (ENSG00000278099.1) SEQ ID NO: 10:
ATATTTACTTGGCAGGGGAGATAACGTGACCACGAAGGTGGTTTTCCCAGGGCTGAGGCTTATT
CATTGTACTCCGGATGTGCTGACCCCTGCGATTTCCCCAAATGTGGGAAACTCGACTGCATAAT
TTGTGGTAGTGGGGGGCTGTGTCCGTGCTTTTCCCTG
RF00003 (U1 family) (ENSG00000277918.1) SEQ ID NO:11:
ATACTTACCTGGCAGGGGAGATACCATGATCACGAATGTGGTTTTCCCAGGGCGAGGCTTATCC
ATTGCACTCCGGATGTGCTGACCCCTGCGATTTCCCCAAATGTGGGAAACTCGACTGCATAATT
TGTGGTAGTGGGGGACTGCGTTCGCGCTTTCCCCTG
RF00003 (U1 family) (ENSG00000270384.1) SEQ ID NO: 12:
CCCCTTATGTGGCGGGGGACATACAGTGGTCAGGAAGGGGGTTCTCCCCGGAGGAGGCTAGT
CCACCACACTTCGGCTCCGCTGACCCCTGCGATTTCTCCACATGCGGGGCCCTCGTCCGCGGT
GGTGTTTGCGCTATCCGGCGGCTGGGTTCGCGCACTCACTCT
RF00003 (U1 family) (ENSG00000270722.1) SEQ ID NO: 13:
ATACTTACCTGGCAGGGGAGATACCATGATCACGAAGGTGGTTTTCCCTGGGCGAGGCTTATC
CATTGCACTCCGGATGTGCTGACCCCTGCGATTTCCCCAAATGTGGGAAACTTGACTGCATAAT
TTGTGGTAGTGGGGGACTGCGTTCGCGCTTTCCCCTG
RF00003 (U1 family) (ENSG00000274428.1) SEQ ID NO: 14:
ATACTTACGTAACAGGAGAAAATACGGCCATGAAGTTGGTGTTTTTCGGGGGCGATTTCTCCAT
TGTACTCAGTATGTGCTGACTGACTCCTGTTACTTCCACATGTGGGGAAACTGGACTGTAATTTG
TGGTGGTGGGGAATTGCGTTCGCTTTCTTCT
RF00003 (U1 family) (ENSG00000274210.1) SEQ ID NO: 15:
ATACTTACCTGGCAGGGGAGATACCATGATCATGAAGGCGCTTTTCTCATGGTCGCGGCTTATC
CATTGCGTTCCAGATGTGCTGACCTCTGCGATTTCCCCAAATGTGGGAAACTCGACTGCATAAT
TTGTGATAGTGGGGGGCTGCGTTCGTGCTTTCCCCTG
RF00003 (U1 family) (ENSG00000206828.1) SEQ ID NO: 16:
ATGTTTATCTGACAGAAGAAATGTTATGATCACGAAGGTGGTTTTTCCAGAGCAAGGCTTAGCAA
TTTTGCTGTGGATGTGCTGACCCCTGTGATTTCCCCGAATGTGGGAATCTCCATTACATAACTTG
TGGCAGTGGGGAACTGTGTCTGTGTTTTCCCCTG
RF00015 (U4 family) (ENSG00000276103.1) SEQ ID NO: 17:
AGTTTTGTGCAGTGGCAGTATCATAGCCAATGAGGTTTGTCCAATGTACAATTATTGCTAATTGA
AAACGTTTCAGGCCAAGCACAGTGGCTCACGCCTATAATCCCAGCACTCTGG
RF00015 (U4 family)(ENSG00000283442.1) SEQ ID NO: 18:
AGCTTTGTACAGTGACAGGATTATAGCCAATGAGGCATGATTATTGCTAATTGAAAACTTTTCCC
AATACCCTGGCATTGGCAATTTTTGACAATCTCTATGGAGA
RF00066 (U7 family) (ENSG00000273629.1) SEQ ID NO: 19:
GAGTGTTACAGCCCTTTTAGAATTTGTCTAGCAAGTTTAGAAAATGATTCAAAGAGACTGAC
RF00015 (U4 family)(ENSG00000277986.1) SEQ ID NO: 20:
AGCTTTGCACAGTGGCAGTATCGTAGCCAATGAGCTTTACCCGAGGCGCGATTATTGCTAGTTA
AATATTTATAAAAACCTTTCGAGCAGCGCCTGAACAAGAATAGGTTCAGAGGAGACTCC
RF00066 (U7 family) (ENSG00000275108.1) SEQ ID NO: 21:
CAGTGTTACAGATCTTTTAGAATTTGTCTAGTACATTTTCCAGTTTTCACCAGGAATCTGCCT
RF00015 (U4 family) (ENSG00000274716.1) SEQ ID NO: 22:
AGCTTTGTGCAGTGGCAGTATCATAGCCAATGAGGTTTATCCGAGGCGTGATTATTGCTAATTG
AAAAATTGAGGAATAGTT
RF00015 (U4 family) (ENSG00000273744.1) SEQ ID NO: 23:
AGCTTTGTGTACTGGCAATATCATAACCAATGAGGTTCATCCAAGACATGATAATTGCTAATGGC
AAACAGCATAAAACCAACTGCC
RF00015 (U4 family) (ENSG00000278374.1) SEQ ID NO: 24:
AGCTTTGCGTGTGGCAGTATCATAGCCAATGAGGTTTATCCGAGGCGTGATTATTGCTAATTGA
AAACAAAATGGAAGACAGGTGACTTTTCCCCTCCCTTGAATAAACACTTCTTCCACACATGGA
RF00015 (U4 family)(ENSG00000274197.1) SEQ ID NO: 25:
AGCTTTGTGCAGTGGCAGTATCGTAGCCAGTGAGGTTTATCGGAGGCGTGATCACTGCTAATTG
AAAACTTTTCTATAAAGGAAC
RF00066 (U7 family) (ENSG00000272215.1) SEQ ID NO: 26:
GAGTGTTGCAGCTCTTACAGAATTTGTCTAGCAGGTTTTCCAGTCTTTACCAGAAATGCCCCC
RF00066 (U7 family) (ENSG00000276376.1) SEQ ID NO: 27:
CAGTGTTACAGCTCTTTTAGAATTTGTTAGGCTGGGCGTACTGGCTCACGCCTATAATCCCAA
RF00066 (U7 family) (ENSG00000275504.1) SEQ ID NO: 28:
GAGTGTTACAGCTCTATTAGAATTTGTCTAGCAGGTTTTCTGGTCTTCACTGGAAAGCACCCCC
RF00003 (U1 family) (ENSG00000275631.1) SEQ ID NO: 29:
ATACTTACCTGGCAGGGCAGATACCATGATCTTAAAGGCAGTTTTCCCAGGGCAAGGCTTATCC
ATTCCACTCTGGATCCATCATAGGGATATGCTGATCCCTGGAATTGCCCCAAATGTGGGAAGCT
CTACTGCAAAATTTTTGGTAGTGAGCGATGGCATTATGCATTCATGTA
RF00066 (U7 family) (ENSG00000275635.1) SEQ ID NO: 30:
CAGTGTTACAGCTCTTTTAGAATTTTTCTAGCAGGTTTTCCAGTTTTCACTGGAACCTCT
RF00066 (U7 family) (ENSG00000275268.1) SEQ ID NO: 31:
CAGTGTTACAGCTCTTTTGGAATTAGTCTAGCAGATTTCTCAGTTTTCACTGGAAACCCTT.

Also preferred for the identification of the MXE2 subtype, the small nuclear RNA expression shows up-regulation compared to the MXE1 subtype for primary small nuclear RNA exhibiting the highest predictive importance for MXE subtype classification, selected from:

RNU5B-1 (ENSG00000200156.1) SEQ ID NO: 32:
ATACTCTGGTTTCTCTTCAGATCGTATAAATCTTTCGCCTTTTACTAAAGATTTCCGTGGAGAGG
AACAACTCTGAGTCTTAAGCTAATTTTTTGAGGCCTTGTTCCGACAAGGCT
RNU12 (ENSG00000276027.1) SEQ ID NO: 33:
ATGCCTTAAACTTATGAGTAAGGAAAATAACGATTCGGGGTGACGCCCGAATCCTCACTGCTAA
TGTGAGACGAATTTTTGAGCGGGTAAAGGTCGCCCTCAAGGTGACCCGCCTACTTTGCGGGAT
GCCTGGGAGTTGCGATCTGCCCG
RNU11 (ENSG00000274978.1) SEQ ID NO: 34:
AAAAAGGGCTTCTGTCGTGAGTGGCACACGTAGGGCAACTCGATTGCTCTGCGTGCGGAATCG
ACATCAAGAGATTTCGGAAGCATAATTTTTTGGTATTTGGGCAGCTGGTGATCGTTGGTCCCGG
CGCCCTT
RNU5E-1 (ENSG00000199347.1) SEQ ID NO: 35:
ATACTCTGGTTTCTCTTCAAATCGTATAAATCTTTCGCCTTTTACTAAAGATTTCCGTGGAGAGAA
ACGAGTGTGAGTCTGAAACCAATTTTTTGAGGCCTTGCGTTTCTTAGCAGGGCT
RNU6-9(ENSG00000207507.1) SEQ ID NO: 36:
GTGCTCGCTTCGGCAGCACATATACTAAAATTGGAACGATACAGAGAAGATTAGCATGGCCCCT
GCGCAAGGATGACACGCAAATTCGTGAAGCGTTCCATATTTTT
RNU5D-1 (ENSG00000200169.1) SEQ ID NO: 37:
ATGCTCTGGTTTCTCTTCAAATCGTATAAATCTTTCGCCTTTTACTAAAGATTTCCGTGGAGAGAA
ACCGTTTTGAGTTTCAAGCAAATTTTTTGAAGCCCTATGTTGGTGGGGTT
RNU5A-1 (ENSG00000199568.1) SEQ ID NO: 38:
ATACTCTGGTTTCTCTTCAGATCGCATAAATCTTTCGCCTTTTACTAAAGATTTCCGTGGAGAGG
AACAACTCTGAGTCTTAACCCAATTTTTTGAGGCCTTGCTTTGGCAAGGCT
RNVU1-15 (ENSG00000207205.1) SEQ ID NO: 39:
ATACTTACTTGGCGGGGGAGATACCATGATCACGAAGGTGGTTTTCTCAGGGCGAGGCTTATC
CGTTATGTTCCGGGTGTACTGACCCCTGCCATTTTCCCCCAATGTGAGGAACTCGACTGCATAA
CTTGTGATAGTAGGGGACTGCGTTCGCGCTTTCCCCTG.

Preferably, additionally in the identification of the MXE2 subtype, the small nuclear RNA expression shows up-regulation compared to the MXE1 subtype for secondary small nuclear RNAs exhibiting complementary predictive importance for MXE subtype classification, selected from:

RNVU1-1 (ENSG00000207340.1) SEQ ID NO: 40:
ATACTTACCTGGCAGGGGAGATACGATGATCACGAAGGTGGTTTTCCCAGGGAGAGGCTTATC
CATTGCACTCCGGATGTGCTGACCCCTGCCGTTTCCCCAAATGTGGGAAACTCGACTGCATAAT
TTGTGGTAGTGGGGGACTGCGTTCGCGCTGTCCTCTG
RNU6ATAC (ENSG00000221676.1) SEQ ID NO: 41:
GTGTTGTATGAAAGGAGAGAAGGTTAGCACTCCCCTTGACAAGGATGGAAGAGGCCCTCGGGC
CTGACAACACGCATACGGTTAAGGCATTGCCACCTACTTCGTGGCATCTAACCATCGTTTTTT
RNVU1-19 (ENSG00000275538.1) SEQ ID NO: 42:
ATACTTACCTGGCAGGGGAGATACTATTATCAAACGAAGGTGGTTTTTCTCAGGGCGAGGGTTA
TCCATTGTGTTCCGGATGTGCTGACCTCTGCGATTTCCCCAAACGTGGGAAACTCGACTGCATA
ATTTGTGGTAGTGGGGGACTGCGTTCGCGCTTTCCTCTG
RNU4-1 (ENSG00000200795.1) SEQ ID NO: 43:
AGCTTTGCGCAGTGGCAGTATCGTAGCCAATGAGGTCTATCCGAGGCGCGATTATTGCTAATTG
AAAACTTTTCCCAATACCCCGCCGTGACGACTTGCAATATAGTCGGCACTGGCAATTTTTGACA
GTCTCTACGGAGA
RNU5F-1 (ENSG00000199377.1) SEQ ID NO: 44:
GATCTCTGGTTTCTCTTCATAACGAATAAATCTTTCGCCTTTTACTAAAGATTTCCGTGGAGAA
AAACAACTATGAGTTTATGGTTAAATTTTTTGAAGTCTTGCCTAGGCAAGGCT
RNU4ATAC (ENSG00000264229.1) SEQ ID NO: 45:
ACCATCCTTTTCTTGGGGTTGCGCTACTGTCCAATGAGCGCATAGTGAGGGCAGTACTGCTAAC
GCCTGAACAACACACCCGCATCAACTAGAGCTTTTGCTTTATTTTGGTGCAATTTTTGGAAAA
RNU6-1 (ENSG00000206625.1) SEQ ID NO: 46:
GTGCTCGCTTCGGCAGCACATATACTAAAATTGGAACGATACAGAGAAGATTAGCATGGCCCCT
GCGCAAGGATGACACGCAAATTCGTGAAGCGTTCCATATTTTT
RNU6-2 (ENSG00000207357.1) SEQ ID NO: 47:
GTGCTCGCTTCGGCAGCACATATACTAAAATTGGAACGATACAGAGAAGATTAGCATGGCCCCT
GCGCAAGGATGACACGCAAATTCGTGAAGCGTTCCATATTTTT.

The ranking as primary and secondary small nuclear RNA expression is performed via stratification of predictive model coefficients for identification of MXE subtypes. Primary events have coefficients greater than or equal to the mean of all absolute coefficients and secondary events have coefficients less than this mean.

The subject with poor progression-free survival is characterized by upregulation of one or more snRNAs selected from RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC, RNVU1-1, RNVU1-15, RNVU1-19 and down-regulation of RNU1-1, RNU1-4, RNU4-2, RNVU1-18, RNVU1-6, RNVU1-7, RF00015, RF00066 and RF00003.

In particular, RNU5E-1, RNU5F-1, RNU6-1, RNU6-2, RNU6-9, RNU6ATAC, RNVU1-1, RNVU1-15, RNVU1-19 are upregulated and RNU1-1, RNU1-4, RNU4-2, RNVU1-18, RNVU1-6, RNVU1-7, RF00015, RF00066 and RF00003 are downregulated in MXE2 subtype, which is associated with poor prognosis.

In particular, the subject having colorectal and/or lung adenocarcinoma with poor progression-free survival is characterized by upregulation of one or more snRNAs selected from RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC and down-regulation of RF00015 and RF00066.

In particular, RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC are upregulated and RF00015 and RF00066 are downregulated in MXE2 subtype, which is associated with poor prognosis.

### Expression of Predictive Protein-Coding Genes

A third embodiment the invention is directed to a cancer subtype identifier, which classifies tumors, based on the expression of predictive protein-coding genes. Preferably, the expression of predictive protein-coding genes shows robust predictive value for identifying the subtypes when the data is resampled multiple times. In each iteration, half of the discovery dataset is randomly resampled, and a lasso-penalized logistic regression model selects relevant genes to predict MXE subtypes. Protein-coding genes selected 90% of the time are deemed stable, with those chosen 95% of the time classified as primary, while the remaining stable genes are classified as secondary.

Preferred for the identification of the MXE1 subtype, is the expression of the predictive protein-coding genes, which is characterized by their up-regulation in the MXE1 subtype as compared to the MXE2 subtype.

More preferred for the identification of the MXE1 subtype, is the expression of the predictive protein-coding genes, which is characterized by their up-regulation in the MXE1 subtype as compared to the MXE2 subtype, wherein the predictive protein-coding genes are selected from the primary group consisting of kelch like family member 21 (KLHL21) (ENSG00000162413.16), mitotic arrest deficient 2 like 2 (MAD2L2) (ENSG00000116670.14), angiotensin II receptor associated protein (AGTRAP) (ENSG00000177674.15), NBL1, DAN family BMP antagonist (NBL1) (ENSG00000158747.14), zinc finger DHHC-type palmitoyltransferase 18 (ZDHHC18) (ENSG00000204160.11), solute carrier family 9 member A1 (SLC9A1) (ENSG00000090020.10), transmembrane protein 222 (TMEM222) (ENSG00000186501.14), platelet activating factor receptor (PTAFR) (ENSG00000169403.11), penta-EF-hand domain containing 1 (PEF1) (ENSG00000162517.12), NHS like 3 (KIAA1522) (ENSG00000162522.10), chromosome 1 open reading frame 122 (C1orf122) (ENSG00000197982.13), bone morphogenetic protein 8a (BMP8A) (ENSG00000183682.7), importin 13 (IPO13) (ENSG00000117408.10), diphthamide biosynthesis 2 (DPH2) (ENSG00000132768.13), ATPase H+ transporting V0 subunit b (ATP6V0B) (ENSG00000117410.13), aldo-keto reductase family 1 member A1 (AKR1A1) (ENSG00000117448.13), leucine rich repeat containing 41 (LRRC41) (ENSG00000132128.16), PDZK1 interacting protein 1 (PDZK1IP1) (ENSG00000162366.7), 24-dehydrocholesterol reductase (DHCR24) (ENSG00000116133.11), CD53 molecule (CD53) (ENSG00000143119.12), WD repeat domain 77 (WDR77) (ENSG00000116455.13), peroxisomal biogenesis factor 11 beta (PEX11B) (ENSG00000131779.10), aph-1 homolog A, gamma-secretase subunit (APH1A) (ENSG00000117362.12), proteasome 20S subunit beta 4 (PSMB4) (ENSG00000159377.10), S100 calcium binding protein A2 (S100A2) (ENSG00000196754.10), jumping translocation breakpoint (JTB) (ENSG00000143543.14), RUN and SH3 domain containing 1 (RUSC1) (ENSG00000160753.15), methyltransferase like 25B (RRNAD1) (ENSG00000143303.11), SLAM family member 8 (SLAMF8) (ENSG00000158714.10), protoporphyrinogen oxidase (PPOX) (ENSG00000143224.17), uridine-cytidine kinase 2 (UCK2) (ENSG00000143179.15), SFT2 domain containing 2 (SFT2D2) (ENSG00000213064.9), transmembrane protein 9 (TMEM9) (ENSG00000116857.16), NUAK family kinase 2 (NUAK2) (ENSG00000163545.8), left-right determination factor 1 (LEFTY1) (ENSG00000243709.1), jumonji domain containing 4 (JMJD4) (ENSG00000081692.12), solute carrier family 35 member F6 (SLC35F6) (ENSG00000213699.8), solute carrier family 5 member 6 (SLC5A6) (ENSG00000138074.14), sorting nexin 17 (SNX17) (ENSG00000115234.10), testis expressed 261 (TEX261) (ENSG00000144043.11), SMYD family member 5 (SMYD5) (ENSG00000135632.11), coiled-coil domain containing 142 (CCDC142) (ENSG00000135637.13), ssemaphorin 4F (SEMA4F) (ENSG00000135622.12), trans-golgi network protein 2 (TGOLN2) (ENSG00000152291.13), transmembrane protein 127 (TMEM127) (ENSG00000135956.8), mitotic spindle organizing protein 2B (MZT2B) (ENSG00000152082.13), Rho guanine nucleotide exchange factor 4 (ARHGEF4) (ENSG00000136002.18), COP9 signalosome subunit 7B (COPS7B) (ENSG00000144524.17), hes family bHLH transcription factor 6 (HES6) (ENSG00000144485.10), G protein-coupled receptor 35 (GPR35) (ENSG00000178623.11), actin related protein 2/3 complex subunit 4 (ARPC4) (ENSG00000241553.12), jagunal homolog 1 (JAGN1) (ENSG00000171135.13), interleukin 17 receptor C (IL17RC) (ENSG00000163702.18), transmembrane protein with metallophosphoesterase domain (TMPPE) (ENSG00000188167.8), ribosomal protein SA (RPSA) (ENSG00000168028.13), ubiquinol-cytochrome c reductase core protein 1 (UQCRC1) (ENSG00000010256.10), DALR anticodon binding domain containing 3 (DALRD3) (ENSG00000178149.16), cytochrome b561 family member D2 (CYB561D2) (ENSG00000114395.10), POC1 centriolar protein A (POC1A) (ENSG00000164087.7), solute carrier family 41 member 3 (SLC41A3) (ENSG00000114544.16), transmembrane protein adipocyte associated 1 (TPRA1) (ENSG00000163870.14), ALG3 alpha-1,3-mannosyltransferase (ALG3) (ENSG00000214160.9), EEF1A lysine methyltransferase 4 (EEF1AKMT4) (ENSG00000284753.1), major facilitator superfamily domain containing 10 (MFSD10) (ENSG00000109736.14), interferon regulatory factor 2 (IRF2) (ENSG00000168310.10), solute carrier family 35 member A4 (SLC35A4) (ENSG00000176087.14), NADH:ubiquinone oxidoreductase subunit A2 (NDUFA2) (ENSG00000131495.8), solute carrier family 36 member 1 (SLC36A1) (ENSG00000123643.12), antioxidant 1 copper chaperone (ATOX1) (ENSG00000177556.11), ADP ribosylation factor like GTPase 10 (ARL10) (ENSG00000175414.6), beta-1,4-galactosyltransferase 7 (B4GALT7) (ENSG00000027847.13), 5-phosphohydroxy-L-lysine phospho-lyase (PHYKPL) (ENSG00000175309.14), H3 clustered histone 2 (HIST1H3B) (ENSG00000274267.1), H1.2 linker histone, cluster member (HIST1H1C) (ENSG00000187837.3), H3 clustered histone 10 (HIST1H3H) (ENSG00000278828.1), olfactory receptor family 2 subfamily I member 1 pseudogene (OR2I1P) (ENSG00000237988.5), RNA polymerase I subunit H (ZNRD1) (ENSG00000066379.14), splicing factor 3b subunit 5 (SF3B5) (ENSG00000169976.6), aquaporin 1 (Colton blood group) (AQP1) (ENSG00000240583.11), receptor activity modifying protein 3 (RAMP3) (ENSG00000122679.8), NOP2/Sun RNA methyltransferase 5 (NSUN5) (ENSG00000130305.16), elastin (ELN) (ENSG00000049540.16), zona pellucida glycoprotein 3 (ZP3) (ENSG00000188372.14), cytochrome P450 family 51 subfamily A member 1 (CYP51A1) (ENSG00000001630.15), solute carrier family 12 member 9 (SLC12A9) (ENSG00000146828.17), monoacylglycerol O-acyltransferase 3 (MOGAT3) (ENSG00000106384.10), RNA polymerase II subunit J2 (POLR2J2) (ENSG00000228049.7), smoothened, frizzled class receptor (SMO) (ENSG00000128602.9), cell cycle regulator of NHEJ (C7orf49) (ENSG00000122783.16), MT-RNR2 like 6 (pseudogene) (MTRNR2L6) (ENSG00000270672.1), Fas activated serine/threonine kinase (FASTK) (ENSG00000164896.19), heparan-alpha-glucosaminide N-acetyltransferase (HGSNAT) (ENSG00000165102.14), grainyhead like transcription factor 2 (GRHL2) (ENSG00000083307.11), F-box and leucine rich repeat protein 6 (FBXL6) (ENSG00000182325.10), ribonuclease P/MRP subunit p25 like (RPP25L) (ENSG00000164967.9), phosphatidylinositol glycan anchor biosynthesis class O (PIGO) (ENSG00000165282.13), haloacid dehalogenase like hydrolase domain containing 3 (HDHD3) (ENSG00000119431.9), WD repeat domain 38 (WDR38) (ENSG00000136918.7), solute carrier family 2 member 8 (SLC2A8) (ENSG00000136856.17), torsin family 2 member A (TOR2A) (ENSG00000160404.17), ST6 N-acetylgalactosaminide alpha-2,6-sialyltransferase 6 (ST6GALNAC6) (ENSG00000160408.14), zinc finger DHHC-type palmitoyltransferase 12 (ZDHHC12) (ENSG00000160446.18), TBC1 domain family member 13 (TBC1D13) (ENSG00000107021.15), protein phosphatase 2 phosphatase activator (PPP2R4) (ENSG00000119383.19), calcium channel flower domain containing 1 (CACFD1) (ENSG00000160325.14), transmembrane protein 141 (TMEM141) (ENSG00000244187.7), transmembrane protein 203 (TMEM203) (ENSG00000187713.6), trypsin like peroxisomal matrix peptidase 1 (TYSND1) (ENSG00000156521.13), leucine rich repeat containing 20 (LRRC20) (ENSG00000172731.13), V-set immunoregulatory receptor (VSIR) (ENSG00000107738.19), SEC24 homolog C, COPII coat complex component (SEC24C) (ENSG00000176986.15), tectonic family member 3 (TCTN3) (ENSG00000119977.20), phosphatidylinositol 4-kinase type 2 alpha (PI4K2A) (ENSG00000155252.13), F-box and WD repeat domain containing 4 (FBXW4) (ENSG00000107829.13), glutathione S-transferase omega 1 (GSTO1) (ENSG00000148834.12), Bet1 golgi vesicular membrane trafficking protein like (BET1L) (ENSG00000177951.17), tetraspanin 4 (TSPAN4) (ENSG00000214063.10), chitinase domain containing 1 (CHID1) (ENSG00000177830.17), tumor suppressing subtransferable candidate 4 (TSSC4) (ENSG00000184281.14), FHF complex subunit HOOK interacting protein 1B (FAM160A2) (ENSG00000051009.10), peroxisomal biogenesis factor 16 (PEX16) (ENSG00000121680.15), solute carrier family 39 member 13 (SLC39A13) (ENSG00000165915.13), cytochrome b561 family member A3 (CYB561A3) (ENSG00000162144.9), transmembrane protein 258 (TMEM258) (ENSG00000134825.15), bestrophin 1 (BEST1) (ENSG00000167995.15), beta-1,3-glucuronyltransferase 3 (B3GAT3) (ENSG00000149541.9), integrator complex subunit 5 (INTS5) (ENSG00000185085.2), cytochrome c oxidase subunit 8A (COX8A) (ENSG00000176340.3), sorting nexin 32 (SNX32) (ENSG00000172803.17), frizzled class receptor 4 (FZD4) (ENSG00000174804.3), solute carrier family 37 member 4 (SLC37A4) (ENSG00000137700.17), NLR family member X1 (NLRX1) (ENSG00000160703.15), F-box and leucine rich repeat protein 14 (FBXL14) (ENSG00000171823.6), non-SMC condensin I complex subunit D2 (NCAPD2) (ENSG00000010292.12), myeloid leukemia factor 2 (MLF2) (ENSG00000089693.10), complement C3a receptor 1 (C3AR1) (ENSG00000171860.4), G protein-coupled receptor class C group 5 member A (GPRC5A) (ENSG00000013588.7), transmembrane protein 106C (TMEM106C) (ENSG00000134291.11), tubulin alpha 1b (TUBA1B) (ENSG00000123416.15), major facilitator superfamily domain containing 5 (MFSD5) (ENSG00000182544.8), ATP synthase membrane subunit C locus 2 (ATP5G2) (ENSG00000135390.17), retinol dehydrogenase 5 (RDH5) (ENSG00000135437.9), premelanosome protein (PMEL) (ENSG00000185664.14), cyclin dependent kinase 4 (CDK4) (ENSG00000135446.16), CTD small phosphatase 2 (CTDSP2) (ENSG00000175215.10), polypeptide N-acetylgalactosaminyltransferase 4 (GALNT4) (ENSG00000257594.3), phospholipase B domain containing 2 (PLBD2) (ENSG00000151176.7), paxillin (PXN) (ENSG00000089159.16), refilin A (FAM101A) (ENSG00000178882.14), transmembrane protein 253 (TMEM253) (ENSG00000232070.8), abhydrolase domain containing 4, N-acyl phospholipase B (ABHD4) (ENSG00000100439.10), proteasome 20S subunit beta 5 (PSMB5) (ENSG00000100804.18), phosphoenolpyruvate carboxykinase 2, mitochondrial (PCK2) (ENSG00000100889.11), ER membrane protein complex subunit 9 (EMC9) (ENSG00000100908.13), ribosomal protein S29 (RPS29) (ENSG00000213741.10), sushi domain containing 6 (SUSD6) (ENSG00000100647.7), acyl-CoA thioesterase 1 (ACOT1) (ENSG00000184227.7), acyl-CoA thioesterase 4 (ACOT4) (ENSG00000177465.4), ATP binding cassette subfamily D member 4 (ABCD4) (ENSG00000119688.20), NPC intracellular cholesterol transporter 2 (NPC2) (ENSG00000119655.10), angel homolog 1 (ANGEL1) (ENSG00000013523.9), CLOCK interacting pacemaker (CIPC) (ENSG00000198894.7), G protein-coupled receptor 68 (GPR68) (ENSG00000119714.10), solute carrier family 25 member 29 (SLC25A29) (ENSG00000197119.12), SIVA1 apoptosis inducing factor (SIVA1) (ENSG00000184990.12), G protein-coupled receptor 176 (GPR176) (ENSG00000166073.10), sorting nexin 22 (SNX22) (ENSG00000157734.13), pleckstrin homology domain containing 02 (PLEKHO2) (ENSG00000241839.9), cartilage intermediate layer protein (CILP) (ENSG00000138615.5), stomatin like 1 (STOML1) (ENSG00000067221.13), COMM domain containing 4 (COMMD4) (ENSG00000140365.15), phosphatidylinositol glycan anchor biosynthesis class Q (PIGQ) (ENSG00000007541.16), transmembrane protein 204 (TMEM204) (ENSG00000131634.13), NME/NM23 nucleoside diphosphate kinase 3 (NME3) (ENSG00000103024.7), NUBP iron-sulfur cluster assembly factor 2, cytosolic (NUBP2) (ENSG00000095906.16), presequence translocase associated motor 16 (PAM16) (ENSG00000217930.7), coronin 7 (CORO7) (ENSG00000262246.5), PAXIP1 associated glutamate rich protein 1 (PAGR1) (ENSG00000280789.1), CDP-diacylglycerol-inositol 3-phosphatidyltransferase (CDIPT) (ENSG00000103502.13), phosphorylase kinase catalytic subunit gamma 2 (PHKG2) (ENSG00000156873.15), ORAI calcium release-activated calcium modulator 3 (ORAI3) (ENSG00000175938.6), serine protease 8 (PRSS8) (ENSG00000052344.15), adhesion G protein-coupled receptor G1 (ADGRG1) (ENSG00000205336.11), chemokine like factor (CKLF) (ENSG00000217555.12), CKLF like MARVEL transmembrane domain containing 3 (CMTM3) (ENSG00000140931.19), transmembrane protein 208 (TMEM208) (ENSG00000168701.18), THAP domain containing 11 (THAP11) (ENSG00000168286.2), proteasome 20S subunit beta 10 (PSMB10) (ENSG00000205220.11), Tax1 binding protein 3 (TAX1BP3) (ENSG00000213977.7), GABA type A receptor-associated protein (GABARAP) (ENSG00000170296.9), transient receptor potential cation channel subfamily V member 2 (TRPV2) (ENSG00000187688.14), N-acetyltransferase domain containing 1 (NATD1) (ENSG00000274180.1), DNA polymerase delta interacting protein 2 (POLDIP2) (ENSG00000004142.11), notchless homolog 1 (NLE1) (ENSG00000073536.17), NFKB inhibitor interacting Ras like 2 (NKIRAS2) (ENSG00000168256.17), N-acetyl-alpha-glucosaminidase (NAGLU) (ENSG00000108784.9), reticulophagy regulator family member 3 (FAM134C) (ENSG00000141699.10), glucose-6-phosphatase catalytic subunit 3 (G6PC3) (ENSG00000141349.8), intercellular adhesion molecule 2 (ICAM2) (ENSG00000108622.10), G protein-coupled receptor class C group 5 member C (GPRC5C) (ENSG00000170412.16), thymidine kinase 1 (TK1) (ENSG00000167900.11), actin gamma 1 (ACTG1) (ENSG00000184009.10), FA core complex associated protein 100 (FAAP100) (ENSG00000185504.16), solute carrier family 25 member 10 (SLC25A10) (ENSG00000183048.11), phospholipid phosphatase 2 (PLPP2) (ENSG00000141934.9), secretory carrier membrane protein 4 (SCAMP4) (ENSG00000227500.9), MOB kinase activator 3A (MOB3A) (ENSG00000172081.13), fucosyltransferase 6 (FUT6) (ENSG00000156413.13), NADH:ubiquinone oxidoreductase subunit A11 (NDUFA11) (ENSG00000174886.12), calcyphosine (CAPS) (ENSG00000105519.15), PET100 cytochrome c oxidase chaperone (C19orf79) (ENSG00000229833.9), CD320 molecule (CD320) (ENSG00000167775.10), sphingosine-1-phosphate receptor 2 (S1PR2) (ENSG00000267534.3), autophagy related 4D cysteine peptidase (ATG4D) (ENSG00000130734.9), phospholipid phosphatase related 2 (PLPPR2) (ENSG00000105520.10), WD repeat domain 83 opposite strand (WDR83OS) (ENSG00000105583.9), notch receptor 3 (NOTCH3) (ENSG00000074181.8), adaptor related protein complex 1 subunit mu 1 (AP1M1) (ENSG00000072958.8), ubiquitin A-52 residue ribosomal protein fusion product 1 (UBA52) (ENSG00000221983.7), kelch like family member 26 (KLHL26) (ENSG00000167487.11), armadillo repeat containing 6 (ARMC6) (ENSG00000105676.13), transmembrane protein 161A (TMEM161A) (ENSG00000064545.14), nuclear receptor 2C2 associated protein (NR2C2AP) (ENSG00000184162.14), MAU2 sister chromatid cohesion factor (MAU2) (ENSG00000129933.20), transmembrane protein 147 (TMEM147) (ENSG00000105677.11), presenilin enhancer, gamma-secretase subunit (PSENEN) (ENSG00000205155.7), cytochrome c oxidase subunit 7A1 (COX7A1) (ENSG00000161281.10), Charcot-Leyden crystal galectin (CLC) (ENSG00000105205.6), zinc finger protein 574 (ZNF574) (ENSG00000105732.12), ETHE1 persulfide dioxygenase (ETHE1) (ENSG00000105755.7), adaptor related protein complex 2 subunit sigma 1 (AP2S1) (ENSG00000042753.11), transient receptor potential cation channel subfamily M member 4 (TRPM4) (ENSG00000130529.15), BCL2 like 12 (BCL2L12) (ENSG00000126453.9), sialic acid binding Ig like lectin 14 (SIGLEC14) (ENSG00000254415.3), formyl peptide receptor 1 (FPR1) (ENSG00000171051.8), protein phosphatase 2 scaffold subunit Aalpha (PPP2R1A) (ENSG00000105568.17), zinc finger protein 320 (ZNF320) (ENSG00000182986.13), ribosomal protein S5 (RPS5) (ENSG00000083845.8), small nuclear ribonucleoprotein polypeptides B and B1 (SNRPB) (ENSG00000125835.18), U-box domain containing 5 (UBOX5) (ENSG00000185019.16), acyl-CoA synthetase short chain family member 1 (ACSS1) (ENSG00000154930.14), glycogen phosphorylase B (PYGB) (ENSG00000100994.11), AAR2 splicing factor (AAR2) (ENSG00000131043.11), acyl-CoA thioesterase 8 (ACOT8) (ENSG00000101473.16), phosphorylated CTD interacting factor 1 (PCIF1) (ENSG00000100982.11), gamma-glutamyltransferase 5 (GGT5) (ENSG00000099998.17), rhomboid domain containing 3 (RHBDD3) (ENSG00000100263.13), galectin 1 (LGALS1) (ENSG00000100097.11), platelet derived growth factor subunit B (PDGFB) (ENSG00000100311.16), centromere protein M (CENPM) (ENSG00000100162.14), translocator protein (TSPO) (ENSG00000100300.17), gem nuclear organelle associated protein 8 (GEMIN8) (ENSG00000046647.13), NADH:ubiquinone oxidoreductase subunit B11 (NDUFB11) (ENSG00000147123.10), ribosomal protein L10 (RPL10) (ENSG00000147403.16), L antigen family member 3 (LAGE3) (ENSG00000196976.7), AC004556.1 (ENSG00000276345.1), peptidyl arginine deiminase 2 (PADI2) (ENSG00000117115.12), phospholipase A2 group IIA (PLA2G2A) (ENSG00000188257.10), collagen type IX alpha 2 chain (COL9A2) (ENSG00000049089.14), cytochrome P450 family 4 subfamily X member 1 (CYP4X1) (ENSG00000186377.7), enoyl-CoA hydratase domain containing 2 (ECHDC2) (ENSG00000121310.16), maestro heat like repeat family member 7 (MROH7) (ENSG00000184313.19), protein kinase cAMP-activated catalytic subunit beta (PRKACB) (ENSG00000142875.19), sterile alpha motif domain containing 13 (SAMD13) (ENSG00000203943.8), coagulation factor III, tissue factor (F3) (ENSG00000117525.13), 3-hydroxy-3-methylglutaryl-CoA synthase 2 (HMGCS2) (ENSG00000134240.11), selenium binding protein 1 (SELENBP1) (ENSG00000143416.20), regulator of G protein signaling 5 (RGS5) (ENSG00000143248.12), cathepsin E (CTSE) (ENSG00000196188.10), immunoglobulin kappa constant (IGKC) (ENSG00000211592.8), chromosome 2 open reading frame 88 (C2orf88) (ENSG00000187699.10), integral membrane protein 2C (ITM2C) (ENSG00000135916.15), FAM3 metabolism regulating signaling molecule D (FAM3D) (ENSG00000198643.6), ABI family member 3 binding protein (ABI3BP) (ENSG00000154175.16), resistin like beta (RETNLB) (ENSG00000163515.6), solute carrier organic anion transporter family member 2A1 (SLCO2A1) (ENSG00000174640.12), mucin 4, cell surface associated (MUC4) (ENSG00000145113.21), chromosome 4 open reading frame 19 (C4orf19) (ENSG00000154274.14), UDP glucuronosyltransferase family 2 member B17 (UGT2B17) (ENSG00000197888.2), joining chain of multimeric IgA and IgM (JCHAIN) (ENSG00000132465.10), N-acylethanolamine acid amidase (NAAA) (ENSG00000138744.14), nuclear receptor subfamily 3 group C member 2 (NR3C2) (ENSG00000151623.14), C-C motif chemokine ligand 28 (CCL28) (ENSG00000151882.11), marginal zone B and B1 cell specific protein (MZB1) (ENSG00000170476.15), solute carrier family 26 member 2 (SLC26A2) (ENSG00000155850.7), CD74 molecule (CD74) (ENSG00000019582.14), chloride intracellular channel 5 (CLIC5) (ENSG00000112782.16), 5′-nucleotidase ecto (NT5E) (ENSG00000135318.11), sphingomyelin phosphodiesterase acid like 3A (SMPDL3A) (ENSG00000172594.12), scinderin (SCIN) (ENSG00000006747.14), histone deacetylase 9 (HDAC9) (ENSG00000048052.21), transient receptor potential cation channel subfamily A member 1 (TRPA1) (ENSG00000104321.10), stathmin 2 (STMN2) (ENSG00000104435.13), glutamic-pyruvic transaminase (GPT) (ENSG00000167701.13), prostaglandin D2 synthase (PTGDS) (ENSG00000107317.12), Rho related BTB domain containing 1 (RHOBTB1) (ENSG00000072422.16), cadherin related family member 1 (CDHR1) (ENSG00000148600.14), 3′-phosphoadenosine 5′-phosphosulfate synthase 2 (PAPSS2) (ENSG00000198682.12), actin filament associated protein 1 like 2 (AFAP1L2) (ENSG00000169129.14), X-ray radiation resistance associated 1 (XRRA1) (ENSG00000166435.15), calpain 5 (CAPN5) (ENSG00000149260.15), coiled-coil domain containing 89 (CCDC89) (ENSG00000179071.4), synaptotagmin like 2 (SYTL2) (ENSG00000137501.17), matrix metallopeptidase 7 (MMP7) (ENSG00000137673.8), neurexophilin and PC-esterase domain family member 1 (NXPE1) (ENSG00000095110.8), neurexophilin and PC-esterase domain family member 4 (NXPE4) (ENSG00000137634.9), tripartite motif containing 29 (TRIM29) (ENSG00000137699.16), V-set and immunoglobulin domain containing 2 (VSIG2) (ENSG00000019102.11), solute carrier family 37 member 2 (SLC37A2) (ENSG00000134955.11), ST3 beta-galactoside alpha-2,3-sialyltransferase 4 (ST3GAL4) (ENSG00000110080.18), BARX homeobox 2 (BARX2) (ENSG00000043039.6), lysophosphatidic acid receptor 6 (LPAR6) (ENSG00000139679.15), heat shock protein family A (Hsp70) member 2 (HSPA2) (ENSG00000126803.9), bradykinin receptor B2 (BDKRB2) (ENSG00000168398.6), creatine kinase B (CKB) (ENSG00000166165.12), calpain 3 (CAPN3) (ENSG00000092529.23), beta-2-microglobulin (B2M) (ENSG00000166710.17), RNA binding fox-1 homolog 1 (RBFOX1) (ENSG00000078328.20), class II major histocompatibility complex transactivator (CIITA) (ENSG00000179583.19), endoplasmic reticulum to nucleus signaling 2 (ERN2) (ENSG00000134398.14), lactate dehydrogenase D (LDHD) (ENSG00000166816.13), carbohydrate sulfotransferase 5 (CHST5) (ENSG00000135702.14), carbonic anhydrase 4 (CA4) (ENSG00000167434.9), axin 2 (AXIN2) (ENSG00000168646.12), secreted and transmembrane 1 (SECTM1) (ENSG00000141574.7), solute carrier family 14 member 1 (Kidd blood group) (SLC14A1) (ENSG00000141469.17), ring finger protein 152 (RNF152) (ENSG00000176641.10), plasmalemma vesicle associated protein (PLVAP) (ENSG00000130300.8), cation channel sperm associated auxiliary subunit gamma (CATSPERG) (ENSG00000099338.22), Fc gamma binding protein (FCGBP) (ENSG00000275395.5), kallikrein 1 (KLK1) (ENSG00000167748.10), glycine receptor alpha 2 (GLRA2) (ENSG00000101958.13), claudin 2 (CLDN2) (ENSG00000165376.10), MT-ND3 (ENSG00000198840.2).

In particular for the identification of the MXE1 subtype, the expression of the predictive protein-coding genes is characterized by their up-regulation in the MXE1 subtype as compared to the MXE2 subtype, wherein the predictive protein-coding genes are selected from the primary group consisting of kelch like family member 21 (KLHL21) (ENSG00000162413.16), mitotic arrest deficient 2 like 2 (MAD2L2) (ENSG00000116670.14), angiotensin II receptor associated protein (AGTRAP) (ENSG00000177674.15), NBL1, DAN family BMP antagonist (NBL1) (ENSG00000158747.14), zinc finger DHHC-type palmitoyltransferase 18 (ZDHHC18) (ENSG00000204160.11), solute carrier family 9 member A1 (SLC9A1) (ENSG00000090020.10), transmembrane protein 222 (TMEM222) (ENSG00000186501.14), platelet activating factor receptor (PTAFR) (ENSG00000169403.11), penta-EF-hand domain containing 1 (PEF1) (ENSG00000162517.12), NHS like 3 (KIAA1522) (ENSG00000162522.10), chromosome 1 open reading frame 122 (C1orf122) (ENSG00000197982.13), bone morphogenetic protein 8a (BMP8A) (ENSG00000183682.7), importin 13 (IPO13) (ENSG00000117408.10), diphthamide biosynthesis 2 (DPH2) (ENSG00000132768.13), ATPase H+ transporting V0 subunit b (ATP6V0B) (ENSG00000117410.13), aldo-keto reductase family 1 member A1 (AKR1A1) (ENSG00000117448.13), leucine rich repeat containing 41 (LRRC41) (ENSG00000132128.16), PDZK1 interacting protein 1 (PDZK1IP1) (ENSG00000162366.7), 24-dehydrocholesterol reductase (DHCR24) (ENSG00000116133.11), CD53 molecule (CD53) (ENSG00000143119.12), WD repeat domain 77 (WDR77) (ENSG00000116455.13), peroxisomal biogenesis factor 11 beta (PEX11B) (ENSG00000131779.10), aph-1 homolog A, gamma-secretase subunit (APH1A) (ENSG00000117362.12), proteasome 20S subunit beta 4 (PSMB4) (ENSG00000159377.10), S100 calcium binding protein A2 (S100A2) (ENSG00000196754.10), jumping translocation breakpoint (JTB) (ENSG00000143543.14), RUN and SH3 domain containing 1 (RUSC1) (ENSG00000160753.15), methyltransferase like 25B (RRNAD1) (ENSG00000143303.11), SLAM family member 8 (SLAMF8) (ENSG00000158714.10), protoporphyrinogen oxidase (PPOX) (ENSG00000143224.17), uridine-cytidine kinase 2 (UCK2) (ENSG00000143179.15), SFT2 domain containing 2 (SFT2D2) (ENSG00000213064.9), transmembrane protein 9 (TMEM9) (ENSG00000116857.16), NUAK family kinase 2 (NUAK2) (ENSG00000163545.8), left-right determination factor 1 (LEFTY1) (ENSG00000243709.1), jumonji domain containing 4 (JMJD4) (ENSG00000081692.12), solute carrier family 35 member F6 (SLC35F6) (ENSG00000213699.8), solute carrier family 5 member 6 (SLC5A6) (ENSG00000138074.14), sorting nexin 17 (SNX17) (ENSG00000115234.10), testis expressed 261 (TEX261) (ENSG00000144043.11), SMYD family member 5 (SMYD5) (ENSG00000135632.11), coiled-coil domain containing 142 (CCDC142) (ENSG00000135637.13), ssemaphorin 4F (SEMA4F) (ENSG00000135622.12), trans-golgi network protein 2 (TGOLN2) (ENSG00000152291.13), transmembrane protein 127 (TMEM127) (ENSG00000135956.8), mitotic spindle organizing protein 2B (MZT2B) (ENSG00000152082.13), Rho guanine nucleotide exchange factor 4 (ARHGEF4) (ENSG00000136002.18), COP9 signalosome subunit 7B (COPS7B) (ENSG00000144524.17), hes family bHLH transcription factor 6 (HES6) (ENSG00000144485.10), G protein-coupled receptor 35 (GPR35) (ENSG00000178623.11), actin related protein 2/3 complex subunit 4 (ARPC4) (ENSG00000241553.12), jagunal homolog 1 (JAGN1) (ENSG00000171135.13), interleukin 17 receptor C (IL17RC) (ENSG00000163702.18), transmembrane protein with metallophosphoesterase domain (TMPPE) (ENSG00000188167.8), ribosomal protein SA (RPSA) (ENSG00000168028.13), ubiquinol-cytochrome c reductase core protein 1 (UQCRC1) (ENSG00000010256.10), DALR anticodon binding domain containing 3 (DALRD3) (ENSG00000178149.16), cytochrome b561 family member D2 (CYB561D2) (ENSG00000114395.10), POC1 centriolar protein A (POC1A) (ENSG00000164087.7), solute carrier family 41 member 3 (SLC41A3) (ENSG00000114544.16), transmembrane protein adipocyte associated 1 (TPRA1) (ENSG00000163870.14), ALG3 alpha-1,3-mannosyltransferase (ALG3) (ENSG00000214160.9), EEF1A lysine methyltransferase 4 (EEF1AKMT4) (ENSG00000284753.1), major facilitator superfamily domain containing 10 (MFSD10) (ENSG00000109736.14), interferon regulatory factor 2 (IRF2) (ENSG00000168310.10), solute carrier family 35 member A4 (SLC35A4) (ENSG00000176087.14), NADH:ubiquinone oxidoreductase subunit A2 (NDUFA2) (ENSG00000131495.8), solute carrier family 36 member 1 (SLC36A1) (ENSG00000123643.12), antioxidant 1 copper chaperone (ATOX1) (ENSG00000177556.11), ADP ribosylation factor like GTPase 10 (ARL10) (ENSG00000175414.6), beta-1,4-galactosyltransferase 7 (B4GALT7) (ENSG00000027847.13), 5-phosphohydroxy-L-lysine phospho-lyase (PHYKPL) (ENSG00000175309.14), H3 clustered histone 2 (HIST1H3B) (ENSG00000274267.1), H1.2 linker histone, cluster member (HIST1H1C) (ENSG00000187837.3), H3 clustered histone 10 (HIST1H3H) (ENSG00000278828.1), olfactory receptor family 2 subfamily I member 1 pseudogene (OR2I1P) (ENSG00000237988.5), RNA polymerase I subunit H (ZNRD1) (ENSG00000066379.14), splicing factor 3b subunit 5 (SF3B5) (ENSG00000169976.6), aquaporin 1 (Colton blood group) (AQP1) (ENSG00000240583.11), receptor activity modifying protein 3 (RAMP3) (ENSG00000122679.8), NOP2/Sun RNA methyltransferase 5 (NSUN5) (ENSG00000130305.16), elastin (ELN) (ENSG00000049540.16), zona pellucida glycoprotein 3 (ZP3) (ENSG00000188372.14), cytochrome P450 family 51 subfamily A member 1 (CYP51A1) (ENSG00000001630.15), solute carrier family 12 member 9 (SLC12A9) (ENSG00000146828.17), monoacylglycerol O-acyltransferase 3 (MOGAT3) (ENSG00000106384.10), RNA polymerase II subunit J2 (POLR2J2) (ENSG00000228049.7), smoothened, frizzled class receptor (SMO) (ENSG00000128602.9), cell cycle regulator of NHEJ (C7orf49) (ENSG00000122783.16), MT-RNR2 like 6 (pseudogene) (MTRNR2L6) (ENSG00000270672.1), Fas activated serine/threonine kinase (FASTK) (ENSG00000164896.19), heparan-alpha-glucosaminide N-acetyltransferase (HGSNAT) (ENSG00000165102.14), grainyhead like transcription factor 2 (GRHL2) (ENSG00000083307.11), F-box and leucine rich repeat protein 6 (FBXL6) (ENSG00000182325.10), ribonuclease P/MRP subunit p25 like (RPP25L) (ENSG00000164967.9), phosphatidylinositol glycan anchor biosynthesis class O (PIGO) (ENSG00000165282.13), haloacid dehalogenase like hydrolase domain containing 3 (HDHD3) (ENSG00000119431.9), WD repeat domain 38 (WDR38) (ENSG00000136918.7), solute carrier family 2 member 8 (SLC2A8) (ENSG00000136856.17), torsin family 2 member A (TOR2A) (ENSG00000160404.17), ST6 N-acetylgalactosaminide alpha-2,6-sialyltransferase 6 (ST6GALNAC6) (ENSG00000160408.14), zinc finger DHHC-type palmitoyltransferase 12 (ZDHHC12) (ENSG00000160446.18), TBC1 domain family member 13 (TBC1D13) (ENSG00000107021.15), protein phosphatase 2 phosphatase activator (PPP2R4) (ENSG00000119383.19), calcium channel flower domain containing 1 (CACFD1) (ENSG00000160325.14), transmembrane protein 141 (TMEM141) (ENSG00000244187.7), transmembrane protein 203 (TMEM203) (ENSG00000187713.6), trypsin like peroxisomal matrix peptidase 1 (TYSND1) (ENSG00000156521.13), leucine rich repeat containing 20 (LRRC20) (ENSG00000172731.13), V-set immunoregulatory receptor (VSIR) (ENSG00000107738.19), SEC24 homolog C, COPII coat complex component (SEC24C) (ENSG00000176986.15), tectonic family member 3 (TCTN3) (ENSG00000119977.20), phosphatidylinositol 4-kinase type 2 alpha (PI4K2A) (ENSG00000155252.13), F-box and WD repeat domain containing 4 (FBXW4) (ENSG00000107829.13), glutathione S-transferase omega 1 (GSTO1) (ENSG00000148834.12), Bet1 golgi vesicular membrane trafficking protein like (BET1L) (ENSG00000177951.17), tetraspanin 4 (TSPAN4) (ENSG00000214063.10), chitinase domain containing 1 (CHID1) (ENSG00000177830.17), tumor suppressing subtransferable candidate 4 (TSSC4) (ENSG00000184281.14), FHF complex subunit HOOK interacting protein 1B (FAM160A2) (ENSG00000051009.10), peroxisomal biogenesis factor 16 (PEX16) (ENSG00000121680.15), solute carrier family 39 member 13 (SLC39A13) (ENSG00000165915.13), cytochrome b561 family member A3 (CYB561A3) (ENSG00000162144.9), transmembrane protein 258 (TMEM258) (ENSG00000134825.15), bestrophin 1 (BEST1) (ENSG00000167995.15), beta-1,3-glucuronyltransferase 3 (B3GAT3) (ENSG00000149541.9), integrator complex subunit 5 (INTS5) (ENSG00000185085.2), cytochrome c oxidase subunit 8A (COX8A) (ENSG00000176340.3), sorting nexin 32 (SNX32) (ENSG00000172803.17), frizzled class receptor 4 (FZD4) (ENSG00000174804.3), solute carrier family 37 member 4 (SLC37A4) (ENSG00000137700.17), NLR family member X1 (NLRX1) (ENSG00000160703.15), F-box and leucine rich repeat protein 14 (FBXL14) (ENSG00000171823.6), non-SMC condensin I complex subunit D2 (NCAPD2) (ENSG00000010292.12), myeloid leukemia factor 2 (MLF2) (ENSG00000089693.10), complement C3a receptor 1 (C3AR1) (ENSG00000171860.4), G protein-coupled receptor class C group 5 member A (GPRC5A) (ENSG00000013588.7), transmembrane protein 106C (TMEM106C) (ENSG00000134291.11), tubulin alpha 1b (TUBA1B) (ENSG00000123416.15), major facilitator superfamily domain containing 5 (MFSD5) (ENSG00000182544.8), ATP synthase membrane subunit C locus 2 (ATP5G2) (ENSG00000135390.17), retinol dehydrogenase 5 (RDH5) (ENSG00000135437.9), premelanosome protein (PMEL) (ENSG00000185664.14), cyclin dependent kinase 4 (CDK4) (ENSG00000135446.16), CTD small phosphatase 2 (CTDSP2) (ENSG00000175215.10), polypeptide N-acetylgalactosaminyltransferase 4 (GALNT4) (ENSG00000257594.3), phospholipase B domain containing 2 (PLBD2) (ENSG00000151176.7), paxillin (PXN) (ENSG00000089159.16), refilin A (FAM101A) (ENSG00000178882.14), transmembrane protein 253 (TMEM253) (ENSG00000232070.8), abhydrolase domain containing 4, N-acyl phospholipase B (ABHD4) (ENSG00000100439.10), proteasome 20S subunit beta 5 (PSMB5) (ENSG00000100804.18), phosphoenolpyruvate carboxykinase 2, mitochondrial (PCK2) (ENSG00000100889.11), ER membrane protein complex subunit 9 (EMC9) (ENSG00000100908.13), ribosomal protein S29 (RPS29) (ENSG00000213741.10), sushi domain containing 6 (SUSD6) (ENSG00000100647.7), acyl-CoA thioesterase 1 (ACOT1) (ENSG00000184227.7), acyl-CoA thioesterase 4 (ACOT4) (ENSG00000177465.4), ATP binding cassette subfamily D member 4 (ABCD4) (ENSG00000119688.20), NPC intracellular cholesterol transporter 2 (NPC2) (ENSG00000119655.10), angel homolog 1 (ANGEL1) (ENSG00000013523.9), CLOCK interacting pacemaker (CIPC) (ENSG00000198894.7), G protein-coupled receptor 68 (GPR68) (ENSG00000119714.10), solute carrier family 25 member 29 (SLC25A29) (ENSG00000197119.12), SIVA1 apoptosis inducing factor (SIVA1) (ENSG00000184990.12), G protein-coupled receptor 176 (GPR176) (ENSG00000166073.10), sorting nexin 22 (SNX22) (ENSG00000157734.13), pleckstrin homology domain containing 02 (PLEKHO2) (ENSG00000241839.9), cartilage intermediate layer protein (CILP) (ENSG00000138615.5), stomatin like 1 (STOML1) (ENSG00000067221.13), COMM domain containing 4 (COMMD4) (ENSG00000140365.15), phosphatidylinositol glycan anchor biosynthesis class Q (PIGQ) (ENSG00000007541.16), transmembrane protein 204 (TMEM204) (ENSG00000131634.13), NME/NM23 nucleoside diphosphate kinase 3 (NME3) (ENSG00000103024.7), NUBP iron-sulfur cluster assembly factor 2, cytosolic (NUBP2) (ENSG00000095906.16), presequence translocase associated motor 16 (PAM16) (ENSG00000217930.7), coronin 7 (CORO7) (ENSG00000262246.5), PAXIP1 associated glutamate rich protein 1 (PAGR1) (ENSG00000280789.1), CDP-diacylglycerol-inositol 3-phosphatidyltransferase (CDIPT) (ENSG00000103502.13), phosphorylase kinase catalytic subunit gamma 2 (PHKG2) (ENSG00000156873.15), ORAI calcium release-activated calcium modulator 3 (ORAI3) (ENSG00000175938.6), serine protease 8 (PRSS8) (ENSG00000052344.15), adhesion G protein-coupled receptor G1 (ADGRG1) (ENSG00000205336.11), chemokine like factor (CKLF) (ENSG00000217555.12), CKLF like MARVEL transmembrane domain containing 3 (CMTM3) (ENSG00000140931.19), transmembrane protein 208 (TMEM208) (ENSG00000168701.18), THAP domain containing 11 (THAP11) (ENSG00000168286.2), proteasome 20S subunit beta 10 (PSMB10) (ENSG00000205220.11), Tax1 binding protein 3 (TAX1BP3) (ENSG00000213977.7), GABA type A receptor-associated protein (GABARAP) (ENSG00000170296.9), transient receptor potential cation channel subfamily V member 2 (TRPV2) (ENSG00000187688.14), N-acetyltransferase domain containing 1 (NATD1) (ENSG00000274180.1), DNA polymerase delta interacting protein 2 (POLDIP2) (ENSG00000004142.11), notchless homolog 1 (NLE1) (ENSG00000073536.17), NFKB inhibitor interacting Ras like 2 (NKIRAS2) (ENSG00000168256.17), N-acetyl-alpha-glucosaminidase (NAGLU) (ENSG00000108784.9), reticulophagy regulator family member 3 (FAM134C) (ENSG00000141699.10), glucose-6-phosphatase catalytic subunit 3 (G6PC3) (ENSG00000141349.8), intercellular adhesion molecule 2 (ICAM2) (ENSG00000108622.10), G protein-coupled receptor class C group 5 member C (GPRC5C) (ENSG00000170412.16), thymidine kinase 1 (TK1) (ENSG00000167900.11), actin gamma 1 (ACTG1) (ENSG00000184009.10), FA core complex associated protein 100 (FAAP100) (ENSG00000185504.16), solute carrier family 25 member 10 (SLC25A10) (ENSG00000183048.11), phospholipid phosphatase 2 (PLPP2) (ENSG00000141934.9), secretory carrier membrane protein 4 (SCAMP4) (ENSG00000227500.9), MOB kinase activator 3A (MOB3A) (ENSG00000172081.13), fucosyltransferase 6 (FUT6) (ENSG00000156413.13), NADH:ubiquinone oxidoreductase subunit A11 (NDUFA11) (ENSG00000174886.12), calcyphosine (CAPS) (ENSG00000105519.15), PET100 cytochrome c oxidase chaperone (C19orf79) (ENSG00000229833.9), CD320 molecule (CD320) (ENSG00000167775.10), sphingosine-1-phosphate receptor 2 (S1PR2) (ENSG00000267534.3), autophagy related 4D cysteine peptidase (ATG4D) (ENSG00000130734.9), phospholipid phosphatase related 2 (PLPPR2) (ENSG00000105520.10), WD repeat domain 83 opposite strand (WDR83OS) (ENSG00000105583.9), notch receptor 3 (NOTCH3) (ENSG00000074181.8), adaptor related protein complex 1 subunit mu 1 (AP1M1) (ENSG00000072958.8), ubiquitin A-52 residue ribosomal protein fusion product 1 (UBA52) (ENSG00000221983.7), kelch like family member 26 (KLHL26) (ENSG00000167487.11), armadillo repeat containing 6 (ARMC6) (ENSG00000105676.13), transmembrane protein 161A (TMEM161A) (ENSG00000064545.14), nuclear receptor 2C2 associated protein (NR2C2AP) (ENSG00000184162.14), MAU2 sister chromatid cohesion factor (MAU2) (ENSG00000129933.20), transmembrane protein 147 (TMEM147) (ENSG00000105677.11), presenilin enhancer, gamma-secretase subunit (PSENEN) (ENSG00000205155.7), cytochrome c oxidase subunit 7A1 (COX7A1) (ENSG00000161281.10), Charcot-Leyden crystal galectin (CLC) (ENSG00000105205.6), zinc finger protein 574 (ZNF574) (ENSG00000105732.12), ETHE1 persulfide dioxygenase (ETHE1) (ENSG00000105755.7), adaptor related protein complex 2 subunit sigma 1 (AP2S1) (ENSG00000042753.11), transient receptor potential cation channel subfamily M member 4 (TRPM4) (ENSG00000130529.15), BCL2 like 12 (BCL2L12) (ENSG00000126453.9), sialic acid binding Ig like lectin 14 (SIGLEC14) (ENSG00000254415.3), formyl peptide receptor 1 (FPR1) (ENSG00000171051.8), protein phosphatase 2 scaffold subunit Aalpha (PPP2R1A) (ENSG00000105568.17), zinc finger protein 320 (ZNF320) (ENSG00000182986.13), ribosomal protein S5 (RPS5) (ENSG00000083845.8), small nuclear ribonucleoprotein polypeptides B and B1 (SNRPB) (ENSG00000125835.18), U-box domain containing 5 (UBOX5) (ENSG00000185019.16), acyl-CoA synthetase short chain family member 1 (ACSS1) (ENSG00000154930.14), glycogen phosphorylase B (PYGB) (ENSG00000100994.11), AAR2 splicing factor (AAR2) (ENSG00000131043.11), acyl-CoA thioesterase 8 (ACOT8) (ENSG00000101473.16), phosphorylated CTD interacting factor 1 (PCIF1) (ENSG00000100982.11), gamma-glutamyltransferase 5 (GGT5) (ENSG00000099998.17), rhomboid domain containing 3 (RHBDD3) (ENSG00000100263.13), galectin 1 (LGALS1) (ENSG00000100097.11), platelet derived growth factor subunit B (PDGFB) (ENSG00000100311.16), centromere protein M (CENPM) (ENSG00000100162.14), translocator protein (TSPO) (ENSG00000100300.17), gem nuclear organelle associated protein 8 (GEMIN8) (ENSG00000046647.13), NADH:ubiquinone oxidoreductase subunit B11 (NDUFB11) (ENSG00000147123.10), ribosomal protein L10 (RPL10) (ENSG00000147403.16), L antigen family member 3 (LAGE3) (ENSG00000196976.7), AC004556.1 (ENSG00000276345.1).

In one embodiment for the identification of the MXE1 subtype, the expression of the predictive protein-coding genes selected from G protein-coupled receptor 35 (GPR35), grainyhead like transcription factor 2 (GRHL2) (ENSG00000083307.11), G protein-coupled receptor class C group 5 member A (GPRC5A) (ENSG00000013588.7), cartilage intermediate layer protein (CILP) (ENSG00000138615.5) and calcyphosine (CAPS) (ENSG00000105519.15) are excluded.

Preferably, additionally the predictive protein-coding genes are selected from the secondary group consisting of solute carrier family 35 member E2B (SLC35E2B) (ENSG00000189339.11), aldo-keto reductase family 7 member A3 (AKR7A3) (ENSG00000162482.4), keratinocyte differentiation factor 1 (KDF1) (ENSG00000175707.8), serine incorporator 2 (SERINC2) (ENSG00000168528.11), transmembrane protein 54 (TMEM54) (ENSG00000121900.18), polyhomeotic homolog 2 (PHC2) (ENSG00000134686.18), solute carrier family 44 member 3 (SLC44A3) (ENSG00000143036.16), glutathione S-transferase mu 3 (GSTM3) (ENSG00000134202.10), H3 clustered histone 13 (HIST2H3D) (ENSG00000183598.3), atypical chemokine receptor 1 (Duffy blood group) (ACKR1) (ENSG00000213088.10), arginyl aminopeptidase (RNPEP) (ENSG00000176393.10), major facilitator superfamily domain containing 4A (MFSD4A) (ENSG00000174514.12), calpain 13 (CAPN13) (ENSG00000162949.16), protein kinase domain containing, cytoplasmic (PKDCC) (ENSG00000162878.12), C-X-C motif chemokine receptor 4 (CXCR4) (ENSG00000121966.6), cytochrome P450 family 27 subfamily A member 1 (CYP27A1) (ENSG00000135929.8), F-box and leucine rich repeat protein 2 (FBXL2) (ENSG00000153558.14), transmembrane protein 129, E3 ubiquitin ligase (TMEM129) (ENSG00000168936.10), family with sequence similarity 53 member C (FAM53C) (ENSG00000120709.10), stimulator of interferon response CGAMP interactor 1 (TMEM173) (ENSG00000184584.12), CD14 molecule (CD14) (ENSG00000170458.13), protocadherin 1 (PCDH1) (ENSG00000156453.13), SH3 domain and tetratricopeptide repeats 2 (SH3TC2) (ENSG00000169247.12), alpha-1,3-mannosyl-glycoprotein 4-beta-N-acetylglucosaminyltransferase B (MGAT4B) (ENSG00000161013.16), sequestosome 1 (SQSTM1) (ENSG00000161011.19), abhydrolase domain containing 16A, phospholipase (ABHD16A) (ENSG00000204427.11), lymphocyte antigen 6 family member G6D (LY6G6D) (ENSG00000244355.7), peptidase inhibitor 16 (PI16) (ENSG00000164530.13), MAD2L1 binding protein (MAD2L1BP) (ENSG00000124688.13), translocation associated membrane protein 2 (TRAM2) (ENSG00000065308.4), rhomboid domain containing 2 (RHBDD2) (ENSG00000005486.16), serpin family E member 1 (SERPINE1) (ENSG00000106366.8), zinc finger C3HC-type containing 1 (ZC3HC1) (ENSG00000091732.15), thromboxane A synthase 1 (TBXAS1) (ENSG00000059377.16), protein O-mannose kinase (POMK) (ENSG00000185900.9), glycosylphosphatidylinositol anchor attachment 1 (GPAA1) (ENSG00000197858.10), zinc finger FYVE-type containing 27 (ZFYVE27) (ENSG00000155256.17), mucin 6, oligomeric mucus/gel-forming (MUC6) (ENSG00000184956.15), Rho GTPase activating protein 1 (ARHGAP1) (ENSG00000175220.11), diacylglycerol lipase alpha (DAGLA) (ENSG00000134780.9), ADP ribosylation factor like GTPase 2 (ARL2) (ENSG00000213465.7), triosephosphate isomerase 1 (TPI1) (ENSG00000111669.14), chromosome 12 open reading frame 57 (C12orf57) (ENSG00000111678.10), EMG1 N1-specific pseudouridine methyltransferase (EMG1) (ENSG00000126749.14), solute carrier family 48 member 1 (SLC48A1) (ENSG00000211584.13), transmembrane BAX inhibitor motif containing 6 (TMBIM6) (ENSG00000139644.12), keratin 6B (KRT6B) (ENSG00000185479.5), OXA1L mitochondrial inner membrane protein (OXA1L) (ENSG00000155463.12), NEDD8 ubiquitin like modifier (NEDD8) (ENSG00000129559.12), coenzyme Q6, monooxygenase (COQ6) (ENSG00000119723.16), kelch like family member 25 (KLHL25) (ENSG00000183655.12), ATP binding cassette subfamily A member 3 (ABCA3) (ENSG00000167972.13), serine protease 21 (PRSS21) (ENSG00000007038.10), ATP binding cassette subfamily C member 6 (ABCC6) (ENSG00000091262.15), coronin 1A (CORO1A) (ENSG00000102879.15), thymidine kinase 2 (TK2) (ENSG00000166548.15), ring finger protein 135 (RNF135) (ENSG00000181481.13), TBC1 domain family member 3L (TBC1D3L) (ENSG00000274512.5), GH3 domain containing (GHDC) (ENSG00000167925.15), homeobox B13 (HOXB13) (ENSG00000159184.7), RNA polymerase II, I and III subunit E (POLR2E) (ENSG00000099817.11), methyl-CpG binding domain protein 3 (MBD3) (ENSG00000071655.17), ubiquinol-cytochrome c reductase, complex III subunit XI (UQCR11) (ENSG00000127540.11), vimentin type intermediate filament associated coiled-coil protein (VMAC) (ENSG00000187650.3), kelch like ECH associated protein 1 (KEAP1) (ENSG00000079999.13), adaptor related protein complex 1 subunit mu 2 (AP1M2) (ENSG00000129354.11), transmembrane p24 trafficking protein 1 (TMED1) (ENSG00000099203.6), BRISC and BRCA1 A complex member 1 (BABAM1) (ENSG00000105393.15), collagen beta (1-O) galactosyltransferase 1 (GLT25D1) (ENSG00000130309.10), zinc finger protein 132 (ZNF132) (ENSG00000131849.11), ectonucleoside triphosphate diphosphohydrolase 6 (ENTPD6) (ENSG00000197586.12), MT-RNR2 like 3 (pseudogene) (MTRNR2L3) (ENSG00000256222.2), solute carrier family 19 member 1 (SLC19A1) (ENSG00000173638.18), claudin 5 (CLDN5) (ENSG00000184113.9), phosphoinositide-3-kinase interacting protein 1 (PIK3IP1) (ENSG00000100100.12), non-SMC condensin II complex subunit H2 (NCAPH2) (ENSG00000025770.18), coiled-coil domain containing 22 (CCDC22) (ENSG00000101997.12), ubiquilin 2 (UBQLN2) (ENSG00000188021.8), transmembrane protein 164 (TMEM164) (ENSG00000157600.11).

The ranking as primary and secondary events is performed via stability scores (max 1, min 0.9) indicating consistent and robust predictive value for identification of MXE subtypes. Primary events have stability scores >=0.95 and secondary events have stability scores <0.95.

Also preferred for the identification of MXE2 subtype is the expression of the predictive protein-coding genes, which is characterized by their up-regulation in the MXE2 subtype as compared to the MXE1 subtype.

Preferably for the identification of MXE2 subtype the predictive protein-coding genes are selected from the primary group consisting of TAR DNA binding protein (TARDBP) (ENSG00000120948.16), heterogeneous nuclear ribonucleoprotein C like 4 (HNRNPCL4) (ENSG00000179412.10), capping actin protein of muscle Z-line subunit beta (CAPZB) (ENSG00000077549.17), heterogeneous nuclear ribonucleoprotein R (HNRNPR) (ENSG00000125944.19), single stranded DNA binding protein 3 (SSBP3) (ENSG00000157216.15), nexilin F-actin binding protein (NEXN) (ENSG00000162614.18), far upstream element binding protein 1 (FUBP1) (ENSG00000162613.16), cellular communication network factor 1 (CYR61) (ENSG00000142871.16), SET like protein (SETSIP) (ENSG00000230667.5), major facilitator superfamily domain containing 14A (MFSD14A) (ENSG00000156875.13), SAS-6 centriolar assembly protein (SASS6) (ENSG00000156876.9), pre-mRNA processing factor 38B (PRPF38B) (ENSG00000134186.11), TATA-box binding protein associated factor 13 (TAF13) (ENSG00000197780.9), RAP1A, member of RAS oncogene family (RAP1A) (ENSG00000116473.14), H2A clustered histone 19 (HIST2H2AA4) (ENSG00000272196.2), H3 clustered histone 15 (HIST2H3A) (ENSG00000203852.3), H4 clustered histone 15 (HIST2H4B) (ENSG00000270276.2), acidic nuclear phosphoprotein 32 family member E (ANP32E) (ENSG00000143401.14), S100 calcium binding protein A10 (S100A10) (ENSG00000197747.8), regulator of G protein signaling 13 (RGS13) (ENSG00000127074.14), ribosomal protein S27a pseudogene 5 (RP11-92K2.2) (ENSG00000231767.4), ubiquitin C-terminal hydrolase L5 (UCHL5) (ENSG00000116750.13), LIM homeobox 9 (LHX9) (ENSG00000143355.15), zinc finger BED-type containing 6 (ZBED6) (ENSG00000257315.2), zinc finger CCCH-type containing 11B (ZC3H11B) (ENSG00000215817.5), ras homolog family member U (RHOU) (ENSG00000116574.5), translin associated factor X (TSNAX) (ENSG00000116918.13), myelin transcription factor 1 like (MYT1L) (ENSG00000186487.18), protein phosphatase 1 catalytic subunit beta (PPP1CB) (ENSG00000213639.9), protein phosphatase, Mg2+/Mn2+ dependent 1B (PPM1B) (ENSG00000138032.20), C1D nuclear receptor corepressor (C1D) (ENSG00000197223.11), poly(rC) binding protein 1 (PCBP1) (ENSG00000169564.6), RANBP2 like and GRIP domain containing 1 (RGPD1) (ENSG00000187627.15), RANBP2 like and GRIP domain containing 2 (RGPD2) (ENSG00000185304.14), RANBP2 like and GRIP domain containing 3 (RGPD3) (ENSG00000153165.18), RANBP2 like and GRIP domain containing 4 (RGPD4) (ENSG00000196862.9), GRIP and coiled-coil domain containing 2 (GCC2) (ENSG00000135968.20), RANBP2 like and GRIP domain containing 5 (RGPD5) (ENSG00000015568.12), RANBP2 like and GRIP domain containing 6 (RGPD6) (ENSG00000183054.11), diazepam binding inhibitor, acyl-CoA binding protein (DBI) (ENSG00000155368.16), RAB6C, member RAS oncogene family (RAB6C) (ENSG00000222014.5), POTE ankyrin domain family member I (POTEI) (ENSG00000196834.11), POTE ankyrin domain family member J (POTEJ) (ENSG00000222038.3), UBX domain protein 4 (UBXN4) (ENSG00000144224.16), ADP ribosylation factor like GTPase 5A (ARL5A) (ENSG00000162980.16), membrane associated ring-CH-type finger 7 (MARCH7) (ENSG00000136536.14), peptidylprolyl isomerase G (PPIG) (ENSG00000138398.15), small RNA binding exonuclease protection factor La (SSB) (ENSG00000138385.15), tousled like kinase 1 (TLK1) (ENSG00000198586.13), corepressor interacting with RBPJ, CIR1 (CIR1) (ENSG00000138433.15), heterogeneous nuclear ribonucleoprotein A3 (HNRNPA3) (ENSG00000170144.20), MOB family member 4, phocein (MOB4) (ENSG00000115540.14), tubulin alpha 4b (TUBA4B) (ENSG00000243910.7), prothymosin alpha (PTMA) (ENSG00000187514.16), myeloma overexpressed 2 pseudogene (MYEOV2) (ENSG00000172428.10), synapsin II (SYN2) (ENSG00000157152.16), LSM3 homolog, U6 small nuclear RNA and mRNA degradation associated (LSM3) (ENSG00000170860.3), protein kinase C delta (PRKCD) (ENSG00000163932.13), MT-RNR2 like 12 (pseudogene) (MTRNR2L12) (ENSG00000269028.3), filamin A interacting protein 1 like (FILIP1L) (ENSG00000168386.18), PEST proteolytic signal containing nuclear protein (PCNP) (ENSG00000081154.11), RAB7A, member RAS oncogene family (RAB7A) (ENSG00000075785.12), WW domain containing transcription regulator 1 (WWTR1) (ENSG00000018408.14), structural maintenance of chromosomes 4 (SMC4) (ENSG00000113810.15), SKI like proto-oncogene (SKIL) (ENSG00000136603.13), cytoplasmic polyadenylation element binding protein 2 (CPEB2) (ENSG00000137449.15), MOB kinase activator 1B (MOB1B) (ENSG00000173542.8), Ras association domain family member 6 (RASSF6) (ENSG00000169435.13), heterogeneous nuclear ribonucleoprotein D like (HNRNPDL) (ENSG00000152795.17), DnaJ heat shock protein family (Hsp40) member B14 (DNAJB14) (ENSG00000164031.16), family with sequence similarity 241 member A (C4orf32) (ENSG00000174749.5), La ribonucleoprotein 7, transcriptional regulator (LARP7) (ENSG00000174720.15), progesterone receptor membrane component 2 (PGRMC2) (ENSG00000164040.16), tryptophan 2,3-dioxygenase (TDO2) (ENSG00000151790.8), palladin, cytoskeletal associated protein (PALLD) (ENSG00000129116.17), 15-hydroxyprostaglandin dehydrogenase (HPGD) (ENSG00000164120.13), inhibitor of growth family member 2 (ING2) (ENSG00000168556.6), cilia and flagella associated protein 97 (CFAP97) (ENSG00000164323.12), NADH:ubiquinone oxidoreductase subunit S6 (NDUFS6) (ENSG00000145494.11), SUB1 regulator of transcription (SUB1) (ENSG00000113387.11), NADH:ubiquinone oxidoreductase complex assembly factor 2 (NDUFAF2) (ENSG00000164182.10), CWC27 spliceosome associated cyclophilin (CWC27) (ENSG00000153015.15), protein geranylgeranyltransferase type I subunit beta (PGGT1B) (ENSG00000164219.9), coiled-coil domain containing 112 (CCDC112) (ENSG00000164221.12), lysyl oxidase (LOX) (ENSG00000113083.13), ubiquitin conjugating enzyme E2 B (UBE2B) (ENSG00000119048.7), RNA binding motif protein 27 (RBM27) (ENSG00000091009.7), H4 clustered histone 11 (HIST1H4J) (ENSG00000197238.4), H4 clustered histone 12 (HIST1H4K) (ENSG00000273542.1), CD2 associated protein (CD2AP) (ENSG00000198087.7), KIAA1586 (KIAA1586) (ENSG00000168116.13), FKBP prolyl isomerase family member 1C (FKBP1C) (ENSG00000198225.5), high mobility group nucleosomal binding domain 3 (HMGN3) (ENSG00000118418.14), SEC63 homolog, protein translocation regulator (SEC63) (ENSG00000025796.13), myristoylated alanine rich protein kinase C substrate (MARCKS) (ENSG00000277443.2), RWD domain containing 1 (RWDD1) (ENSG00000111832.12), NUS1 dehydrodolichyl diphosphate synthase subunit (NUS1) (ENSG00000153989.7), cellular communication network factor 2 (CTGF) (ENSG00000118523.5), BCL2 associated transcription factor 1 (BCLAF1) (ENSG00000029363.16), LTV1 ribosome biogenesis factor (LTV1) (ENSG00000135521.8), TGF-beta activated kinase 1 (MAP3K7) binding protein 2 (TAB2) (ENSG00000055208.18), peptidylprolyl isomerase like 4 (PPIL4) (ENSG00000131013.3), RB associated KRAB zinc finger (RBAK) (ENSG00000146587.17), transmembrane protein 106B (TMEM106B) (ENSG00000106460.18), aryl hydrocarbon receptor (AHR) (ENSG00000106546.13), transformer 2 alpha homolog (TRA2A) (ENSG00000164548.10), sperm microtubule inner protein 4 (C7orf31) (ENSG00000153790.11), heterogeneous nuclear ribonucleoprotein A2/B1 (HNRNPA2B1) (ENSG00000122566.20), zinc and ring finger 2 (ZNRF2) (ENSG00000180233.10), ITPR interacting domain containing 1 (CCDC129) (ENSG00000180347.13), POU class 6 homeobox 2 (POU6F2) (ENSG00000106536.19), RAS like proto-oncogene A (RALA) (ENSG00000006451.7), inhibin subunit beta A (INHBA) (ENSG00000122641.10), zinc finger protein 680 (ZNF680) (ENSG00000173041.11), fibrinogen like 2 (FGL2) (ENSG00000127951.6), A-kinase anchoring protein 9 (AKAP9) (ENSG00000127914.16), family with sequence similarity 133 member B (FAM133B) (ENSG00000234545.7), DnaJ heat shock protein family (Hsp40) member C2 (DNAJC2) (ENSG00000105821.14), NADH:ubiquinone oxidoreductase subunit A5 (NDUFA5) (ENSG00000128609.14), chromosome 7 open reading frame 73 (C7orf73) (ENSG00000243317.7), protein kinase AMP-activated non-catalytic subunit gamma 2 (PRKAG2) (ENSG00000106617.13), neurofilament medium chain (NEFM) (ENSG00000104722.13), dynactin subunit 6 (DCTN6) (ENSG00000104671.7), CCAAT enhancer binding protein delta (CEBPD) (ENSG00000221869.4), syntrophin gamma 1 (SNTG1) (ENSG00000147481.15), RAB2A, member RAS oncogene family (RAB2A) (ENSG00000104388.14), translocation associated membrane protein 1 (TRAM1) (ENSG00000067167.7), zinc finger C2HC-type containing 1A (ZC2HC1A) (ENSG00000104427.11), tumor protein D52 (TPD52) (ENSG00000076554.15), ribosomal biogenesis factor (C8orf59) (ENSG00000176731.11), ubiquinol-cytochrome c reductase binding protein (UQCRB) (ENSG00000156467.9), RNA polymerase II, I and III subunit K (POLR2K) (ENSG00000147669.10), poly(A) binding protein cytoplasmic 1 (PABPC1) (ENSG00000070756.15), glycine decarboxylase (GLDC) (ENSG00000178445.9), zinc finger DHHC-type palmitoyltransferase 21 (ZDHHC21) (ENSG00000175893.11), PC4 and SRSF1 interacting protein 1 (PSIP1) (ENSG00000164985.14), caspase activity and apoptosis inhibitor 1 (CAAP1) (ENSG00000120159.11), TATA-box binding protein associated factor 1 like (TAF1L) (ENSG00000122728.6), carnosine N-methyltransferase 1 (CARNMT1) (ENSG00000156017.12), riboflavin kinase (RFK) (ENSG00000135002.11), RecQ mediated genome instability 1 (RMI1) (ENSG00000178966.16), acidic nuclear phosphoprotein 32 family member B (ANP32B) (ENSG00000136938.8), actin binding transcription modulator (FAM206A) (ENSG00000119328.11), TATA-box binding protein associated factor 3 (TAF3) (ENSG00000165632.7), chondroitin sulfate N-acetylgalactosaminyltransferase 2 (CSGALNACT2) (ENSG00000169826.7), ubiquitin conjugating enzyme E2 D1 (UBE2D1) (ENSG00000072401.14), VPS26 retromer complex component A (VPS26A) (ENSG00000122958.14), eukaryotic translation initiation factor 5A like 1 (EIF5AL1) (ENSG00000253626.3), kinesin family member 20B (KIF20B) (ENSG00000138182.14), FRA10A associated CGG repeat 1 (FRA10AC1) (ENSG00000148690.11), survival motor neuron domain containing 1 (SMNDC1) (ENSG00000119953.12), coiled-coil domain containing 186 (CCDC186) (ENSG00000165813.18), pleckstrin homology like domain family A member 2 (PHLDA2) (ENSG00000181649.6), MT-RNR2 like 8 (pseudogene) (MTRNR2L8) (ENSG00000255823.4), lin-7 homolog C, crumbs cell polarity complex component (LIN7C) (ENSG00000148943.11), glycine-N-acyltransferase like 1 (GLYATL1) (ENSG00000166840.13), membrane spanning 4-domains A6E (MS4A6E) (ENSG00000166926.8), heterogeneous nuclear ribonucleoprotein U like 2 (HNRNPUL2) (ENSG00000214753.2), FKBP prolyl isomerase 2 (FKBP2) (ENSG00000173486.12), spliceosome associated factor 1, recruiter of U4/U6.U5 tri-snRNP (SART1) (ENSG00000175467.14), remodeling and spacing factor 1 (RSF1) (ENSG00000048649.13), cysteine and histidine rich domain containing 1 (CHORDC1) (ENSG00000110172.11), ankyrin repeat domain 49 (ANKRD49) (ENSG00000168876.8), family with sequence similarity 76 member B (FAM76B) (ENSG00000077458.12), coiled-coil domain containing 82 (CCDC82) (ENSG00000149231.12), transmembrane protein 123 (TMEM123) (ENSG00000152558.14), defective in cullin neddylation 1 domain containing 5 (DCUN1D5) (ENSG00000137692.11), RNA binding motif protein 7 (RBM7) (ENSG00000076053.10), H2A.X variant histone (H2AFX) (ENSG00000188486.3), parathymosin (PTMS) (ENSG00000159335.15), basic helix-loop-helix family member e41 (BHLHE41) (ENSG00000123095.5), ERGIC and golgi 2 (ERGIC2) (ENSG00000087502.17), glucoside xylosyltransferase 1 (GXYLT1) (ENSG00000151233.10), zinc finger CCHC-type and RNA binding motif containing 1 (ZCRB1) (ENSG00000139168.7), twinfilin actin binding protein 1 (TWF1) (ENSG00000151239.13), activating transcription factor 1 (ATF1) (ENSG00000123268.8), oxysterol binding protein like 8 (OSBPL8) (ENSG00000091039.16), RNA 5′-phosphate and 3′-OH ligase 1 (C12orf29) (ENSG00000133641.17), centrosomal protein 290 (CEP290) (ENSG00000198707.14), early endosome antigen 1 (EEA1) (ENSG00000102189.16), small nuclear ribonucleoprotein polypeptide F (SNRPF) (ENSG00000139343.10), nuclear transcription factor Y subunit beta (NFYB) (ENSG00000120837.7), mitochondrial calcium uptake 2 (MICU2) (ENSG00000165487.13), poly(A) binding protein cytoplasmic 3 (PABPC3) (ENSG00000151846.8), casein kinase 1 alpha 1 like (CSNK1A1L) (ENSG00000180138.7), laccase domain containing 1 (LACC1) (ENSG00000179630.10), GPALPP motifs containing 1 (GPALPP1) (ENSG00000133114.17), tumor protein, translationally-controlled 1 (TPT1) (ENSG00000133112.16), zinc finger CCCH-type containing 13 (ZC3H13) (ENSG00000123200.16), integral membrane protein 2B (ITM2B) (ENSG00000136156.12), cytoskeleton associated protein 2 (CKAP2) (ENSG00000136108.14), mitotic spindle organizing protein 1 (MZT1) (ENSG00000204899.5), KLF transcription factor 5 (KLF5) (ENSG00000102554.13), potassium channel tetramerization domain containing 12 (KCTD12) (ENSG00000178695.5), coiled-coil domain containing 168 (CCDC168) (ENSG00000175820.3), arginine and glutamate rich 1 (ARGLU1) (ENSG00000134884.13), serine palmitoyltransferase small subunit A (SPTSSA) (ENSG00000165389.6), ras homolog family member J (RHOJ) (ENSG00000126785.12), golgin A6 family like 6 (GOLGA6L6) (ENSG00000277322.1), golgin A6 family like 2 (GOLGA6L2) (ENSG00000174450.11), ER membrane protein complex subunit 7 (EMC7) (ENSG00000134153.9), eukaryotic translation initiation factor 3 subunit J (EIF3J) (ENSG00000104131.12), COP9 signalosome subunit 2 (COPS2) (ENSG00000166200.14), RAB27A, member RAS oncogene family (RAB27A) (ENSG00000069974.15), SAFB like transcription modulator (SLTM) (ENSG00000137776.16), reticulocalbin 2 (RCN2) (ENSG00000117906.13), methenyltetrahydrofolate synthetase (MTHFS) (ENSG00000136371.10), zinc finger AN1-type containing 6 (ZFAND6) (ENSG00000086666.18), nuclear receptor subfamily 2 group F member 2 (NR2F2) (ENSG00000185551.14), centrosomal protein 20 (FOPNL) (ENSG00000133393.12), centriolar coiled-coil protein 110 (CCP110) (ENSG00000103540.16), nuclear pore complex interacting protein family member B5 (NPIPB5) (ENSG00000243716.10), eukaryotic translation initiation factor 3 subunit C like (EIF3CL) (ENSG00000205609.12), terminal nucleotidyltransferase 4B (PAPD5) (ENSG00000121274.12), tubulin polymerization promoting protein family member 3 (TPPP3) (ENSG00000159713.10), NIN1 (RPN12) binding protein 1 homolog (NOB1) (ENSG00000141101.12), contactin associated protein family member 4 (CNTNAP4) (ENSG00000152910.18), MAF bZIP transcription factor (MAF) (ENSG00000178573.6), CBFA2/RUNX1 partner transcriptional co-repressor 3 (CBFA2T3) (ENSG00000129993.14), ankyrin repeat domain containing 11 (ANKRD11) (ENSG00000167522.15), ER membrane protein complex subunit 6 (TMEM93) (ENSG00000127774.6), lysine demethylase 6B (KDM6B) (ENSG00000132510.10), ribosomal protein L26 (RPL26) (ENSG00000161970.13), SUZ12 polycomb repressive complex 2 subunit (SUZ12) (ENSG00000178691.10), insulin like growth factor binding protein 4 (IGFBP4) (ENSG00000141753.6), keratin 10 (KRT10) (ENSG00000186395.7), transducer of ERBB2, 1 (TOB1) (ENSG00000141232.4), nascent polypeptide associated complex subunit alpha 2 (NACA2) (ENSG00000253506.2), SRY-box transcription factor 9 (SOX9) (ENSG00000125398.6), ring finger protein 138 (RNF138) (ENSG00000134758.13), immediate early response 3 interacting protein 1 (IER3IP1) (ENSG00000134049.5), methyl-CpG binding domain protein 2 (MBD2) (ENSG00000134046.11), SEC11 homolog C, signal peptidase complex subunit (SEC11C) (ENSG00000166562.8), coiled-coil domain containing 102B (CCDC102B) (ENSG00000150636.17), zinc finger and BTB domain containing 7A (ZBTB7A) (ENSG00000178951.8), scaffold attachment factor B2 (SAFB2) (ENSG00000130254.11), scaffold attachment factor B (SAFB) (ENSG00000160633.12), tubulin beta 4A class IVa (TUBB4A) (ENSG00000104833.11), RAB11B, member RAS oncogene family (RAB11B) (ENSG00000185236.11), solute carrier family 1 member 6 (SLC1A6) (ENSG00000105143.12), KLF transcription factor 2 (KLF2) (ENSG00000127528.5), zinc finger protein 708 (ZNF708) (ENSG00000182141.10), PAF1 homolog, Paf1/RNA polymerase II complex component (PAF1) (ENSG00000006712.14), transforming growth factor beta 1 (TGFB1) (ENSG00000105329.9), novel protein, readthrough between CEACAM5-CEACAM6 (AC011513.3) (ENSG00000267881.1), CD177 molecule (CD177) (ENSG00000204936.9), potassium voltage-gated channel subfamily A member 7 (KCNA7) (ENSG00000104848.1), ribosomal protein L28 (RPL28) (ENSG00000108107.14), zinc finger protein 580 (ZNF580) (ENSG00000213015.8), ESF1 nucleolar pre-rRNA processing protein homolog (ESF1) (ENSG00000089048.14), charged multivesicular body protein 4B (CHMP4B) (ENSG00000101421.3), DNA topoisomerase I (TOP1) (ENSG00000198900.5), prefoldin subunit 4 (PFDN4) (ENSG00000101132.9), PRELI domain containing 3B (PRELID3B) (ENSG00000101166.15), synaptonemal complex protein 2 (SYCP2) (ENSG00000196074.12), TATA-box binding protein associated factor 4 (TAF4) (ENSG00000130699.18), cholinergic receptor nicotinic alpha 4 subunit (CHRNA4) (ENSG00000101204.16), SRY-box transcription factor 18 (SOX18) (ENSG00000203883.6), histone H2B type F-S-like (CH507-42P11.1) (ENSG00000274559.3), ATP synthase peripheral stalk subunit F6 (ATP5J) (ENSG00000154723.12), GA binding protein transcription factor subunit alpha (GABPA) (ENSG00000154727.10), SR-related CTD associated factor 4 (SCAF4) (ENSG00000156304.14), H2B clustered histone 12 like (H2BFS) (ENSG00000234289.5), BCLAF1 And THRAP3 family member 3 (CXorf23) (ENSG00000173681.16), proprotein convertase subtilisin/kexin type 1 inhibitor (PCSK1N) (ENSG00000102109.8), MT-RNR2 like 10 (pseudogene) (MTRNR2L10) (ENSG00000256045.2), ATRX chromatin remodeler (ATRX) (ENSG00000085224.21), transcription elongation factor A like 5 (TCEAL5) (ENSG00000204065.2), glutamate dehydrogenase 2 (GLUD2) (ENSG00000182890.4), hypoxanthine phosphoribosyltransferase 1 (HPRT1) (ENSG00000165704.14), oxidoreductase core subunit 6 (MT-ND6) (ENSG00000198695.2), enolase 1 (ENO1) (ENSG00000074800.14), solute carrier family 2 member 1 (SLC2A1) (ENSG00000117394.21), adenylate kinase 4 (AK4) (ENSG00000162433.14), tuftelin 1 (TUFT1) (ENSG00000143367.15), activating transcription factor 3 (ATF3) (ENSG00000162772.16), regenerating family member 1 alpha (REG1A) (ENSG00000115386.5), dual specificity phosphatase 2 (DUSP2) (ENSG00000158050.4), pyruvate dehydrogenase kinase 1 (PDK1) (ENSG00000152256.13), basic helix-loop-helix family member e40 (BHLHE40) (ENSG00000134107.4), C-X-C motif chemokine ligand 8 (CXCL8) (ENSG00000169429.10), secreted phosphoprotein 1 (SPP1) (ENSG00000118785.13), secreted frizzled related protein 2 (SFRP2) (ENSG00000145423.4), natriuretic peptide receptor 3 (NPR3) (ENSG00000113389.15), stanniocalcin 2 (STC2) (ENSG00000113739.10), immediate early response 3 (IER3) (ENSG00000137331.11), heat shock protein family A (Hsp70) member 1B (HSPA1B) (ENSG00000204388.6), alpha-2-glycoprotein 1, zinc-binding (AZGP1) (ENSG00000160862.12), potassium voltage-gated channel subfamily H member 2 (KCNH2) (ENSG00000055118.14), BCL2 interacting protein 3 like (BNIP3L) (ENSG00000104765.15), PLAG1 zinc finger (PLAG1) (ENSG00000181690.7), N-myc downstream regulated 1 (NDRG1) (ENSG00000104419.14), carbonic anhydrase 9 (CA9) (ENSG00000107159.12), UDP-N-acetylglucosamine pyrophosphorylase 1 like 1 (UAP1L1) (ENSG00000197355.10), prolyl 4-hydroxylase subunit alpha 1 (P4HA1) (ENSG00000122884.12), interferon induced transmembrane protein 1 (IFITM1) (ENSG00000185885.15), interferon induced transmembrane protein 3 (IFITM3) (ENSG00000142089.15), cleavage stimulation factor subunit 3 (CSTF3) (ENSG00000176102.11), protein kinase C and casein kinase substrate in neurons 3 (PACSIN3) (ENSG00000165912.15), fatty acid desaturase 2 (FADS2) (ENSG00000134824.13), fatty acid desaturase 1 (FADS1) (ENSG00000149485.18), enolase 2 (ENO2) (ENSG00000111674.8), COPI coat complex subunit zeta 1 (COPZ1) (ENSG00000111481.9), syntaxin 2 (STX2) (ENSG00000111450.13), endoplasmic reticulum oxidoreductase 1 alpha (ERO1A) (ENSG00000197930.12), dipeptidase 1 (DPEP1) (ENSG00000015413.9), lectin, mannose binding 1 (LMAN1) (ENSG00000074695.5), kallikrein related peptidase 6 (KLK6) (ENSG00000167755.14), troponin T1, slow skeletal type (TNNT1) (ENSG00000105048.16), ribophorin II (RPN2) (ENSG00000118705.16), WAP four-disulfide core domain 3 (WFDC3) (ENSG00000124116.18), deoxynucleotidyltransferase terminal interacting protein 1 (DNTTIP1) (ENSG00000101457.12), ubiquitin conjugating enzyme E2 C (UBE2C) (ENSG00000175063.16), dolichyl-phosphate mannosyltransferase subunit 1, catalytic (DPM1) (ENSG00000000419.12).

Preferably for the identification of MXE2 subtype the predictive protein-coding genes are selected from the primary group consisting of TAR DNA binding protein (TARDBP) (ENSG00000120948.16), heterogeneous nuclear ribonucleoprotein C like 4 (HNRNPCL4) (ENSG00000179412.10), capping actin protein of muscle Z-line subunit beta (CAPZB) (ENSG00000077549.17), heterogeneous nuclear ribonucleoprotein R (HNRNPR) (ENSG00000125944.19), single stranded DNA binding protein 3 (SSBP3) (ENSG00000157216.15), nexilin F-actin binding protein (NEXN) (ENSG00000162614.18), far upstream element binding protein 1 (FUBP1) (ENSG00000162613.16), cellular communication network factor 1 (CYR61) (ENSG00000142871.16), SET like protein (SETSIP) (ENSG00000230667.5), major facilitator superfamily domain containing 14A (MFSD14A) (ENSG00000156875.13), SAS-6 centriolar assembly protein (SASS6) (ENSG00000156876.9), pre-mRNA processing factor 38B (PRPF38B) (ENSG00000134186.11), TATA-box binding protein associated factor 13 (TAF13) (ENSG00000197780.9), RAP1A, member of RAS oncogene family (RAP1A) (ENSG00000116473.14), H2A clustered histone 19 (HIST2H2AA4) (ENSG00000272196.2), H3 clustered histone 15 (HIST2H3A) (ENSG00000203852.3), H4 clustered histone 15 (HIST2H4B) (ENSG00000270276.2), acidic nuclear phosphoprotein 32 family member E (ANP32E) (ENSG00000143401.14), S100 calcium binding protein A10 (S100A10) (ENSG00000197747.8), regulator of G protein signaling 13 (RGS13) (ENSG00000127074.14), ribosomal protein S27a pseudogene 5 (RP11-92K2.2) (ENSG00000231767.4), ubiquitin C-terminal hydrolase L5 (UCHL5) (ENSG00000116750.13), LIM homeobox 9 (LHX9) (ENSG00000143355.15), zinc finger BED-type containing 6 (ZBED6) (ENSG00000257315.2), zinc finger CCCH-type containing 11B (ZC3H11B) (ENSG00000215817.5), ras homolog family member U (RHOU) (ENSG00000116574.5), translin associated factor X (TSNAX) (ENSG00000116918.13), myelin transcription factor 1 like (MYT1L) (ENSG00000186487.18), protein phosphatase 1 catalytic subunit beta (PPP1CB) (ENSG00000213639.9), protein phosphatase, Mg2+/Mn2+ dependent 1B (PPM1B) (ENSG00000138032.20), C1D nuclear receptor corepressor (C1D) (ENSG00000197223.11), poly(rC) binding protein 1 (PCBP1) (ENSG00000169564.6), RANBP2 like and GRIP domain containing 1 (RGPD1) (ENSG00000187627.15), RANBP2 like and GRIP domain containing 2 (RGPD2) (ENSG00000185304.14), RANBP2 like and GRIP domain containing 3 (RGPD3) (ENSG00000153165.18), RANBP2 like and GRIP domain containing 4 (RGPD4) (ENSG00000196862.9), GRIP and coiled-coil domain containing 2 (GCC2) (ENSG00000135968.20), RANBP2 like and GRIP domain containing 5 (RGPD5) (ENSG00000015568.12), RANBP2 like and GRIP domain containing 6 (RGPD6) (ENSG00000183054.11), diazepam binding inhibitor, acyl-CoA binding protein (DBI) (ENSG00000155368.16), RAB6C, member RAS oncogene family (RAB6C) (ENSG00000222014.5), POTE ankyrin domain family member I (POTEI) (ENSG00000196834.11), POTE ankyrin domain family member J (POTEJ) (ENSG00000222038.3), UBX domain protein 4 (UBXN4) (ENSG00000144224.16), ADP ribosylation factor like GTPase 5A (ARL5A) (ENSG00000162980.16), membrane associated ring-CH-type finger 7 (MARCH7) (ENSG00000136536.14), peptidylprolyl isomerase G (PPIG) (ENSG00000138398.15), small RNA binding exonuclease protection factor La (SSB) (ENSG00000138385.15), tousled like kinase 1 (TLK1) (ENSG00000198586.13), corepressor interacting with RBPJ, CIR1 (CIR1) (ENSG00000138433.15), heterogeneous nuclear ribonucleoprotein A3 (HNRNPA3) (ENSG00000170144.20), MOB family member 4, phocein (MOB4) (ENSG00000115540.14), tubulin alpha 4b (TUBA4B) (ENSG00000243910.7), prothymosin alpha (PTMA) (ENSG00000187514.16), myeloma overexpressed 2 pseudogene (MYEOV2) (ENSG00000172428.10), synapsin II (SYN2) (ENSG00000157152.16), LSM3 homolog, U6 small nuclear RNA and mRNA degradation associated (LSM3) (ENSG00000170860.3), protein kinase C delta (PRKCD) (ENSG00000163932.13), MT-RNR2 like 12 (pseudogene) (MTRNR2L12) (ENSG00000269028.3), filamin A interacting protein 1 like (FILIP1L) (ENSG00000168386.18), PEST proteolytic signal containing nuclear protein (PCNP) (ENSG00000081154.11), RAB7A, member RAS oncogene family (RAB7A) (ENSG00000075785.12), WW domain containing transcription regulator 1 (WWTR1) (ENSG00000018408.14), structural maintenance of chromosomes 4 (SMC4) (ENSG00000113810.15), SKI like proto-oncogene (SKIL) (ENSG00000136603.13), cytoplasmic polyadenylation element binding protein 2 (CPEB2) (ENSG00000137449.15), MOB kinase activator 1B (MOB1B) (ENSG00000173542.8), Ras association domain family member 6 (RASSF6) (ENSG00000169435.13), heterogeneous nuclear ribonucleoprotein D like (HNRNPDL) (ENSG00000152795.17), DnaJ heat shock protein family (Hsp40) member B14 (DNAJB14) (ENSG00000164031.16), family with sequence similarity 241 member A (C4orf32) (ENSG00000174749.5), La ribonucleoprotein 7, transcriptional regulator (LARP7) (ENSG00000174720.15), progesterone receptor membrane component 2 (PGRMC2) (ENSG00000164040.16), tryptophan 2,3-dioxygenase (TDO2) (ENSG00000151790.8), palladin, cytoskeletal associated protein (PALLD) (ENSG00000129116.17), 15-hydroxyprostaglandin dehydrogenase (HPGD) (ENSG00000164120.13), inhibitor of growth family member 2 (ING2) (ENSG00000168556.6), cilia and flagella associated protein 97 (CFAP97) (ENSG00000164323.12), NADH:ubiquinone oxidoreductase subunit S6 (NDUFS6) (ENSG00000145494.11), SUB1 regulator of transcription (SUB1) (ENSG00000113387.11), NADH:ubiquinone oxidoreductase complex assembly factor 2 (NDUFAF2) (ENSG00000164182.10), CWC27 spliceosome associated cyclophilin (CWC27) (ENSG00000153015.15), protein geranylgeranyltransferase type I subunit beta (PGGT1B) (ENSG00000164219.9), coiled-coil domain containing 112 (CCDC112) (ENSG00000164221.12), lysyl oxidase (LOX) (ENSG00000113083.13), ubiquitin conjugating enzyme E2 B (UBE2B) (ENSG00000119048.7), RNA binding motif protein 27 (RBM27) (ENSG00000091009.7), H4 clustered histone 11 (HIST1H4J) (ENSG00000197238.4), H4 clustered histone 12 (HIST1H4K) (ENSG00000273542.1), CD2 associated protein (CD2AP) (ENSG00000198087.7), KIAA1586 (KIAA1586) (ENSG00000168116.13), FKBP prolyl isomerase family member 1C (FKBP1C) (ENSG00000198225.5), high mobility group nucleosomal binding domain 3 (HMGN3) (ENSG00000118418.14), SEC63 homolog, protein translocation regulator (SEC63) (ENSG00000025796.13), myristoylated alanine rich protein kinase C substrate (MARCKS) (ENSG00000277443.2), RWD domain containing 1 (RWDD1) (ENSG00000111832.12), NUS1 dehydrodolichyl diphosphate synthase subunit (NUS1) (ENSG00000153989.7), cellular communication network factor 2 (CTGF) (ENSG00000118523.5), BCL2 associated transcription factor 1 (BCLAF1) (ENSG00000029363.16), LTV1 ribosome biogenesis factor (LTV1) (ENSG00000135521.8), TGF-beta activated kinase 1 (MAP3K7) binding protein 2 (TAB2) (ENSG00000055208.18), peptidylprolyl isomerase like 4 (PPIL4) (ENSG00000131013.3), RB associated KRAB zinc finger (RBAK) (ENSG00000146587.17), transmembrane protein 106B (TMEM106B) (ENSG00000106460.18), aryl hydrocarbon receptor (AHR) (ENSG00000106546.13), transformer 2 alpha homolog (TRA2A) (ENSG00000164548.10), sperm microtubule inner protein 4 (C7orf31) (ENSG00000153790.11), heterogeneous nuclear ribonucleoprotein A2/B1 (HNRNPA2B1) (ENSG00000122566.20), zinc and ring finger 2 (ZNRF2) (ENSG00000180233.10), ITPR interacting domain containing 1 (CCDC129) (ENSG00000180347.13), POU class 6 homeobox 2 (POU6F2) (ENSG00000106536.19), RAS like proto-oncogene A (RALA) (ENSG00000006451.7), inhibin subunit beta A (INHBA) (ENSG00000122641.10), zinc finger protein 680 (ZNF680) (ENSG00000173041.11), fibrinogen like 2 (FGL2) (ENSG00000127951.6), A-kinase anchoring protein 9 (AKAP9) (ENSG00000127914.16), family with sequence similarity 133 member B (FAM133B) (ENSG00000234545.7), DnaJ heat shock protein family (Hsp40) member C2 (DNAJC2) (ENSG00000105821.14), NADH:ubiquinone oxidoreductase subunit A5 (NDUFA5) (ENSG00000128609.14), chromosome 7 open reading frame 73 (C7orf73) (ENSG00000243317.7), protein kinase AMP-activated non-catalytic subunit gamma 2 (PRKAG2) (ENSG00000106617.13), neurofilament medium chain (NEFM) (ENSG00000104722.13), dynactin subunit 6 (DCTN6) (ENSG00000104671.7), CCAAT enhancer binding protein delta (CEBPD) (ENSG00000221869.4), syntrophin gamma 1 (SNTG1) (ENSG00000147481.15), RAB2A, member RAS oncogene family (RAB2A) (ENSG00000104388.14), translocation associated membrane protein 1 (TRAM1) (ENSG00000067167.7), zinc finger C2HC-type containing 1A (ZC2HC1A) (ENSG00000104427.11), tumor protein D52 (TPD52) (ENSG00000076554.15), ribosomal biogenesis factor (C8orf59) (ENSG00000176731.11), ubiquinol-cytochrome c reductase binding protein (UQCRB) (ENSG00000156467.9), RNA polymerase II, I and III subunit K (POLR2K) (ENSG00000147669.10), poly(A) binding protein cytoplasmic 1 (PABPC1) (ENSG00000070756.15), glycine decarboxylase (GLDC) (ENSG00000178445.9), zinc finger DHHC-type palmitoyltransferase 21 (ZDHHC21) (ENSG00000175893.11), PC4 and SRSF1 interacting protein 1 (PSIP1) (ENSG00000164985.14), caspase activity and apoptosis inhibitor 1 (CAAP1) (ENSG00000120159.11), TATA-box binding protein associated factor 1 like (TAF1L) (ENSG00000122728.6), carnosine N-methyltransferase 1 (CARNMT1) (ENSG00000156017.12), riboflavin kinase (RFK) (ENSG00000135002.11), RecQ mediated genome instability 1 (RMI1) (ENSG00000178966.16), acidic nuclear phosphoprotein 32 family member B (ANP32B) (ENSG00000136938.8), actin binding transcription modulator (FAM206A) (ENSG00000119328.11), TATA-box binding protein associated factor 3 (TAF3) (ENSG00000165632.7), chondroitin sulfate N-acetylgalactosaminyltransferase 2 (CSGALNACT2) (ENSG00000169826.7), ubiquitin conjugating enzyme E2 D1 (UBE2D1) (ENSG00000072401.14), VPS26 retromer complex component A (VPS26A) (ENSG00000122958.14), eukaryotic translation initiation factor 5A like 1 (EIF5AL1) (ENSG00000253626.3), kinesin family member 20B (KIF20B) (ENSG00000138182.14), FRA10A associated CGG repeat 1 (FRA10AC1) (ENSG00000148690.11), survival motor neuron domain containing 1 (SMNDC1) (ENSG00000119953.12), coiled-coil domain containing 186 (CCDC186) (ENSG00000165813.18), pleckstrin homology like domain family A member 2 (PHLDA2) (ENSG00000181649.6), MT-RNR2 like 8 (pseudogene) (MTRNR2L8) (ENSG00000255823.4), lin-7 homolog C, crumbs cell polarity complex component (LIN7C) (ENSG00000148943.11), glycine-N-acyltransferase like 1 (GLYATL1) (ENSG00000166840.13), membrane spanning 4-domains A6E (MS4A6E) (ENSG00000166926.8), heterogeneous nuclear ribonucleoprotein U like 2 (HNRNPUL2) (ENSG00000214753.2), FKBP prolyl isomerase 2 (FKBP2) (ENSG00000173486.12), spliceosome associated factor 1, recruiter of U4/U6.U5 tri-snRNP (SART1) (ENSG00000175467.14), remodeling and spacing factor 1 (RSF1) (ENSG00000048649.13), cysteine and histidine rich domain containing 1 (CHORDC1) (ENSG00000110172.11), ankyrin repeat domain 49 (ANKRD49) (ENSG00000168876.8), family with sequence similarity 76 member B (FAM76B) (ENSG00000077458.12), coiled-coil domain containing 82 (CCDC82) (ENSG00000149231.12), transmembrane protein 123 (TMEM123) (ENSG00000152558.14), defective in cullin neddylation 1 domain containing 5 (DCUN1D5) (ENSG00000137692.11), RNA binding motif protein 7 (RBM7) (ENSG00000076053.10), H2A.X variant histone (H2AFX) (ENSG00000188486.3), parathymosin (PTMS) (ENSG00000159335.15), basic helix-loop-helix family member e41 (BHLHE41) (ENSG00000123095.5), ERGIC and golgi 2 (ERGIC2) (ENSG00000087502.17), glucoside xylosyltransferase 1 (GXYLT1) (ENSG00000151233.10), zinc finger CCHC-type and RNA binding motif containing 1 (ZCRB1) (ENSG00000139168.7), twinfilin actin binding protein 1 (TWF1) (ENSG00000151239.13), activating transcription factor 1 (ATF1) (ENSG00000123268.8), oxysterol binding protein like 8 (OSBPL8) (ENSG00000091039.16), RNA 5′-phosphate and 3′-OH ligase 1 (C12orf29) (ENSG00000133641.17), centrosomal protein 290 (CEP290) (ENSG00000198707.14), early endosome antigen 1 (EEA1) (ENSG00000102189.16), small nuclear ribonucleoprotein polypeptide F (SNRPF) (ENSG00000139343.10), nuclear transcription factor Y subunit beta (NFYB) (ENSG00000120837.7), mitochondrial calcium uptake 2 (MICU2) (ENSG00000165487.13), poly(A) binding protein cytoplasmic 3 (PABPC3) (ENSG00000151846.8), casein kinase 1 alpha 1 like (CSNK1A1L) (ENSG00000180138.7), laccase domain containing 1 (LACC1) (ENSG00000179630.10), GPALPP motifs containing 1 (GPALPP1) (ENSG00000133114.17), tumor protein, translationally-controlled 1 (TPT1) (ENSG00000133112.16), zinc finger CCCH-type containing 13 (ZC3H13) (ENSG00000123200.16), integral membrane protein 2B (ITM2B) (ENSG00000136156.12), cytoskeleton associated protein 2 (CKAP2) (ENSG00000136108.14), mitotic spindle organizing protein 1 (MZT1) (ENSG00000204899.5), KLF transcription factor 5 (KLF5) (ENSG00000102554.13), potassium channel tetramerization domain containing 12 (KCTD12) (ENSG00000178695.5), coiled-coil domain containing 168 (CCDC168) (ENSG00000175820.3), arginine and glutamate rich 1 (ARGLU1) (ENSG00000134884.13), serine palmitoyltransferase small subunit A (SPTSSA) (ENSG00000165389.6), ras homolog family member J (RHOJ) (ENSG00000126785.12), golgin A6 family like 6 (GOLGA6L6) (ENSG00000277322.1), golgin A6 family like 2 (GOLGA6L2) (ENSG00000174450.11), ER membrane protein complex subunit 7 (EMC7) (ENSG00000134153.9), eukaryotic translation initiation factor 3 subunit J (EIF3J) (ENSG00000104131.12), COP9 signalosome subunit 2 (COPS2) (ENSG00000166200.14), RAB27A, member RAS oncogene family (RAB27A) (ENSG00000069974.15), SAFB like transcription modulator (SLTM) (ENSG00000137776.16), reticulocalbin 2 (RCN2) (ENSG00000117906.13), methenyltetrahydrofolate synthetase (MTHFS) (ENSG00000136371.10), zinc finger AN1-type containing 6 (ZFAND6) (ENSG00000086666.18), nuclear receptor subfamily 2 group F member 2 (NR2F2) (ENSG00000185551.14), centrosomal protein 20 (FOPNL) (ENSG00000133393.12), centriolar coiled-coil protein 110 (CCP110) (ENSG00000103540.16), nuclear pore complex interacting protein family member B5 (NPIPB5) (ENSG00000243716.10), eukaryotic translation initiation factor 3 subunit C like (EIF3CL) (ENSG00000205609.12), terminal nucleotidyltransferase 4B (PAPD5) (ENSG00000121274.12), tubulin polymerization promoting protein family member 3 (TPPP3) (ENSG00000159713.10), NIN1 (RPN12) binding protein 1 homolog (NOB1) (ENSG00000141101.12), contactin associated protein family member 4 (CNTNAP4) (ENSG00000152910.18), MAF bZIP transcription factor (MAF) (ENSG00000178573.6), CBFA2/RUNX1 partner transcriptional co-repressor 3 (CBFA2T3) (ENSG00000129993.14), ankyrin repeat domain containing 11 (ANKRD11) (ENSG00000167522.15), ER membrane protein complex subunit 6 (TMEM93) (ENSG00000127774.6), lysine demethylase 6B (KDM6B) (ENSG00000132510.10), ribosomal protein L26 (RPL26) (ENSG00000161970.13), SUZ12 polycomb repressive complex 2 subunit (SUZ12) (ENSG00000178691.10), insulin like growth factor binding protein 4 (IGFBP4) (ENSG00000141753.6), keratin 10 (KRT10) (ENSG00000186395.7), transducer of ERBB2, 1 (TOB1) (ENSG00000141232.4), nascent polypeptide associated complex subunit alpha 2 (NACA2) (ENSG00000253506.2), SRY-box transcription factor 9 (SOX9) (ENSG00000125398.6), ring finger protein 138 (RNF138) (ENSG00000134758.13), immediate early response 3 interacting protein 1 (IER3IP1) (ENSG00000134049.5), methyl-CpG binding domain protein 2 (MBD2) (ENSG00000134046.11), SEC11 homolog C, signal peptidase complex subunit (SEC11C) (ENSG00000166562.8), coiled-coil domain containing 102B (CCDC102B) (ENSG00000150636.17), zinc finger and BTB domain containing 7A (ZBTB7A) (ENSG00000178951.8), scaffold attachment factor B2 (SAFB2) (ENSG00000130254.11), scaffold attachment factor B (SAFB) (ENSG00000160633.12), tubulin beta 4A class IVa (TUBB4A) (ENSG00000104833.11), RAB11B, member RAS oncogene family (RAB11B) (ENSG00000185236.11), solute carrier family 1 member 6 (SLC1A6) (ENSG00000105143.12), KLF transcription factor 2 (KLF2) (ENSG00000127528.5), zinc finger protein 708 (ZNF708) (ENSG00000182141.10), PAF1 homolog, Paf1/RNA polymerase II complex component (PAF1) (ENSG00000006712.14), transforming growth factor beta 1 (TGFB1) (ENSG00000105329.9), novel protein, readthrough between CEACAM5-CEACAM6 (AC011513.3) (ENSG00000267881.1), CD177 molecule (CD177) (ENSG00000204936.9), potassium voltage-gated channel subfamily A member 7 (KCNA7) (ENSG00000104848.1), ribosomal protein L28 (RPL28) (ENSG00000108107.14), zinc finger protein 580 (ZNF580) (ENSG00000213015.8), ESF1 nucleolar pre-rRNA processing protein homolog (ESF1) (ENSG00000089048.14), charged multivesicular body protein 4B (CHMP4B) (ENSG00000101421.3), DNA topoisomerase I (TOP1) (ENSG00000198900.5), prefoldin subunit 4 (PFDN4) (ENSG00000101132.9), PRELI domain containing 3B (PRELID3B) (ENSG00000101166.15), synaptonemal complex protein 2 (SYCP2) (ENSG00000196074.12), TATA-box binding protein associated factor 4 (TAF4) (ENSG00000130699.18), cholinergic receptor nicotinic alpha 4 subunit (CHRNA4) (ENSG00000101204.16), SRY-box transcription factor 18 (SOX18) (ENSG00000203883.6), histone H2B type F-S-like (CH507-42P11.1) (ENSG00000274559.3), ATP synthase peripheral stalk subunit F6 (ATP5J) (ENSG00000154723.12), GA binding protein transcription factor subunit alpha (GABPA) (ENSG00000154727.10), SR-related CTD associated factor 4 (SCAF4) (ENSG00000156304.14), H2B clustered histone 12 like (H2BFS) (ENSG00000234289.5), BCLAF1 And THRAP3 family member 3 (CXorf23) (ENSG00000173681.16), proprotein convertase subtilisin/kexin type 1 inhibitor (PCSK1N) (ENSG00000102109.8), MT-RNR2 like 10 (pseudogene) (MTRNR2L10) (ENSG00000256045.2), ATRX chromatin remodeler (ATRX) (ENSG00000085224.21), transcription elongation factor A like 5 (TCEAL5) (ENSG00000204065.2), glutamate dehydrogenase 2 (GLUD2) (ENSG00000182890.4), hypoxanthine phosphoribosyltransferase 1 (HPRT1) (ENSG00000165704.14), oxidoreductase core subunit 6 (MT-ND6) (ENSG00000198695.2).

In one embodiment for the identification of the MXE2 subtype, the expression of the predictive protein-coding genes selected from nexilin F-actin binding protein (NEXN) (ENSG00000162614.18), regulator of G protein signaling 13 (RGS13) (ENSG00000127074.14), myelin transcription factor 1 like (MYT1L) (ENSG00000186487.18), neurofilament medium chain (NEFM) (ENSG00000104722.13), casein kinase 1 alpha 1 like (CSNK1A1L) (ENSG00000180138.7), contactin associated protein family member 4 (CNTNAP4) (ENSG00000152910.18), nascent polypeptide associated complex subunit alpha 2 (NACA2) (ENSG00000253506.2), proprotein convertase subtilisin/kexin type 1 inhibitor (PCSKIN) (ENSG00000102109.8) and transcription elongation factor A like 5 (TCEAL5) (ENSG00000204065.2) are excluded.

Preferably, additionally, the predictive protein-coding genes are selected from the secondary group consisting of KH RNA binding domain containing, signal transduction associated 1 (KHDRBS1) (ENSG00000121774.17), Y-box binding protein 1 (YBX1) (ENSG00000065978.18), hook microtubule tethering protein 1 (HOOK1) (ENSG00000134709.10), chloride channel accessory 4 (CLCA4) (ENSG00000016602.9), coagulation factor V (F5) (ENSG00000198734.10), glutaredoxin 2 (GLRX2) (ENSG00000023572.9), cell division cycle 73 (CDC73) (ENSG00000134371.11), ribosomal RNA processing 15 homolog (RRP15) (ENSG00000067533.5), olfactory receptor family 2 subfamily M member 3 (OR2M3) (ENSG00000228198.3), DEAD-box helicase 1 (DDX1) (ENSG00000079785.14), mediator of cell motility 1 (MEMO1) (ENSG00000162959.13), ZFP36 ring finger protein like 2 (ZFP36L2) (ENSG00000152518.7), epithelial cell adhesion molecule (EPCAM) (ENSG00000119888.10), actin related protein 2 (ACTR2) (ENSG00000138071.13), ETAA1 activator of ATR kinase (ETAA1) (ENSG00000143971.8), pre-mRNA processing factor 40 homolog A (PRPF40A) (ENSG00000196504.15), NFE2 like bZIP transcription factor 2 (NFE2L2) (ENSG00000116044.15), golgin A4 (GOLGA4) (ENSG00000144674.16), mitochondrial matrix import factor 23 (CCDC58) (ENSG00000160124.9), defective in cullin neddylation 1 domain containing 1 (DCUN1D1) (ENSG00000043093.13), protein phosphatase 1 regulatory inhibitor subunit 2 (PPP1R2) (ENSG00000184203.7), heparan sulfate-glucosamine 3-sulfotransferase 1 (HS3ST1) (ENSG00000002587.9), factor interacting with PAPOLA and CPSF1 (FIP1L1) (ENSG00000145216.15), heterogeneous nuclear ribonucleoprotein D (HNRNPD) (ENSG00000138668.18), paired like homeodomain 2 (PITX2) (ENSG00000164093.16), chromosome 4 open reading frame 33 (C4orf33) (ENSG00000151470.12), RWD domain containing 4 (RWDD4) (ENSG00000182552.14), homer scaffold protein 1 (HOMER1) (ENSG00000152413.14), LysM domain containing 3 (LYSMD3) (ENSG00000176018.12), chromodomain helicase DNA binding protein 1 (CHD1) (ENSG00000153922.10), chromosome 5 open reading frame 15 (C5orf15) (ENSG00000113583.7), protocadherin beta 16 (PCDHB16) (ENSG00000272674.3), mannosidase endo-alpha (MANEA) (ENSG00000172469.15), PNN interacting serine and arginine rich protein (PNISR) (ENSG00000132424.14), anterior gradient 3, protein disulphide isomerase family member (AGR3) (ENSG00000173467.8), homeobox A7 (HOXA7) (ENSG00000122592.7), homeobox A9 (HOXA9) (ENSG00000078399.17), THAP domain containing 5 (THAP5) (ENSG00000177683.13), caveolin 2 (CAV2) (ENSG00000105971.14), zinc finger protein 800 (ZNF800) (ENSG00000048405.9), testis expressed 15, meiosis and synapsis associated (TEX15) (ENSG00000133863.8), MAK16 homolog (MAK16) (ENSG00000198042.10), transcriptional and immune response regulator (C8orf4) (ENSG00000176907.4), snail family transcriptional repressor 2 (SNAI2) (ENSG00000019549.10), lysophospholipase 1 (LYPLA1) (ENSG00000120992.17), XK related 4 (XKR4) (ENSG00000206579.8), potassium voltage-gated channel modifier subfamily V member 1 (KCNV1) (ENSG00000164794.8), HHLA1 neighbor of OC90 (HHLA1) (ENSG00000132297.11), DNA topoisomerase I mitochondrial (TOP1MT) (ENSG00000184428.12), regulatory factor X3 (RFX3) (ENSG00000080298.15), cyclin dependent kinase inhibitor 2A (CDKN2A) (ENSG00000147889.17), intraflagellar transport 74 (IFT74) (ENSG00000096872.15), nuclear receptor subfamily 4 group A member 3 (NR4A3) (ENSG00000119508.17), pre-mRNA processing factor 18 (PRPF18) (ENSG00000165630.13), zinc finger protein 37A (ZNF37A) (ENSG00000075407.18), RAB11 family interacting protein 2 (RAB11FIP2) (ENSG00000107560.10), G protein-coupled receptor 26 (GPR26) (ENSG00000154478.3), small VCP interacting protein (SVIP) (ENSG00000198168.8), electron transfer flavoprotein regulatory factor 1 (LYRM5) (ENSG00000205707.10), H3.5 histone (HF3C) (ENSG00000188375.4), solute carrier family 26 member 10 (SLC26A10) (ENSG00000135502.17), RAP1B, member of RAS oncogene family (RAP1B) (ENSG00000127314.17), TBC1 domain family member 15 (TBC1D15) (ENSG00000121749.15), MGAT4 family member C (MGAT4C) (ENSG00000182050.13), nuclear receptor corepressor 2 (NCOR2) (ENSG00000196498.13), nuclear FMR1 interacting protein 1 (NUFIP1) (ENSG00000083635.7), ubiquitin protein ligase E3A (UBE3A) (ENSG00000114062.18), gamma-aminobutyric acid type A receptor subunit gamma3 (GABRG3) (ENSG00000182256.12), sprouty related EVH1 domain containing 1 (SPRED1) (ENSG00000166068.12), LEO1 homolog, Paf1/RNA polymerase II complex component (LEO1) (ENSG00000166477.12), unc-13 homolog C (UNC13C) (ENSG00000137766.16), RAR related orphan receptor A (RORA) (ENSG00000069667.15), thyroid hormone receptor interactor 4 (TRIP4) (ENSG00000103671.9), solute carrier organic anion transporter family member 3A1 (SLCO3A1) (ENSG00000176463.13), mitochondrial ribosomal protein S34 (MRPS34) (ENSG00000074071.14), homeobox B2 (HOXB2) (ENSG00000173917.10), chromobox 4 (CBX4) (ENSG00000141582.14), occludin/ELL domain containing 1 (OCEL1) (ENSG00000099330.8), cartilage oligomeric matrix protein (COMP) (ENSG00000105664.10), zinc finger protein 254 (ZNF254) (ENSG00000213096.10), FXYD domain containing ion transport regulator 5 (FXYD5) (ENSG00000089327.14), coiled-coil domain containing 9 (CCDC9) (ENSG00000105321.13), myosin heavy chain 14 (MYH14) (ENSG00000105357.16), CCR4-NOT transcription complex subunit 3 (CNOT3) (ENSG00000088038.18), eukaryotic translation initiation factor 2 subunit beta (EIF2S2) (ENSG00000125977.6), solute carrier family 12 member 5 (SLC12A5) (ENSG00000124140.13), cathepsin Z (CTSZ) (ENSG00000101160.13), macrophage migration inhibitory factor (MIF) (ENSG00000240972.1), TSPY like 2 (TSPYL2) (ENSG00000184205.14), peptidylprolyl cis/trans isomerase, NIMA-interacting 4 (PIN4) (ENSG00000102309.12), transcription elongation factor A like 6 (TCEAL6) (ENSG00000204071.10).

In one embodiment for the identification of the MXE2 subtype, the expression of the predictive protein-coding genes from the secondary group, selected from XK related 4 (XKR4), chloride channel accessory 4 (CLCA4) (ENSG00000016602.9), MGAT4 family member C (MGAT4C) (ENSG00000182050.13) and epithelial cell adhesion molecule (EPCAM) (ENSG00000119888.10) are excluded.

The ranking as primary and secondary events is performed via stability scores (max 1, min 0.9) indicating consistent and robust predictive value for identification of MXE subtypes. Primary events have stability scores >=0.95 and secondary events have stability scores <0.95.

The invention also relates to a method for predicting a prognosis for a subject suffering from cancer the method comprising using a cancer subtype identifier based on PSI values based on an occurrence of at least one mutually exclusive exon usage event, and/or expression of small nuclear RNAs and/or expression of protein-coding genes comprising the steps:
a) collecting a sample from a tumor and/or body fluid and/or body excretion of a subject;
b) determining at least one of
   the PSI values based on the occurrence of at least one mutually exclusive exon usage event,
   deregulation of the expression of small nuclear RNAs,
   expression of subtype predictive protein-coding genes,
   combinations thereof; and
c) conversion of values of step b) into a rating scale to indicate the disease prognosis.

In step a) a sample from a tumor in a subject having cancer is collected. Step a) is non-therapeutic.

Suitable samples contain tissues or cells from the tumor of a subject having a cancer of epithelial cell origin. The sample preferably comprises a biopsy sample, such as a tumor biopsy, a primary tissue, a metastatic tissue. In particular, the specimen can be obtained by needle biopsy, image-guided biopsy, surgical (excisional) biopsy, shave/punch biopsy, endoscopic biopsy, laparoscopic biopsy and combinations thereof.

In a first embodiment, in step b) of the method according to the invention the PSI values based on the occurrence of at least one mutually exclusive exon usage event, and/or deregulation of the expression of small nuclear RNAs and/or expression of subtype predictive protein-coding genes is determined.

The PSI value represents the percentage of exon inclusion and is calculated/determined by dividing the number of reads from the upstream and downstream splice junctions of the alternative exon and/or from the alternative exon itself (referred to as inclusion reads) by the total number of reads from all splice junctions within the splice site, including the reads derived from the splice junction that connects the exon upstream of the alternative exon to the exon downstream of the alternative exon (referred as skipping reads) and inclusion reads. The effective length for the isoform is used for normalization of inclusion and skipping reads which is a mathematical transformation of sequencing read length and exon length.

PSI values, for example, can be estimated from RNA-Seq data using computational tools such as rMATS-turbo (see e.g, github.com/Xinglab/rmats-turbo) or MISO (miso.readthedocs.io/en/fastmiso/).

In a second embodiment, in step b) of the method according to the invention the expression of small nuclear RNAs is determined by molecular methods including but not limited to RNA sequencing in a targeted or non-targeted way, using primers, probes and antisense nucleotides designed to specific RNA or DNA sequences to detect and quantify gene expression through amplification or hybridization assays.

In a second embodiment, in step b) of the method according to the invention the expression of predictive protein-coding genes is determined by molecular methods including but not limited to RNA sequencing in a targeted or non-targeted way, using primers, probes and antisense nucleotides designed to specific RNA or DNA sequences to detect and quantify gene expression through amplification or hybridization assays. In the context of protein-coding genes, “expression” may also include translation and protein expression can be quantified specific antibodies against marker proteins.

In step c) the values of step b) are converted into a rating scale indicating the disease prognosis.

Preferably, in step c) according to the invention the conversion of PSI values to indicate the disease prognosis classifies the tumors as MXE1 subtype or MXE2 subtype.

Preferably, in step c) according to the invention the conversion of expression values of small nuclear RNAs to indicate the disease prognosis classifies the tumors as MXE1 subtype or MXE2 subtype.

Preferably, in step c) according to the invention the conversion of expression values of predictive protein-coding genes to indicate the disease prognosis by classifying tumors as MXE1 subtype or MXE2 subtype.

Preferably, the values of the conversion of PSI, expression of snRNAs and/or expression of protein-coding genes indicate the disease prognosis and is used to decide whether the subject is subjected to a treatment by anti-tumor therapy and/or surveillance of progress of the disease.

There are several ways to convert the PSI values, expression of snRNAs and/or expression of protein-coding genes to indicate the disease prognosis.

One option is that the calculated PSI values, expression of snRNAs and/or expression of protein-coding genes are converted to MXE probabilities by mathematical transformation. Preferably, the calculated PSI values, expression of snRNAs and/or expression of protein-coding genes are converted to MXE probabilities estimates by mathematical transformation of the dot product between PSI values and their respective weights derived from the reference data set as follows:

F denotes the vector representations for PSI values and/or variance stabilized gene expression values for deregulated snRNAs or subtype-predictive protein coding genes of a new sample. θ indicates the weights associated with F in regard to the MXE subtypes, as estimated from logistic regression models trained on the reference data set.

$$F = \begin{matrix}{F}_{1} \\ {F}_{2} \\ {F}_{n}\end{matrix} {θ}_{MXE \{ 1 \vert 2 \}} = \begin{matrix}{Weight}_{1 MXE \{ 1 \vert 2 \}} \\ {Weight}_{2 MXE \{ 1 \vert 2 \}} \\ {Weight}_{nMXE \{ 1 \vert 2 \}}\end{matrix}$$

z is the linear combination of the PSI values or variance stabilized gene expression values for deregulated snRNAs and/or subtype-predictive protein coding genes and their corresponding weights and β₀ is the intercept derived from the reference data set for each MXE subtype:

$${z}_{MXE \{ 1 \vert 2 \}} = F \cdot {θ}_{MxE \{ 1 \vert 2 \}} = {\sum }_{i = 1}^{n} {F}_{i} {Weight}_{iMXE \{ 1 \vert 2 \}} + {β}_{{0}_{MXE \{ 1 \vert 2 \}}}$$

z is converted into a probability for each MXE subtype using the logistic function where e is a mathematical constant (~2.718):

$$P ( MXE \{ 1 \vert 2 \} ) = \frac{1}{1 + {e}^{- {z}_{MXE \{ 1 \vert 2 \}}}}$$

Another possible approach involves measuring the distance between a sample's PSI value and/or variance stabilized gene expression values for deregulated snRNAs and/or subtype-predictive protein coding genes and the median PSI or variance stabilized gene expression values for deregulated snRNAs and/or subtype-predictive protein coding genes for each MXE subtype in the reference dataset. By ranking these distances, the MXE with the lowest distance can be assigned to the sample. For n number of features, the MXE can be assigned as follows:

$${d}_{MXE 1} = {\sum }_{i = 1}^{n} \sqrt{{( med ( {F}_{{i}_{MXE 1 reference}} ) - {F}_{sample} )}^{2}}$$

$${d}_{MXE 2} = {\sum }_{i = 1}^{n} \sqrt{{( med ( {F}_{{i}_{MXE 2 reference}} ) - {F}_{sample} )}^{2}}$$

$$MXE = min ( {d}_{MXE 1} , {d}_{MXE 2} )$$

Alternatively, Pearson correlation between the features of the test sample and the feature centroids from the reference dataset can be used instead of a distance metric.

A further approach involves selecting pairs of PSI values and/or expression values from deregulated snRNAs or subtype-predictive protein coding genes whose values and/or expression levels switch ranking consistently between the two prognostic groups by using “top scoring pairs” (TSP) algorithm. The relationship can be converted to binary vectors for supervised model-based classification.

Advantageously, in the methods of the present invention it is possible to establish a rating scale by using at least one of the PSI values based on the occurrence of at least one mutually exclusive exon usage event, the expression of small nuclear RNAs the expression of protein-coding genes, combinations thereof. Advantageously, this rating scale can be used to decide whether the subject is likely to experience an increased progression of the disease. In particular, the PSI values based on the occurrence of at least one mutually exclusive exon usage event, and/or the expression of small nuclear RNAs and/or the expression of protein-coding genes can be used to classify the tumors as MXE1 subtype or MXE2 subtype. Further, it can be decided whether a subject should be subjected to a treatment by anti tumor therapy and/or surveillance of progress of the disease. This method allows a decision at a very early stage of the disease, where markers used in state of the art staging protocols (like e.g. the occurrence of other tumor markers or metastases), usually are not yet detectible.

MXE1 and MXE2 subtypes are independent of CMS and tumor staging and can be combined with conventional clinical classifications to refine them.

In another preferred embodiment, the conversion of values of step b) to indicate the disease prognosis is combined with tumor classification subtypes other than MXE1 subtype and MXE2 subtype. It is preferably combined with at least one tumor classification subtypes selected from CMS and immune infiltrate.

The consensus molecular subtype (CMS) classification system divides colon cancers into 4 distinct groups, called subtypes, each with distinct biological and molecular characteristics: CMS1, CMS2, CMS3 and CMS4. Gene expression profiles from tumors have been used to establish the subgroups. CMS1 (MSI Immune) tumors have microsatellite instability and are heavily infiltrated with immune cells. CMS2 (Canonical) is an epithelial subtype with significant upregulation of WNT and MYC downstream targets, and chromosomal instability (CIN) is a distinguishing feature of this subtype. CMS3 (Metabolic), like CMS2, has epithelial characteristics but exhibits less CIN. In addition, the CMS3 subtype is enriched for KRAS mutations and shows de-regulation of genes involved in metabolism. CMS4 (Mesenchymal) is a mesenchymal subtype and exhibits activation of pathways that regulate epithelial-mesenchymal transition (EMT) and stemness.

Immune cell infiltrate has been associated with tumor progression. Immune cells that infiltrated the tumor center correlated with clinicopathological characteristics and prognosis. The immune risk scores were an independent prognostic indicator and were found to predict cervical cancer prognosis as well as the effects of chemoradiotherapy. Immune cells that migrate from the blood into the tumor, e.g., T-cells, B-cells, natural killer (NK) cells, or macrophages, are defined as tumor-infiltrating immune cells. The density patterns of different immune cell markers (namely CD3, CD4, CD8, CD20, CD57, CD68, and CD163) may be used as classification system to predict the prognosis of patients having cancer.

Another embodiment of the invention is a method for treating a subject suffering from cancer the method comprising using a cancer subtype identifier based on PSI values based on an occurrence of at least one mutually exclusive exon event, and/or the deregulation of expression of small nuclear RNAs and/or deregulation of expression of subtype predictive protein-coding genes comprising the steps:
a′) collecting a sample from a tumor and/or body fluid and/or body excretion of a subject;
b′) determining at least one of
   the PSI values based on the occurrence of at least one mutually exclusive exon usage event,
   the deregulation of the expression of small nuclear RNAs,
   expression of subtype predictive protein-coding genes,
   combinations thereof;
c′) conversion of the values of step b) into a rating scale to indicate the disease prognosis by classifying the tumors as MXE1 subtype or MXE2 subtype; and
d′) subjecting the subject to an anti tumor treatment if the rating scale indicates that the subject is likely to experience increased progression of the disease.

Advantageously, in the method of the present invention it is possible to establish a rating scale by using the PSI values based on the occurrence of at least one mutually exclusive exon usage event, and/or the expression of small nuclear RNAs and/or the expression of protein-coding genes. Advantageously, this rating scale can be used to decide, whether the subject is likely to experience an increased progression of the disease. In particular, the PSI values based on the occurrence of at least one mutually exclusive exon usage event, and/or the expression of small nuclear RNAs and/or the expression of protein-coding genes can be used to classify the tumors as MXE1 subtype or MXE2 subtype. Further, it can be decided whether a subject should be subjected to a treatment by anti tumor therapy and/or surveillance of progress of the disease. This method allows a decision at a very early stage of the disease, where markers used in state of the art staging protocols (like e.g. the occurrence of other tumor markers or metastases), usually are not yet detectible.

There is a great variety of anti tumor therapies that are well known to a person skilled in the art. Based on specific factors like nature of the cancer, the stage of disease etc. the skilled person can decide which therapy is appropriate for treatment of a subject in need of an anti tumor therapy e.g. surgery, radiotherapy and/or chemotherapy.

A further embodiment of the invention relates to a kit for predicting the prognosis for a subject suffering from colorectal cancer using at least one of PSI values based on an occurrence of at least one mutually exclusive exon usage event, deregulation of the expression of small nuclear RNAs, expression of subtype predictive protein-coding genes, combinations thereof comprising a composition for determining the PSI value, the expression of small nuclear RNAs and the expression protein-coding genes.

Furthermore, the kit may comprise reagents and/or devices for preparing a sample such as collecting a tissue and/or excising a sample from the tissue; for spotting test samples on a suitable surface, such as nitrocellulose strips or glass slides; for performing immuno-quantitation such as labeled antibodies, or reagents for labeling antibodies; instructions for performing a method of the invention; etc.

The components of the kit may, optionally, be packaged in one or more containers.

Optionally, the kit according to the invention comprises suitable buffers; one or more containers or packaging material; and/or instructions for performing the method. The reagents of the kit may be in containers in which the reagents are stable, e.g. in lyophilized form or stabilized liquids. The reagents may also be in single use form, e.g., in single dosage form.

Another embodiment of the invention is the use of at least one of PSI values based on the occurrence of at least one mutually exclusive exon usage event, the expression of small nuclear RNAs, the expression of predictive protein-coding genes, combination thereof as a marker of the prognosis for a subject suffering from cancer.

### Exemplary Embodiments

1. A cancer subtype identifier, which classifies tumors based on at least one of
PSI values based on the occurrence of at least one mutually exclusive exon usage event,
expression of small nuclear RNAs,
expression of predictive protein-coding genes,
a combination thereof.

2. A cancer subtype identifier, according to embodiment 1, which classifies tumors based on at least one of
PSI (percent spliced-in) values based on the occurrence of at least one mutually exclusive exon usage event,
expression of small nuclear RNAs,
expression of predictive protein-coding genes,
a combination thereof,
wherein the tumor is classified as MXE1 subtype and MXE2 subtype.

3. A cancer subtype identifier, according to embodiment 1 or 2, which classifies tumors based on at least one of
PSI (percent spliced-in) values based on the occurrence of at least one mutually exclusive exon usage event,
expression of small nuclear RNAs,
expression of predictive protein-coding genes,
a combination thereof,
wherein the tumor is classified as MXE1 subtype and MXE2 subtype, wherein
the MXE1 subtype is characterized by
i) a distinct combination of PSI values from mutually exclusive exon usage events,
ii) a distinct combination of expressions of small nuclear RNAs
iii) a distinct combination of protein coding genes,
a combination of i) and ii), i) and iii), ii) and iii) and i), ii) and iii),
wherein MXE1 subtype is associated with a good prognosis compared to the MXE2 subtype. and/or
the MXE2 subtype is characterized by
iv) a distinct combination of PSI values from mutually exclusive exon usage events,
v) a distinct combination of expressions of small nuclear RNAs
vi) a distinct combination of protein coding genes,
a combination of iv) and v), iv) and vi), v) and vi) and iv), v) and vi),
wherein the MXE2 subtype is associated with a poor prognosis compared to the MXE1 subtype.
wherein i) is different from iv), ii) is different from v), iii) is different from vi).

4. The cancer subtype identifier according to embodiment 1 2 or 3, which classifies tumors based on at least one of
PSI (percent spliced-in) values based on the occurrence of at least one mutually exclusive exon usage event,
expression of small nuclear RNAs,
a combination thereof,
wherein the tumor is classified as MXE1 subtype and MXE2 subtype,
optionally the cancer subtype identifier classifies tumors additionally based on expression of predictive protein-coding genes.

5. The cancer subtype identifier according to any of the preceding embodiments, wherein the cancer is selected from lung cancer, breast cancer, gastric cancer, esophageal cancer, skin cancer, ovarian cancer, cervical cancer, liver cancer, bladder cancer, kidney cancer, pancreatic cancer, prostate cancer, thyroid cancer, head cancer, neck cancer, oral cancer, laryngeal cancer, and colorectal cancer, preferably colorectal cancer and non-small cell lung cancer (lung adenocarcinoma).

6. The cancer subtype identifier according to any of the preceding embodiments, wherein the small nuclear RNAs are selected from U1 small nuclear 1 RNA (RNU1-1); U12 small nuclear RNA (RNU12); U4 small nuclear 2 RNA (RNU4-2); U5A small nuclear 1 RNA (RNU5A-1); U5B small nuclear 1 RNA (RNU5B-1); U5D small nuclear 1 RNA (RNU5D-1); U5E small nuclear 1 RNA (RNUSE-1); U6 small nuclear 9 RNA (RNU6-9); U6atac small nuclear RNA (RNU6ATAC); variant U1 small nuclear 1 RNA (RNVU1-1); variant U1 small nuclear 15 RNA (RNVU1-15); variant U1 small nuclear 18 RNA (RNVU1-18); variant U1 small nuclear 19 RNA (RNVU1-19); variant U1 small nuclear 6 RNA (RNVU1-6); variant U1 small nuclear 7 RNA (RNVU1-7); U11 small nuclear RNA (RNU11); U1 small nuclear 4 RNA (RNU1-4); U4 small nuclear 1 RNA (RNU4-1); U4atac small nuclear RNA (RNU4ATAC); U5F small nuclear 1 RNA (RNU5F-1); U6 small nuclear 1 RNA (RNU6-1); U6 small nuclear 2 RNA (RNU6-2); U1 small nuclear RNA (RF00003); U4 small nuclear RNA (RF00015) and U7 small nuclear RNA (RF00066).

7. The cancer subtype identifier according to any of the preceding embodiments, wherein the small nuclear RNAs are selected from RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC, RNVU1-1, RNVU1-15, RNVU1-19 are upregulated and wherein the small nuclear RNAs are selected from RNU1-1, RNU1-4, RNU4-2, RNVU1-18, RNVU1-6, RNVU1-7, RF00015, RF00066 and RF00003 are downregulated.

8. The cancer subtype identifier according to any of the preceding embodiments, wherein In particular, RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC, RNVU1-1, RNVU1-15, RNVU1-19 are upregulated and RNU1-1, RNU1-4, RNU4-2, RNVU1-18, RNVU1-6, RNVU1-7, RF00015, RF00066 and RF00003 are downregulated in MXE2 subtype, which is associated with poor prognosis and/or wherein RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC are upregulated and RF00015 and RF00066 are downregulated in MXE2 subtype, which is associated with poor prognosis.

9. The cancer subtype identifier according to any of the preceding embodiments, wherein the tumor is classified as MXE1 subtype and MXE2 subtype.

10. The cancer subtype identifier according any of the preceding embodiments, wherein for identification of MXE1 subtype at least one primary mutually exclusive exon usage event is present, characterized by differential splicing of exons that are incorporated into different transcripts, as exon 1 and exon 2, wherein the mutually exclusive splicing events are optionally selected from exon1 (with genomic coordinates chr21: 45480466-45480520) of gene collagen type XVIII alpha 1 chain (COL18A1) (ENSG00000182871.14), compared to exon2 (with genomic coordinates chr21: 45480699-45480858); exon1 (with genomic coordinates chr19: 33012646-33012724) of gene rhophilin Rho GTPase binding protein 2 (RHPN2) (ENSG00000131941.7), compared to exon2 (with genomic coordinates chr19: 33021570-33021646); exon1 (with genomic coordinates chr19: 10986188-10986593) of gene SWI/SNF related, matrix associated, actin dependent regulator of chromatin, subfamily a, member 4 (SMARCA4) (ENSG00000127616.18), compared to exon2 (with genomic coordinates chr19: 10986904-10987003); exon1 (with genomic coordinates chr17: 69304668-69304810) of gene ATP binding cassette subfamily A member 5 (ABCA5) (ENSG00000154265.15), compared to exon2 (with genomic coordinates chr17: 69306724-69306954); exon1 (with genomic coordinates chr11: 66564087-66564146) of gene cathepsin F (CTSF) (ENSG00000174080.10), compared to exon2 (with genomic coordinates chr11: 66564557-66564648); exon1 (with genomic coordinates chr1: 200844781-200844869) of gene calmodulin regulated spectrin associated protein family member 2 (CAMSAP2) (ENSG00000118200.14), compared to exon2 (with genomic coordinates chr1: 200848031-200850234); exon1 (with genomic coordinates chr2: 188988080-188988134) of gene collagen type III alpha 1 chain (COL3A1) (ENSG00000168542.14), compared to exon2 (with genomic coordinates chr2: 188989395-188989449); exon1 (with genomic coordinates chr1: 54205294-54205410) of gene mitochondrial ribosomal protein L37 (MRPL37) (ENSG00000116221.15), compared to exon2 (with genomic coordinates chr1: 54209945-54210131); exon1 (with genomic coordinates chr6: 149836084-149836297) of gene LDL receptor related protein 11 (LRP11) (ENSG00000120256.9), compared to exon2 (with genomic coordinates chr6: 149837337-149837463); exon1 (with genomic coordinates chrX: 41140655-41140771) of gene ubiquitin specific peptidase 9 X-linked (USP9X) (ENSG00000124486.12), compared to exon2 (with genomic coordinates chrX: 41140965-41141217); exon1 (with genomic coordinates chr6: 31642828-31643115) of gene BAG cochaperone 6 (BAG6) (ENSG00000204463.12), compared to exon2 (with genomic coordinates chr6: 31644081-31644194); exon1 (with genomic coordinates chr6: 33303954-33303989) of gene TAP binding protein (TAPBP) (ENSG00000231925.11), compared to exon2 (with genomic coordinates chr6: 33304127-33304217); exon1 (with genomic coordinates chr7: 34966893-34966971) of gene dpy-19 like C-mannosyltransferase 1 (DPY19L1) (ENSG00000173852.14), compared to exon2 (with genomic coordinates chr7: 34969432-34969532); exon1 (with genomic coordinates chr2: 24299908-24300171) of gene intersectin 2 (ITSN2) (ENSG00000198399.14), compared to exon2 (with genomic coordinates chr2: 24301153-24301239); exon1 (with genomic coordinates chr10: 112438335-112438389) of gene zinc finger DHHC-type palmitoyltransferase 6 (ZDHHC6) (ENSG00000023041.11), compared to exon2 (with genomic coordinates chr10: 112440533-112440695); exon1 (with genomic coordinates chr10: 112438335-112438389) of gene zinc finger DHHC-type palmitoyltransferase 6 (ZDHHC6) (ENSG00000023041.11), compared to exon2 (with genomic coordinates chr10: 112442191-112442351).

11. The cancer subtype identifier according to any of the preceding embodiments,
wherein for the identification of MXE1 subtype at least one mutually exclusive exon usage event is present, characterized by differential splicing of exons that are incorporated into different transcripts, as exon1 and exon2, wherein the mutually exclusive splicing events, preferably are selected from exon1 (with genomic coordinates chr21: 45480466-45480520) of gene collagen type XVIII alpha 1 chain (COL18A1) (ENSG00000182871.14), compared to exon2 (with genomic coordinates chr21: 45480699-45480858); exon1 (with genomic coordinates chr19: 33012646-33012724) of gene rhophilin Rho GTPase binding protein 2 (RHPN2) (ENSG00000131941.7), compared to exon2 (with genomic coordinates chr19: 33021570-33021646); exon1 (with genomic coordinates chr19: 10986188-10986593) of gene SWI/SNF related, matrix associated, actin dependent regulator of chromatin, subfamily a, member 4 (SMARCA4) (ENSG00000127616.18), compared to exon2 (with genomic coordinates chr19: 10986904-10987003); exon1 (with genomic coordinates chr17: 69304668-69304810) of gene ATP binding cassette subfamily A member 5 (ABCA5) (ENSG00000154265.15), compared to exon2 (with genomic coordinates chr17: 69306724-69306954); exon1 (with genomic coordinates chr11: 66564087-66564146) of gene cathepsin F (CTSF) (ENSG00000174080.10), compared to exon2 (with genomic coordinates chr11: 66564557-66564648); exon1 (with genomic coordinates chr1: 200844781-200844869) of gene calmodulin regulated spectrin associated protein family member 2 (CAMSAP2) (ENSG00000118200.14), compared to exon2 (with genomic coordinates chr1: 200848031-200850234); exon1 (with genomic coordinates chr2: 188988080-188988134) of gene collagen type III alpha 1 chain (COL3A1) (ENSG00000168542.14), compared to exon2 (with genomic coordinates chr2: 188989395-188989449); exon1 (with genomic coordinates chr1: 54205294-54205410) of gene mitochondrial ribosomal protein L37 (MRPL37) (ENSG00000116221.15), compared to exon2 (with genomic coordinates chr1: 54209945-54210131); exon1 (with genomic coordinates chr6: 149836084-149836297) of gene LDL receptor related protein 11 (LRP11) (ENSG00000120256.9), compared to exon2 (with genomic coordinates chr6: 149837337-149837463); exon1 (with genomic coordinates chrX: 41140655-41140771) of gene ubiquitin specific peptidase 9 X-linked (USP9X) (ENSG00000124486.12), compared to exon2 (with genomic coordinates chrX: 41140965-41141217); exon1 (with genomic coordinates chr6: 31642828-31643115) of gene BAG cochaperone 6 (BAG6) (ENSG00000204463.12), compared to exon2 (with genomic coordinates chr6: 31644081-31644194); exon1 (with genomic coordinates chr6: 33303954-33303989) of gene TAP binding protein (TAPBP) (ENSG00000231925.11), compared to exon2 (with genomic coordinates chr6: 33304127-33304217); exon1 (with genomic coordinates chr7: 34966893-34966971) of gene dpy-19 like C-mannosyltransferase 1 (DPY19L1) (ENSG00000173852.14), compared to exon2 (with genomic coordinates chr7: 34969432-34969532); exon1 (with genomic coordinates chr2: 24299908-24300171) of gene intersectin 2 (ITSN2) (ENSG00000198399.14), compared to exon2 (with genomic coordinates chr2: 24301153-24301239); exon1 (with genomic coordinates chr10: 112438335-112438389) of gene zinc finger DHHC-type palmitoyltransferase 6 (ZDHHC6) (ENSG00000023041.11), compared to exon2 (with genomic coordinates chr10: 112440533-112440695); exon1 (with genomic coordinates chr10: 112438335-112438389) of gene zinc finger DHHC-type palmitoyltransferase 6 (ZDHHC6) (ENSG00000023041.11), compared to exon2 (with genomic coordinates chr10: 112442191-112442351); exon1 (with genomic coordinates chr19: 49862043-49862105) of gene polynucleotide kinase 3′-phosphatase (PNKP) (ENSG00000039650.11), compared to exon2 (with genomic coordinates chr19: 49862184-49862281); exon1 (with genomic coordinates chr19: 47372729-47372923) of gene DExH-box helicase 34 (DHX34) (ENSG00000134815.18), compared to exon2 (with genomic coordinates chr19: 47373598-47373700); exon1 (with genomic coordinates chr15: 65701050-65701192) of gene DENN domain containing 4A (DENND4A) (ENSG00000174485.15), compared to exon2 (with genomic coordinates chr15: 65701761-65701890); exon1 (with genomic coordinates chr17: 67145742-67145890) of gene helicase with zinc finger (HELZ) (ENSG00000198265.11), compared to exon2 (with genomic coordinates chr17: 67149866-67149985); exon1 (with genomic coordinates chr11: 2527927-2528018) of gene potassium voltage-gated channel subfamily Q member 1 (KCNQ1) (ENSG00000053918.16), compared to exon2 (with genomic coordinates chr11: 2587569-2587692); exon1 (with genomic coordinates chr5: 9050409-9050457) of gene semaphorin 5A (SEMA5A) (ENSG00000112902.11), compared to exon2 (with genomic coordinates chr5: 9051872-9052028); exon1 (with genomic coordinates chr20: 10641112-10641244) of gene jagged canonical Notch ligand 1 (JAG1) (ENSG00000101384.12), compared to exon2 (with genomic coordinates chr20: 10641459-10641693).

12. The cancer subtype identifier according any of the preceding embodiments, wherein for the identification of MXE2 subtype at least one primary mutually exclusive exon usage event is present, characterized by differential splicing of exons that are incorporated into different transcripts, as exon1 and exon2, wherein the mutually exclusive splicing events are optionally selected from exon1 (with genomic coordinates chr20: 48988490-48988662) of gene ADP ribosylation factor guanine nucleotide exchange factor 2 (ARFGEF2) (ENSG00000124198.8), compared to exon2 (with genomic coordinates chr20: 48989284-48989436); exon1 (with genomic coordinates chr18: 62266700-62266749) of gene RAB11 Binding and LisH domain (KIAA1468) (ENSG00000134444.14), compared to exon2 (with genomic coordinates chr18: 62268868-62268948); exon1 (with genomic coordinates chr16: 30081258-30081310) of gene protein phosphatase 4 catalytic subunit (PPP4C) (ENSG00000149923.13), compared to exon2 (with genomic coordinates chr16: 30082483-30082534); exon1 (with genomic coordinates chr15: 34255261-34255392) of gene solute carrier family 12 member 6 (SLC12A6) (ENSG00000140199.11), compared to exon2 (with genomic coordinates chr15: 34258812-34258944); exon1 (with genomic coordinates chr14: 22905605-22905659) of gene RNA binding motif protein 23 (RBM23) (ENSG00000100461.17), compared to exon2 (with genomic coordinates chr14: 22908332-22908380); exon1 (with genomic coordinates chr12: 7142085-7142139) of gene calsyntenin 3 (CLSTN3) (ENSG00000139182.13), compared to exon2 (with genomic coordinates chr12: 7142868-7143026); exon1 (with genomic coordinates chr20: 13534021-13534141) of gene taspase 1 (TASP1) (ENSG00000089123.15), compared to exon2 (with genomic coordinates chr20: 13559007-13559114); exon1 (with genomic coordinates chr8: 52630469-52630528) of gene RB1 inducible coiled-coil 1 (RB1CC1) (ENSG00000023287.12), compared to exon2 (with genomic coordinates chr8: 52634920-52634968); exon1 (with genomic coordinates chr1: 150156065-150156206) of gene pleckstrin homology domain containing 01 (PLEKHO1) (ENSG00000023902.13), compared to exon2 (with genomic coordinates chr1: 150157384-150157486); exon1 (with genomic coordinates chr2: 32607454-32607643) of gene baculoviral IAP repeat containing 6 (BIRC6) (ENSG00000115760.13), compared to exon2 (with genomic coordinates chr2: 32611447-32611582); exon1 (with genomic coordinates chr11: 9430874-9431003) of gene importin 7 (IPO7) (ENSG00000205339.9), compared to exon2 (with genomic coordinates chr11: 9433569-9433636); exon1 (with genomic coordinates chr17: 82081163-82081352) of gene fatty acid synthase (FASN) (ENSG00000169710.8), compared to exon2 (with genomic coordinates chr17: 82081600-82081843); exon1 (with genomic coordinates chr6: 157873101-157873176) of gene sorting nexin 9 (SNX9) (ENSG00000130340.15), compared to exon2 (with genomic coordinates chr6: 157875050-157875176); exon1 (with genomic coordinates chr7: 5390484-5390628) of gene trinucleotide repeat containing 18 (TNRC18) (ENSG00000182095.14), compared to exon2 (with genomic coordinates chr7: 5394439-5394595); exon1 (with genomic coordinates chr17: 50195922-50195976) of gene collagen type I alpha 1 chain (COL1A1) (ENSG00000108821.13), compared to exon2 (with genomic coordinates chr17: 50196154-50196199).

13. The cancer subtype identifier according any of the preceding embodiments, wherein for the identification of MXE2 subtype at least one mutually exclusive exon usage event is present, characterized by differential splicing of exons that are incorporated into different transcripts, as exon1 and exon2, wherein the mutually exclusive splicing events preferably are selected from exon1 (with genomic coordinates chr20: 48988490-48988662) of gene ADP ribosylation factor guanine nucleotide exchange factor 2 (ARFGEF2) (ENSG00000124198.8), compared to exon2 (with genomic coordinates chr20: 48989284-48989436); exon1 (with genomic coordinates chr18: 62266700-62266749) of gene RAB11 Binding and LisH domain (KIAA1468) (ENSG00000134444.14), compared to exon2 (with genomic coordinates chr18: 62268868-62268948); exon1 (with genomic coordinates chr16: 30081258-30081310) of gene protein phosphatase 4 catalytic subunit (PPP4C) (ENSG00000149923.13), compared to exon2 (with genomic coordinates chr16: 30082483-30082534); exon1 (with genomic coordinates chr15: 34255261-34255392) of gene solute carrier family 12 member 6 (SLC12A6) (ENSG00000140199.11), compared to exon2 (with genomic coordinates chr15: 34258812-34258944); exon1 (with genomic coordinates chr14: 22905605-22905659) of gene RNA binding motif protein 23 (RBM23) (ENSG00000100461.17), compared to exon2 (with genomic coordinates chr14: 22908332-22908380); exon1 (with genomic coordinates chr12: 7142085-7142139) of gene calsyntenin 3 (CLSTN3) (ENSG00000139182.13), compared to exon2 (with genomic coordinates chr12: 7142868-7143026); exon1 (with genomic coordinates chr20: 13534021-13534141) of gene taspase 1 (TASP1) (ENSG00000089123.15), compared to exon2 (with genomic coordinates chr20: 13559007-13559114); exon1 (with genomic coordinates chr8: 52630469-52630528) of gene RB1 inducible coiled-coil 1 (RB1CC1) (ENSG00000023287.12), compared to exon2 (with genomic coordinates chr8: 52634920-52634968); exon1 (with genomic coordinates chr1: 150156065-150156206) of gene pleckstrin homology domain containing 01 (PLEKHO1) (ENSG00000023902.13), compared to exon2 (with genomic coordinates chr1: 150157384-150157486); exon1 (with genomic coordinates chr2: 32607454-32607643) of gene baculoviral IAP repeat containing 6 (BIRC6) (ENSG00000115760.13), compared to exon2 (with genomic coordinates chr2: 32611447-32611582); exon1 (with genomic coordinates chr11: 9430874-9431003) of gene importin 7 (IPO7) (ENSG00000205339.9), compared to exon2 (with genomic coordinates chr11: 9433569-9433636); exon1 (with genomic coordinates chr17: 82081163-82081352) of gene fatty acid synthase (FASN) (ENSG00000169710.8), compared to exon2 (with genomic coordinates chr17: 82081600-82081843); exon1 (with genomic coordinates chr6: 157873101-157873176) of gene sorting nexin 9 (SNX9) (ENSG00000130340.15), compared to exon2 (with genomic coordinates chr6: 157875050-157875176); exon1 (with genomic coordinates chr7: 5390484-5390628) of gene trinucleotide repeat containing 18 (TNRC18) (ENSG00000182095.14), compared to exon2 (with genomic coordinates chr7: 5394439-5394595); exon1 (with genomic coordinates chr17: 50195922-50195976) of gene collagen type I alpha 1 chain (COL1A1) (ENSG00000108821.13), compared to exon2 (with genomic coordinates chr17: 50196154-50196199); exon1 (with genomic coordinates chr19: 55091649-55091700) of gene protein phosphatase 1 regulatory subunit 12C (PPP1R12C) (ENSG00000125503.12), compared to exon2 (with genomic coordinates chr19: 55091858-55091909); exon1 (with genomic coordinates chr17: 69301138-69301286) of gene ATP binding cassette subfamily A member 5 (ABCA5) (ENSG00000154265.15), compared to exon2 (with genomic coordinates chr17: 69302717-69302906); exon1 (with genomic coordinates chr4: 148152468-148152613) of gene nuclear receptor subfamily 3 group C member 2 (NR3C2) (ENSG00000151623.14), compared to exon2 (with genomic coordinates chr4: 148154550-148154901); exon1 (with genomic coordinates chr9: 120453455-120453873) of gene CDK5 regulatory subunit associated protein 2 (CDK5RAP2) (ENSG00000136861.17), compared to exon2 (with genomic coordinates chr9: 120460571-120460667).

14. The cancer subtype identifier according to any of the preceding embodiments, wherein for the identification of the MXE1 subtype primary small nuclear RNA expression shows up-regulation as compared to the MXE2 subtype exhibiting the highest predictive importance for MXE subtype classification selected from

RNU4-2 (ENSG00000202538.1) SEQ ID NO: 1:
AGCTTTGCGCAGTGGCAGTATCGTAGCCAATGAGGTTTATCCGAGGCGCGATTATTGCTA
ATTGAAAACTTTTCCCAATACCCCGCCATGACGACTTGAAATATAGTCGGCATTGGCAATT
TTTGACAGTCTCTACGGAGA
RNVU1-7 (ENSG00000206585.1) SEQ ID NO: 2:
ATACTTACCTGGCAGGGGAGATACCATGATCACGAAGGTGGTTTTCCCAGGGCGAGGCT
TATCCATTGCACTCCGGATGTGCTGACCCCTGCGATTTCCCCAAATGTGGGAAACTCGAC
TGCATAATTTGTGGTAGTGGGGGACTGCGTCCGCGCTTTCCCCTG
RNVU1-6 (ENSG00000201558.1) SEQ ID NO: 3:
ATACTTACCTGGCAGGAGAGATACCCTGGTCACGAAGGTGGTTTTTCCAGAGCGAGTCTT
ATCCATTGCACTCCGGATCTGCTGACCCCTGCAGTTTCCCCAAATGTGGGAAACTTGACT
GTATAATTTGTGGCAGTGGTAGACTGCATTCGCGCTTTCCCCTG, preferably

15. The cancer subtype identifier according any of the preceding embodiments, wherein for the identification of the MXE1 subtype the small nuclear RNA expression shows up-regulation as compared to the MXE2 subtype:

RNU1-1(ENSG00000206652.1) SEQ ID No: 6:
ATACTTACCTGGCAGGGGAGATACCATGATCACGAAGGTGGTTTTCCCAGGGCGAGGCT
TATCCATTGCACTCCGGATGTGCTGACCCCTGCGATTTCCCCAAATGTGGGAAACTCGAC
TGCATAATTTGTGGTAGTGGGGGACTGCGTTCGCGCTTTCCCCTG
RNU1-4 (ENSG00000207389.1) SEQ ID NO: 4:
ATACTTACCTGGCAGGGGAGATACCATGATCACGAAGGTGGTTTTCCCAGGGCGAGGCT
TATCCATTGCACTCCGGATGTGCTGACCCCTGCGATTTCCCCAAATGTGGGAAACTCGAC
TGCATAATTTGTGGTAGTGGGGGACTGCGTTCGCGCTTTCCCCTG
RNVU1-18(ENSG00000206737.1) SEQ ID NO: 5:
ATACTTACCTGGCAGGGGAGATACCATGATCACGAAGGTGGTTTTCCCAGGGCGAGGCT
TATCCATTGCACTCCGGATGTGCTGACCCCTGCGATTTCCCCAAATGTGGGAAACTCGAC
TGCATAATTTGTGGTAGTGGGGGACTGCGTTCGCGCTTTCCCCTG
RF00003 (U1 family) (ENSG00000273727.1) SEQ ID NO:7:
ATACTTATGTAACAGGAGAAAATACGGCCATGAAGTTGGTGTTTTTCGGGGGGGGTTTTT
CCATTGTACTCAGTATGTGCTGACTGACTCCTGTTACTTCCACATGTGGGGAAACTGGAC
TGTAATTTGTGGTGGTGGGGAATTGCGTTCGCGCTTTCTTCT
RF00003 (U1 family) (ENSG00000273516.1) SEQ ID NO: 8 :
CCACTTACCTGGCAGGAAAGAGACCGTGGTCACGAAGGGGGTTCTCCCAGAGTGAAGCT
TCTTCATCGCACTCTAGAGTTGCTGATCTCTGTGATTTCTTCAATGTGGGAAACGGTGTTT
GTGCTAGAAGAGGGCTGCGCTCTCTA
RF00003 (U1 family) (ENSG00000275291.1) SEQ ID NO: 9:
ATACTTACGTAACAGGAGAAAATACGGCCATGAAGTTGGTGTTTCTCGGGGGCGATTTCT
CCATTGTACTCAGTATGTGCTGACTGACTCCTGTTACTTCCACATGTGGGGAAACTGGAC
TGTAATTTGTGGTGGTGGGGAATTGCGTTCGCGCTTTCTTCT
RF00003 (U1 family) (ENSG00000278099.1) SEQ ID NO 10:
ATATTTACTTGGCAGGGGAGATAACGTGACCACGAAGGTGGTTTTCCCAGGGCTGAGGC
TTATTCATTGTACTCCGGATGTGCTGACCCCTGCGATTTCCCCAAATGTGGGAAACTCGA
CTGCATAATTTGTGGTAGTGGGGGGCTGTGTCCGTGCTTTTCCCTG
RF00003 (U1 family) (ENSG00000277918.1) SEQ ID NO: 11:
ATACTTACCTGGCAGGGGAGATACCATGATCACGAATGTGGTTTTCCCAGGGCGAGGCT
TATCCATTGCACTCCGGATGTGCTGACCCCTGCGATTTCCCCAAATGTGGGAAACTCGAC
TGCATAATTTGTGGTAGTGGGGGACTGCGTTCGCGCTTTCCCCTG
RF00003 (U1 family) (ENSG00000270384.1) SEQ ID NO: 12:
CCCCTTATGTGGCGGGGGACATACAGTGGTCAGGAAGGGGGTTCTCCCCGGAGGAGGC
TAGTCCACCACACTTCGGCTCCGCTGACCCCTGCGATTTCTCCACATGCGGGGCCCTCG
TCCGCGGTGGTGTTTGCGCTATCCGGCGGCTGGGTTCGCGCACTCACTCT
RF00003 (U1 family) (ENSG00000270722.1) SEQ ID NO: 13:
ATACTTACCTGGCAGGGGAGATACCATGATCACGAAGGTGGTTTTCCCTGGGCGAGGCT
TATCCATTGCACTCCGGATGTGCTGACCCCTGCGATTTCCCCAAATGTGGGAAACTTGAC
TGCATAATTTGTGGTAGTGGGGGACTGCGTTCGCGCTTTCCCCTG
RNVU1-6(ENSG00000201558.1) SEQ ID NO: 3:
ATACTTACCTGGCAGGAGAGATACCCTGGTCACGAAGGTGGTTTTTCCAGAGCGAGTCTT
ATCCATTGCACTCCGGATCTGCTGACCCCTGCAGTTTCCCCAAATGTGGGAAACTTGACT
GTATAATTTGTGGCAGTGGTAGACTGCATTCGCGCTTTCCCCTG
RF00003 (U1 family) (ENSG00000274428.1) SEQ ID NO: 14:
ATACTTACGTAACAGGAGAAAATACGGCCATGAAGTTGGTGTTTTTCGGGGGCGATTTCT
CCATTGTACTCAGTATGTGCTGACTGACTCCTGTTACTTCCACATGTGGGGAAACTGGAC
TGTAATTTGTGGTGGTGGGGAATTGCGTTCGCTTTCTTCT
RNVU1-7(ENSG00000206585.1) SEQ ID NO: 2:
ATACTTACCTGGCAGGGGAGATACCATGATCACGAAGGTGGTTTTCCCAGGGCGAGGCT
TATCCATTGCACTCCGGATGTGCTGACCCCTGCGATTTCCCCAAATGTGGGAAACTCGAC
TGCATAATTTGTGGTAGTGGGGGACTGCGTCCGCGCTTTCCCCTG
RF00003 (U1 family) (ENSG00000274210.1) SEQ ID NO: 15:
ATACTTACCTGGCAGGGGAGATACCATGATCATGAAGGCGCTTTTCTCATGGTCGCGGCT
TATCCATTGCGTTCCAGATGTGCTGACCTCTGCGATTTCCCCAAATGTGGGAAACTCGAC
TGCATAATTTGTGATAGTGGGGGGCTGCGTTCGTGCTTTCCCCTG
RF00003 (U1 family) (ENSG00000206828.1) SEQ ID NO: 16:
ATGTTTATCTGACAGAAGAAATGTTATGATCACGAAGGTGGTTTTTCCAGAGCAAGGCTTA
GCAATTTTGCTGTGGATGTGCTGACCCCTGTGATTTCCCCGAATGTGGGAATCTCCATTA
CATAACTTGTGGCAGTGGGGAACTGTGTCTGTGTTTTCCCCTG
RF00015 (U4 family) (ENSG00000276103.1) SEQ ID NO: 17:
AGTTTTGTGCAGTGGCAGTATCATAGCCAATGAGGTTTGTCCAATGTACAATTATTGCTAA
TTGAAAACGTTTCAGGCCAAGCACAGTGGCTCACGCCTATAATCCCAGCACTCTGG
RF00015 (U4 family)(ENSG00000283442.1) SEQ ID NO: 18:
AGCTTTGTACAGTGACAGGATTATAGCCAATGAGGCATGATTATTGCTAATTGAAAACTTT
TCCCAATACCCTGGCATTGGCAATTTTTGACAATCTCTATGGAGA
RF00066 (U7 family) (ENSG00000273629.1) SEQ ID NO: 19:
GAGTGTTACAGCCCTTTTAGAATTTGTCTAGCAAGTTTAGAAAATGATTCAAAGAGACTGA
C
RF00015 (U4 family)(ENSG00000277986.1) SEQ ID NO: 20:
AGCTTTGCACAGTGGCAGTATCGTAGCCAATGAGCTTTACCCGAGGCGCGATTATTGCTA
GTTAAATATTTATAAAAACCTTTCGAGCAGCGCCTGAACAAGAATAGGTTCAGAGGAGAC
TCC
RF00066 (U7 family) (ENSG00000275108.1) SEQ ID NO: 21:
CAGTGTTACAGATCTTTTAGAATTTGTCTAGTACATTTTCCAGTTTTCACCAGGAATCTGC
CT
RF00015 (U4 family)(ENSG00000274716.1) SEQ ID NO: 22:
AGCTTTGTGCAGTGGCAGTATCATAGCCAATGAGGTTTATCCGAGGCGTGATTATTGCTA
ATTGAAAAATTGAGGAATAGTT
RF00015 (U4 family)(ENSG00000273744.1) SEQ ID NO: 23:
AGCTTTGTGTACTGGCAATATCATAACCAATGAGGTTCATCCAAGACATGATAATTGCTAA
TGGCAAACAGCATAAAACCAACTGCC
RF00015 (U4 family)(ENSG00000278374.1) SEQ ID NO: 24:
AGCTTTGCGTGTGGCAGTATCATAGCCAATGAGGTTTATCCGAGGCGTGATTATTGCTAA
TTGAAAACAAAATGGAAGACAGGTGACTTTTCCCCTCCCTTGAATAAACACTTCTTCCACA
CATGGA
RF00015 (U4 family)(ENSG00000274197.1) SEQ ID NO: 25:
AGCTTTGTGCAGTGGCAGTATCGTAGCCAGTGAGGTTTATCGGAGGCGTGATCACTGCT
AATTGAAAACTTTTCTATAAAGGAAC
RF00066 (U7 family) (ENSG00000272215.1) SEQ ID NO: 26:
GAGTGTTGCAGCTCTTACAGAATTTGTCTAGCAGGTTTTCCAGTCTTTACCAGAAATGCCC
CC
RNU4-2(ENSG00000202538.1) SEQ ID NO:1:
AGCTTTGCGCAGTGGCAGTATCGTAGCCAATGAGGTTTATCCGAGGCGCGATTATTGCTA
ATTGAAAACTTTTCCCAATACCCCGCCATGACGACTTGAAATATAGTCGGCATTGGCAATT
TTTGACAGTCTCTACGGAGA
RF00066 (U7 family) (ENSG00000276376.1) SEQ ID NO: 27:
CAGTGTTACAGCTCTTTTAGAATTTGTTAGGCTGGGCGTACTGGCTCACGCCTATAATCC
CAA
RF00066 (U7 family) (ENSG00000275504.1) SEQ ID NO: 28:
GAGTGTTACAGCTCTATTAGAATTTGTCTAGCAGGTTTTCTGGTCTTCACTGGAAAGCACC
CCC
RF00003 (U1 family) (ENSG00000275631.1) SEQ ID NO: 29:
ATACTTACCTGGCAGGGCAGATACCATGATCTTAAAGGCAGTTTTCCCAGGGCAAGGCTT
ATCCATTCCACTCTGGATCCATCATAGGGATATGCTGATCCCTGGAATTGCCCCAAATGT
GGGAAGCTCTACTGCAAAATTTTTGGTAGTGAGCGATGGCATTATGCATTCATGTA
RF00066 (U7 family) ENSG00000275635.1) SEQ ID NO: 30:
CAGTGTTACAGCTCTTTTAGAATTTTTCTAGCAGGTTTTCCAGTTTTCACTGGAACCTCT
RF00066 (U7 family) (ENSG00000275268.1) SEQ ID NO: 31:
CAGTGTTACAGCTCTTTTGGAATTAGTCTAGCAGATTTCTCAGTTTTCACTGGAAACCCTT.

16. The cancer subtype identifier according to any of the preceding embodiments, wherein for the identification of the MXE2 subtype the primary small nuclear RNA expression shows up-regulation as compared to the MXE1 subtype exhibiting the highest predictive importance for MXE subtype classification selected from

RNU5B-1 (ENSG00000200156.1) SEQ ID NO: 32:
ATACTCTGGTTTCTCTTCAGATCGTATAAATCTTTCGCCTTTTACTAAAGATTTCCGTGGAG
AGGAACAACTCTGAGTCTTAAGCTAATTTTTTGAGGCCTTGTTCCGACAAGGCT
RNU12 (ENSG00000276027.1) SEQ ID NO: 33:
ATGCCTTAAACTTATGAGTAAGGAAAATAACGATTCGGGGTGACGCCCGAATCCTCACTG
CTAATGTGAGACGAATTTTTGAGCGGGTAAAGGTCGCCCTCAAGGTGACCCGCCTACTTT
GCGGGATGCCTGGGAGTTGCGATCTGCCCG
RNU11 (ENSG00000274978.1) SEQ ID NO: 34:
AAAAAGGGCTTCTGTCGTGAGTGGCACACGTAGGGCAACTCGATTGCTCTGCGTGCGGA
ATCGACATCAAGAGATTTCGGAAGCATAATTTTTTGGTATTTGGGCAGCTGGTGATCGTT
GGTCCCGGCGCCCTT
RNU5E-1 (ENSG00000199347.1) SEQ ID NO: 35:
ATACTCTGGTTTCTCTTCAAATCGTATAAATCTTTCGCCTTTTACTAAAGATTTCCGTGGAG
AGAAACGAGTGTGAGTCTGAAACCAATTTTTTGAGGCCTTGCGTTTCTTAGCAGGGCT
RNU6-9(ENSG00000207507.1) SEQ ID NO: 36:
GTGCTCGCTTCGGCAGCACATATACTAAAATTGGAACGATACAGAGAAGATTAGCATGGC
CCCTGCGCAAGGATGACACGCAAATTCGTGAAGCGTTCCATATTTTT
RNU5D-1 (ENSG00000200169.1) SEQ ID NO: 37:
ATGCTCTGGTTTCTCTTCAAATCGTATAAATCTTTCGCCTTTTACTAAAGATTTCCGTGGAG
AGAAACCGTTTTGAGTTTCAAGCAAATTTTTTGAAGCCCTATGTTGGTGGGGTT
RNU5A-1 (ENSG00000199568.1) SEQ ID NO: 38:
ATACTCTGGTTTCTCTTCAGATCGCATAAATCTTTCGCCTTTTACTAAAGATTTCCGTGGA
GAGGAACAACTCTGAGTCTTAACCCAATTTTTTGAGGCCTTGCTTTGGCAAGGCT
RNVU1-15 (ENSG00000207205.1) SEQ ID NO: 39:
ATACTTACTTGGCGGGGGAGATACCATGATCACGAAGGTGGTTTTCTCAGGGCGAGGCT
TATCCGTTATGTTCCGGGTGTACTGACCCCTGCCATTTTCCCCCAATGTGAGGAACTCGA
CTGCATAACTTGTGATAGTAGGGGACTGCGTTCGCGCTTTCCCCTG.

17. The cancer subtype identifier according to any of the preceding embodiments, wherein for the identification of the MXE2 subtype the small nuclear RNA expression shows up-regulation as compared to the MXE1 subtype:

RNU5E-1 (ENSG00000199347.1) SEQ ID NO: 35:
ATACTCTGGTTTCTCTTCAAATCGTATAAATCTTTCGCCTTTTACTAAAGATTTCCGTGGAG
AGAAACGAGTGTGAGTCTGAAACCAATTTTTTGAGGCCTTGCGTTTCTTAGCAGGGCT
RNU11 (ENSG00000274978.1) SEQ ID NO: 34:
AAAAAGGGCTTCTGTCGTGAGTGGCACACGTAGGGCAACTCGATTGCTCTGCGTGCGGA
ATCGACATCAAGAGATTTCGGAAGCATAATTTTTTGGTATTTGGGCAGCTGGTGATCGTT
GGTCCCGGCGCCCTT
RNU5F-1 (ENSG00000199377.1) SEQ ID NO: 44:
GATCTCTGGTTTCTCTTCATAACGAATAAATCTTTCGCCTTTTACTAAAGATTTCCGTGGA
GAAAAACAACTATGAGTTTATGGTTAAATTTTTTGAAGTCTTGCCTAGGCAAGGCT
RNU5D-1 (ENSG00000200169.1) SEQ ID NO: 37:
ATGCTCTGGTTTCTCTTCAAATCGTATAAATCTTTCGCCTTTTACTAAAGATTTCCGTGGAG
AGAAACCGTTTTGAGTTTCAAGCAAATTTTTTGAAGCCCTATGTTGGTGGGGTT
RNVU1-19 (ENSG00000275538.1) SEQ ID NO: 42:
ATACTTACCTGGCAGGGGAGATACTATTATCAAACGAAGGTGGTTTTTCTCAGGGCGAGG
GTTATCCATTGTGTTCCGGATGTGCTGACCTCTGCGATTTCCCCAAACGTGGGAAACTCG
ACTGCATAATTTGTGGTAGTGGGGGACTGCGTTCGCGCTTTCCTCTG
RNVU1-15 (ENSG00000207205.1) SEQ ID : 39:
ATACTTACTTGGCGGGGGAGATACCATGATCACGAAGGTGGTTTTCTCAGGGCGAGGCT
TATCCGTTATGTTCCGGGTGTACTGACCCCTGCCATTTTCCCCCAATGTGAGGAACTCGA
CTGCATAACTTGTGATAGTAGGGGACTGCGTTCGCGCTTTCCCCTG
RNVU1-1 (ENSG00000207340.1) SEQ ID NO:40 :
ATACTTACCTGGCAGGGGAGATACGATGATCACGAAGGTGGTTTTCCCAGGGAGAGGCT
TATCCATTGCACTCCGGATGTGCTGACCCCTGCCGTTTCCCCAAATGTGGGAAACTCGAC
TGCATAATTTGTGGTAGTGGGGGACTGCGTTCGCGCTGTCCTCTG
RNU4ATAC (ENSG00000264229.1) SEQ ID NO: 45:
ACCATCCTTTTCTTGGGGTTGCGCTACTGTCCAATGAGCGCATAGTGAGGGCAGTACTGC
TAACGCCTGAACAACACACCCGCATCAACTAGAGCTTTTGCTTTATTTTGGTGCAATTTTT
GGAAAA
RNU6ATAC (ENSG00000221676.1) SEQ ID NO: 41:
GTGTTGTATGAAAGGAGAGAAGGTTAGCACTCCCCTTGACAAGGATGGAAGAGGCCCTC
GGGCCTGACAACACGCATACGGTTAAGGCATTGCCACCTACTTCGTGGCATCTAACCATC
GTTTTTT
RNU5A-1 (ENSG00000199568.1) SEQ ID NO: 38:
ATACTCTGGTTTCTCTTCAGATCGCATAAATCTTTCGCCTTTTACTAAAGATTTCCGTGGA
GAGGAACAACTCTGAGTCTTAACCCAATTTTTTGAGGCCTTGCTTTGGCAAGGCT
RNU5B-1 (ENSG00000200156.1) SEQ ID NO: 32:
ATACTCTGGTTTCTCTTCAGATCGTATAAATCTTTCGCCTTTTACTAAAGATTTCCGTGGAG
AGGAACAACTCTGAGTCTTAAGCTAATTTTTTGAGGCCTTGTTCCGACAAGGCT
RNU6-1 (ENSG00000206625.1) SEQ ID NO 46:
GTGCTCGCTTCGGCAGCACATATACTAAAATTGGAACGATACAGAGAAGATTAGCATGGC
CCCTGCGCAAGGATGACACGCAAATTCGTGAAGCGTTCCATATTTTT
RNU6-9(ENSG00000207507.1) SEQ ID NO: 36:
GTGCTCGCTTCGGCAGCACATATACTAAAATTGGAACGATACAGAGAAGATTAGCATGGC
CCCTGCGCAAGGATGACACGCAAATTCGTGAAGCGTTCCATATTTTT
RNU6-2 (ENSG00000207357.1) SEQ ID NO: 47:
GTGCTCGCTTCGGCAGCACATATACTAAAATTGGAACGATACAGAGAAGATTAGCATGGC
CCCTGCGCAAGGATGACACGCAAATTCGTGAAGCGTTCCATATTTTT
RNU4-1 (ENSG00000200795.1) SEQ ID NO: 43:
AGCTTTGCGCAGTGGCAGTATCGTAGCCAATGAGGTCTATCCGAGGCGCGATTATTGCT
AATTGAAAACTTTTCCCAATACCCCGCCGTGACGACTTGCAATATAGTCGGCACTGGCAA
TTTTTGACAGTCTCTACGGAGA
RNU12 (ENSG00000276027.1) SEQ ID NO: 33:
ATGCCTTAAACTTATGAGTAAGGAAAATAACGATTCGGGGTGACGCCCGAATCCTCACTG
CTAATGTGAGACGAATTTTTGAGCGGGTAAAGGTCGCCCTCAAGGTGACCCGCCTACTTT
GCGGGATGCCTGGGAGTTGCGATCTGCCCG.

18. The cancer subtype identifier according any of the preceding embodiments, wherein for the identification of the MXE1 subtype the expression of the predictive protein-coding genes is characterized by up-regulation in the MXE1 subtype as compared to a MXE2 subtype of primary group, selected from the primary group consisting of kelch like family member 21 (KLHL21) (ENSG00000162413.16), mitotic arrest deficient 2 like 2 (MAD2L2) (ENSG00000116670.14), angiotensin II receptor associated protein (AGTRAP) (ENSG00000177674.15), NBL1, DAN family BMP antagonist (NBL1) (ENSG00000158747.14), zinc finger DHHC-type palmitoyltransferase 18 (ZDHHC18) (ENSG00000204160.11), solute carrier family 9 member A1 (SLC9A1) (ENSG00000090020.10), transmembrane protein 222 (TMEM222) (ENSG00000186501.14), platelet activating factor receptor (PTAFR) (ENSG00000169403.11), penta-EF-hand domain containing 1 (PEF1) (ENSG00000162517.12), NHS like 3 (KIAA1522) (ENSG00000162522.10), chromosome 1 open reading frame 122 (C1orf122) (ENSG00000197982.13), bone morphogenetic protein 8a (BMP8A) (ENSG00000183682.7), importin 13 (IPO13) (ENSG00000117408.10), diphthamide biosynthesis 2 (DPH2) (ENSG00000132768.13), ATPase H+ transporting V0 subunit b (ATP6V0B) (ENSG00000117410.13), aldo-keto reductase family 1 member A1 (AKR1A1) (ENSG00000117448.13), leucine rich repeat containing 41 (LRRC41) (ENSG00000132128.16), PDZK1 interacting protein 1 (PDZK1IP1) (ENSG00000162366.7), 24-dehydrocholesterol reductase (DHCR24) (ENSG00000116133.11), CD53 molecule (CD53) (ENSG00000143119.12), WD repeat domain 77 (WDR77) (ENSG00000116455.13), peroxisomal biogenesis factor 11 beta (PEX11B) (ENSG00000131779.10), aph-1 homolog A, gamma-secretase subunit (APH1A) (ENSG00000117362.12), proteasome 20S subunit beta 4 (PSMB4) (ENSG00000159377.10), S100 calcium binding protein A2 (S100A2) (ENSG00000196754.10), jumping translocation breakpoint (JTB) (ENSG00000143543.14), RUN and SH3 domain containing 1 (RUSC1) (ENSG00000160753.15), methyltransferase like 25B (RRNAD1) (ENSG00000143303.11), SLAM family member 8 (SLAMF8) (ENSG00000158714.10), protoporphyrinogen oxidase (PPOX) (ENSG00000143224.17), uridine-cytidine kinase 2 (UCK2) (ENSG00000143179.15), SFT2 domain containing 2 (SFT2D2) (ENSG00000213064.9), transmembrane protein 9 (TMEM9) (ENSG00000116857.16), NUAK family kinase 2 (NUAK2) (ENSG00000163545.8), left-right determination factor 1 (LEFTY1) (ENSG00000243709.1), jumonji domain containing 4 (JMJD4) (ENSG00000081692.12), solute carrier family 35 member F6 (SLC35F6) (ENSG00000213699.8), solute carrier family 5 member 6 (SLC5A6) (ENSG00000138074.14), sorting nexin 17 (SNX17) (ENSG00000115234.10), testis expressed 261 (TEX261) (ENSG00000144043.11), SMYD family member 5 (SMYD5) (ENSG00000135632.11), coiled-coil domain containing 142 (CCDC142) (ENSG00000135637.13), ssemaphorin 4F (SEMA4F) (ENSG00000135622.12), trans-golgi network protein 2 (TGOLN2) (ENSG00000152291.13), transmembrane protein 127 (TMEM127) (ENSG00000135956.8), mitotic spindle organizing protein 2B (MZT2B) (ENSG00000152082.13), Rho guanine nucleotide exchange factor 4 (ARHGEF4) (ENSG00000136002.18), COP9 signalosome subunit 7B (COPS7B) (ENSG00000144524.17), hes family bHLH transcription factor 6 (HES6) (ENSG00000144485.10), G protein-coupled receptor 35 (GPR35) (ENSG00000178623.11), actin related protein 2/3 complex subunit 4 (ARPC4) (ENSG00000241553.12), jagunal homolog 1 (JAGN1) (ENSG00000171135.13), interleukin 17 receptor C (IL17RC) (ENSG00000163702.18), transmembrane protein with metallophosphoesterase domain (TMPPE) (ENSG00000188167.8), ribosomal protein SA (RPSA) (ENSG00000168028.13), ubiquinol-cytochrome c reductase core protein 1 (UQCRC1) (ENSG00000010256.10), DALR anticodon binding domain containing 3 (DALRD3) (ENSG00000178149.16), cytochrome b561 family member D2 (CYB561D2) (ENSG00000114395.10), POC1 centriolar protein A (POC1A) (ENSG00000164087.7), solute carrier family 41 member 3 (SLC41A3) (ENSG00000114544.16), transmembrane protein adipocyte associated 1 (TPRA1) (ENSG00000163870.14), ALG3 alpha-1,3-mannosyltransferase (ALG3) (ENSG00000214160.9), EEF1A lysine methyltransferase 4 (EEF1AKMT4) (ENSG00000284753.1), major facilitator superfamily domain containing 10 (MFSD10) (ENSG00000109736.14), interferon regulatory factor 2 (IRF2) (ENSG00000168310.10), solute carrier family 35 member A4 (SLC35A4) (ENSG00000176087.14), NADH:ubiquinone oxidoreductase subunit A2 (NDUFA2) (ENSG00000131495.8), solute carrier family 36 member 1 (SLC36A1) (ENSG00000123643.12), antioxidant 1 copper chaperone (ATOX1) (ENSG00000177556.11), ADP ribosylation factor like GTPase 10 (ARL10) (ENSG00000175414.6), beta-1,4-galactosyltransferase 7 (B4GALT7) (ENSG00000027847.13), 5-phosphohydroxy-L-lysine phospho-lyase (PHYKPL) (ENSG00000175309.14), H3 clustered histone 2 (HIST1H3B) (ENSG00000274267.1), H1.2 linker histone, cluster member (HIST1H1C) (ENSG00000187837.3), H3 clustered histone 10 (HIST1H3H) (ENSG00000278828.1), olfactory receptor family 2 subfamily I member 1 pseudogene (OR2I1P) (ENSG00000237988.5), RNA polymerase I subunit H (ZNRD1) (ENSG00000066379.14), splicing factor 3b subunit 5 (SF3B5) (ENSG00000169976.6), aquaporin 1 (Colton blood group) (AQP1) (ENSG00000240583.11), receptor activity modifying protein 3 (RAMP3) (ENSG00000122679.8), NOP2/Sun RNA methyltransferase 5 (NSUN5) (ENSG00000130305.16), elastin (ELN) (ENSG00000049540.16), zona pellucida glycoprotein 3 (ZP3) (ENSG00000188372.14), cytochrome P450 family 51 subfamily A member 1 (CYP51A1) (ENSG00000001630.15), solute carrier family 12 member 9 (SLC12A9) (ENSG00000146828.17), monoacylglycerol O-acyltransferase 3 (MOGAT3) (ENSG00000106384.10), RNA polymerase II subunit J2 (POLR2J2) (ENSG00000228049.7), smoothened, frizzled class receptor (SMO) (ENSG00000128602.9), cell cycle regulator of NHEJ (C7orf49) (ENSG00000122783.16), MT-RNR2 like 6 (pseudogene) (MTRNR2L6) (ENSG00000270672.1), Fas activated serine/threonine kinase (FASTK) (ENSG00000164896.19), heparan-alpha-glucosaminide N-acetyltransferase (HGSNAT) (ENSG00000165102.14), grainyhead like transcription factor 2 (GRHL2) (ENSG00000083307.11), F-box and leucine rich repeat protein 6 (FBXL6) (ENSG00000182325.10), ribonuclease P/MRP subunit p25 like (RPP25L) (ENSG00000164967.9), phosphatidylinositol glycan anchor biosynthesis class O (PIGO) (ENSG00000165282.13), haloacid dehalogenase like hydrolase domain containing 3 (HDHD3) (ENSG00000119431.9), WD repeat domain 38 (WDR38) (ENSG00000136918.7), solute carrier family 2 member 8 (SLC2A8) (ENSG00000136856.17), torsin family 2 member A (TOR2A) (ENSG00000160404.17), ST6 N-acetylgalactosaminide alpha-2,6-sialyltransferase 6 (ST6GALNAC6) (ENSG00000160408.14), zinc finger DHHC-type palmitoyltransferase 12 (ZDHHC12) (ENSG00000160446.18), TBC1 domain family member 13 (TBC1D13) (ENSG00000107021.15), protein phosphatase 2 phosphatase activator (PPP2R4) (ENSG00000119383.19), calcium channel flower domain containing 1 (CACFD1) (ENSG00000160325.14), transmembrane protein 141 (TMEM141) (ENSG00000244187.7), transmembrane protein 203 (TMEM203) (ENSG00000187713.6), trypsin like peroxisomal matrix peptidase 1 (TYSND1) (ENSG00000156521.13), leucine rich repeat containing 20 (LRRC20) (ENSG00000172731.13), V-set immunoregulatory receptor (VSIR) (ENSG00000107738.19), SEC24 homolog C, COPII coat complex component (SEC24C) (ENSG00000176986.15), tectonic family member 3 (TCTN3) (ENSG00000119977.20), phosphatidylinositol 4-kinase type 2 alpha (PI4K2A) (ENSG00000155252.13), F-box and WD repeat domain containing 4 (FBXW4) (ENSG00000107829.13), glutathione S-transferase omega 1 (GSTO1) (ENSG00000148834.12), Bet1 golgi vesicular membrane trafficking protein like (BET1L) (ENSG00000177951.17), tetraspanin 4 (TSPAN4) (ENSG00000214063.10), chitinase domain containing 1 (CHID1) (ENSG00000177830.17), tumor suppressing subtransferable candidate 4 (TSSC4) (ENSG00000184281.14), FHF complex subunit HOOK interacting protein 1B (FAM160A2) (ENSG00000051009.10), peroxisomal biogenesis factor 16 (PEX16) (ENSG00000121680.15), solute carrier family 39 member 13 (SLC39A13) (ENSG00000165915.13), cytochrome b561 family member A3 (CYB561A3) (ENSG00000162144.9), transmembrane protein 258 (TMEM258) (ENSG00000134825.15), bestrophin 1 (BEST1) (ENSG00000167995.15), beta-1,3-glucuronyltransferase 3 (B3GAT3) (ENSG00000149541.9), integrator complex subunit 5 (INTS5) (ENSG00000185085.2), cytochrome c oxidase subunit 8A (COX8A) (ENSG00000176340.3), sorting nexin 32 (SNX32) (ENSG00000172803.17), frizzled class receptor 4 (FZD4) (ENSG00000174804.3), solute carrier family 37 member 4 (SLC37A4) (ENSG00000137700.17), NLR family member X1 (NLRX1) (ENSG00000160703.15), F-box and leucine rich repeat protein 14 (FBXL14) (ENSG00000171823.6), non-SMC condensin I complex subunit D2 (NCAPD2) (ENSG00000010292.12), myeloid leukemia factor 2 (MLF2) (ENSG00000089693.10), complement C3a receptor 1 (C3AR1) (ENSG00000171860.4), G protein-coupled receptor class C group 5 member A (GPRC5A) (ENSG00000013588.7), transmembrane protein 106C (TMEM106C) (ENSG00000134291.11), tubulin alpha 1b (TUBA1B) (ENSG00000123416.15), major facilitator superfamily domain containing 5 (MFSD5) (ENSG00000182544.8), ATP synthase membrane subunit C locus 2 (ATP5G2) (ENSG00000135390.17), retinol dehydrogenase 5 (RDH5) (ENSG00000135437.9), premelanosome protein (PMEL) (ENSG00000185664.14), cyclin dependent kinase 4 (CDK4) (ENSG00000135446.16), CTD small phosphatase 2 (CTDSP2) (ENSG00000175215.10), polypeptide N-acetylgalactosaminyltransferase 4 (GALNT4) (ENSG00000257594.3), phospholipase B domain containing 2 (PLBD2) (ENSG00000151176.7), paxillin (PXN) (ENSG00000089159.16), refilin A (FAM101A) (ENSG00000178882.14), transmembrane protein 253 (TMEM253) (ENSG00000232070.8), abhydrolase domain containing 4, N-acyl phospholipase B (ABHD4) (ENSG00000100439.10), proteasome 20S subunit beta 5 (PSMB5) (ENSG00000100804.18), phosphoenolpyruvate carboxykinase 2, mitochondrial (PCK2) (ENSG00000100889.11), ER membrane protein complex subunit 9 (EMC9) (ENSG00000100908.13), ribosomal protein S29 (RPS29) (ENSG00000213741.10), sushi domain containing 6 (SUSD6) (ENSG00000100647.7), acyl-CoA thioesterase 1 (ACOT1) (ENSG00000184227.7), acyl-CoA thioesterase 4 (ACOT4) (ENSG00000177465.4), ATP binding cassette subfamily D member 4 (ABCD4) (ENSG00000119688.20), NPC intracellular cholesterol transporter 2 (NPC2) (ENSG00000119655.10), angel homolog 1 (ANGEL1) (ENSG00000013523.9), CLOCK interacting pacemaker (CIPC) (ENSG00000198894.7), G protein-coupled receptor 68 (GPR68) (ENSG00000119714.10), solute carrier family 25 member 29 (SLC25A29) (ENSG00000197119.12), SIVA1 apoptosis inducing factor (SIVA1) (ENSG00000184990.12), G protein-coupled receptor 176 (GPR176) (ENSG00000166073.10), sorting nexin 22 (SNX22) (ENSG00000157734.13), pleckstrin homology domain containing O2 (PLEKHO2) (ENSG00000241839.9), cartilage intermediate layer protein (CILP) (ENSG00000138615.5), stomatin like 1 (STOML1) (ENSG00000067221.13), COMM domain containing 4 (COMMD4) (ENSG00000140365.15), phosphatidylinositol glycan anchor biosynthesis class Q (PIGQ) (ENSG00000007541.16), transmembrane protein 204 (TMEM204) (ENSG00000131634.13), NME/NM23 nucleoside diphosphate kinase 3 (NME3) (ENSG00000103024.7), NUBP iron-sulfur cluster assembly factor 2, cytosolic (NUBP2) (ENSG00000095906.16), presequence translocase associated motor 16 (PAM16) (ENSG00000217930.7), coronin 7 (CORO7) (ENSG00000262246.5), PAXIP1 associated glutamate rich protein 1 (PAGR1) (ENSG00000280789.1), CDP-diacylglycerol-inositol 3-phosphatidyltransferase (CDIPT) (ENSG00000103502.13), phosphorylase kinase catalytic subunit gamma 2 (PHKG2) (ENSG00000156873.15), ORAI calcium release-activated calcium modulator 3 (ORAI3) (ENSG00000175938.6), serine protease 8 (PRSS8) (ENSG00000052344.15), adhesion G protein-coupled receptor G1 (ADGRG1) (ENSG00000205336.11), chemokine like factor (CKLF) (ENSG00000217555.12), CKLF like MARVEL transmembrane domain containing 3 (CMTM3) (ENSG00000140931.19), transmembrane protein 208 (TMEM208) (ENSG00000168701.18), THAP domain containing 11 (THAP11) (ENSG00000168286.2), proteasome 20S subunit beta 10 (PSMB10) (ENSG00000205220.11), Tax1 binding protein 3 (TAX1BP3) (ENSG00000213977.7), GABA type A receptor-associated protein (GABARAP) (ENSG00000170296.9), transient receptor potential cation channel subfamily V member 2 (TRPV2) (ENSG00000187688.14), N-acetyltransferase domain containing 1 (NATD1) (ENSG00000274180.1), DNA polymerase delta interacting protein 2 (POLDIP2) (ENSG00000004142.11), notchless homolog 1 (NLE1) (ENSG00000073536.17), NFKB inhibitor interacting Ras like 2 (NKIRAS2) (ENSG00000168256.17), N-acetyl-alpha-glucosaminidase (NAGLU) (ENSG00000108784.9), reticulophagy regulator family member 3 (FAM134C) (ENSG00000141699.10), glucose-6-phosphatase catalytic subunit 3 (G6PC3) (ENSG00000141349.8), intercellular adhesion molecule 2 (ICAM2) (ENSG00000108622.10), G protein-coupled receptor class C group 5 member C (GPRC5C) (ENSG00000170412.16), thymidine kinase 1 (TK1) (ENSG00000167900.11), actin gamma 1 (ACTG1) (ENSG00000184009.10), FA core complex associated protein 100 (FAAP100) (ENSG00000185504.16), solute carrier family 25 member 10 (SLC25A10) (ENSG00000183048.11), phospholipid phosphatase 2 (PLPP2) (ENSG00000141934.9), secretory carrier membrane protein 4 (SCAMP4) (ENSG00000227500.9), MOB kinase activator 3A (MOB3A) (ENSG00000172081.13), fucosyltransferase 6 (FUT6) (ENSG00000156413.13), NADH:ubiquinone oxidoreductase subunit A11 (NDUFA11) (ENSG00000174886.12), calcyphosine (CAPS) (ENSG00000105519.15), PET100 cytochrome c oxidase chaperone (C19orf79) (ENSG00000229833.9), CD320 molecule (CD320) (ENSG00000167775.10), sphingosine-1-phosphate receptor 2 (S1PR2) (ENSG00000267534.3), autophagy related 4D cysteine peptidase (ATG4D) (ENSG00000130734.9), phospholipid phosphatase related 2 (PLPPR2) (ENSG00000105520.10), WD repeat domain 83 opposite strand (WDR83OS) (ENSG00000105583.9), notch receptor 3 (NOTCH3) (ENSG00000074181.8), adaptor related protein complex 1 subunit mu 1 (AP1M1) (ENSG00000072958.8), ubiquitin A-52 residue ribosomal protein fusion product 1 (UBA52) (ENSG00000221983.7), kelch like family member 26 (KLHL26) (ENSG00000167487.11), armadillo repeat containing 6 (ARMC6) (ENSG00000105676.13), transmembrane protein 161A (TMEM161A) (ENSG00000064545.14), nuclear receptor 2C2 associated protein (NR2C2AP) (ENSG00000184162.14), MAU2 sister chromatid cohesion factor (MAU2) (ENSG00000129933.20), transmembrane protein 147 (TMEM147) (ENSG00000105677.11), presenilin enhancer, gamma-secretase subunit (PSENEN) (ENSG00000205155.7), cytochrome c oxidase subunit 7A1 (COX7A1) (ENSG00000161281.10), Charcot-Leyden crystal galectin (CLC) (ENSG00000105205.6), zinc finger protein 574 (ZNF574) (ENSG00000105732.12), ETHE1 persulfide dioxygenase (ETHE1) (ENSG00000105755.7), adaptor related protein complex 2 subunit sigma 1 (AP2S1) (ENSG00000042753.11), transient receptor potential cation channel subfamily M member 4 (TRPM4) (ENSG00000130529.15), BCL2 like 12 (BCL2L12) (ENSG00000126453.9), sialic acid binding Ig like lectin 14 (SIGLEC14) (ENSG00000254415.3), formyl peptide receptor 1 (FPR1) (ENSG00000171051.8), protein phosphatase 2 scaffold subunit Aalpha (PPP2R1A) (ENSG00000105568.17), zinc finger protein 320 (ZNF320) (ENSG00000182986.13), ribosomal protein S5 (RPS5) (ENSG00000083845.8), small nuclear ribonucleoprotein polypeptides B and B1 (SNRPB) (ENSG00000125835.18), U-box domain containing 5 (UBOX5) (ENSG00000185019.16), acyl-CoA synthetase short chain family member 1 (ACSS1) (ENSG00000154930.14), glycogen phosphorylase B (PYGB) (ENSG00000100994.11), AAR2 splicing factor (AAR2) (ENSG00000131043.11), acyl-CoA thioesterase 8 (ACOT8) (ENSG00000101473.16), phosphorylated CTD interacting factor 1 (PCIF1) (ENSG00000100982.11), gamma-glutamyltransferase 5 (GGT5) (ENSG00000099998.17), rhomboid domain containing 3 (RHBDD3) (ENSG00000100263.13), galectin 1 (LGALS1) (ENSG00000100097.11), platelet derived growth factor subunit B (PDGFB) (ENSG00000100311.16), centromere protein M (CENPM) (ENSG00000100162.14), translocator protein (TSPO) (ENSG00000100300.17), gem nuclear organelle associated protein 8 (GEMIN8) (ENSG00000046647.13), NADH:ubiquinone oxidoreductase subunit B11 (NDUFB11) (ENSG00000147123.10), ribosomal protein L10 (RPL10) (ENSG00000147403.16), L antigen family member 3 (LAGE3) (ENSG00000196976.7), AC004556.1 (ENSG00000276345.1), peptidyl arginine deiminase 2 (PADI2) (ENSG00000117115.12), phospholipase A2 group IIA (PLA2G2A) (ENSG00000188257.10), collagen type IX alpha 2 chain (COL9A2) (ENSG00000049089.14), cytochrome P450 family 4 subfamily X member 1 (CYP4X1) (ENSG00000186377.7), enoyl-CoA hydratase domain containing 2 (ECHDC2) (ENSG00000121310.16), maestro heat like repeat family member 7 (MROH7) (ENSG00000184313.19), protein kinase cAMP-activated catalytic subunit beta (PRKACB) (ENSG00000142875.19), sterile alpha motif domain containing 13 (SAMD13) (ENSG00000203943.8), coagulation factor III, tissue factor (F3) (ENSG00000117525.13), 3-hydroxy-3-methylglutaryl-CoA synthase 2 (HMGCS2) (ENSG00000134240.11), selenium binding protein 1 (SELENBP1) (ENSG00000143416.20), regulator of G protein signaling 5 (RGS5) (ENSG00000143248.12), cathepsin E (CTSE) (ENSG00000196188.10), immunoglobulin kappa constant (IGKC) (ENSG00000211592.8), chromosome 2 open reading frame 88 (C2orf88) (ENSG00000187699.10), integral membrane protein 2C (ITM2C) (ENSG00000135916.15), FAM3 metabolism regulating signaling molecule D (FAM3D) (ENSG00000198643.6), ABI family member 3 binding protein (ABI3BP) (ENSG00000154175.16), resistin like beta (RETNLB) (ENSG00000163515.6), solute carrier organic anion transporter family member 2A1 (SLCO2A1) (ENSG00000174640.12), mucin 4, cell surface associated (MUC4) (ENSG00000145113.21), chromosome 4 open reading frame 19 (C4orf19) (ENSG00000154274.14), UDP glucuronosyltransferase family 2 member B17 (UGT2B17) (ENSG00000197888.2), joining chain of multimeric IgA and IgM (JCHAIN) (ENSG00000132465.10), N-acylethanolamine acid amidase (NAAA) (ENSG00000138744.14), nuclear receptor subfamily 3 group C member 2 (NR3C2) (ENSG00000151623.14), C-C motif chemokine ligand 28 (CCL28) (ENSG00000151882.11), marginal zone B and B1 cell specific protein (MZB1) (ENSG00000170476.15), solute carrier family 26 member 2 (SLC26A2) (ENSG00000155850.7), CD74 molecule (CD74) (ENSG00000019582.14), chloride intracellular channel 5 (CLIC5) (ENSG00000112782.16), 5′-nucleotidase ecto (NT5E) (ENSG00000135318.11), sphingomyelin phosphodiesterase acid like 3A (SMPDL3A) (ENSG00000172594.12), scinderin (SCIN) (ENSG00000006747.14), histone deacetylase 9 (HDAC9) (ENSG00000048052.21), transient receptor potential cation channel subfamily A member 1 (TRPA1) (ENSG00000104321.10), stathmin 2 (STMN2) (ENSG00000104435.13), glutamic-pyruvic transaminase (GPT) (ENSG00000167701.13), prostaglandin D2 synthase (PTGDS) (ENSG00000107317.12), Rho related BTB domain containing 1 (RHOBTB1) (ENSG00000072422.16), cadherin related family member 1 (CDHR1) (ENSG00000148600.14), 3′-phosphoadenosine 5′-phosphosulfate synthase 2 (PAPSS2) (ENSG00000198682.12), actin filament associated protein 1 like 2 (AFAP1L2) (ENSG00000169129.14), X-ray radiation resistance associated 1 (XRRA1) (ENSG00000166435.15), calpain 5 (CAPN5) (ENSG00000149260.15), coiled-coil domain containing 89 (CCDC89) (ENSG00000179071.4), synaptotagmin like 2 (SYTL2) (ENSG00000137501.17), matrix metallopeptidase 7 (MMP7) (ENSG00000137673.8), neurexophilin and PC-esterase domain family member 1 (NXPE1) (ENSG00000095110.8), neurexophilin and PC-esterase domain family member 4 (NXPE4) (ENSG00000137634.9), tripartite motif containing 29 (TRIM29) (ENSG00000137699.16), V-set and immunoglobulin domain containing 2 (VSIG2) (ENSG00000019102.11), solute carrier family 37 member 2 (SLC37A2) (ENSG00000134955.11), ST3 beta-galactoside alpha-2,3-sialyltransferase 4 (ST3GAL4) (ENSG00000110080.18), BARX homeobox 2 (BARX2) (ENSG00000043039.6), lysophosphatidic acid receptor 6 (LPAR6) (ENSG00000139679.15), heat shock protein family A (Hsp70) member 2 (HSPA2) (ENSG00000126803.9), bradykinin receptor B2 (BDKRB2) (ENSG00000168398.6), creatine kinase B (CKB) (ENSG00000166165.12), calpain 3 (CAPN3) (ENSG00000092529.23), beta-2-microglobulin (B2M) (ENSG00000166710.17), RNA binding fox-1 homolog 1 (RBFOX1) (ENSG00000078328.20), class II major histocompatibility complex transactivator (CIITA) (ENSG00000179583.19), endoplasmic reticulum to nucleus signaling 2 (ERN2) (ENSG00000134398.14), lactate dehydrogenase D (LDHD) (ENSG00000166816.13), carbohydrate sulfotransferase 5 (CHST5) (ENSG00000135702.14), carbonic anhydrase 4 (CA4) (ENSG00000167434.9), axin 2 (AXIN2) (ENSG00000168646.12), secreted and transmembrane 1 (SECTM1) (ENSG00000141574.7), solute carrier family 14 member 1 (Kidd blood group) (SLC14A1) (ENSG00000141469.17), ring finger protein 152 (RNF152) (ENSG00000176641.10), plasmalemma vesicle associated protein (PLVAP) (ENSG00000130300.8), cation channel sperm associated auxiliary subunit gamma (CATSPERG) (ENSG00000099338.22), Fc gamma binding protein (FCGBP) (ENSG00000275395.5), kallikrein 1 (KLK1) (ENSG00000167748.10), glycine receptor alpha 2 (GLRA2) (ENSG00000101958.13), claudin 2 (CLDN2) (ENSG00000165376.10), MT-ND3 (ENSG00000198840.2).

19. The cancer subtype identifier according any of the preceding embodiments, wherein for the identification of the MXE1 subtype the expression of the predictive protein-coding genes is characterized by up-regulation in the MXE1 subtype as compared to a MXE2 subtype of primary group, selected from kelch like family member 21 (KLHL21) (ENSG00000162413.16), mitotic arrest deficient 2 like 2 (MAD2L2) (ENSG00000116670.14), angiotensin II receptor associated protein (AGTRAP) (ENSG00000177674.15), NBL1, DAN family BMP antagonist (NBL1) (ENSG00000158747.14), zinc finger DHHC-type palmitoyltransferase 18 (ZDHHC18) (ENSG00000204160.11), solute carrier family 9 member A1 (SLC9A1) (ENSG00000090020.10), transmembrane protein 222 (TMEM222) (ENSG00000186501.14), platelet activating factor receptor (PTAFR) (ENSG00000169403.11), penta-EF-hand domain containing 1 (PEF1) (ENSG00000162517.12), NHS like 3 (KIAA1522) (ENSG00000162522.10), chromosome 1 open reading frame 122 (C1orf122) (ENSG00000197982.13), bone morphogenetic protein 8a (BMP8A) (ENSG00000183682.7), importin 13 (IPO13) (ENSG00000117408.10), diphthamide biosynthesis 2 (DPH2) (ENSG00000132768.13), ATPase H+ transporting V0 subunit b (ATP6V0B) (ENSG00000117410.13), aldo-keto reductase family 1 member A1 (AKR1A1) (ENSG00000117448.13), leucine rich repeat containing 41 (LRRC41) (ENSG00000132128.16), PDZK1 interacting protein 1 (PDZK1IP1) (ENSG00000162366.7), 24-dehydrocholesterol reductase (DHCR24) (ENSG00000116133.11), CD53 molecule (CD53) (ENSG00000143119.12), WD repeat domain 77 (WDR77) (ENSG00000116455.13), peroxisomal biogenesis factor 11 beta (PEX11B) (ENSG00000131779.10), aph-1 homolog A, gamma-secretase subunit (APH1A) (ENSG00000117362.12), proteasome 20S subunit beta 4 (PSMB4) (ENSG00000159377.10), S100 calcium binding protein A2 (S100A2) (ENSG00000196754.10), jumping translocation breakpoint (JTB) (ENSG00000143543.14), RUN and SH3 domain containing 1 (RUSC1) (ENSG00000160753.15), methyltransferase like 25B (RRNAD1) (ENSG00000143303.11), SLAM family member 8 (SLAMF8) (ENSG00000158714.10), protoporphyrinogen oxidase (PPOX) (ENSG00000143224.17), uridine-cytidine kinase 2 (UCK2) (ENSG00000143179.15), SFT2 domain containing 2 (SFT2D2) (ENSG00000213064.9), transmembrane protein 9 (TMEM9) (ENSG00000116857.16), NUAK family kinase 2 (NUAK2) (ENSG00000163545.8), left-right determination factor 1 (LEFTY1) (ENSG00000243709.1), jumonji domain containing 4 (JMJD4) (ENSG00000081692.12), solute carrier family 35 member F6 (SLC35F6) (ENSG00000213699.8), solute carrier family 5 member 6 (SLC5A6) (ENSG00000138074.14), sorting nexin 17 (SNX17) (ENSG00000115234.10), testis expressed 261 (TEX261) (ENSG00000144043.11), SMYD family member 5 (SMYD5) (ENSG00000135632.11), coiled-coil domain containing 142 (CCDC142) (ENSG00000135637.13), ssemaphorin 4F (SEMA4F) (ENSG00000135622.12), trans-golgi network protein 2 (TGOLN2) (ENSG00000152291.13), transmembrane protein 127 (TMEM127) (ENSG00000135956.8), mitotic spindle organizing protein 2B (MZT2B) (ENSG00000152082.13), Rho guanine nucleotide exchange factor 4 (ARHGEF4) (ENSG00000136002.18), COP9 signalosome subunit 7B (COPS7B) (ENSG00000144524.17), hes family bHLH transcription factor 6 (HES6) (ENSG00000144485.10), G protein-coupled receptor 35 (GPR35) (ENSG00000178623.11), actin related protein 2/3 complex subunit 4 (ARPC4) (ENSG00000241553.12), jagunal homolog 1 (JAGN1) (ENSG00000171135.13), interleukin 17 receptor C (IL17RC) (ENSG00000163702.18), transmembrane protein with metallophosphoesterase domain (TMPPE) (ENSG00000188167.8), ribosomal protein SA (RPSA) (ENSG00000168028.13), ubiquinol-cytochrome c reductase core protein 1 (UQCRC1) (ENSG00000010256.10), DALR anticodon binding domain containing 3 (DALRD3) (ENSG00000178149.16), cytochrome b561 family member D2 (CYB561D2) (ENSG00000114395.10), POC1 centriolar protein A (POC1A) (ENSG00000164087.7), solute carrier family 41 member 3 (SLC41A3) (ENSG00000114544.16), transmembrane protein adipocyte associated 1 (TPRA1) (ENSG00000163870.14), ALG3 alpha-1,3-mannosyltransferase (ALG3) (ENSG00000214160.9), EEF1A lysine methyltransferase 4 (EEF1AKMT4) (ENSG00000284753.1), major facilitator superfamily domain containing 10 (MFSD10) (ENSG00000109736.14), interferon regulatory factor 2 (IRF2) (ENSG00000168310.10), solute carrier family 35 member A4 (SLC35A4) (ENSG00000176087.14), NADH:ubiquinone oxidoreductase subunit A2 (NDUFA2) (ENSG00000131495.8), solute carrier family 36 member 1 (SLC36A1) (ENSG00000123643.12), antioxidant 1 copper chaperone (ATOX1) (ENSG00000177556.11), ADP ribosylation factor like GTPase 10 (ARL10) (ENSG00000175414.6), beta-1,4-galactosyltransferase 7 (B4GALT7) (ENSG00000027847.13), 5-phosphohydroxy-L-lysine phospho-lyase (PHYKPL) (ENSG00000175309.14), H3 clustered histone 2 (HIST1H3B) (ENSG00000274267.1), H1.2 linker histone, cluster member (HIST1H1C) (ENSG00000187837.3), H3 clustered histone 10 (HIST1H3H) (ENSG00000278828.1), olfactory receptor family 2 subfamily I member 1 pseudogene (OR2I1P) (ENSG00000237988.5), RNA polymerase I subunit H (ZNRD1) (ENSG00000066379.14), splicing factor 3b subunit 5 (SF3B5) (ENSG00000169976.6), aquaporin 1 (Colton blood group) (AQP1) (ENSG00000240583.11), receptor activity modifying protein 3 (RAMP3) (ENSG00000122679.8), NOP2/Sun RNA methyltransferase 5 (NSUN5) (ENSG00000130305.16), elastin (ELN) (ENSG00000049540.16), zona pellucida glycoprotein 3 (ZP3) (ENSG00000188372.14), cytochrome P450 family 51 subfamily A member 1 (CYP51A1) (ENSG00000001630.15), solute carrier family 12 member 9 (SLC12A9) (ENSG00000146828.17), monoacylglycerol O-acyltransferase 3 (MOGAT3) (ENSG00000106384.10), RNA polymerase II subunit J2 (POLR2J2) (ENSG00000228049.7), smoothened, frizzled class receptor (SMO) (ENSG00000128602.9), cell cycle regulator of NHEJ (C7orf49) (ENSG00000122783.16), MT-RNR2 like 6 (pseudogene) (MTRNR2L6) (ENSG00000270672.1), Fas activated serine/threonine kinase (FASTK) (ENSG00000164896.19), heparan-alpha-glucosaminide N-acetyltransferase (HGSNAT) (ENSG00000165102.14), grainyhead like transcription factor 2 (GRHL2) (ENSG00000083307.11), F-box and leucine rich repeat protein 6 (FBXL6) (ENSG00000182325.10), ribonuclease P/MRP subunit p25 like (RPP25L) (ENSG00000164967.9), phosphatidylinositol glycan anchor biosynthesis class O (PIGO) (ENSG00000165282.13), haloacid dehalogenase like hydrolase domain containing 3 (HDHD3) (ENSG00000119431.9), WD repeat domain 38 (WDR38) (ENSG00000136918.7), solute carrier family 2 member 8 (SLC2A8) (ENSG00000136856.17), torsin family 2 member A (TOR2A) (ENSG00000160404.17), ST6 N-acetylgalactosaminide alpha-2,6-sialyltransferase 6 (ST6GALNAC6) (ENSG00000160408.14), zinc finger DHHC-type palmitoyltransferase 12 (ZDHHC12) (ENSG00000160446.18), TBC1 domain family member 13 (TBC1D13) (ENSG00000107021.15), protein phosphatase 2 phosphatase activator (PPP2R4) (ENSG00000119383.19), calcium channel flower domain containing 1 (CACFD1) (ENSG00000160325.14), transmembrane protein 141 (TMEM141) (ENSG00000244187.7), transmembrane protein 203 (TMEM203) (ENSG00000187713.6), trypsin like peroxisomal matrix peptidase 1 (TYSND1) (ENSG00000156521.13), leucine rich repeat containing 20 (LRRC20) (ENSG00000172731.13), V-set immunoregulatory receptor (VSIR) (ENSG00000107738.19), SEC24 homolog C, COPII coat complex component (SEC24C) (ENSG00000176986.15), tectonic family member 3 (TCTN3) (ENSG00000119977.20), phosphatidylinositol 4-kinase type 2 alpha (PI4K2A) (ENSG00000155252.13), F-box and WD repeat domain containing 4 (FBXW4) (ENSG00000107829.13), glutathione S-transferase omega 1 (GSTO1) (ENSG00000148834.12), Bet1 golgi vesicular membrane trafficking protein like (BET1L) (ENSG00000177951.17), tetraspanin 4 (TSPAN4) (ENSG00000214063.10), chitinase domain containing 1 (CHID1) (ENSG00000177830.17), tumor suppressing subtransferable candidate 4 (TSSC4) (ENSG00000184281.14), FHF complex subunit HOOK interacting protein 1B (FAM160A2) (ENSG00000051009.10), peroxisomal biogenesis factor 16 (PEX16) (ENSG00000121680.15), solute carrier family 39 member 13 (SLC39A13) (ENSG00000165915.13), cytochrome b561 family member A3 (CYB561A3) (ENSG00000162144.9), transmembrane protein 258 (TMEM258) (ENSG00000134825.15), bestrophin 1 (BEST1) (ENSG00000167995.15), beta-1,3-glucuronyltransferase 3 (B3GAT3) (ENSG00000149541.9), integrator complex subunit 5 (INTS5) (ENSG00000185085.2), cytochrome c oxidase subunit 8A (COX8A) (ENSG00000176340.3), sorting nexin 32 (SNX32) (ENSG00000172803.17), frizzled class receptor 4 (FZD4) (ENSG00000174804.3), solute carrier family 37 member 4 (SLC37A4) (ENSG00000137700.17), NLR family member X1 (NLRX1) (ENSG00000160703.15), F-box and leucine rich repeat protein 14 (FBXL14) (ENSG00000171823.6), non-SMC condensin I complex subunit D2 (NCAPD2) (ENSG00000010292.12), myeloid leukemia factor 2 (MLF2) (ENSG00000089693.10), complement C3a receptor 1 (C3AR1) (ENSG00000171860.4), G protein-coupled receptor class C group 5 member A (GPRC5A) (ENSG00000013588.7), transmembrane protein 106C (TMEM106C) (ENSG00000134291.11), tubulin alpha 1b (TUBA1B) (ENSG00000123416.15), major facilitator superfamily domain containing 5 (MFSD5) (ENSG00000182544.8), ATP synthase membrane subunit C locus 2 (ATP5G2) (ENSG00000135390.17), retinol dehydrogenase 5 (RDH5) (ENSG00000135437.9), premelanosome protein (PMEL) (ENSG00000185664.14), cyclin dependent kinase 4 (CDK4) (ENSG00000135446.16), CTD small phosphatase 2 (CTDSP2) (ENSG00000175215.10), polypeptide N-acetylgalactosaminyltransferase 4 (GALNT4) (ENSG00000257594.3), phospholipase B domain containing 2 (PLBD2) (ENSG00000151176.7), paxillin (PXN) (ENSG00000089159.16), refilin A (FAM101A) (ENSG00000178882.14), transmembrane protein 253 (TMEM253) (ENSG00000232070.8), abhydrolase domain containing 4, N-acyl phospholipase B (ABHD4) (ENSG00000100439.10), proteasome 20S subunit beta 5 (PSMB5) (ENSG00000100804.18), phosphoenolpyruvate carboxykinase 2, mitochondrial (PCK2) (ENSG00000100889.11), ER membrane protein complex subunit 9 (EMC9) (ENSG00000100908.13), ribosomal protein S29 (RPS29) (ENSG00000213741.10), sushi domain containing 6 (SUSD6) (ENSG00000100647.7), acyl-CoA thioesterase 1 (ACOT1) (ENSG00000184227.7), acyl-CoA thioesterase 4 (ACOT4) (ENSG00000177465.4), ATP binding cassette subfamily D member 4 (ABCD4) (ENSG00000119688.20), NPC intracellular cholesterol transporter 2 (NPC2) (ENSG00000119655.10), angel homolog 1 (ANGEL1) (ENSG00000013523.9), CLOCK interacting pacemaker (CIPC) (ENSG00000198894.7), G protein-coupled receptor 68 (GPR68) (ENSG00000119714.10), solute carrier family 25 member 29 (SLC25A29) (ENSG00000197119.12), SIVA1 apoptosis inducing factor (SIVA1) (ENSG00000184990.12), G protein-coupled receptor 176 (GPR176) (ENSG00000166073.10), sorting nexin 22 (SNX22) (ENSG00000157734.13), pleckstrin homology domain containing O2 (PLEKHO2) (ENSG00000241839.9), cartilage intermediate layer protein (CILP) (ENSG00000138615.5), stomatin like 1 (STOML1) (ENSG00000067221.13), COMM domain containing 4 (COMMD4) (ENSG00000140365.15), phosphatidylinositol glycan anchor biosynthesis class Q (PIGQ) (ENSG00000007541.16), transmembrane protein 204 (TMEM204) (ENSG00000131634.13), NME/NM23 nucleoside diphosphate kinase 3 (NME3) (ENSG00000103024.7), NUBP iron-sulfur cluster assembly factor 2, cytosolic (NUBP2) (ENSG00000095906.16), presequence translocase associated motor 16 (PAM16) (ENSG00000217930.7), coronin 7 (CORO7) (ENSG00000262246.5), PAXIP1 associated glutamate rich protein 1 (PAGR1) (ENSG00000280789.1), CDP-diacylglycerol-inositol 3-phosphatidyltransferase (CDIPT) (ENSG00000103502.13), phosphorylase kinase catalytic subunit gamma 2 (PHKG2) (ENSG00000156873.15), ORAI calcium release-activated calcium modulator 3 (ORAI3) (ENSG00000175938.6), serine protease 8 (PRSS8) (ENSG00000052344.15), adhesion G protein-coupled receptor G1 (ADGRG1) (ENSG00000205336.11), chemokine like factor (CKLF) (ENSG00000217555.12), CKLF like MARVEL transmembrane domain containing 3 (CMTM3) (ENSG00000140931.19), transmembrane protein 208 (TMEM208) (ENSG00000168701.18), THAP domain containing 11 (THAP11) (ENSG00000168286.2), proteasome 20S subunit beta 10 (PSMB10) (ENSG00000205220.11), Tax1 binding protein 3 (TAX1BP3) (ENSG00000213977.7), GABA type A receptor-associated protein (GABARAP) (ENSG00000170296.9), transient receptor potential cation channel subfamily V member 2 (TRPV2) (ENSG00000187688.14), N-acetyltransferase domain containing 1 (NATD1) (ENSG00000274180.1), DNA polymerase delta interacting protein 2 (POLDIP2) (ENSG00000004142.11), notchless homolog 1 (NLE1) (ENSG00000073536.17), NFKB inhibitor interacting Ras like 2 (NKIRAS2) (ENSG00000168256.17), N-acetyl-alpha-glucosaminidase (NAGLU) (ENSG00000108784.9), reticulophagy regulator family member 3 (FAM134C) (ENSG00000141699.10), glucose-6-phosphatase catalytic subunit 3 (G6PC3) (ENSG00000141349.8), intercellular adhesion molecule 2 (ICAM2) (ENSG00000108622.10), G protein-coupled receptor class C group 5 member C (GPRC5C) (ENSG00000170412.16), thymidine kinase 1 (TK1) (ENSG00000167900.11), actin gamma 1 (ACTG1) (ENSG00000184009.10), FA core complex associated protein 100 (FAAP100) (ENSG00000185504.16), solute carrier family 25 member 10 (SLC25A10) (ENSG00000183048.11), phospholipid phosphatase 2 (PLPP2) (ENSG00000141934.9), secretory carrier membrane protein 4 (SCAMP4) (ENSG00000227500.9), MOB kinase activator 3A (MOB3A) (ENSG00000172081.13), fucosyltransferase 6 (FUT6) (ENSG00000156413.13), NADH:ubiquinone oxidoreductase subunit A11 (NDUFA11) (ENSG00000174886.12), calcyphosine (CAPS) (ENSG00000105519.15), PET100 cytochrome c oxidase chaperone (C19orf79) (ENSG00000229833.9), CD320 molecule (CD320) (ENSG00000167775.10), sphingosine-1-phosphate receptor 2 (S1PR2) (ENSG00000267534.3), autophagy related 4D cysteine peptidase (ATG4D) (ENSG00000130734.9), phospholipid phosphatase related 2 (PLPPR2) (ENSG00000105520.10), WD repeat domain 83 opposite strand (WDR83OS) (ENSG00000105583.9), notch receptor 3 (NOTCH3) (ENSG00000074181.8), adaptor related protein complex 1 subunit mu 1 (AP1M1) (ENSG00000072958.8), ubiquitin A-52 residue ribosomal protein fusion product 1 (UBA52) (ENSG00000221983.7), kelch like family member 26 (KLHL26) (ENSG00000167487.11), armadillo repeat containing 6 (ARMC6) (ENSG00000105676.13), transmembrane protein 161A (TMEM161A) (ENSG00000064545.14), nuclear receptor 2C2 associated protein (NR2C2AP) (ENSG00000184162.14), MAU2 sister chromatid cohesion factor (MAU2) (ENSG00000129933.20), transmembrane protein 147 (TMEM147) (ENSG00000105677.11), presenilin enhancer, gamma-secretase subunit (PSENEN) (ENSG00000205155.7), cytochrome c oxidase subunit 7A1 (COX7A1) (ENSG00000161281.10), Charcot-Leyden crystal galectin (CLC) (ENSG00000105205.6), zinc finger protein 574 (ZNF574) (ENSG00000105732.12), ETHE1 persulfide dioxygenase (ETHE1) (ENSG00000105755.7), adaptor related protein complex 2 subunit sigma 1 (AP2S1) (ENSG00000042753.11), transient receptor potential cation channel subfamily M member 4 (TRPM4) (ENSG00000130529.15), BCL2 like 12 (BCL2L12) (ENSG00000126453.9), sialic acid binding Ig like lectin 14 (SIGLEC14) (ENSG00000254415.3), formyl peptide receptor 1 (FPR1) (ENSG00000171051.8), protein phosphatase 2 scaffold subunit Aalpha (PPP2R1A) (ENSG00000105568.17), zinc finger protein 320 (ZNF320) (ENSG00000182986.13), ribosomal protein S5 (RPS5) (ENSG00000083845.8), small nuclear ribonucleoprotein polypeptides B and B1 (SNRPB) (ENSG00000125835.18), U-box domain containing 5 (UBOX5) (ENSG00000185019.16), acyl-CoA synthetase short chain family member 1 (ACSS1) (ENSG00000154930.14), glycogen phosphorylase B (PYGB) (ENSG00000100994.11), AAR2 splicing factor (AAR2) (ENSG00000131043.11), acyl-CoA thioesterase 8 (ACOT8) (ENSG00000101473.16), phosphorylated CTD interacting factor 1 (PCIF1) (ENSG00000100982.11), gamma-glutamyltransferase 5 (GGT5) (ENSG00000099998.17), rhomboid domain containing 3 (RHBDD3) (ENSG00000100263.13), galectin 1 (LGALS1) (ENSG00000100097.11), platelet derived growth factor subunit B (PDGFB) (ENSG00000100311.16), centromere protein M (CENPM) (ENSG00000100162.14), translocator protein (TSPO) (ENSG00000100300.17), gem nuclear organelle associated protein 8 (GEMIN8) (ENSG00000046647.13), NADH:ubiquinone oxidoreductase subunit B11 (NDUFB11) (ENSG00000147123.10), ribosomal protein L10 (RPL10) (ENSG00000147403.16), L antigen family member 3 (LAGE3) (ENSG00000196976.7), AC004556.1 (ENSG00000276345.1), preferably wherein for the identification of the MXE1 subtype the expression of the predictive protein-coding genes is characterized by their up-regulation in the MXE1 subtype as compared to the MXE2 subtype, preferably selected from kelch like family member 21 (KLHL21) (ENSG00000162413.16), mitotic arrest deficient 2 like 2 (MAD2L2) (ENSG00000116670.14), angiotensin II receptor associated protein (AGTRAP) (ENSG00000177674.15), NBL1, DAN family BMP antagonist (NBL1) (ENSG00000158747.14), zinc finger DHHC-type palmitoyltransferase 18 (ZDHHC18) (ENSG00000204160.11), solute carrier family 9 member A1 (SLC9A1) (ENSG00000090020.10), transmembrane protein 222 (TMEM222) (ENSG00000186501.14), platelet activating factor receptor (PTAFR) (ENSG00000169403.11), penta-EF-hand domain containing 1 (PEF1) (ENSG00000162517.12), NHS like 3 (KIAA1522) (ENSG00000162522.10), chromosome 1 open reading frame 122 (C1orf122) (ENSG00000197982.13), bone morphogenetic protein 8a (BMP8A) (ENSG00000183682.7), importin 13 (IPO13) (ENSG00000117408.10), diphthamide biosynthesis 2 (DPH2) (ENSG00000132768.13), ATPase H+ transporting V0 subunit b (ATP6V0B) (ENSG00000117410.13), aldo-keto reductase family 1 member A1 (AKR1A1) (ENSG00000117448.13), leucine rich repeat containing 41 (LRRC41) (ENSG00000132128.16), PDZK1 interacting protein 1 (PDZK1IP1) (ENSG00000162366.7), 24-dehydrocholesterol reductase (DHCR24) (ENSG00000116133.11), CD53 molecule (CD53) (ENSG00000143119.12), WD repeat domain 77 (WDR77) (ENSG00000116455.13), peroxisomal biogenesis factor 11 beta (PEX11B) (ENSG00000131779.10), aph-1 homolog A, gamma-secretase subunit (APH1A) (ENSG00000117362.12), proteasome 20S subunit beta 4 (PSMB4) (ENSG00000159377.10), S100 calcium binding protein A2 (S100A2) (ENSG00000196754.10), jumping translocation breakpoint (JTB) (ENSG00000143543.14), RUN and SH3 domain containing 1 (RUSC1) (ENSG00000160753.15), methyltransferase like 25B (RRNAD1) (ENSG00000143303.11), SLAM family member 8 (SLAMF8) (ENSG00000158714.10), protoporphyrinogen oxidase (PPOX) (ENSG00000143224.17), uridine-cytidine kinase 2 (UCK2) (ENSG00000143179.15), SFT2 domain containing 2 (SFT2D2) (ENSG00000213064.9), transmembrane protein 9 (TMEM9) (ENSG00000116857.16), NUAK family kinase 2 (NUAK2) (ENSG00000163545.8), left-right determination factor 1 (LEFTY1) (ENSG00000243709.1), jumonji domain containing 4 (JMJD4) (ENSG00000081692.12), solute carrier family 35 member F6 (SLC35F6) (ENSG00000213699.8), solute carrier family 5 member 6 (SLC5A6) (ENSG00000138074.14), sorting nexin 17 (SNX17) (ENSG00000115234.10), testis expressed 261 (TEX261) (ENSG00000144043.11), SMYD family member 5 (SMYD5) (ENSG00000135632.11), coiled-coil domain containing 142 (CCDC142) (ENSG00000135637.13), ssemaphorin 4F (SEMA4F) (ENSG00000135622.12), trans-golgi network protein 2 (TGOLN2) (ENSG00000152291.13), transmembrane protein 127 (TMEM127) (ENSG00000135956.8), mitotic spindle organizing protein 2B (MZT2B) (ENSG00000152082.13), Rho guanine nucleotide exchange factor 4 (ARHGEF4) (ENSG00000136002.18), COP9 signalosome subunit 7B (COPS7B) (ENSG00000144524.17), hes family bHLH transcription factor 6 (HES6) (ENSG00000144485.10), G protein-coupled receptor 35 (GPR35) (ENSG00000178623.11), actin related protein 2/3 complex subunit 4 (ARPC4) (ENSG00000241553.12), jagunal homolog 1 (JAGN1) (ENSG00000171135.13), interleukin 17 receptor C (IL17RC) (ENSG00000163702.18), transmembrane protein with metallophosphoesterase domain (TMPPE) (ENSG00000188167.8), ribosomal protein SA (RPSA) (ENSG00000168028.13), ubiquinol-cytochrome c reductase core protein 1 (UQCRC1) (ENSG00000010256.10), DALR anticodon binding domain containing 3 (DALRD3) (ENSG00000178149.16), cytochrome b561 family member D2 (CYB561D2) (ENSG00000114395.10), POC1 centriolar protein A (POC1A) (ENSG00000164087.7), solute carrier family 41 member 3 (SLC41A3) (ENSG00000114544.16), transmembrane protein adipocyte associated 1 (TPRA1) (ENSG00000163870.14), ALG3 alpha-1,3-mannosyltransferase (ALG3) (ENSG00000214160.9), EEF1A lysine methyltransferase 4 (EEF1AKMT4) (ENSG00000284753.1), major facilitator superfamily domain containing 10 (MFSD10) (ENSG00000109736.14), interferon regulatory factor 2 (IRF2) (ENSG00000168310.10), solute carrier family 35 member A4 (SLC35A4) (ENSG00000176087.14), NADH:ubiquinone oxidoreductase subunit A2 (NDUFA2) (ENSG00000131495.8), solute carrier family 36 member 1 (SLC36A1) (ENSG00000123643.12), antioxidant 1 copper chaperone (ATOX1) (ENSG00000177556.11), ADP ribosylation factor like GTPase 10 (ARL10) (ENSG00000175414.6), beta-1,4-galactosyltransferase 7 (B4GALT7) (ENSG00000027847.13), 5-phosphohydroxy-L-lysine phospho-lyase (PHYKPL) (ENSG00000175309.14), H3 clustered histone 2 (HIST1H3B) (ENSG00000274267.1), H1.2 linker histone, cluster member (HIST1H1C) (ENSG00000187837.3), H3 clustered histone 10 (HIST1H3H) (ENSG00000278828.1), olfactory receptor family 2 subfamily I member 1 pseudogene (OR2I1P) (ENSG00000237988.5), RNA polymerase I subunit H (ZNRD1) (ENSG00000066379.14), splicing factor 3b subunit 5 (SF3B5) (ENSG00000169976.6), aquaporin 1 (Colton blood group) (AQP1) (ENSG00000240583.11), receptor activity modifying protein 3 (RAMP3) (ENSG00000122679.8), NOP2/Sun RNA methyltransferase 5 (NSUN5) (ENSG00000130305.16), elastin (ELN) (ENSG00000049540.16), zona pellucida glycoprotein 3 (ZP3) (ENSG00000188372.14), cytochrome P450 family 51 subfamily A member 1 (CYP51A1) (ENSG00000001630.15), solute carrier family 12 member 9 (SLC12A9) (ENSG00000146828.17), monoacylglycerol O-acyltransferase 3 (MOGAT3) (ENSG00000106384.10), RNA polymerase II subunit J2 (POLR2J2) (ENSG00000228049.7), smoothened, frizzled class receptor (SMO) (ENSG00000128602.9), cell cycle regulator of NHEJ (C7orf49) (ENSG00000122783.16), MT-RNR2 like 6 (pseudogene) (MTRNR2L6) (ENSG00000270672.1), Fas activated serine/threonine kinase (FASTK) (ENSG00000164896.19), heparan-alpha-glucosaminide N-acetyltransferase (HGSNAT) (ENSG00000165102.14), grainyhead like transcription factor 2 (GRHL2) (ENSG00000083307.11), F-box and leucine rich repeat protein 6 (FBXL6) (ENSG00000182325.10), ribonuclease P/MRP subunit p25 like (RPP25L) (ENSG00000164967.9), phosphatidylinositol glycan anchor biosynthesis class O (PIGO) (ENSG00000165282.13), haloacid dehalogenase like hydrolase domain containing 3 (HDHD3) (ENSG00000119431.9), WD repeat domain 38 (WDR38) (ENSG00000136918.7), solute carrier family 2 member 8 (SLC2A8) (ENSG00000136856.17), torsin family 2 member A (TOR2A) (ENSG00000160404.17), ST6 N-acetylgalactosaminide alpha-2,6-sialyltransferase 6 (ST6GALNAC6) (ENSG00000160408.14), zinc finger DHHC-type palmitoyltransferase 12 (ZDHHC12) (ENSG00000160446.18), TBC1 domain family member 13 (TBC1D13) (ENSG00000107021.15), protein phosphatase 2 phosphatase activator (PPP2R4) (ENSG00000119383.19), calcium channel flower domain containing 1 (CACFD1) (ENSG00000160325.14), transmembrane protein 141 (TMEM141) (ENSG00000244187.7), transmembrane protein 203 (TMEM203) (ENSG00000187713.6), trypsin like peroxisomal matrix peptidase 1 (TYSND1) (ENSG00000156521.13), leucine rich repeat containing 20 (LRRC20) (ENSG00000172731.13), V-set immunoregulatory receptor (VSIR) (ENSG00000107738.19), SEC24 homolog C, COPII coat complex component (SEC24C) (ENSG00000176986.15), tectonic family member 3 (TCTN3) (ENSG00000119977.20), phosphatidylinositol 4-kinase type 2 alpha (PI4K2A) (ENSG00000155252.13), F-box and WD repeat domain containing 4 (FBXW4) (ENSG00000107829.13), glutathione S-transferase omega 1 (GSTO1) (ENSG00000148834.12), Bet1 golgi vesicular membrane trafficking protein like (BET1L) (ENSG00000177951.17), tetraspanin 4 (TSPAN4) (ENSG00000214063.10), chitinase domain containing 1 (CHID1) (ENSG00000177830.17), tumor suppressing subtransferable candidate 4 (TSSC4) (ENSG00000184281.14), FHF complex subunit HOOK interacting protein 1B (FAM160A2) (ENSG00000051009.10), peroxisomal biogenesis factor 16 (PEX16) (ENSG00000121680.15), solute carrier family 39 member 13 (SLC39A13) (ENSG00000165915.13), cytochrome b561 family member A3 (CYB561A3) (ENSG00000162144.9), transmembrane protein 258 (TMEM258) (ENSG00000134825.15), bestrophin 1 (BEST1) (ENSG00000167995.15), beta-1,3-glucuronyltransferase 3 (B3GAT3) (ENSG00000149541.9), integrator complex subunit 5 (INTS5) (ENSG00000185085.2), cytochrome c oxidase subunit 8A (COX8A) (ENSG00000176340.3), sorting nexin 32 (SNX32) (ENSG00000172803.17), frizzled class receptor 4 (FZD4) (ENSG00000174804.3), solute carrier family 37 member 4 (SLC37A4) (ENSG00000137700.17), NLR family member X1 (NLRX1) (ENSG00000160703.15), F-box and leucine rich repeat protein 14 (FBXL14) (ENSG00000171823.6), non-SMC condensin I complex subunit D2 (NCAPD2) (ENSG00000010292.12), myeloid leukemia factor 2 (MLF2) (ENSG00000089693.10), complement C3a receptor 1 (C3AR1) (ENSG00000171860.4), G protein-coupled receptor class C group 5 member A (GPRC5A) (ENSG00000013588.7), transmembrane protein 106C (TMEM106C) (ENSG00000134291.11), tubulin alpha 1b (TUBA1B) (ENSG00000123416.15), major facilitator superfamily domain containing 5 (MFSD5) (ENSG00000182544.8), ATP synthase membrane subunit C locus 2 (ATP5G2) (ENSG00000135390.17), retinol dehydrogenase 5 (RDH5) (ENSG00000135437.9), premelanosome protein (PMEL) (ENSG00000185664.14), cyclin dependent kinase 4 (CDK4) (ENSG00000135446.16), CTD small phosphatase 2 (CTDSP2) (ENSG00000175215.10), polypeptide N-acetylgalactosaminyltransferase 4 (GALNT4) (ENSG00000257594.3), phospholipase B domain containing 2 (PLBD2) (ENSG00000151176.7), paxillin (PXN) (ENSG00000089159.16), refilin A (FAM101A) (ENSG00000178882.14), transmembrane protein 253 (TMEM253) (ENSG00000232070.8), abhydrolase domain containing 4, N-acyl phospholipase B (ABHD4) (ENSG00000100439.10), proteasome 20S subunit beta 5 (PSMB5) (ENSG00000100804.18), phosphoenolpyruvate carboxykinase 2, mitochondrial (PCK2) (ENSG00000100889.11), ER membrane protein complex subunit 9 (EMC9) (ENSG00000100908.13), ribosomal protein S29 (RPS29) (ENSG00000213741.10), sushi domain containing 6 (SUSD6) (ENSG00000100647.7), acyl-CoA thioesterase 1 (ACOT1) (ENSG00000184227.7), acyl-CoA thioesterase 4 (ACOT4) (ENSG00000177465.4), ATP binding cassette subfamily D member 4 (ABCD4) (ENSG00000119688.20), NPC intracellular cholesterol transporter 2 (NPC2) (ENSG00000119655.10), angel homolog 1 (ANGEL1) (ENSG00000013523.9), CLOCK interacting pacemaker (CIPC) (ENSG00000198894.7), G protein-coupled receptor 68 (GPR68) (ENSG00000119714.10), solute carrier family 25 member 29 (SLC25A29) (ENSG00000197119.12), SIVA1 apoptosis inducing factor (SIVA1) (ENSG00000184990.12), G protein-coupled receptor 176 (GPR176) (ENSG00000166073.10), sorting nexin 22 (SNX22) (ENSG00000157734.13), pleckstrin homology domain containing O2 (PLEKHO2) (ENSG00000241839.9), cartilage intermediate layer protein (CILP) (ENSG00000138615.5), stomatin like 1 (STOML1) (ENSG00000067221.13), COMM domain containing 4 (COMMD4) (ENSG00000140365.15), phosphatidylinositol glycan anchor biosynthesis class Q (PIGQ) (ENSG00000007541.16), transmembrane protein 204 (TMEM204) (ENSG00000131634.13), NME/NM23 nucleoside diphosphate kinase 3 (NME3) (ENSG00000103024.7), NUBP iron-sulfur cluster assembly factor 2, cytosolic (NUBP2) (ENSG00000095906.16), presequence translocase associated motor 16 (PAM16) (ENSG00000217930.7), coronin 7 (CORO7) (ENSG00000262246.5), PAXIP1 associated glutamate rich protein 1 (PAGR1) (ENSG00000280789.1), CDP-diacylglycerol-inositol 3-phosphatidyltransferase (CDIPT) (ENSG00000103502.13), phosphorylase kinase catalytic subunit gamma 2 (PHKG2) (ENSG00000156873.15), ORAI calcium release-activated calcium modulator 3 (ORAI3) (ENSG00000175938.6), serine protease 8 (PRSS8) (ENSG00000052344.15), adhesion G protein-coupled receptor G1 (ADGRG1) (ENSG00000205336.11), chemokine like factor (CKLF) (ENSG00000217555.12), CKLF like MARVEL transmembrane domain containing 3 (CMTM3) (ENSG00000140931.19), transmembrane protein 208 (TMEM208) (ENSG00000168701.18), THAP domain containing 11 (THAP11) (ENSG00000168286.2), proteasome 20S subunit beta 10 (PSMB10) (ENSG00000205220.11), Tax1 binding protein 3 (TAX1BP3) (ENSG00000213977.7), GABA type A receptor-associated protein (GABARAP) (ENSG00000170296.9), transient receptor potential cation channel subfamily V member 2 (TRPV2) (ENSG00000187688.14), N-acetyltransferase domain containing 1 (NATD1) (ENSG00000274180.1), DNA polymerase delta interacting protein 2 (POLDIP2) (ENSG00000004142.11), notchless homolog 1 (NLE1) (ENSG00000073536.17), NFKB inhibitor interacting Ras like 2 (NKIRAS2) (ENSG00000168256.17), N-acetyl-alpha-glucosaminidase (NAGLU) (ENSG00000108784.9), reticulophagy regulator family member 3 (FAM134C) (ENSG00000141699.10), glucose-6-phosphatase catalytic subunit 3 (G6PC3) (ENSG00000141349.8), intercellular adhesion molecule 2 (ICAM2) (ENSG00000108622.10), G protein-coupled receptor class C group 5 member C (GPRC5C) (ENSG00000170412.16), thymidine kinase 1 (TK1) (ENSG00000167900.11), actin gamma 1 (ACTG1) (ENSG00000184009.10), FA core complex associated protein 100 (FAAP100) (ENSG00000185504.16), solute carrier family 25 member 10 (SLC25A10) (ENSG00000183048.11), phospholipid phosphatase 2 (PLPP2) (ENSG00000141934.9), secretory carrier membrane protein 4 (SCAMP4) (ENSG00000227500.9), MOB kinase activator 3A (MOB3A) (ENSG00000172081.13), fucosyltransferase 6 (FUT6) (ENSG00000156413.13), NADH:ubiquinone oxidoreductase subunit A11 (NDUFA11) (ENSG00000174886.12), calcyphosine (CAPS) (ENSG00000105519.15), PET100 cytochrome c oxidase chaperone (C19orf79) (ENSG00000229833.9), CD320 molecule (CD320) (ENSG00000167775.10), sphingosine-1-phosphate receptor 2 (S1PR2) (ENSG00000267534.3), autophagy related 4D cysteine peptidase (ATG4D) (ENSG00000130734.9), phospholipid phosphatase related 2 (PLPPR2) (ENSG00000105520.10), WD repeat domain 83 opposite strand (WDR83OS) (ENSG00000105583.9), notch receptor 3 (NOTCH3) (ENSG00000074181.8), adaptor related protein complex 1 subunit mu 1 (AP1M1) (ENSG00000072958.8), ubiquitin A-52 residue ribosomal protein fusion product 1 (UBA52) (ENSG00000221983.7), kelch like family member 26 (KLHL26) (ENSG00000167487.11), armadillo repeat containing 6 (ARMC6) (ENSG00000105676.13), transmembrane protein 161A (TMEM161A) (ENSG00000064545.14), nuclear receptor 2C2 associated protein (NR2C2AP) (ENSG00000184162.14), MAU2 sister chromatid cohesion factor (MAU2) (ENSG00000129933.20), transmembrane protein 147 (TMEM147) (ENSG00000105677.11), presenilin enhancer, gamma-secretase subunit (PSENEN) (ENSG00000205155.7), cytochrome c oxidase subunit 7A1 (COX7A1) (ENSG00000161281.10), Charcot-Leyden crystal galectin (CLC) (ENSG00000105205.6), zinc finger protein 574 (ZNF574) (ENSG00000105732.12), ETHE1 persulfide dioxygenase (ETHE1) (ENSG00000105755.7), adaptor related protein complex 2 subunit sigma 1 (AP2S1) (ENSG00000042753.11), transient receptor potential cation channel subfamily M member 4 (TRPM4) (ENSG00000130529.15), BCL2 like 12 (BCL2L12) (ENSG00000126453.9), sialic acid binding Ig like lectin 14 (SIGLEC14) (ENSG00000254415.3), formyl peptide receptor 1 (FPR1) (ENSG00000171051.8), protein phosphatase 2 scaffold subunit Aalpha (PPP2R1A) (ENSG00000105568.17), zinc finger protein 320 (ZNF320) (ENSG00000182986.13), ribosomal protein S5 (RPS5) (ENSG00000083845.8), small nuclear ribonucleoprotein polypeptides B and B1 (SNRPB) (ENSG00000125835.18), U-box domain containing 5 (UBOX5) (ENSG00000185019.16), acyl-CoA synthetase short chain family member 1 (ACSS1) (ENSG00000154930.14), glycogen phosphorylase B (PYGB) (ENSG00000100994.11), AAR2 splicing factor (AAR2) (ENSG00000131043.11), acyl-CoA thioesterase 8 (ACOT8) (ENSG00000101473.16), phosphorylated CTD interacting factor 1 (PCIF1) (ENSG00000100982.11), gamma-glutamyltransferase 5 (GGT5) (ENSG00000099998.17), rhomboid domain containing 3 (RHBDD3) (ENSG00000100263.13), galectin 1 (LGALS1) (ENSG00000100097.11), platelet derived growth factor subunit B (PDGFB) (ENSG00000100311.16), centromere protein M (CENPM) (ENSG00000100162.14), translocator protein (TSPO) (ENSG00000100300.17), gem nuclear organelle associated protein 8 (GEMIN8) (ENSG00000046647.13), NADH:ubiquinone oxidoreductase subunit B11 (NDUFB11) (ENSG00000147123.10), ribosomal protein L10 (RPL10) (ENSG00000147403.16), L antigen family member 3 (LAGE3) (ENSG00000196976.7), AC004556.1 (ENSG00000276345.1), solute carrier family 35 member E2B (SLC35E2B) (ENSG00000189339.11), aldo-keto reductase family 7 member A3 (AKR7A3) (ENSG00000162482.4), keratinocyte differentiation factor 1 (KDF1) (ENSG00000175707.8), serine incorporator 2 (SERINC2) (ENSG00000168528.11), transmembrane protein 54 (TMEM54) (ENSG00000121900.18), polyhomeotic homolog 2 (PHC2) (ENSG00000134686.18), solute carrier family 44 member 3 (SLC44A3) (ENSG00000143036.16), glutathione S-transferase mu 3 (GSTM3) (ENSG00000134202.10), H3 clustered histone 13 (HIST2H3D) (ENSG00000183598.3), atypical chemokine receptor 1 (Duffy blood group) (ACKR1) (ENSG00000213088.10), arginyl aminopeptidase (RNPEP) (ENSG00000176393.10), major facilitator superfamily domain containing 4A (MFSD4A) (ENSG00000174514.12), calpain 13 (CAPN13) (ENSG00000162949.16), protein kinase domain containing, cytoplasmic (PKDCC) (ENSG00000162878.12), C-X-C motif chemokine receptor 4 (CXCR4) (ENSG00000121966.6), cytochrome P450 family 27 subfamily A member 1 (CYP27A1) (ENSG00000135929.8), F-box and leucine rich repeat protein 2 (FBXL2) (ENSG00000153558.14), transmembrane protein 129, E3 ubiquitin ligase (TMEM129) (ENSG00000168936.10), family with sequence similarity 53 member C (FAM53C) (ENSG00000120709.10), stimulator of interferon response CGAMP interactor 1 (TMEM173) (ENSG00000184584.12), CD14 molecule (CD14) (ENSG00000170458.13), protocadherin 1 (PCDH1) (ENSG00000156453.13), SH3 domain and tetratricopeptide repeats 2 (SH3TC2) (ENSG00000169247.12), alpha-1,3-mannosyl-glycoprotein 4-beta-N-acetylglucosaminyltransferase B (MGAT4B) (ENSG00000161013.16), sequestosome 1 (SQSTM1) (ENSG00000161011.19), abhydrolase domain containing 16A, phospholipase (ABHD16A) (ENSG00000204427.11), lymphocyte antigen 6 family member G6D (LY6G6D) (ENSG00000244355.7), peptidase inhibitor 16 (PI16) (ENSG00000164530.13), MAD2L1 binding protein (MAD2L1BP) (ENSG00000124688.13), translocation associated membrane protein 2 (TRAM2) (ENSG00000065308.4), rhomboid domain containing 2 (RHBDD2) (ENSG00000005486.16), serpin family E member 1 (SERPINE1) (ENSG00000106366.8), zinc finger C3HC-type containing 1 (ZC3HC1) (ENSG00000091732.15), thromboxane A synthase 1 (TBXAS1) (ENSG00000059377.16), protein O-mannose kinase (POMK) (ENSG00000185900.9), glycosylphosphatidylinositol anchor attachment 1 (GPAA1) (ENSG00000197858.10), zinc finger FYVE-type containing 27 (ZFYVE27) (ENSG00000155256.17), mucin 6, oligomeric mucus/gel-forming (MUC6) (ENSG00000184956.15), Rho GTPase activating protein 1 (ARHGAP1) (ENSG00000175220.11), diacylglycerol lipase alpha (DAGLA) (ENSG00000134780.9), ADP ribosylation factor like GTPase 2 (ARL2) (ENSG00000213465.7), triosephosphate isomerase 1 (TPI1) (ENSG00000111669.14), chromosome 12 open reading frame 57 (C12orf57) (ENSG00000111678.10), EMG1 N1-specific pseudouridine methyltransferase (EMG1) (ENSG00000126749.14), solute carrier family 48 member 1 (SLC48A1) (ENSG00000211584.13), transmembrane BAX inhibitor motif containing 6 (TMBIM6) (ENSG00000139644.12), keratin 6B (KRT6B) (ENSG00000185479.5), OXA1L mitochondrial inner membrane protein (OXA1L) (ENSG00000155463.12), NEDD8 ubiquitin like modifier (NEDD8) (ENSG00000129559.12), coenzyme Q6, monooxygenase (COQ6) (ENSG00000119723.16), kelch like family member 25 (KLHL25) (ENSG00000183655.12), ATP binding cassette subfamily A member 3 (ABCA3) (ENSG00000167972.13), serine protease 21 (PRSS21) (ENSG00000007038.10), ATP binding cassette subfamily C member 6 (ABCC6) (ENSG00000091262.15), coronin 1A (CORO1A) (ENSG00000102879.15), thymidine kinase 2 (TK2) (ENSG00000166548.15), ring finger protein 135 (RNF135) (ENSG00000181481.13), TBC1 domain family member 3L (TBC1D3L) (ENSG00000274512.5), GH3 domain containing (GHDC) (ENSG00000167925.15), homeobox B13 (HOXB13) (ENSG00000159184.7), RNA polymerase II, I and III subunit E (POLR2E) (ENSG00000099817.11), methyl-CpG binding domain protein 3 (MBD3) (ENSG00000071655.17), ubiquinol-cytochrome c reductase, complex III subunit XI (UQCR11) (ENSG00000127540.11), vimentin type intermediate filament associated coiled-coil protein (VMAC) (ENSG00000187650.3), kelch like ECH associated protein 1 (KEAP1) (ENSG00000079999.13), adaptor related protein complex 1 subunit mu 2 (AP1M2) (ENSG00000129354.11), transmembrane p24 trafficking protein 1 (TMED1) (ENSG00000099203.6), BRISC and BRCA1 A complex member 1 (BABAM1) (ENSG00000105393.15), collagen beta (1-O) galactosyltransferase 1 (GLT25D1) (ENSG00000130309.10), zinc finger protein 132 (ZNF132) (ENSG00000131849.11), ectonucleoside triphosphate diphosphohydrolase 6 (ENTPD6) (ENSG00000197586.12), MT-RNR2 like 3 (pseudogene) (MTRNR2L3) (ENSG00000256222.2), solute carrier family 19 member 1 (SLC19A1) (ENSG00000173638.18), claudin 5 (CLDN5) (ENSG00000184113.9), phosphoinositide-3-kinase interacting protein 1 (PIK3IP1) (ENSG00000100100.12), non-SMC condensin II complex subunit H2 (NCAPH2) (ENSG00000025770.18), coiled-coil domain containing 22 (CCDC22) (ENSG00000101997.12), ubiquilin 2 (UBQLN2) (ENSG00000188021.8), transmembrane protein 164 (TMEM164) (ENSG00000157600.11).

20. The cancer subtype identifier according any of the preceding embodiments, wherein for the identification of MXE2 subtype the expression of the predictive protein-coding genes is characterized by their up-regulation in the MXE2 subtype as compared to the MXE1 subtype of primary group selected from TAR DNA binding protein (TARDBP) (ENSG00000120948.16), heterogeneous nuclear ribonucleoprotein C like 4 (HNRNPCL4) (ENSG00000179412.10), capping actin protein of muscle Z-line subunit beta (CAPZB) (ENSG00000077549.17), heterogeneous nuclear ribonucleoprotein R (HNRNPR) (ENSG00000125944.19), single stranded DNA binding protein 3 (SSBP3) (ENSG00000157216.15), nexilin F-actin binding protein (NEXN) (ENSG00000162614.18), far upstream element binding protein 1 (FUBP1) (ENSG00000162613.16), cellular communication network factor 1 (CYR61) (ENSG00000142871.16), SET like protein (SETSIP) (ENSG00000230667.5), major facilitator superfamily domain containing 14A (MFSD14A) (ENSG00000156875.13), SAS-6 centriolar assembly protein (SASS6) (ENSG00000156876.9), pre-mRNA processing factor 38B (PRPF38B) (ENSG00000134186.11), TATA-box binding protein associated factor 13 (TAF13) (ENSG00000197780.9), RAP1A, member of RAS oncogene family (RAP1A) (ENSG00000116473.14), H2A clustered histone 19 (HIST2H2AA4) (ENSG00000272196.2), H3 clustered histone 15 (HIST2H3A) (ENSG00000203852.3), H4 clustered histone 15 (HIST2H4B) (ENSG00000270276.2), acidic nuclear phosphoprotein 32 family member E (ANP32E) (ENSG00000143401.14), S100 calcium binding protein A10 (S100A10) (ENSG00000197747.8), regulator of G protein signaling 13 (RGS13) (ENSG00000127074.14), ribosomal protein S27a pseudogene 5 (RP11-92K2.2) (ENSG00000231767.4), ubiquitin C-terminal hydrolase L5 (UCHL5) (ENSG00000116750.13), LIM homeobox 9 (LHX9) (ENSG00000143355.15), zinc finger BED-type containing 6 (ZBED6) (ENSG00000257315.2), zinc finger CCCH-type containing 11B (ZC3H11B) (ENSG00000215817.5), ras homolog family member U (RHOU) (ENSG00000116574.5), translin associated factor X (TSNAX) (ENSG00000116918.13), myelin transcription factor 1 like (MYT1L) (ENSG00000186487.18), protein phosphatase 1 catalytic subunit beta (PPP1CB) (ENSG00000213639.9), protein phosphatase, Mg2+/Mn2+ dependent 1B (PPM1B) (ENSG00000138032.20), C1D nuclear receptor corepressor (C1D) (ENSG00000197223.11), poly(rC) binding protein 1 (PCBP1) (ENSG00000169564.6), RANBP2 like and GRIP domain containing 1 (RGPD1) (ENSG00000187627.15), RANBP2 like and GRIP domain containing 2 (RGPD2) (ENSG00000185304.14), RANBP2 like and GRIP domain containing 3 (RGPD3) (ENSG00000153165.18), RANBP2 like and GRIP domain containing 4 (RGPD4) (ENSG00000196862.9), GRIP and coiled-coil domain containing 2 (GCC2) (ENSG00000135968.20), RANBP2 like and GRIP domain containing 5 (RGPD5) (ENSG00000015568.12), RANBP2 like and GRIP domain containing 6 (RGPD6) (ENSG00000183054.11), diazepam binding inhibitor, acyl-CoA binding protein (DBI) (ENSG00000155368.16), RAB6C, member RAS oncogene family (RAB6C) (ENSG00000222014.5), POTE ankyrin domain family member I (POTEI) (ENSG00000196834.11), POTE ankyrin domain family member J (POTEJ) (ENSG00000222038.3), UBX domain protein 4 (UBXN4) (ENSG00000144224.16), ADP ribosylation factor like GTPase 5A (ARL5A) (ENSG00000162980.16), membrane associated ring-CH-type finger 7 (MARCH7) (ENSG00000136536.14), peptidylprolyl isomerase G (PPIG) (ENSG00000138398.15), small RNA binding exonuclease protection factor La (SSB) (ENSG00000138385.15), tousled like kinase 1 (TLK1) (ENSG00000198586.13), corepressor interacting with RBPJ, CIR1 (CIR1) (ENSG00000138433.15), heterogeneous nuclear ribonucleoprotein A3 (HNRNPA3) (ENSG00000170144.20), MOB family member 4, phocein (MOB4) (ENSG00000115540.14), tubulin alpha 4b (TUBA4B) (ENSG00000243910.7), prothymosin alpha (PTMA) (ENSG00000187514.16), myeloma overexpressed 2 pseudogene (MYEOV2) (ENSG00000172428.10), synapsin II (SYN2) (ENSG00000157152.16), LSM3 homolog, U6 small nuclear RNA and mRNA degradation associated (LSM3) (ENSG00000170860.3), protein kinase C delta (PRKCD) (ENSG00000163932.13), MT-RNR2 like 12 (pseudogene) (MTRNR2L12) (ENSG00000269028.3), filamin A interacting protein 1 like (FILIP1L) (ENSG00000168386.18), PEST proteolytic signal containing nuclear protein (PCNP) (ENSG00000081154.11), RAB7A, member RAS oncogene family (RAB7A) (ENSG00000075785.12), WW domain containing transcription regulator 1 (WWTR1) (ENSG00000018408.14), structural maintenance of chromosomes 4 (SMC4) (ENSG00000113810.15), SKI like proto-oncogene (SKIL) (ENSG00000136603.13), cytoplasmic polyadenylation element binding protein 2 (CPEB2) (ENSG00000137449.15), MOB kinase activator 1B (MOB1B) (ENSG00000173542.8), Ras association domain family member 6 (RASSF6) (ENSG00000169435.13), heterogeneous nuclear ribonucleoprotein D like (HNRNPDL) (ENSG00000152795.17), DnaJ heat shock protein family (Hsp40) member B14 (DNAJB14) (ENSG00000164031.16), family with sequence similarity 241 member A (C4orf32) (ENSG00000174749.5), La ribonucleoprotein 7, transcriptional regulator (LARP7) (ENSG00000174720.15), progesterone receptor membrane component 2 (PGRMC2) (ENSG00000164040.16), tryptophan 2,3-dioxygenase (TDO2) (ENSG00000151790.8), palladin, cytoskeletal associated protein (PALLD) (ENSG00000129116.17), 15-hydroxyprostaglandin dehydrogenase (HPGD) (ENSG00000164120.13), inhibitor of growth family member 2 (ING2) (ENSG00000168556.6), cilia and flagella associated protein 97 (CFAP97) (ENSG00000164323.12), NADH:ubiquinone oxidoreductase subunit S6 (NDUFS6) (ENSG00000145494.11), SUB1 regulator of transcription (SUB1) (ENSG00000113387.11), NADH:ubiquinone oxidoreductase complex assembly factor 2 (NDUFAF2) (ENSG00000164182.10), CWC27 spliceosome associated cyclophilin (CWC27) (ENSG00000153015.15), protein geranylgeranyltransferase type I subunit beta (PGGT1B) (ENSG00000164219.9), coiled-coil domain containing 112 (CCDC112) (ENSG00000164221.12), lysyl oxidase (LOX) (ENSG00000113083.13), ubiquitin conjugating enzyme E2 B (UBE2B) (ENSG00000119048.7), RNA binding motif protein 27 (RBM27) (ENSG00000091009.7), H4 clustered histone 11 (HIST1H4J) (ENSG00000197238.4), H4 clustered histone 12 (HIST1H4K) (ENSG00000273542.1), CD2 associated protein (CD2AP) (ENSG00000198087.7), KIAA1586 (KIAA1586) (ENSG00000168116.13), FKBP prolyl isomerase family member 1C (FKBP1C) (ENSG00000198225.5), high mobility group nucleosomal binding domain 3 (HMGN3) (ENSG00000118418.14), SEC63 homolog, protein translocation regulator (SEC63) (ENSG00000025796.13), myristoylated alanine rich protein kinase C substrate (MARCKS) (ENSG00000277443.2), RWD domain containing 1 (RWDD1) (ENSG00000111832.12), NUS1 dehydrodolichyl diphosphate synthase subunit (NUS1) (ENSG00000153989.7), cellular communication network factor 2 (CTGF) (ENSG00000118523.5), BCL2 associated transcription factor 1 (BCLAF1) (ENSG00000029363.16), LTV1 ribosome biogenesis factor (LTV1) (ENSG00000135521.8), TGF-beta activated kinase 1 (MAP3K7) binding protein 2 (TAB2) (ENSG00000055208.18), peptidylprolyl isomerase like 4 (PPIL4) (ENSG00000131013.3), RB associated KRAB zinc finger (RBAK) (ENSG00000146587.17), transmembrane protein 106B (TMEM106B) (ENSG00000106460.18), aryl hydrocarbon receptor (AHR) (ENSG00000106546.13), transformer 2 alpha homolog (TRA2A) (ENSG00000164548.10), sperm microtubule inner protein 4 (C7orf31) (ENSG00000153790.11), heterogeneous nuclear ribonucleoprotein A2/B1 (HNRNPA2B1) (ENSG00000122566.20), zinc and ring finger 2 (ZNRF2) (ENSG00000180233.10), ITPR interacting domain containing 1 (CCDC129) (ENSG00000180347.13), POU class 6 homeobox 2 (POU6F2) (ENSG00000106536.19), RAS like proto-oncogene A (RALA) (ENSG00000006451.7), inhibin subunit beta A (INHBA) (ENSG00000122641.10), zinc finger protein 680 (ZNF680) (ENSG00000173041.11), fibrinogen like 2 (FGL2) (ENSG00000127951.6), A-kinase anchoring protein 9 (AKAP9) (ENSG00000127914.16), family with sequence similarity 133 member B (FAM133B) (ENSG00000234545.7), DnaJ heat shock protein family (Hsp40) member C2 (DNAJC2) (ENSG00000105821.14), NADH:ubiquinone oxidoreductase subunit A5 (NDUFA5) (ENSG00000128609.14), chromosome 7 open reading frame 73 (C7orf73) (ENSG00000243317.7), protein kinase AMP-activated non-catalytic subunit gamma 2 (PRKAG2) (ENSG00000106617.13), neurofilament medium chain (NEFM) (ENSG00000104722.13), dynactin subunit 6 (DCTN6) (ENSG00000104671.7), CCAAT enhancer binding protein delta (CEBPD) (ENSG00000221869.4), syntrophin gamma 1 (SNTG1) (ENSG00000147481.15), RAB2A, member RAS oncogene family (RAB2A) (ENSG00000104388.14), translocation associated membrane protein 1 (TRAM1) (ENSG00000067167.7), zinc finger C2HC-type containing 1A (ZC2HC1A) (ENSG00000104427.11), tumor protein D52 (TPD52) (ENSG00000076554.15), ribosomal biogenesis factor (C8orf59) (ENSG00000176731.11), ubiquinol-cytochrome c reductase binding protein (UQCRB) (ENSG00000156467.9), RNA polymerase II, I and III subunit K (POLR2K) (ENSG00000147669.10), poly(A) binding protein cytoplasmic 1 (PABPC1) (ENSG00000070756.15), glycine decarboxylase (GLDC) (ENSG00000178445.9), zinc finger DHHC-type palmitoyltransferase 21 (ZDHHC21) (ENSG00000175893.11), PC4 and SRSF1 interacting protein 1 (PSIP1) (ENSG00000164985.14), caspase activity and apoptosis inhibitor 1 (CAAP1) (ENSG00000120159.11), TATA-box binding protein associated factor 1 like (TAF1L) (ENSG00000122728.6), carnosine N-methyltransferase 1 (CARNMT1) (ENSG00000156017.12), riboflavin kinase (RFK) (ENSG00000135002.11), RecQ mediated genome instability 1 (RMI1) (ENSG00000178966.16), acidic nuclear phosphoprotein 32 family member B (ANP32B) (ENSG00000136938.8), actin binding transcription modulator (FAM206A) (ENSG00000119328.11), TATA-box binding protein associated factor 3 (TAF3) (ENSG00000165632.7), chondroitin sulfate N-acetylgalactosaminyltransferase 2 (CSGALNACT2) (ENSG00000169826.7), ubiquitin conjugating enzyme E2 D1 (UBE2D1) (ENSG00000072401.14), VPS26 retromer complex component A (VPS26A) (ENSG00000122958.14), eukaryotic translation initiation factor 5A like 1 (EIF5AL1) (ENSG00000253626.3), kinesin family member 20B (KIF20B) (ENSG00000138182.14), FRA10A associated CGG repeat 1 (FRA10AC1) (ENSG00000148690.11), survival motor neuron domain containing 1 (SMNDC1) (ENSG00000119953.12), coiled-coil domain containing 186 (CCDC186) (ENSG00000165813.18), pleckstrin homology like domain family A member 2 (PHLDA2) (ENSG00000181649.6), MT-RNR2 like 8 (pseudogene) (MTRNR2L8) (ENSG00000255823.4), lin-7 homolog C, crumbs cell polarity complex component (LIN7C) (ENSG00000148943.11), glycine-N-acyltransferase like 1 (GLYATL1) (ENSG00000166840.13), membrane spanning 4-domains A6E (MS4A6E) (ENSG00000166926.8), heterogeneous nuclear ribonucleoprotein U like 2 (HNRNPUL2) (ENSG00000214753.2), FKBP prolyl isomerase 2 (FKBP2) (ENSG00000173486.12), spliceosome associated factor 1, recruiter of U4/U6.U5 tri-snRNP (SART1) (ENSG00000175467.14), remodeling and spacing factor 1 (RSF1) (ENSG00000048649.13), cysteine and histidine rich domain containing 1 (CHORDC1) (ENSG00000110172.11), ankyrin repeat domain 49 (ANKRD49) (ENSG00000168876.8), family with sequence similarity 76 member B (FAM76B) (ENSG00000077458.12), coiled-coil domain containing 82 (CCDC82) (ENSG00000149231.12), transmembrane protein 123 (TMEM123) (ENSG00000152558.14), defective in cullin neddylation 1 domain containing 5 (DCUN1D5) (ENSG00000137692.11), RNA binding motif protein 7 (RBM7) (ENSG00000076053.10), H2A.X variant histone (H2AFX) (ENSG00000188486.3), parathymosin (PTMS) (ENSG00000159335.15), basic helix-loop-helix family member e41 (BHLHE41) (ENSG00000123095.5), ERGIC and golgi 2 (ERGIC2) (ENSG00000087502.17), glucoside xylosyltransferase 1 (GXYLT1) (ENSG00000151233.10), zinc finger CCHC-type and RNA binding motif containing 1 (ZCRB1) (ENSG00000139168.7), twinfilin actin binding protein 1 (TWF1) (ENSG00000151239.13), activating transcription factor 1 (ATF1) (ENSG00000123268.8), oxysterol binding protein like 8 (OSBPL8) (ENSG00000091039.16), RNA 5′-phosphate and 3′-OH ligase 1 (C12orf29) (ENSG00000133641.17), centrosomal protein 290 (CEP290) (ENSG00000198707.14), early endosome antigen 1 (EEA1) (ENSG00000102189.16), small nuclear ribonucleoprotein polypeptide F (SNRPF) (ENSG00000139343.10), nuclear transcription factor Y subunit beta (NFYB) (ENSG00000120837.7), mitochondrial calcium uptake 2 (MICU2) (ENSG00000165487.13), poly(A) binding protein cytoplasmic 3 (PABPC3) (ENSG00000151846.8), casein kinase 1 alpha 1 like (CSNK1A1L) (ENSG00000180138.7), laccase domain containing 1 (LACC1) (ENSG00000179630.10), GPALPP motifs containing 1 (GPALPP1) (ENSG00000133114.17), tumor protein, translationally-controlled 1 (TPT1) (ENSG00000133112.16), zinc finger CCCH-type containing 13 (ZC3H13) (ENSG00000123200.16), integral membrane protein 2B (ITM2B) (ENSG00000136156.12), cytoskeleton associated protein 2 (CKAP2) (ENSG00000136108.14), mitotic spindle organizing protein 1 (MZT1) (ENSG00000204899.5), KLF transcription factor 5 (KLF5) (ENSG00000102554.13), potassium channel tetramerization domain containing 12 (KCTD12) (ENSG00000178695.5), coiled-coil domain containing 168 (CCDC168) (ENSG00000175820.3), arginine and glutamate rich 1 (ARGLU1) (ENSG00000134884.13), serine palmitoyltransferase small subunit A (SPTSSA) (ENSG00000165389.6), ras homolog family member J (RHOJ) (ENSG00000126785.12), golgin A6 family like 6 (GOLGA6L6) (ENSG00000277322.1), golgin A6 family like 2 (GOLGA6L2) (ENSG00000174450.11), ER membrane protein complex subunit 7 (EMC7) (ENSG00000134153.9), eukaryotic translation initiation factor 3 subunit J (EIF3J) (ENSG00000104131.12), COP9 signalosome subunit 2 (COPS2) (ENSG00000166200.14), RAB27A, member RAS oncogene family (RAB27A) (ENSG00000069974.15), SAFB like transcription modulator (SLTM) (ENSG00000137776.16), reticulocalbin 2 (RCN2) (ENSG00000117906.13), methenyltetrahydrofolate synthetase (MTHFS) (ENSG00000136371.10), zinc finger AN1-type containing 6 (ZFAND6) (ENSG00000086666.18), nuclear receptor subfamily 2 group F member 2 (NR2F2) (ENSG00000185551.14), centrosomal protein 20 (FOPNL) (ENSG00000133393.12), centriolar coiled-coil protein 110 (CCP110) (ENSG00000103540.16), nuclear pore complex interacting protein family member B5 (NPIPB5) (ENSG00000243716.10), eukaryotic translation initiation factor 3 subunit C like (EIF3CL) (ENSG00000205609.12), terminal nucleotidyltransferase 4B (PAPD5) (ENSG00000121274.12), tubulin polymerization promoting protein family member 3 (TPPP3) (ENSG00000159713.10), NIN1 (RPN12) binding protein 1 homolog (NOB1) (ENSG00000141101.12), contactin associated protein family member 4 (CNTNAP4) (ENSG00000152910.18), MAF bZIP transcription factor (MAF) (ENSG00000178573.6), CBFA2/RUNX1 partner transcriptional co-repressor 3 (CBFA2T3) (ENSG00000129993.14), ankyrin repeat domain containing 11 (ANKRD11) (ENSG00000167522.15), ER membrane protein complex subunit 6 (TMEM93) (ENSG00000127774.6), lysine demethylase 6B (KDM6B) (ENSG00000132510.10), ribosomal protein L26 (RPL26) (ENSG00000161970.13), SUZ12 polycomb repressive complex 2 subunit (SUZ12) (ENSG00000178691.10), insulin like growth factor binding protein 4 (IGFBP4) (ENSG00000141753.6), keratin 10 (KRT10) (ENSG00000186395.7), transducer of ERBB2, 1 (TOB1) (ENSG00000141232.4), nascent polypeptide associated complex subunit alpha 2 (NACA2) (ENSG00000253506.2), SRY-box transcription factor 9 (SOX9) (ENSG00000125398.6), ring finger protein 138 (RNF138) (ENSG00000134758.13), immediate early response 3 interacting protein 1 (IER3IP1) (ENSG00000134049.5), methyl-CpG binding domain protein 2 (MBD2) (ENSG00000134046.11), SEC11 homolog C, signal peptidase complex subunit (SEC11C) (ENSG00000166562.8), coiled-coil domain containing 102B (CCDC102B) (ENSG00000150636.17), zinc finger and BTB domain containing 7A (ZBTB7A) (ENSG00000178951.8), scaffold attachment factor B2 (SAFB2) (ENSG00000130254.11), scaffold attachment factor B (SAFB) (ENSG00000160633.12), tubulin beta 4A class IVa (TUBB4A) (ENSG00000104833.11), RAB11B, member RAS oncogene family (RAB11B) (ENSG00000185236.11), solute carrier family 1 member 6 (SLC1A6) (ENSG00000105143.12), KLF transcription factor 2 (KLF2) (ENSG00000127528.5), zinc finger protein 708 (ZNF708) (ENSG00000182141.10), PAF1 homolog, Paf1/RNA polymerase II complex component (PAF1) (ENSG00000006712.14), transforming growth factor beta 1 (TGFB1) (ENSG00000105329.9), novel protein, readthrough between CEACAM5-CEACAM6 (AC011513.3) (ENSG00000267881.1), CD177 molecule (CD177) (ENSG00000204936.9), potassium voltage-gated channel subfamily A member 7 (KCNA7) (ENSG00000104848.1), ribosomal protein L28 (RPL28) (ENSG00000108107.14), zinc finger protein 580 (ZNF580) (ENSG00000213015.8), ESF1 nucleolar pre-rRNA processing protein homolog (ESF1) (ENSG00000089048.14), charged multivesicular body protein 4B (CHMP4B) (ENSG00000101421.3), DNA topoisomerase I (TOP1) (ENSG00000198900.5), prefoldin subunit 4 (PFDN4) (ENSG00000101132.9), PRELI domain containing 3B (PRELID3B) (ENSG00000101166.15), synaptonemal complex protein 2 (SYCP2) (ENSG00000196074.12), TATA-box binding protein associated factor 4 (TAF4) (ENSG00000130699.18), cholinergic receptor nicotinic alpha 4 subunit (CHRNA4) (ENSG00000101204.16), SRY-box transcription factor 18 (SOX18) (ENSG00000203883.6), histone H2B type F-S-like (CH507-42P11.1) (ENSG00000274559.3), ATP synthase peripheral stalk subunit F6 (ATP5J) (ENSG00000154723.12), GA binding protein transcription factor subunit alpha (GABPA) (ENSG00000154727.10), SR-related CTD associated factor 4 (SCAF4) (ENSG00000156304.14), H2B clustered histone 12 like (H2BFS) (ENSG00000234289.5), BCLAF1 And THRAP3 family member 3 (CXorf23) (ENSG00000173681.16), proprotein convertase subtilisin/kexin type 1 inhibitor (PCSKIN) (ENSG00000102109.8), MT-RNR2 like 10 (pseudogene) (MTRNR2L10) (ENSG00000256045.2), ATRX chromatin remodeler (ATRX) (ENSG00000085224.21), transcription elongation factor A like 5 (TCEAL5) (ENSG00000204065.2), glutamate dehydrogenase 2 (GLUD2) (ENSG00000182890.4), hypoxanthine phosphoribosyltransferase 1 (HPRT1) (ENSG00000165704.14), oxidoreductase core subunit 6 (MT-ND6) (ENSG00000198695.2), enolase 1 (ENO1) (ENSG00000074800.14), solute carrier family 2 member 1 (SLC2A1) (ENSG00000117394.21), adenylate kinase 4 (AK4) (ENSG00000162433.14), tuftelin 1 (TUFT1) (ENSG00000143367.15), activating transcription factor 3 (ATF3) (ENSG00000162772.16), regenerating family member 1 alpha (REG1A) (ENSG00000115386.5), dual specificity phosphatase 2 (DUSP2) (ENSG00000158050.4), pyruvate dehydrogenase kinase 1 (PDK1) (ENSG00000152256.13), basic helix-loop-helix family member e40 (BHLHE40) (ENSG00000134107.4), C-X-C motif chemokine ligand 8 (CXCL8) (ENSG00000169429.10), secreted phosphoprotein 1 (SPP1) (ENSG00000118785.13), secreted frizzled related protein 2 (SFRP2) (ENSG00000145423.4), natriuretic peptide receptor 3 (NPR3) (ENSG00000113389.15), stanniocalcin 2 (STC2) (ENSG00000113739.10), immediate early response 3 (IER3) (ENSG00000137331.11), heat shock protein family A (Hsp70) member 1B (HSPA1B) (ENSG00000204388.6), alpha-2-glycoprotein 1, zinc-binding (AZGP1) (ENSG00000160862.12), potassium voltage-gated channel subfamily H member 2 (KCNH2) (ENSG00000055118.14), BCL2 interacting protein 3 like (BNIP3L) (ENSG00000104765.15), PLAG1 zinc finger (PLAG1) (ENSG00000181690.7), N-myc downstream regulated 1 (NDRG1) (ENSG00000104419.14), carbonic anhydrase 9 (CA9) (ENSG00000107159.12), UDP-N-acetylglucosamine pyrophosphorylase 1 like 1 (UAP1L1) (ENSG00000197355.10), prolyl 4-hydroxylase subunit alpha 1 (P4HA1) (ENSG00000122884.12), interferon induced transmembrane protein 1 (IFITM1) (ENSG00000185885.15), interferon induced transmembrane protein 3 (IFITM3) (ENSG00000142089.15), cleavage stimulation factor subunit 3 (CSTF3) (ENSG00000176102.11), protein kinase C and casein kinase substrate in neurons 3 (PACSIN3) (ENSG00000165912.15), fatty acid desaturase 2 (FADS2) (ENSG00000134824.13), fatty acid desaturase 1 (FADS1) (ENSG00000149485.18), enolase 2 (ENO2) (ENSG00000111674.8), COPI coat complex subunit zeta 1 (COPZ1) (ENSG00000111481.9), syntaxin 2 (STX2) (ENSG00000111450.13), endoplasmic reticulum oxidoreductase 1 alpha (ERO1A) (ENSG00000197930.12), dipeptidase 1 (DPEP1) (ENSG00000015413.9), lectin, mannose binding 1 (LMAN1) (ENSG00000074695.5), kallikrein related peptidase 6 (KLK6) (ENSG00000167755.14), troponin T1, slow skeletal type (TNNT1) (ENSG00000105048.16), ribophorin II (RPN2) (ENSG00000118705.16), WAP four-disulfide core domain 3 (WFDC3) (ENSG00000124116.18), deoxynucleotidyltransferase terminal interacting protein 1 (DNTTIP1) (ENSG00000101457.12), ubiquitin conjugating enzyme E2 C (UBE2C) (ENSG00000175063.16), dolichyl-phosphate mannosyltransferase subunit 1, catalytic (DPM1) (ENSG00000000419.12).

21. The cancer subtype identifier according any of the preceding embodiments, wherein for the identification of MXE2 subtype the expression of the predictive protein-coding genes is characterized by their up-regulation in the MXE2 subtype as compared to the MXE1 subtype of primary group, selected from of TAR DNA binding protein (TARDBP) (ENSG00000120948.16), heterogeneous nuclear ribonucleoprotein C like 4 (HNRNPCL4) (ENSG00000179412.10), capping actin protein of muscle Z-line subunit beta (CAPZB) (ENSG00000077549.17), heterogeneous nuclear ribonucleoprotein R (HNRNPR) (ENSG00000125944.19), single stranded DNA binding protein 3 (SSBP3) (ENSG00000157216.15), nexilin F-actin binding protein (NEXN) (ENSG00000162614.18), far upstream element binding protein 1 (FUBP1) (ENSG00000162613.16), cellular communication network factor 1 (CYR61) (ENSG00000142871.16), SET like protein (SETSIP) (ENSG00000230667.5), major facilitator superfamily domain containing 14A (MFSD14A) (ENSG00000156875.13), SAS-6 centriolar assembly protein (SASS6) (ENSG00000156876.9), pre-mRNA processing factor 38B (PRPF38B) (ENSG00000134186.11), TATA-box binding protein associated factor 13 (TAF13) (ENSG00000197780.9), RAP1A, member of RAS oncogene family (RAP1A) (ENSG00000116473.14), H2A clustered histone 19 (HIST2H2AA4) (ENSG00000272196.2), H3 clustered histone 15 (HIST2H3A) (ENSG00000203852.3), H4 clustered histone 15 (HIST2H4B) (ENSG00000270276.2), acidic nuclear phosphoprotein 32 family member E (ANP32E) (ENSG00000143401.14), S100 calcium binding protein A10 (S100A10) (ENSG00000197747.8), regulator of G protein signaling 13 (RGS13) (ENSG00000127074.14), ribosomal protein S27a pseudogene 5 (RP11-92K2.2) (ENSG00000231767.4), ubiquitin C-terminal hydrolase L5 (UCHL5) (ENSG00000116750.13), LIM homeobox 9 (LHX9) (ENSG00000143355.15), zinc finger BED-type containing 6 (ZBED6) (ENSG00000257315.2), zinc finger CCCH-type containing 11B (ZC3H11B) (ENSG00000215817.5), ras homolog family member U (RHOU) (ENSG00000116574.5), translin associated factor X (TSNAX) (ENSG00000116918.13), myelin transcription factor 1 like (MYT1L) (ENSG00000186487.18), protein phosphatase 1 catalytic subunit beta (PPP1CB) (ENSG00000213639.9), protein phosphatase, Mg2+/Mn2+ dependent 1B (PPM1B) (ENSG00000138032.20), C1D nuclear receptor corepressor (C1D) (ENSG00000197223.11), poly(rC) binding protein 1 (PCBP1) (ENSG00000169564.6), RANBP2 like and GRIP domain containing 1 (RGPD1) (ENSG00000187627.15), RANBP2 like and GRIP domain containing 2 (RGPD2) (ENSG00000185304.14), RANBP2 like and GRIP domain containing 3 (RGPD3) (ENSG00000153165.18), RANBP2 like and GRIP domain containing 4 (RGPD4) (ENSG00000196862.9), GRIP and coiled-coil domain containing 2 (GCC2) (ENSG00000135968.20), RANBP2 like and GRIP domain containing 5 (RGPD5) (ENSG00000015568.12), RANBP2 like and GRIP domain containing 6 (RGPD6) (ENSG00000183054.11), diazepam binding inhibitor, acyl-CoA binding protein (DBI) (ENSG00000155368.16), RAB6C, member RAS oncogene family (RAB6C) (ENSG00000222014.5), POTE ankyrin domain family member I (POTEI) (ENSG00000196834.11), POTE ankyrin domain family member J (POTEJ) (ENSG00000222038.3), UBX domain protein 4 (UBXN4) (ENSG00000144224.16), ADP ribosylation factor like GTPase 5A (ARL5A) (ENSG00000162980.16), membrane associated ring-CH-type finger 7 (MARCH7) (ENSG00000136536.14), peptidylprolyl isomerase G (PPIG) (ENSG00000138398.15), small RNA binding exonuclease protection factor La (SSB) (ENSG00000138385.15), tousled like kinase 1 (TLK1) (ENSG00000198586.13), corepressor interacting with RBPJ, CIR1 (CIR1) (ENSG00000138433.15), heterogeneous nuclear ribonucleoprotein A3 (HNRNPA3) (ENSG00000170144.20), MOB family member 4, phocein (MOB4) (ENSG00000115540.14), tubulin alpha 4b (TUBA4B) (ENSG00000243910.7), prothymosin alpha (PTMA) (ENSG00000187514.16), myeloma overexpressed 2 pseudogene (MYEOV2) (ENSG00000172428.10), synapsin II (SYN2) (ENSG00000157152.16), LSM3 homolog, U6 small nuclear RNA and mRNA degradation associated (LSM3) (ENSG00000170860.3), protein kinase C delta (PRKCD) (ENSG00000163932.13), MT-RNR2 like 12 (pseudogene) (MTRNR2L12) (ENSG00000269028.3), filamin A interacting protein 1 like (FILIP1L) (ENSG00000168386.18), PEST proteolytic signal containing nuclear protein (PCNP) (ENSG00000081154.11), RAB7A, member RAS oncogene family (RAB7A) (ENSG00000075785.12), WW domain containing transcription regulator 1 (WWTR1) (ENSG00000018408.14), structural maintenance of chromosomes 4 (SMC4) (ENSG00000113810.15), SKI like proto-oncogene (SKIL) (ENSG00000136603.13), cytoplasmic polyadenylation element binding protein 2 (CPEB2) (ENSG00000137449.15), MOB kinase activator 1B (MOB1B) (ENSG00000173542.8), Ras association domain family member 6 (RASSF6) (ENSG00000169435.13), heterogeneous nuclear ribonucleoprotein D like (HNRNPDL) (ENSG00000152795.17), DnaJ heat shock protein family (Hsp40) member B14 (DNAJB14) (ENSG00000164031.16), family with sequence similarity 241 member A (C4orf32) (ENSG00000174749.5), La ribonucleoprotein 7, transcriptional regulator (LARP7) (ENSG00000174720.15), progesterone receptor membrane component 2 (PGRMC2) (ENSG00000164040.16), tryptophan 2,3-dioxygenase (TDO2) (ENSG00000151790.8), palladin, cytoskeletal associated protein (PALLD) (ENSG00000129116.17), 15-hydroxyprostaglandin dehydrogenase (HPGD) (ENSG00000164120.13), inhibitor of growth family member 2 (ING2) (ENSG00000168556.6), cilia and flagella associated protein 97 (CFAP97) (ENSG00000164323.12), NADH:ubiquinone oxidoreductase subunit S6 (NDUFS6) (ENSG00000145494.11), SUB1 regulator of transcription (SUB1) (ENSG00000113387.11), NADH:ubiquinone oxidoreductase complex assembly factor 2 (NDUFAF2) (ENSG00000164182.10), CWC27 spliceosome associated cyclophilin (CWC27) (ENSG00000153015.15), protein geranylgeranyltransferase type I subunit beta (PGGT1B) (ENSG00000164219.9), coiled-coil domain containing 112 (CCDC112) (ENSG00000164221.12), lysyl oxidase (LOX) (ENSG00000113083.13), ubiquitin conjugating enzyme E2 B (UBE2B) (ENSG00000119048.7), RNA binding motif protein 27 (RBM27) (ENSG00000091009.7), H4 clustered histone 11 (HIST1H4J) (ENSG00000197238.4), H4 clustered histone 12 (HIST1H4K) (ENSG00000273542.1), CD2 associated protein (CD2AP) (ENSG00000198087.7), KIAA1586 (KIAA1586) (ENSG00000168116.13), FKBP prolyl isomerase family member 1C (FKBP1C) (ENSG00000198225.5), high mobility group nucleosomal binding domain 3 (HMGN3) (ENSG00000118418.14), SEC63 homolog, protein translocation regulator (SEC63) (ENSG00000025796.13), myristoylated alanine rich protein kinase C substrate (MARCKS) (ENSG00000277443.2), RWD domain containing 1 (RWDD1) (ENSG00000111832.12), NUS1 dehydrodolichyl diphosphate synthase subunit (NUS1) (ENSG00000153989.7), cellular communication network factor 2 (CTGF) (ENSG00000118523.5), BCL2 associated transcription factor 1 (BCLAF1) (ENSG00000029363.16), LTV1 ribosome biogenesis factor (LTV1) (ENSG00000135521.8), TGF-beta activated kinase 1 (MAP3K7) binding protein 2 (TAB2) (ENSG00000055208.18), peptidylprolyl isomerase like 4 (PPIL4) (ENSG00000131013.3), RB associated KRAB zinc finger (RBAK) (ENSG00000146587.17), transmembrane protein 106B (TMEM106B) (ENSG00000106460.18), aryl hydrocarbon receptor (AHR) (ENSG00000106546.13), transformer 2 alpha homolog (TRA2A) (ENSG00000164548.10), sperm microtubule inner protein 4 (C7orf31) (ENSG00000153790.11), heterogeneous nuclear ribonucleoprotein A2/B1 (HNRNPA2B1) (ENSG00000122566.20), zinc and ring finger 2 (ZNRF2) (ENSG00000180233.10), ITPR interacting domain containing 1 (CCDC129) (ENSG00000180347.13), POU class 6 homeobox 2 (POU6F2) (ENSG00000106536.19), RAS like proto-oncogene A (RALA) (ENSG00000006451.7), inhibin subunit beta A (INHBA) (ENSG00000122641.10), zinc finger protein 680 (ZNF680) (ENSG00000173041.11), fibrinogen like 2 (FGL2) (ENSG00000127951.6), A-kinase anchoring protein 9 (AKAP9) (ENSG00000127914.16), family with sequence similarity 133 member B (FAM133B) (ENSG00000234545.7), DnaJ heat shock protein family (Hsp40) member C2 (DNAJC2) (ENSG00000105821.14), NADH:ubiquinone oxidoreductase subunit A5 (NDUFA5) (ENSG00000128609.14), chromosome 7 open reading frame 73 (C7orf73) (ENSG00000243317.7), protein kinase AMP-activated non-catalytic subunit gamma 2 (PRKAG2) (ENSG00000106617.13), neurofilament medium chain (NEFM) (ENSG00000104722.13), dynactin subunit 6 (DCTN6) (ENSG00000104671.7), CCAAT enhancer binding protein delta (CEBPD) (ENSG00000221869.4), syntrophin gamma 1 (SNTG1) (ENSG00000147481.15), RAB2A, member RAS oncogene family (RAB2A) (ENSG00000104388.14), translocation associated membrane protein 1 (TRAM1) (ENSG00000067167.7), zinc finger C2HC-type containing 1A (ZC2HC1A) (ENSG00000104427.11), tumor protein D52 (TPD52) (ENSG00000076554.15), ribosomal biogenesis factor (C8orf59) (ENSG00000176731.11), ubiquinol-cytochrome c reductase binding protein (UQCRB) (ENSG00000156467.9), RNA polymerase II, I and III subunit K (POLR2K) (ENSG00000147669.10), poly(A) binding protein cytoplasmic 1 (PABPC1) (ENSG00000070756.15), glycine decarboxylase (GLDC) (ENSG00000178445.9), zinc finger DHHC-type palmitoyltransferase 21 (ZDHHC21) (ENSG00000175893.11), PC4 and SRSF1 interacting protein 1 (PSIP1) (ENSG00000164985.14), caspase activity and apoptosis inhibitor 1 (CAAP1) (ENSG00000120159.11), TATA-box binding protein associated factor 1 like (TAF1L) (ENSG00000122728.6), carnosine N-methyltransferase 1 (CARNMT1) (ENSG00000156017.12), riboflavin kinase (RFK) (ENSG00000135002.11), RecQ mediated genome instability 1 (RMI1) (ENSG00000178966.16), acidic nuclear phosphoprotein 32 family member B (ANP32B) (ENSG00000136938.8), actin binding transcription modulator (FAM206A) (ENSG00000119328.11), TATA-box binding protein associated factor 3 (TAF3) (ENSG00000165632.7), chondroitin sulfate N-acetylgalactosaminyltransferase 2 (CSGALNACT2) (ENSG00000169826.7), ubiquitin conjugating enzyme E2 D1 (UBE2D1) (ENSG00000072401.14), VPS26 retromer complex component A (VPS26A) (ENSG00000122958.14), eukaryotic translation initiation factor 5A like 1 (EIF5AL1) (ENSG00000253626.3), kinesin family member 20B (KIF20B) (ENSG00000138182.14), FRA10A associated CGG repeat 1 (FRA10AC1) (ENSG00000148690.11), survival motor neuron domain containing 1 (SMNDC1) (ENSG00000119953.12), coiled-coil domain containing 186 (CCDC186) (ENSG00000165813.18), pleckstrin homology like domain family A member 2 (PHLDA2) (ENSG00000181649.6), MT-RNR2 like 8 (pseudogene) (MTRNR2L8) (ENSG00000255823.4), lin-7 homolog C, crumbs cell polarity complex component (LIN7C) (ENSG00000148943.11), glycine-N-acyltransferase like 1 (GLYATL1) (ENSG00000166840.13), membrane spanning 4-domains A6E (MS4A6E) (ENSG00000166926.8), heterogeneous nuclear ribonucleoprotein U like 2 (HNRNPUL2) (ENSG00000214753.2), FKBP prolyl isomerase 2 (FKBP2) (ENSG00000173486.12), spliceosome associated factor 1, recruiter of U4/U6.U5 tri-snRNP (SART1) (ENSG00000175467.14), remodeling and spacing factor 1 (RSF1) (ENSG00000048649.13), cysteine and histidine rich domain containing 1 (CHORDC1) (ENSG00000110172.11), ankyrin repeat domain 49 (ANKRD49) (ENSG00000168876.8), family with sequence similarity 76 member B (FAM76B) (ENSG00000077458.12), coiled-coil domain containing 82 (CCDC82) (ENSG00000149231.12), transmembrane protein 123 (TMEM123) (ENSG00000152558.14), defective in cullin neddylation 1 domain containing 5 (DCUN1D5) (ENSG00000137692.11), RNA binding motif protein 7 (RBM7) (ENSG00000076053.10), H2A.X variant histone (H2AFX) (ENSG00000188486.3), parathymosin (PTMS) (ENSG00000159335.15), basic helix-loop-helix family member e41 (BHLHE41) (ENSG00000123095.5), ERGIC and golgi 2 (ERGIC2) (ENSG00000087502.17), glucoside xylosyltransferase 1 (GXYLT1) (ENSG00000151233.10), zinc finger CCHC-type and RNA binding motif containing 1 (ZCRB1) (ENSG00000139168.7), twinfilin actin binding protein 1 (TWF1) (ENSG00000151239.13), activating transcription factor 1 (ATF1) (ENSG00000123268.8), oxysterol binding protein like 8 (OSBPL8) (ENSG00000091039.16), RNA 5′-phosphate and 3′-OH ligase 1 (C12orf29) (ENSG00000133641.17), centrosomal protein 290 (CEP290) (ENSG00000198707.14), early endosome antigen 1 (EEA1) (ENSG00000102189.16), small nuclear ribonucleoprotein polypeptide F (SNRPF) (ENSG00000139343.10), nuclear transcription factor Y subunit beta (NFYB) (ENSG00000120837.7), mitochondrial calcium uptake 2 (MICU2) (ENSG00000165487.13), poly(A) binding protein cytoplasmic 3 (PABPC3) (ENSG00000151846.8), casein kinase 1 alpha 1 like (CSNK1A1L) (ENSG00000180138.7), laccase domain containing 1 (LACC1) (ENSG00000179630.10), GPALPP motifs containing 1 (GPALPP1) (ENSG00000133114.17), tumor protein, translationally-controlled 1 (TPT1) (ENSG00000133112.16), zinc finger CCCH-type containing 13 (ZC3H13) (ENSG00000123200.16), integral membrane protein 2B (ITM2B) (ENSG00000136156.12), cytoskeleton associated protein 2 (CKAP2) (ENSG00000136108.14), mitotic spindle organizing protein 1 (MZT1) (ENSG00000204899.5), KLF transcription factor 5 (KLF5) (ENSG00000102554.13), potassium channel tetramerization domain containing 12 (KCTD12) (ENSG00000178695.5), coiled-coil domain containing 168 (CCDC168) (ENSG00000175820.3), arginine and glutamate rich 1 (ARGLU1) (ENSG00000134884.13), serine palmitoyltransferase small subunit A (SPTSSA) (ENSG00000165389.6), ras homolog family member J (RHOJ) (ENSG00000126785.12), golgin A6 family like 6 (GOLGA6L6) (ENSG00000277322.1), golgin A6 family like 2 (GOLGA6L2) (ENSG00000174450.11), ER membrane protein complex subunit 7 (EMC7) (ENSG00000134153.9), eukaryotic translation initiation factor 3 subunit J (EIF3J) (ENSG00000104131.12), COP9 signalosome subunit 2 (COPS2) (ENSG00000166200.14), RAB27A, member RAS oncogene family (RAB27A) (ENSG00000069974.15), SAFB like transcription modulator (SLTM) (ENSG00000137776.16), reticulocalbin 2 (RCN2) (ENSG00000117906.13), methenyltetrahydrofolate synthetase (MTHFS) (ENSG00000136371.10), zinc finger AN1-type containing 6 (ZFAND6) (ENSG00000086666.18), nuclear receptor subfamily 2 group F member 2 (NR2F2) (ENSG00000185551.14), centrosomal protein 20 (FOPNL) (ENSG00000133393.12), centriolar coiled-coil protein 110 (CCP110) (ENSG00000103540.16), nuclear pore complex interacting protein family member B5 (NPIPB5) (ENSG00000243716.10), eukaryotic translation initiation factor 3 subunit C like (EIF3CL) (ENSG00000205609.12), terminal nucleotidyltransferase 4B (PAPD5) (ENSG00000121274.12), tubulin polymerization promoting protein family member 3 (TPPP3) (ENSG00000159713.10), NIN1 (RPN12) binding protein 1 homolog (NOB1) (ENSG00000141101.12), contactin associated protein family member 4 (CNTNAP4) (ENSG00000152910.18), MAF bZIP transcription factor (MAF) (ENSG00000178573.6), CBFA2/RUNX1 partner transcriptional co-repressor 3 (CBFA2T3) (ENSG00000129993.14), ankyrin repeat domain containing 11 (ANKRD11) (ENSG00000167522.15), ER membrane protein complex subunit 6 (TMEM93) (ENSG00000127774.6), lysine demethylase 6B (KDM6B) (ENSG00000132510.10), ribosomal protein L26 (RPL26) (ENSG00000161970.13), SUZ12 polycomb repressive complex 2 subunit (SUZ12) (ENSG00000178691.10), insulin like growth factor binding protein 4 (IGFBP4) (ENSG00000141753.6), keratin 10 (KRT10) (ENSG00000186395.7), transducer of ERBB2, 1 (TOB1) (ENSG00000141232.4), nascent polypeptide associated complex subunit alpha 2 (NACA2) (ENSG00000253506.2), SRY-box transcription factor 9 (SOX9) (ENSG00000125398.6), ring finger protein 138 (RNF138) (ENSG00000134758.13), immediate early response 3 interacting protein 1 (IER3IP1) (ENSG00000134049.5), methyl-CpG binding domain protein 2 (MBD2) (ENSG00000134046.11), SEC11 homolog C, signal peptidase complex subunit (SEC11C) (ENSG00000166562.8), coiled-coil domain containing 102B (CCDC102B) (ENSG00000150636.17), zinc finger and BTB domain containing 7A (ZBTB7A) (ENSG00000178951.8), scaffold attachment factor B2 (SAFB2) (ENSG00000130254.11), scaffold attachment factor B (SAFB) (ENSG00000160633.12), tubulin beta 4A class IVa (TUBB4A) (ENSG00000104833.11), RAB11B, member RAS oncogene family (RAB11B) (ENSG00000185236.11), solute carrier family 1 member 6 (SLC1A6) (ENSG00000105143.12), KLF transcription factor 2 (KLF2) (ENSG00000127528.5), zinc finger protein 708 (ZNF708) (ENSG00000182141.10), PAF1 homolog, Paf1/RNA polymerase II complex component (PAF1) (ENSG00000006712.14), transforming growth factor beta 1 (TGFB1) (ENSG00000105329.9), novel protein, readthrough between CEACAM5-CEACAM6 (AC011513.3) (ENSG00000267881.1), CD177 molecule (CD177) (ENSG00000204936.9), potassium voltage-gated channel subfamily A member 7 (KCNA7) (ENSG00000104848.1), ribosomal protein L28 (RPL28) (ENSG00000108107.14), zinc finger protein 580 (ZNF580) (ENSG00000213015.8), ESF1 nucleolar pre-rRNA processing protein homolog (ESF1) (ENSG00000089048.14), charged multivesicular body protein 4B (CHMP4B) (ENSG00000101421.3), DNA topoisomerase I (TOP1) (ENSG00000198900.5), prefoldin subunit 4 (PFDN4) (ENSG00000101132.9), PRELI domain containing 3B (PRELID3B) (ENSG00000101166.15), synaptonemal complex protein 2 (SYCP2) (ENSG00000196074.12), TATA-box binding protein associated factor 4 (TAF4) (ENSG00000130699.18), cholinergic receptor nicotinic alpha 4 subunit (CHRNA4) (ENSG00000101204.16), SRY-box transcription factor 18 (SOX18) (ENSG00000203883.6), histone H2B type F-S-like (CH507-42P11.1) (ENSG00000274559.3), ATP synthase peripheral stalk subunit F6 (ATP5J) (ENSG00000154723.12), GA binding protein transcription factor subunit alpha (GABPA) (ENSG00000154727.10), SR-related CTD associated factor 4 (SCAF4) (ENSG00000156304.14), H2B clustered histone 12 like (H2BFS) (ENSG00000234289.5), BCLAF1 And THRAP3 family member 3 (CXorf23) (ENSG00000173681.16), proprotein convertase subtilisin/kexin type 1 inhibitor (PCSKIN) (ENSG00000102109.8), MT-RNR2 like 10 (pseudogene) (MTRNR2L10) (ENSG00000256045.2), ATRX chromatin remodeler (ATRX) (ENSG00000085224.21), transcription elongation factor A like 5 (TCEAL5) (ENSG00000204065.2), glutamate dehydrogenase 2 (GLUD2) (ENSG00000182890.4), hypoxanthine phosphoribosyltransferase 1 (HPRT1) (ENSG00000165704.14), oxidoreductase core subunit 6 (MT-ND6) (ENSG00000198695.2), preferably,
wherein for the identification of MXE2 subtype the expression of the predictive protein-coding genes is characterized by their up-regulation in the MXE2 subtype as compared to the MXE1 subtype, preferably selected from TAR DNA binding protein (TARDBP) (ENSG00000120948.16), heterogeneous nuclear ribonucleoprotein C like 4 (HNRNPCL4) (ENSG00000179412.10), capping actin protein of muscle Z-line subunit beta (CAPZB) (ENSG00000077549.17), heterogeneous nuclear ribonucleoprotein R (HNRNPR) (ENSG00000125944.19), single stranded DNA binding protein 3 (SSBP3) (ENSG00000157216.15), nexilin F-actin binding protein (NEXN) (ENSG00000162614.18), far upstream element binding protein 1 (FUBP1) (ENSG00000162613.16), cellular communication network factor 1 (CYR61) (ENSG00000142871.16), SET like protein (SETSIP) (ENSG00000230667.5), major facilitator superfamily domain containing 14A (MFSD14A) (ENSG00000156875.13), SAS-6 centriolar assembly protein (SASS6) (ENSG00000156876.9), pre-mRNA processing factor 38B (PRPF38B) (ENSG00000134186.11), TATA-box binding protein associated factor 13 (TAF13) (ENSG00000197780.9), RAP1A, member of RAS oncogene family (RAP1A) (ENSG00000116473.14), H2A clustered histone 19 (HIST2H2AA4) (ENSG00000272196.2), H3 clustered histone 15 (HIST2H3A) (ENSG00000203852.3), H4 clustered histone 15 (HIST2H4B) (ENSG00000270276.2), acidic nuclear phosphoprotein 32 family member E (ANP32E) (ENSG00000143401.14), S100 calcium binding protein A10 (S100A10) (ENSG00000197747.8), regulator of G protein signaling 13 (RGS13) (ENSG00000127074.14), ribosomal protein S27a pseudogene 5 (RP11-92K2.2) (ENSG00000231767.4), ubiquitin C-terminal hydrolase L5 (UCHL5) (ENSG00000116750.13), LIM homeobox 9 (LHX9) (ENSG00000143355.15), zinc finger BED-type containing 6 (ZBED6) (ENSG00000257315.2), zinc finger CCCH-type containing 11B (ZC3H11B) (ENSG00000215817.5), ras homolog family member U (RHOU) (ENSG00000116574.5), translin associated factor X (TSNAX) (ENSG00000116918.13), myelin transcription factor 1 like (MYT1L) (ENSG00000186487.18), protein phosphatase 1 catalytic subunit beta (PPP1CB) (ENSG00000213639.9), protein phosphatase, Mg2+/Mn2+ dependent 1B (PPM1B) (ENSG00000138032.20), C1D nuclear receptor corepressor (C1D) (ENSG00000197223.11), poly(rC) binding protein 1 (PCBP1) (ENSG00000169564.6), RANBP2 like and GRIP domain containing 1 (RGPD1) (ENSG00000187627.15), RANBP2 like and GRIP domain containing 2 (RGPD2) (ENSG00000185304.14), RANBP2 like and GRIP domain containing 3 (RGPD3) (ENSG00000153165.18), RANBP2 like and GRIP domain containing 4 (RGPD4) (ENSG00000196862.9), GRIP and coiled-coil domain containing 2 (GCC2) (ENSG00000135968.20), RANBP2 like and GRIP domain containing 5 (RGPD5) (ENSG00000015568.12), RANBP2 like and GRIP domain containing 6 (RGPD6) (ENSG00000183054.11), diazepam binding inhibitor, acyl-CoA binding protein (DBI) (ENSG00000155368.16), RAB6C, member RAS oncogene family (RAB6C) (ENSG00000222014.5), POTE ankyrin domain family member I (POTEI) (ENSG00000196834.11), POTE ankyrin domain family member J (POTEJ) (ENSG00000222038.3), UBX domain protein 4 (UBXN4) (ENSG00000144224.16), ADP ribosylation factor like GTPase 5A (ARL5A) (ENSG00000162980.16), membrane associated ring-CH-type finger 7 (MARCH7) (ENSG00000136536.14), peptidylprolyl isomerase G (PPIG) (ENSG00000138398.15), small RNA binding exonuclease protection factor La (SSB) (ENSG00000138385.15), tousled like kinase 1 (TLK1) (ENSG00000198586.13), corepressor interacting with RBPJ, CIR1 (CIR1) (ENSG00000138433.15), heterogeneous nuclear ribonucleoprotein A3 (HNRNPA3) (ENSG00000170144.20), MOB family member 4, phocein (MOB4) (ENSG00000115540.14), tubulin alpha 4b (TUBA4B) (ENSG00000243910.7), prothymosin alpha (PTMA) (ENSG00000187514.16), myeloma overexpressed 2 pseudogene (MYEOV2) (ENSG00000172428.10), synapsin II (SYN2) (ENSG00000157152.16), LSM3 homolog, U6 small nuclear RNA and mRNA degradation associated (LSM3) (ENSG00000170860.3), protein kinase C delta (PRKCD) (ENSG00000163932.13), MT-RNR2 like 12 (pseudogene) (MTRNR2L12) (ENSG00000269028.3), filamin A interacting protein 1 like (FILIP1L) (ENSG00000168386.18), PEST proteolytic signal containing nuclear protein (PCNP) (ENSG00000081154.11), RAB7A, member RAS oncogene family (RAB7A) (ENSG00000075785.12), WW domain containing transcription regulator 1 (WWTR1) (ENSG00000018408.14), structural maintenance of chromosomes 4 (SMC4) (ENSG00000113810.15), SKI like proto-oncogene (SKIL) (ENSG00000136603.13), cytoplasmic polyadenylation element binding protein 2 (CPEB2) (ENSG00000137449.15), MOB kinase activator 1B (MOB1B) (ENSG00000173542.8), Ras association domain family member 6 (RASSF6) (ENSG00000169435.13), heterogeneous nuclear ribonucleoprotein D like (HNRNPDL) (ENSG00000152795.17), DnaJ heat shock protein family (Hsp40) member B14 (DNAJB14) (ENSG00000164031.16), family with sequence similarity 241 member A (C4orf32) (ENSG00000174749.5), La ribonucleoprotein 7, transcriptional regulator (LARP7) (ENSG00000174720.15), progesterone receptor membrane component 2 (PGRMC2) (ENSG00000164040.16), tryptophan 2,3-dioxygenase (TDO2) (ENSG00000151790.8), palladin, cytoskeletal associated protein (PALLD) (ENSG00000129116.17), 15-hydroxyprostaglandin dehydrogenase (HPGD) (ENSG00000164120.13), inhibitor of growth family member 2 (ING2) (ENSG00000168556.6), cilia and flagella associated protein 97 (CFAP97) (ENSG00000164323.12), NADH:ubiquinone oxidoreductase subunit S6 (NDUFS6) (ENSG00000145494.11), SUB1 regulator of transcription (SUB1) (ENSG00000113387.11), NADH:ubiquinone oxidoreductase complex assembly factor 2 (NDUFAF2) (ENSG00000164182.10), CWC27 spliceosome associated cyclophilin (CWC27) (ENSG00000153015.15), protein geranylgeranyltransferase type I subunit beta (PGGT1B) (ENSG00000164219.9), coiled-coil domain containing 112 (CCDC112) (ENSG00000164221.12), lysyl oxidase (LOX) (ENSG00000113083.13), ubiquitin conjugating enzyme E2 B (UBE2B) (ENSG00000119048.7), RNA binding motif protein 27 (RBM27) (ENSG00000091009.7), H4 clustered histone 11 (HIST1H4J) (ENSG00000197238.4), H4 clustered histone 12 (HIST1H4K) (ENSG00000273542.1), CD2 associated protein (CD2AP) (ENSG00000198087.7), KIAA1586 (KIAA1586) (ENSG00000168116.13), FKBP prolyl isomerase family member 1C (FKBP1C) (ENSG00000198225.5), high mobility group nucleosomal binding domain 3 (HMGN3) (ENSG00000118418.14), SEC63 homolog, protein translocation regulator (SEC63) (ENSG00000025796.13), myristoylated alanine rich protein kinase C substrate (MARCKS) (ENSG00000277443.2), RWD domain containing 1 (RWDD1) (ENSG00000111832.12), NUS1 dehydrodolichyl diphosphate synthase subunit (NUS1) (ENSG00000153989.7), cellular communication network factor 2 (CTGF) (ENSG00000118523.5), BCL2 associated transcription factor 1 (BCLAF1) (ENSG00000029363.16), LTV1 ribosome biogenesis factor (LTV1) (ENSG00000135521.8), TGF-beta activated kinase 1 (MAP3K7) binding protein 2 (TAB2) (ENSG00000055208.18), peptidylprolyl isomerase like 4 (PPIL4) (ENSG00000131013.3), RB associated KRAB zinc finger (RBAK) (ENSG00000146587.17), transmembrane protein 106B (TMEM106B) (ENSG00000106460.18), aryl hydrocarbon receptor (AHR) (ENSG00000106546.13), transformer 2 alpha homolog (TRA2A) (ENSG00000164548.10), sperm microtubule inner protein 4 (C7orf31) (ENSG00000153790.11), heterogeneous nuclear ribonucleoprotein A2/B1 (HNRNPA2B1) (ENSG00000122566.20), zinc and ring finger 2 (ZNRF2) (ENSG00000180233.10), ITPR interacting domain containing 1 (CCDC129) (ENSG00000180347.13), POU class 6 homeobox 2 (POU6F2) (ENSG00000106536.19), RAS like proto-oncogene A (RALA) (ENSG00000006451.7), inhibin subunit beta A (INHBA) (ENSG00000122641.10), zinc finger protein 680 (ZNF680) (ENSG00000173041.11), fibrinogen like 2 (FGL2) (ENSG00000127951.6), A-kinase anchoring protein 9 (AKAP9) (ENSG00000127914.16), family with sequence similarity 133 member B (FAM133B) (ENSG00000234545.7), DnaJ heat shock protein family (Hsp40) member C2 (DNAJC2) (ENSG00000105821.14), NADH:ubiquinone oxidoreductase subunit A5 (NDUFA5) (ENSG00000128609.14), chromosome 7 open reading frame 73 (C7orf73) (ENSG00000243317.7), protein kinase AMP-activated non-catalytic subunit gamma 2 (PRKAG2) (ENSG00000106617.13), neurofilament medium chain (NEFM) (ENSG00000104722.13), dynactin subunit 6 (DCTN6) (ENSG00000104671.7), CCAAT enhancer binding protein delta (CEBPD) (ENSG00000221869.4), syntrophin gamma 1 (SNTG1) (ENSG00000147481.15), RAB2A, member RAS oncogene family (RAB2A) (ENSG00000104388.14), translocation associated membrane protein 1 (TRAM1) (ENSG00000067167.7), zinc finger C2HC-type containing 1A (ZC2HC1A) (ENSG00000104427.11), tumor protein D52 (TPD52) (ENSG00000076554.15), ribosomal biogenesis factor (C8orf59) (ENSG00000176731.11), ubiquinol-cytochrome c reductase binding protein (UQCRB) (ENSG00000156467.9), RNA polymerase II, I and III subunit K (POLR2K) (ENSG00000147669.10), poly(A) binding protein cytoplasmic 1 (PABPC1) (ENSG00000070756.15), glycine decarboxylase (GLDC) (ENSG00000178445.9), zinc finger DHHC-type palmitoyltransferase 21 (ZDHHC21) (ENSG00000175893.11), PC4 and SRSF1 interacting protein 1 (PSIP1) (ENSG00000164985.14), caspase activity and apoptosis inhibitor 1 (CAAP1) (ENSG00000120159.11), TATA-box binding protein associated factor 1 like (TAF1L) (ENSG00000122728.6), carnosine N-methyltransferase 1 (CARNMT1) (ENSG00000156017.12), riboflavin kinase (RFK) (ENSG00000135002.11), RecQ mediated genome instability 1 (RMI1) (ENSG00000178966.16), acidic nuclear phosphoprotein 32 family member B (ANP32B) (ENSG00000136938.8), actin binding transcription modulator (FAM206A) (ENSG00000119328.11), TATA-box binding protein associated factor 3 (TAF3) (ENSG00000165632.7), chondroitin sulfate N-acetylgalactosaminyltransferase 2 (CSGALNACT2) (ENSG00000169826.7), ubiquitin conjugating enzyme E2 D1 (UBE2D1) (ENSG00000072401.14), VPS26 retromer complex component A (VPS26A) (ENSG00000122958.14), eukaryotic translation initiation factor 5A like 1 (EIF5AL1) (ENSG00000253626.3), kinesin family member 20B (KIF20B) (ENSG00000138182.14), FRA10A associated CGG repeat 1 (FRA10AC1) (ENSG00000148690.11), survival motor neuron domain containing 1 (SMNDC1) (ENSG00000119953.12), coiled-coil domain containing 186 (CCDC186) (ENSG00000165813.18), pleckstrin homology like domain family A member 2 (PHLDA2) (ENSG00000181649.6), MT-RNR2 like 8 (pseudogene) (MTRNR2L8) (ENSG00000255823.4), lin-7 homolog C, crumbs cell polarity complex component (LIN7C) (ENSG00000148943.11), glycine-N-acyltransferase like 1 (GLYATL1) (ENSG00000166840.13), membrane spanning 4-domains A6E (MS4A6E) (ENSG00000166926.8), heterogeneous nuclear ribonucleoprotein U like 2 (HNRNPUL2) (ENSG00000214753.2), FKBP prolyl isomerase 2 (FKBP2) (ENSG00000173486.12), spliceosome associated factor 1, recruiter of U4/U6.U5 tri-snRNP (SART1) (ENSG00000175467.14), remodeling and spacing factor 1 (RSF1) (ENSG00000048649.13), cysteine and histidine rich domain containing 1 (CHORDC1) (ENSG00000110172.11), ankyrin repeat domain 49 (ANKRD49) (ENSG00000168876.8), family with sequence similarity 76 member B (FAM76B) (ENSG00000077458.12), coiled-coil domain containing 82 (CCDC82) (ENSG00000149231.12), transmembrane protein 123 (TMEM123) (ENSG00000152558.14), defective in cullin neddylation 1 domain containing 5 (DCUN1D5) (ENSG00000137692.11), RNA binding motif protein 7 (RBM7) (ENSG00000076053.10), H2A.X variant histone (H2AFX) (ENSG00000188486.3), parathymosin (PTMS) (ENSG00000159335.15), basic helix-loop-helix family member e41 (BHLHE41) (ENSG00000123095.5), ERGIC and golgi 2 (ERGIC2) (ENSG00000087502.17), glucoside xylosyltransferase 1 (GXYLT1) (ENSG00000151233.10), zinc finger CCHC-type and RNA binding motif containing 1 (ZCRB1) (ENSG00000139168.7), twinfilin actin binding protein 1 (TWF1) (ENSG00000151239.13), activating transcription factor 1 (ATF1) (ENSG00000123268.8), oxysterol binding protein like 8 (OSBPL8) (ENSG00000091039.16), RNA 5′-phosphate and 3′-OH ligase 1 (C12orf29) (ENSG00000133641.17), centrosomal protein 290 (CEP290) (ENSG00000198707.14), early endosome antigen 1 (EEA1) (ENSG00000102189.16), small nuclear ribonucleoprotein polypeptide F (SNRPF) (ENSG00000139343.10), nuclear transcription factor Y subunit beta (NFYB) (ENSG00000120837.7), mitochondrial calcium uptake 2 (MICU2) (ENSG00000165487.13), poly(A) binding protein cytoplasmic 3 (PABPC3) (ENSG00000151846.8), casein kinase 1 alpha 1 like (CSNK1A1L) (ENSG00000180138.7), laccase domain containing 1 (LACC1) (ENSG00000179630.10), GPALPP motifs containing 1 (GPALPP1) (ENSG00000133114.17), tumor protein, translationally-controlled 1 (TPT1) (ENSG00000133112.16), zinc finger CCCH-type containing 13 (ZC3H13) (ENSG00000123200.16), integral membrane protein 2B (ITM2B) (ENSG00000136156.12), cytoskeleton associated protein 2 (CKAP2) (ENSG00000136108.14), mitotic spindle organizing protein 1 (MZT1) (ENSG00000204899.5), KLF transcription factor 5 (KLF5) (ENSG00000102554.13), potassium channel tetramerization domain containing 12 (KCTD12) (ENSG00000178695.5), coiled-coil domain containing 168 (CCDC168) (ENSG00000175820.3), arginine and glutamate rich 1 (ARGLU1) (ENSG00000134884.13), serine palmitoyltransferase small subunit A (SPTSSA) (ENSG00000165389.6), ras homolog family member J (RHOJ) (ENSG00000126785.12), golgin A6 family like 6 (GOLGA6L6) (ENSG00000277322.1), golgin A6 family like 2 (GOLGA6L2) (ENSG00000174450.11), ER membrane protein complex subunit 7 (EMC7) (ENSG00000134153.9), eukaryotic translation initiation factor 3 subunit J (EIF3J) (ENSG00000104131.12), COP9 signalosome subunit 2 (COPS2) (ENSG00000166200.14), RAB27A, member RAS oncogene family (RAB27A) (ENSG00000069974.15), SAFB like transcription modulator (SLTM) (ENSG00000137776.16), reticulocalbin 2 (RCN2) (ENSG00000117906.13), methenyltetrahydrofolate synthetase (MTHFS) (ENSG00000136371.10), zinc finger AN1-type containing 6 (ZFAND6) (ENSG00000086666.18), nuclear receptor subfamily 2 group F member 2 (NR2F2) (ENSG00000185551.14), centrosomal protein 20 (FOPNL) (ENSG00000133393.12), centriolar coiled-coil protein 110 (CCP110) (ENSG00000103540.16), nuclear pore complex interacting protein family member B5 (NPIPB5) (ENSG00000243716.10), eukaryotic translation initiation factor 3 subunit C like (EIF3CL) (ENSG00000205609.12), terminal nucleotidyltransferase 4B (PAPD5) (ENSG00000121274.12), tubulin polymerization promoting protein family member 3 (TPPP3) (ENSG00000159713.10), NIN1 (RPN12) binding protein 1 homolog (NOB1) (ENSG00000141101.12), contactin associated protein family member 4 (CNTNAP4) (ENSG00000152910.18), MAF bZIP transcription factor (MAF) (ENSG00000178573.6), CBFA2/RUNX1 partner transcriptional co-repressor 3 (CBFA2T3) (ENSG00000129993.14), ankyrin repeat domain containing 11 (ANKRD11) (ENSG00000167522.15), ER membrane protein complex subunit 6 (TMEM93) (ENSG00000127774.6), lysine demethylase 6B (KDM6B) (ENSG00000132510.10), ribosomal protein L26 (RPL26) (ENSG00000161970.13), SUZ12 polycomb repressive complex 2 subunit (SUZ12) (ENSG00000178691.10), insulin like growth factor binding protein 4 (IGFBP4) (ENSG00000141753.6), keratin 10 (KRT10) (ENSG00000186395.7), transducer of ERBB2, 1 (TOB1) (ENSG00000141232.4), nascent polypeptide associated complex subunit alpha 2 (NACA2) (ENSG00000253506.2), SRY-box transcription factor 9 (SOX9) (ENSG00000125398.6), ring finger protein 138 (RNF138) (ENSG00000134758.13), immediate early response 3 interacting protein 1 (IER3IP1) (ENSG00000134049.5), methyl-CpG binding domain protein 2 (MBD2) (ENSG00000134046.11), SEC11 homolog C, signal peptidase complex subunit (SEC11C) (ENSG00000166562.8), coiled-coil domain containing 102B (CCDC102B) (ENSG00000150636.17), zinc finger and BTB domain containing 7A (ZBTB7A) (ENSG00000178951.8), scaffold attachment factor B2 (SAFB2) (ENSG00000130254.11), scaffold attachment factor B (SAFB) (ENSG00000160633.12), tubulin beta 4A class IVa (TUBB4A) (ENSG00000104833.11), RAB11B, member RAS oncogene family (RAB11B) (ENSG00000185236.11), solute carrier family 1 member 6 (SLC1A6) (ENSG00000105143.12), KLF transcription factor 2 (KLF2) (ENSG00000127528.5), zinc finger protein 708 (ZNF708) (ENSG00000182141.10), PAF1 homolog, Paf1/RNA polymerase II complex component (PAF1) (ENSG00000006712.14), transforming growth factor beta 1 (TGFB1) (ENSG00000105329.9), novel protein, readthrough between CEACAM5-CEACAM6 (AC011513.3) (ENSG00000267881.1), CD177 molecule (CD177) (ENSG00000204936.9), potassium voltage-gated channel subfamily A member 7 (KCNA7) (ENSG00000104848.1), ribosomal protein L28 (RPL28) (ENSG00000108107.14), zinc finger protein 580 (ZNF580) (ENSG00000213015.8), ESF1 nucleolar pre-rRNA processing protein homolog (ESF1) (ENSG00000089048.14), charged multivesicular body protein 4B (CHMP4B) (ENSG00000101421.3), DNA topoisomerase I (TOP1) (ENSG00000198900.5), prefoldin subunit 4 (PFDN4) (ENSG00000101132.9), PRELI domain containing 3B (PRELID3B) (ENSG00000101166.15), synaptonemal complex protein 2 (SYCP2) (ENSG00000196074.12), TATA-box binding protein associated factor 4 (TAF4) (ENSG00000130699.18), cholinergic receptor nicotinic alpha 4 subunit (CHRNA4) (ENSG00000101204.16), SRY-box transcription factor 18 (SOX18) (ENSG00000203883.6), histone H2B type F-S-like (CH507-42P11.1) (ENSG00000274559.3), ATP synthase peripheral stalk subunit F6 (ATP5J) (ENSG00000154723.12), GA binding protein transcription factor subunit alpha (GABPA) (ENSG00000154727.10), SR-related CTD associated factor 4 (SCAF4) (ENSG00000156304.14), H2B clustered histone 12 like (H2BFS) (ENSG00000234289.5), BCLAF1 And THRAP3 family member 3 (CXorf23) (ENSG00000173681.16), proprotein convertase subtilisin/kexin type 1 inhibitor (PCSKIN) (ENSG00000102109.8), MT-RNR2 like 10 (pseudogene) (MTRNR2L10) (ENSG00000256045.2), ATRX chromatin remodeler (ATRX) (ENSG00000085224.21), transcription elongation factor A like 5 (TCEAL5) (ENSG00000204065.2), glutamate dehydrogenase 2 (GLUD2) (ENSG00000182890.4), hypoxanthine phosphoribosyltransferase 1 (HPRT1) (ENSG00000165704.14), oxidoreductase core subunit 6 (MT-ND6) (ENSG00000198695.2), KH RNA binding domain containing, signal transduction associated 1 (KHDRBS1) (ENSG00000121774.17), Y-box binding protein 1 (YBX1) (ENSG00000065978.18), hook microtubule tethering protein 1 (HOOK1) (ENSG00000134709.10), chloride channel accessory 4 (CLCA4) (ENSG00000016602.9), coagulation factor V (F5) (ENSG00000198734.10), glutaredoxin 2 (GLRX2) (ENSG00000023572.9), cell division cycle 73 (CDC73) (ENSG00000134371.11), ribosomal RNA processing 15 homolog (RRP15) (ENSG00000067533.5), olfactory receptor family 2 subfamily M member 3 (OR2M3) (ENSG00000228198.3), DEAD-box helicase 1 (DDX1) (ENSG00000079785.14), mediator of cell motility 1 (MEMO1) (ENSG00000162959.13), ZFP36 ring finger protein like 2 (ZFP36L2) (ENSG00000152518.7), epithelial cell adhesion molecule (EPCAM) (ENSG00000119888.10), actin related protein 2 (ACTR2) (ENSG00000138071.13), ETAA1 activator of ATR kinase (ETAA1) (ENSG00000143971.8), pre-mRNA processing factor 40 homolog A (PRPF40A) (ENSG00000196504.15), NFE2 like bZIP transcription factor 2 (NFE2L2) (ENSG00000116044.15), golgin A4 (GOLGA4) (ENSG00000144674.16), mitochondrial matrix import factor 23 (CCDC58) (ENSG00000160124.9), defective in cullin neddylation 1 domain containing 1 (DCUN1D1) (ENSG00000043093.13), protein phosphatase 1 regulatory inhibitor subunit 2 (PPP1R2) (ENSG00000184203.7), heparan sulfate-glucosamine 3-sulfotransferase 1 (HS3ST1) (ENSG00000002587.9), factor interacting with PAPOLA and CPSF1 (FIP1L1) (ENSG00000145216.15), heterogeneous nuclear ribonucleoprotein D (HNRNPD) (ENSG00000138668.18), paired like homeodomain 2 (PITX2) (ENSG00000164093.16), chromosome 4 open reading frame 33 (C4orf33) (ENSG00000151470.12), RWD domain containing 4 (RWDD4) (ENSG00000182552.14), homer scaffold protein 1 (HOMER1) (ENSG00000152413.14), LysM domain containing 3 (LYSMD3) (ENSG00000176018.12), chromodomain helicase DNA binding protein 1 (CHD1) (ENSG00000153922.10), chromosome 5 open reading frame 15 (C5orf15) (ENSG00000113583.7), protocadherin beta 16 (PCDHB16) (ENSG00000272674.3), mannosidase endo-alpha (MANEA) (ENSG00000172469.15), PNN interacting serine and arginine rich protein (PNISR) (ENSG00000132424.14), anterior gradient 3, protein disulphide isomerase family member (AGR3) (ENSG00000173467.8), homeobox A7 (HOXA7) (ENSG00000122592.7), homeobox A9 (HOXA9) (ENSG00000078399.17), THAP domain containing 5 (THAP5) (ENSG00000177683.13), caveolin 2 (CAV2) (ENSG00000105971.14), zinc finger protein 800 (ZNF800) (ENSG00000048405.9), testis expressed 15, meiosis and synapsis associated (TEX15) (ENSG00000133863.8), MAK16 homolog (MAK16) (ENSG00000198042.10), transcriptional and immune response regulator (C8orf4) (ENSG00000176907.4), snail family transcriptional repressor 2 (SNAI2) (ENSG00000019549.10), lysophospholipase 1 (LYPLA1) (ENSG00000120992.17), XK related 4 (XKR4) (ENSG00000206579.8), potassium voltage-gated channel modifier subfamily V member 1 (KCNV1) (ENSG00000164794.8), HHLA1 neighbor of OC90 (HHLA1) (ENSG00000132297.11), DNA topoisomerase I mitochondrial (TOP1MT) (ENSG00000184428.12), regulatory factor X3 (RFX3) (ENSG00000080298.15), cyclin dependent kinase inhibitor 2A (CDKN2A) (ENSG00000147889.17), intraflagellar transport 74 (IFT74) (ENSG00000096872.15), nuclear receptor subfamily 4 group A member 3 (NR4A3) (ENSG00000119508.17), pre-mRNA processing factor 18 (PRPF18) (ENSG00000165630.13), zinc finger protein 37A (ZNF37A) (ENSG00000075407.18), RAB11 family interacting protein 2 (RAB11FIP2) (ENSG00000107560.10), G protein-coupled receptor 26 (GPR26) (ENSG00000154478.3), small VCP interacting protein (SVIP) (ENSG00000198168.8), electron transfer flavoprotein regulatory factor 1 (LYRM5) (ENSG00000205707.10), H3.5 histone (HF3C) (ENSG00000188375.4), solute carrier family 26 member 10 (SLC26A10) (ENSG00000135502.17), RAP1B, member of RAS oncogene family (RAP1B) (ENSG00000127314.17), TBC1 domain family member 15 (TBC1D15) (ENSG00000121749.15), MGAT4 family member C (MGAT4C) (ENSG00000182050.13), nuclear receptor corepressor 2 (NCOR2) (ENSG00000196498.13), nuclear FMR1 interacting protein 1 (NUFIP1) (ENSG00000083635.7), ubiquitin protein ligase E3A (UBE3A) (ENSG00000114062.18), gamma-aminobutyric acid type A receptor subunit gamma3 (GABRG3) (ENSG00000182256.12), sprouty related EVH1 domain containing 1 (SPRED1) (ENSG00000166068.12), LEO1 homolog, Paf1/RNA polymerase II complex component (LEO1) (ENSG00000166477.12), unc-13 homolog C (UNC13C) (ENSG00000137766.16), RAR related orphan receptor A (RORA) (ENSG00000069667.15), thyroid hormone receptor interactor 4 (TRIP4) (ENSG00000103671.9), solute carrier organic anion transporter family member 3A1 (SLCO3A1) (ENSG00000176463.13), mitochondrial ribosomal protein S34 (MRPS34) (ENSG00000074071.14), homeobox B2 (HOXB2) (ENSG00000173917.10), chromobox 4 (CBX4) (ENSG00000141582.14), occludin/ELL domain containing 1 (OCEL1) (ENSG00000099330.8), cartilage oligomeric matrix protein (COMP) (ENSG00000105664.10), zinc finger protein 254 (ZNF254) (ENSG00000213096.10), FXYD domain containing ion transport regulator 5 (FXYD5) (ENSG00000089327.14), coiled-coil domain containing 9 (CCDC9) (ENSG00000105321.13), myosin heavy chain 14 (MYH14) (ENSG00000105357.16), CCR4-NOT transcription complex subunit 3 (CNOT3) (ENSG00000088038.18), eukaryotic translation initiation factor 2 subunit beta (EIF2S2) (ENSG00000125977.6), solute carrier family 12 member 5 (SLC12A5) (ENSG00000124140.13), cathepsin Z (CTSZ) (ENSG00000101160.13), macrophage migration inhibitory factor (MIF) (ENSG00000240972.1), TSPY like 2 (TSPYL2) (ENSG00000184205.14), peptidylprolyl cis/trans isomerase, NIMA-interacting 4 (PIN4) (ENSG00000102309.12), transcription elongation factor A like 6 (TCEAL6) (ENSG00000204071.10).

22. The cancer subtype identifier according to any of the preceding embodiments, wherein for distinction of MXE1 from MXE2, exon1 is more spliced-in (the PSI value is higher) than exon2 in MXE1 subtype for the exons as defined in embodiment 10 and/or 11.

23. The cancer subtype identifier according to any of the preceding embodiments, wherein for distinction of MXE1 from MXE2, exon1 is more spliced-in (the PSI value is higher) than exon2 in MXE2 subtype for the exons as defined in embodiment 12 and/or 13.

24. The cancer subtype identifier according to any of the preceding embodiments, wherein in MXE1 subtype, primarily exon1 (with genomic coordinates chr21: 45480466-45480520) of gene collagen type XVIII alpha 1 chain (COL18A1) (ENSG00000182871.14), is 18.3%+/−0.9 (SE) more spliced-in (the PSI value is higher) compared to exon2 (with genomic coordinates chr21: 45480699-45480858); exon1 (with genomic coordinates chr19: 33012646-33012724) of gene rhophilin Rho GTPase binding protein 2 (RHPN2) (ENSG00000131941.7), is 12.2%+/−0.6 (SE) more spliced-in (the PSI value is higher) compared to exon2 (with genomic coordinates chr19: 33021570-33021646); exon1 (with genomic coordinates chr19: 10986188-10986593) of gene SWI/SNF related, matrix associated, actin dependent regulator of chromatin, subfamily a, member 4 (SMARCA4) (ENSG00000127616.18), is 16.8%+/−0.9 (SE) more spliced-in (the PSI value is higher) compared to exon2 (with genomic coordinates chr19: 10986904-10987003); exon1 (with genomic coordinates chr17: 69304668-69304810) of gene ATP binding cassette subfamily A member 5 (ABCA5) (ENSG00000154265.15), is 23%+/−1.7 (SE) more spliced-in (the PSI value is higher) compared to exon2 (with genomic coordinates chr17: 69306724-69306954); exon1 (with genomic coordinates chr11: 66564087-66564146) of gene cathepsin F (CTSF) (ENSG00000174080.10), is 20.7%+/−1.1 (SE) more spliced-in (the PSI value is higher) compared to exon2 (with genomic coordinates chr11: 66564557-66564648); exon1 (with genomic coordinates chr1: 200844781-200844869) of gene calmodulin regulated spectrin associated protein family member 2 (CAMSAP2) (ENSG00000118200.14), is 16.7%+/−1.2 (SE) more spliced-in (the PSI value is higher) compared to exon2 (with genomic coordinates chr1: 200848031-200850234); exon1 (with genomic coordinates chr2: 188988080-188988134) of gene collagen type III alpha 1 chain (COL3A1) (ENSG00000168542.14), is 15.8%+/−0.6 (SE) more spliced-in (the PSI value is higher) compared to exon2 (with genomic coordinates chr2: 188989395-188989449); exon1 (with genomic coordinates chr1: 54205294-54205410) of gene mitochondrial ribosomal protein L37 (MRPL37) (ENSG00000116221.15), is 13.7%+/−0.5 (SE) more spliced-in (the PSI value is higher) compared to exon2 (with genomic coordinates chr1: 54209945-54210131); exon1 (with genomic coordinates chr6: 149836084-149836297) of gene LDL receptor related protein 11 (LRP11) (ENSG00000120256.9), is 13.6%+/−0.6 (SE) more spliced-in (the PSI value is higher) compared to exon2 (with genomic coordinates chr6: 149837337-149837463); exon1 (with genomic coordinates chrX: 41140655-41140771) of gene ubiquitin specific peptidase 9 X-linked (USP9X) (ENSG00000124486.12), is 16.4%+/−0.7 (SE) more spliced-in (the PSI value is higher) compared to exon2 (with genomic coordinates chrX: 41140965-41141217); exon1 (with genomic coordinates chr6: 31642828-31643115) of gene BAG cochaperone 6 (BAG6) (ENSG00000204463.12), is 14.8%+/−0.9 (SE) more spliced-in (the PSI value is higher) compared to exon2 (with genomic coordinates chr6: 31644081-31644194); exon1 (with genomic coordinates chr6: 33303954-33303989) of gene TAP binding protein (TAPBP) (ENSG00000231925.11), is 13.4%+/−0.6 (SE) more spliced-in (the PSI value is higher) compared to exon2 (with genomic coordinates chr6: 33304127-33304217); exon1 (with genomic coordinates chr7: 34966893-34966971) of gene dpy-19 like C-mannosyltransferase 1 (DPY19L1) (ENSG00000173852.14), is 18.3%+/−1.3 (SE) more spliced-in (the PSI value is higher) compared to exon2 (with genomic coordinates chr7: 34969432-34969532); exon1 (with genomic coordinates chr2: 24299908-24300171) of gene intersectin 2 (ITSN2) (ENSG00000198399.14), is 14.9%+/−0.7 (SE) more spliced-in (the PSI value is higher) compared to exon2 (with genomic coordinates chr2: 24301153-24301239); exon1 (with genomic coordinates chr10: 112438335-112438389) of gene zinc finger DHHC-type palmitoyltransferase 6 (ZDHHC6) (ENSG00000023041.11), is 16.4%+/−0.8 (SE) more spliced-in (the PSI value is higher) compared to exon2 (with genomic coordinates chr10: 112440533-112440695); exon1 (with genomic coordinates chr10: 112438335-112438389) of gene zinc finger DHHC-type palmitoyltransferase 6 (ZDHHC6) (ENSG00000023041.11), is 15.9%+/−0.8 (SE) more spliced-in (the PSI value is higher) compared to exon2 (with genomic coordinates chr10: 112442191-112442351), preferably
wherein in MXE1 subtype, exon1 (with genomic coordinates chr21: 45480466-45480520) of gene collagen type XVIII alpha 1 chain (COL18A1) (ENSG00000182871.14), is 18.3%+/−0.9 (SE) more spliced-in (the PSI value is higher) compared to exon2 (with genomic coordinates chr21: 45480699-45480858); exon1 (with genomic coordinates chr19: 33012646-33012724) of gene rhophilin Rho GTPase binding protein 2 (RHPN2) (ENSG00000131941.7), is 12.2%+/−0.6 (SE) more spliced-in (the PSI value is higher) compared to exon2 (with genomic coordinates chr19: 33021570-33021646); exon1 (with genomic coordinates chr19: 10986188-10986593) of gene SWI/SNF related, matrix associated, actin dependent regulator of chromatin, subfamily a, member 4 (SMARCA4) (ENSG00000127616.18), is 16.8%+/−0.9 (SE) more spliced-in (the PSI value is higher) compared to exon2 (with genomic coordinates chr19: 10986904-10987003); exon1 (with genomic coordinates chr17: 69304668-69304810) of gene ATP binding cassette subfamily A member 5 (ABCA5) (ENSG00000154265.15), is 23%+/−1.7 (SE) more spliced-in (the PSI value is higher) compared to exon2 (with genomic coordinates chr17: 69306724-69306954); exon1 (with genomic coordinates chr11: 66564087-66564146) of gene cathepsin F (CTSF) (ENSG00000174080.10), is 20.7%+/−1.1 (SE) more spliced-in (the PSI value is higher) compared to exon2 (with genomic coordinates chr11: 66564557-66564648); exon1 (with genomic coordinates chr1: 200844781-200844869) of gene calmodulin regulated spectrin associated protein family member 2 (CAMSAP2) (ENSG00000118200.14), is 16.7%+/−1.2 (SE) more spliced-in (the PSI value is higher) compared to exon2 (with genomic coordinates chr1: 200848031-200850234); exon1 (with genomic coordinates chr2: 188988080-188988134) of gene collagen type III alpha 1 chain (COL3A1) (ENSG00000168542.14), is 15.8%+/−0.6 (SE) more spliced-in (the PSI value is higher) compared to exon2 (with genomic coordinates chr2: 188989395-188989449); exon1 (with genomic coordinates chr1: 54205294-54205410) of gene mitochondrial ribosomal protein L37 (MRPL37) (ENSG00000116221.15), is 13.7%+/−0.5 (SE) more spliced-in (the PSI value is higher) compared to exon2 (with genomic coordinates chr1: 54209945-54210131); exon1 (with genomic coordinates chr6: 149836084-149836297) of gene LDL receptor related protein 11 (LRP11) (ENSG00000120256.9), is 13.6%+/−0.6 (SE) more spliced-in (the PSI value is higher) compared to exon2 (with genomic coordinates chr6: 149837337-149837463); exon1 (with genomic coordinates chrX: 41140655-41140771) of gene ubiquitin specific peptidase 9 X-linked (USP9X) (ENSG00000124486.12), is 16.4%+/−0.7 (SE) more spliced-in (the PSI value is higher) compared to exon2 (with genomic coordinates chrX: 41140965-41141217); exon1 (with genomic coordinates chr6: 31642828-31643115) of gene BAG cochaperone 6 (BAG6) (ENSG00000204463.12), is 14.8%+/−0.9 (SE) more spliced-in (the PSI value is higher) compared to exon2 (with genomic coordinates chr6: 31644081-31644194); exon1 (with genomic coordinates chr6: 33303954-33303989) of gene TAP binding protein (TAPBP) (ENSG00000231925.11), is 13.4%+/−0.6 (SE) more spliced-in (the PSI value is higher) compared to exon2 (with genomic coordinates chr6: 33304127-33304217); exon1 (with genomic coordinates chr7: 34966893-34966971) of gene dpy-19 like C-mannosyltransferase 1 (DPY19L1) (ENSG00000173852.14), is 18.3%+/−1.3 (SE) more spliced-in (the PSI value is higher) compared to exon2 (with genomic coordinates chr7: 34969432-34969532); exon1 (with genomic coordinates chr2: 24299908-24300171) of gene intersectin 2 (ITSN2) (ENSG00000198399.14), is 14.9%+/−0.7 (SE) more spliced-in (the PSI value is higher) compared to exon2 (with genomic coordinates chr2: 24301153-24301239); exon1 (with genomic coordinates chr10: 112438335-112438389) of gene zinc finger DHHC-type palmitoyltransferase 6 (ZDHHC6) (ENSG00000023041.11), is 16.4%+/−0.8 (SE) more spliced-in (the PSI value is higher) compared to exon2 (with genomic coordinates chr10: 112440533-112440695); exon1 (with genomic coordinates chr10: 112438335-112438389) of gene zinc finger DHHC-type palmitoyltransferase 6 (ZDHHC6) (ENSG00000023041.11), is 15.9%+/−0.8 (SE) more spliced-in (the PSI value is higher) compared to exon2 (with genomic coordinates chr10: 112442191-112442351); exon1 (with genomic coordinates chr19: 49862043-49862105) of gene polynucleotide kinase 3′-phosphatase (PNKP) (ENSG00000039650.11), is 14.9%+/−0.9 (SE) more spliced-in (the PSI value is higher) compared to exon2 (with genomic coordinates chr19: 49862184-49862281); exon1 (with genomic coordinates chr19: 47372729-47372923) of gene DExH-box helicase 34 (DHX34) (ENSG00000134815.18), is 15%+/−1 (SE) more spliced-in (the PSI value is higher) compared to exon2 (with genomic coordinates chr19: 47373598-47373700); exon1 (with genomic coordinates chr15: 65701050-65701192) of gene DENN domain containing 4A (DENND4A) (ENSG00000174485.15), is 15%+/−1.1 (SE) more spliced-in (the PSI value is higher) compared to exon2 (with genomic coordinates chr15: 65701761-65701890); exon1 (with genomic coordinates chr17: 67145742-67145890) of gene helicase with zinc finger (HELZ) (ENSG00000198265.11), is 14.2%+/−0.8 (SE) more spliced-in (the PSI value is higher) compared to exon2 (with genomic coordinates chr17: 67149866-67149985); exon1 (with genomic coordinates chr11: 2527927-2528018) of gene potassium voltage-gated channel subfamily Q member 1 (KCNQ1) (ENSG00000053918.16), is 12.3%+/−0.8 (SE) more spliced-in (the PSI value is higher) compared to exon2 (with genomic coordinates chr11: 2587569-2587692); exon1 (with genomic coordinates chr5: 9050409-9050457) of gene semaphorin 5A (SEMA5A) (ENSG00000112902.11), is 12.6%+/−1.2 (SE) more spliced-in (the PSI value is higher) compared to exon2 (with genomic coordinates chr5: 9051872-9052028); exon1 (with genomic coordinates chr20: 10641112-10641244) of gene jagged canonical Notch ligand 1 (JAG1) (ENSG00000101384.12), is 13.3%+/−0.6 (SE) more spliced-in (the PSI value is higher) compared to exon2 (with genomic coordinates chr20: 10641459-10641693).

25. The cancer subtype identifier according to any of the preceding embodiments, wherein in MXE2 subtype primarily, exon1 (with genomic coordinates chr20: 48988490-48988662) of gene ADP ribosylation factor guanine nucleotide exchange factor 2 (ARFGEF2) (ENSG00000124198.8), is 15%+/−0.6 (SE) more spliced-in (the PSI value is higher) compared to exon2 (with genomic coordinates chr20: 48989284-48989436); exon1 (with genomic coordinates chr18: 62266700-62266749) of gene RAB11 binding and LisH domain (KIAA1468) (ENSG00000134444.14), is 15.8%+/−1 (SE) more spliced-in (the PSI value is higher) compared to exon2 (with genomic coordinates chr18: 62268868-62268948); exon1 (with genomic coordinates chr16: 30081258-30081310) of gene protein phosphatase 4 catalytic subunit (PPP4C) (ENSG00000149923.13), is 16.1%+/−0.8 (SE) more spliced-in (the PSI value is higher) compared to exon2 (with genomic coordinates chr16: 30082483-30082534); exon1 (with genomic coordinates chr15: 34255261-34255392) of gene solute carrier family 12 member 6 (SLC12A6) (ENSG00000140199.11), is 14.1%+/−1 (SE) more spliced-in (the PSI value is higher) compared to exon2 (with genomic coordinates chr15: 34258812-34258944); exon1 (with genomic coordinates chr14: 22905605-22905659) of gene RNA binding motif protein 23 (RBM23) (ENSG00000100461.17), is 19.2%+/−1.2 (SE) more spliced-in (the PSI value is higher) compared to exon2 (with genomic coordinates chr14: 22908332-22908380); exon1 (with genomic coordinates chr12: 7142085-7142139) of gene calsyntenin 3 (CLSTN3) (ENSG00000139182.13), is 17.2%+/−1 (SE) more spliced-in (the PSI value is higher) compared to exon2 (with genomic coordinates chr12: 7142868-7143026); exon1 (with genomic coordinates chr20: 13534021-13534141) of gene taspase 1 (TASP1) (ENSG00000089123.15), is 14.1%+/−0.8 (SE) more spliced-in (the PSI value is higher) compared to exon2 (with genomic coordinates chr20: 13559007-13559114); exon1 (with genomic coordinates chr8: 52630469-52630528) of gene RB1 inducible coiled-coil 1 (RB1CC1) (ENSG00000023287.12), is 14.1%+/−0.8 (SE) more spliced-in (the PSI value is higher) compared to exon2 (with genomic coordinates chr8: 52634920-52634968); exon1 (with genomic coordinates chr1: 150156065-150156206) of gene pleckstrin homology domain containing 01 (PLEKHO1) (ENSG00000023902.13), is 12.8%+/−0.7 (SE) more spliced-in (the PSI value is higher) compared to exon2 (with genomic coordinates chr1: 150157384-150157486); exon1 (with genomic coordinates chr2: 32607454-32607643) of gene baculoviral IAP repeat containing 6 (BIRC6) (ENSG00000115760.13), is 13.4%+/−0.6 (SE) more spliced-in (the PSI value is higher) compared to exon2 (with genomic coordinates chr2: 32611447-32611582); exon1 (with genomic coordinates chr11: 9430874-9431003) of gene importin 7 (IPO7) (ENSG00000205339.9), is 14.4%+/−0.6 (SE) more spliced-in (the PSI value is higher) compared to exon2 (with genomic coordinates chr11: 9433569-9433636); exon1 (with genomic coordinates chr17: 82081163-82081352) of gene fatty acid synthase (FASN) (ENSG00000169710.8), is 13.5%+/−0.7 (SE) more spliced-in (the PSI value is higher) compared to exon2 (with genomic coordinates chr17: 82081600-82081843); exon1 (with genomic coordinates chr6: 157873101-157873176) of gene sorting nexin 9 (SNX9) (ENSG00000130340.15), is 15.5%+/−0.6 (SE) more spliced-in (the PSI value is higher) compared to exon2 (with genomic coordinates chr6: 157875050-157875176); exon1 (with genomic coordinates chr7: 5390484-5390628) of gene trinucleotide repeat containing 18 (TNRC18) (ENSG00000182095.14), is 21.6%+/−1 (SE) more spliced-in (the PSI value is higher) compared to exon2 (with genomic coordinates chr7: 5394439-5394595); exon1 (with genomic coordinates chr17: 50195922-50195976) of gene collagen type I alpha 1 chain (COL1A1) (ENSG00000108821.13), is 13%+/−0.4 (SE) more spliced-in (the PSI value is higher) compared to exon2 (with genomic coordinates chr17: 50196154-50196199), preferably
wherein in MXE2 subtype, exon1 (with genomic coordinates chr20: 48988490-48988662) of gene ADP ribosylation factor guanine nucleotide exchange factor 2 (ARFGEF2) (ENSG00000124198.8), is 15%+/−0.6 (SE) more spliced-in (the PSI value is higher) compared to exon2 (with genomic coordinates chr20: 48989284-48989436); exon1 (with genomic coordinates chr18: 62266700-62266749) of gene RAB11 binding and LisH domain (KIAA1468) (ENSG00000134444.14), is 15.8%+/−1 (SE) more spliced-in (the PSI value is higher) compared to exon2 (with genomic coordinates chr18: 62268868-62268948); exon1 (with genomic coordinates chr16: 30081258-30081310) of gene protein phosphatase 4 catalytic subunit (PPP4C) (ENSG00000149923.13), is 16.1%+/−0.8 (SE) more spliced-in (the PSI value is higher) compared to exon2 (with genomic coordinates chr16: 30082483-30082534); exon1 (with genomic coordinates chr15: 34255261-34255392) of gene solute carrier family 12 member 6 (SLC12A6) (ENSG00000140199.11), is 14.1%+/−1 (SE) more spliced-in (the PSI value is higher) compared to exon2 (with genomic coordinates chr15: 34258812-34258944); exon1 (with genomic coordinates chr14: 22905605-22905659) of gene RNA binding motif protein 23 (RBM23) (ENSG00000100461.17), is 19.2%+/−1.2 (SE) more spliced-in (the PSI value is higher) compared to exon2 (with genomic coordinates chr14: 22908332-22908380); exon1 (with genomic coordinates chr12: 7142085-7142139) of gene calsyntenin 3 (CLSTN3) (ENSG00000139182.13), is 17.2%+/−1 (SE) more spliced-in compared to exon2 (with genomic coordinates chr12: 7142868-7143026); exon1 (with genomic coordinates chr20: 13534021-13534141) of gene taspase 1 (TASP1) (ENSG00000089123.15), is 14.1%+/−0.8 (SE) more spliced-in (the PSI value is higher) compared to exon2 (with genomic coordinates chr20: 13559007-13559114); exon1 (with genomic coordinates chr8: 52630469-52630528) of gene RB1 inducible coiled-coil 1 (RB1CC1) (ENSG00000023287.12), is 14.1%+/−0.8 (SE) more spliced-in (the PSI value is higher) compared to exon2 (with genomic coordinates chr8: 52634920-52634968); exon1 (with genomic coordinates chr1: 150156065-150156206) of gene pleckstrin homology domain containing 01 (PLEKHO1) (ENSG00000023902.13), is 12.8%+/−0.7 (SE) more spliced-in (the PSI value is higher) compared to exon2 (with genomic coordinates chr1: 150157384-150157486); exon1 (with genomic coordinates chr2: 32607454-32607643) of gene baculoviral IAP repeat containing 6 (BIRC6) (ENSG00000115760.13), is 13.4%+/−0.6 (SE) more spliced-in (the PSI value is higher) compared to exon2 (with genomic coordinates chr2: 32611447-32611582); exon1 (with genomic coordinates chr11: 9430874-9431003) of gene importin 7 (IPO7) (ENSG00000205339.9), is 14.4%+/−0.6 (SE) more spliced-in (the PSI value is higher) compared to exon2 (with genomic coordinates chr11: 9433569-9433636); exon1 (with genomic coordinates chr17: 82081163-82081352) of gene fatty acid synthase (FASN) (ENSG00000169710.8), is 13.5%+/−0.7 (SE) more spliced-in (the PSI value is higher) compared to exon2 (with genomic coordinates chr17: 82081600-82081843); exon1 (with genomic coordinates chr6: 157873101-157873176) of gene sorting nexin 9 (SNX9) (ENSG00000130340.15), is 15.5%+/−0.6 (SE) more spliced-in (the PSI value is higher) compared to exon2 (with genomic coordinates chr6: 157875050-157875176); exon1 (with genomic coordinates chr7: 5390484-5390628) of gene trinucleotide repeat containing 18 (TNRC18) (ENSG00000182095.14), is 21.6%+/−1 (SE) more spliced-in (the PSI value is higher) compared to exon2 (with genomic coordinates chr7: 5394439-5394595); exon1 (with genomic coordinates chr17: 50195922-50195976) of gene collagen type I alpha 1 chain (COL1A1) (ENSG00000108821.13), is 13%+/−0.4 (SE) more spliced-in (the PSI value is higher) compared to exon2 (with genomic coordinates chr17: 50196154-50196199); exon1 (with genomic coordinates chr19: 55091649-55091700) of gene protein phosphatase 1 regulatory subunit 12C (PPP1R12C) (ENSG00000125503.12), is 15.3%+/−1 (SE) more spliced-in (the PSI value is higher) compared to exon2 (with genomic coordinates chr19: 55091858-55091909); exon1 (with genomic coordinates chr17: 69301138-69301286) of gene ATP binding cassette subfamily A member 5 (ABCA5) (ENSG00000154265.15), is 17.6%+/−1.5 (SE) more spliced-in (the PSI value is higher) compared to exon2 (with genomic coordinates chr17: 69302717-69302906); exon1 (with genomic coordinates chr4: 148152468-148152613) of gene nuclear receptor subfamily 3 group C member 2 (NR3C2) (ENSG00000151623.14), is 12.2%+/−1.5 (SE) more spliced-in (the PSI value is higher) compared to exon2 (with genomic coordinates chr4: 148154550-148154901); exon1 (with genomic coordinates chr9: 120453455-120453873) of gene CDK5 regulatory subunit associated protein 2 (CDK5RAP2) (ENSG00000136861.17), is 15%+/−0.9 (SE) more spliced-in (the PSI value is higher) compared to exon2 (with genomic coordinates chr9: 120460571-120460667).

26. The cancer subtype identifier according to any of the preceding embodiments, which classifies tumors by determining
the PSI values based on the occurrence of at least one mutually exclusive usage exon event,
the expression of small nuclear RNAs and
the expression of predictive protein-coding genes.

27. A to method for classifying tumors using a cancer subtype identifier based on at least one
PSI values based on the occurrence of at least one mutually exclusive exon usage event,
expression of small nuclear RNAs
expression of predictive protein-coding genes,
a combination thereof.

28. A method according to embodiment 26 or 27, for classifying tumors using a cancer subtype identifier based on at least one
PSI values based on the occurrence of at least one mutually exclusive exon usage event,
expression of small nuclear RNAs
expression of predictive protein-coding genes,
a combination thereof,
   wherein the tumor is classified as MXE1 subtype and MXE2 subtype

29. A method according to embodiment 26, 27 or 28, for classifying tumors using a cancer subtype identifier based on at least one
PSI (percent spliced-in) values based on the occurrence of at least one mutually exclusive exon usage event,
expression of small nuclear RNAs,
expression of predictive protein-coding genes,
a combination thereof,
   wherein the tumor is classified as MXE1 subtype and MXE2 subtype, wherein
   the MXE1 subtype is characterized by
   i) a distinct combination of PSI values from mutually exclusive exon usage events,
   ii) a distinct combination of expressions of small nuclear RNAs
   iii) a distinct combination of protein coding genes,
   a combination of i) and ii), i) and iii), ii) and iii) and i), ii) and iii),
   wherein MXE1 subtype is associated with a good prognosis compared to the MXE2 subtype.
   and/or
   the MXE2 subtype is characterized by
   iv) a distinct combination of PSI values from mutually exclusive exon usage events,
   v) a distinct combination of expressions of small nuclear RNAs
   vi) a distinct combination of protein coding genes,
a combination of iv) and v), iv) and vi), v) and vi) and iv), v) and vi),
wherein the MXE2 subtype is associated with a poor prognosis compared to the MXE1 subtype.
wherein i) is different from iv), ii) is different from v), iii) is different from vi).

30. A method according to embodiment 26 to 28, for classifying tumors using a cancer subtype identifier based on at least one
PSI (percent spliced-in) values based on the occurrence of at least one mutually exclusive exon usage event,
expression of small nuclear RNAs,
a combination thereof,
   wherein the tumor is classified as MXE1 subtype and MXE2 subtype,
   optionally the cancer subtype identifier classifies tumors additionally based on expression of predictive protein-coding genes.

31. A method for predicting the prognosis for a subject suffering from colorectal cancer using the PSI values based on the occurrence of at least one mutually exclusive exon usage event, and/or the expression of small nuclear RNAs and/or the expression of predictive protein-coding genes comprising the steps:
a) collecting a sample from a tumor and/or body fluid and/or body excretion of a subject;
b) determining of at least one of
the PSI values based on the occurrence of at least one mutually exclusive exon usage event, the deregulation of the expression of small nuclear RNAs,
expression of subtype predictive protein-coding genes,
combinations thereof,
c) conversion of values of step b) into a rating scale to indicate the disease prognosis.

32. The method according to of embodiment 31, wherein in step c) the conversion of values of step b) to indicate the disease prognosis classifies the tumors as MXE1 subtype or MXE2 subtype.

33. The method of according to any of embodiments 31 or 32, wherein in step c) the conversion of the values of step b) to indicate the disease prognosis is used to decide whether the subject is subjected to a treatment by anti-tumor therapy and/or surveillance of progress of the disease.

34. The method of according to any of embodiments 31 to 33, wherein in step c) the conversion of the values of step b) to indicate the disease prognosis is combined with tumor classification subtypes other than MXE1 subtype and MXE2 subtype, preferably is combined with at least one tumor classification subtypes selected from CMS and immune infiltrate.

35. A method for treating a subject suffering from cancer using the PSI values based on the occurrence of at least one mutually exclusive exon event, and/or the deregulation of expression of small nuclear RNAs and/or the deregulation of expression of subtype predictive protein-coding genes comprising the steps:
a′) collecting a sample from a tumor and/or bodily fluid and/or body excretion of a subject;
b′) determining of at least one of
the PSI values based on the occurrence of at least one mutually exclusive exon usage event, the deregulation of the expression of small nuclear RNAs,
expression of subtype predictive protein-coding genes,
combinations thereof;
c′) conversion of the values of step b) into a rating scale to indicate the disease prognosis by classifying the tumors as MXE1 subtype or MXE2 subtype;
d′) subjecting the subject to an anti tumor treatment if the rating scale indicates that the subject is likely to experience increased progression of the disease.

36. Biomarker (composition) for predicting the prognosis for a subject suffering from cancer, in particular for lung cancer and colorectal cancer, selected from the group consisting of U1 small nuclear 1 RNA (RNU1-1); U12 small nuclear RNA (RNU12); U4 small nuclear 2 RNA (RNU4-2); U5A small nuclear 1 RNA (RNU5A-1); U5B small nuclear 1 RNA (RNU5B-1); U5D small nuclear 1 RNA (RNU5D-1); U5E small nuclear 1 RNA (RNUSE-1); U6 small nuclear 9 RNA (RNU6-9); U6atac small nuclear RNA (RNU6ATAC); variant U1 small nuclear 1 RNA (RNVU1-1); variant U1 small nuclear 15 RNA (RNVU1-15); variant U1 small nuclear 18 RNA (RNVU1-18); variant U1 small nuclear 19 RNA (RNVU1-19); variant U1 small nuclear 6 RNA (RNVU1-6); variant U1 small nuclear 7 RNA (RNVU1-7); U11 small nuclear RNA (RNU11); U1 small nuclear 4 RNA (RNU1-4); U4 small nuclear 1 RNA (RNU4-1); U4atac small nuclear RNA (RNU4ATAC); U5F small nuclear 1 RNA (RNU5F-1); U6 small nuclear 1 RNA (RNU6-1); U6 small nuclear 2 RNA (RNU6-2); U1 small nuclear RNA (RF00003); U4 small nuclear RNA (RF00015) and U7 small nuclear RNA (RF00066), in particular RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC, RNVU1-1, RNVU1-15, RNVU1-19 are upregulated in MXE2 subtype compared to MXE1 subtype and RNU1-1, RNU1-4, RNU4-2, RNVU1-18, RNVU1-6, RNVU1-7, RF00015, RF00066 and RF00003 are downregulated in MXE2 subtype compared to MXE1 subtype.

37. Biomarker (composition) for predicting the prognosis for a subject suffering from cancer, in particular non-small lung cancer (adenocarcinoma), selected from RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC, U4 and U7, in particular RNU11, RNU12, RNU4-1, RNU4ATAC, RNU5A-1, RNU5B-1, RNU5D-1, RNU5E-1, RNU5F-1, RNU6-1, RNU6-2, RNU6-9, RNU6ATAC are upregulated in MXE2 subtype compared to MXE1 subtype and RF00015 and RF00066 are downregulated in MXE2 subtype compared to MXE1 subtype.

38. A kit for predicting the prognosis for a subject suffering from cancer using at least one of the PSI values based on the occurrence of at least one mutually exclusive exon usage event, the deregulation of the expression of small nuclear RNAs, the expression of subtype predictive protein-coding genes, combinations thereof, preferably at least one exon skipping event or one biomarker as defined in claim 22 or 23, comprising means for determining the PSI value, the expression of small nuclear RNAs and the expression protein-coding genes.

39. A kit for predicting the prognosis for a subject suffering from cancer using the cancer subtype identifier as defined in embodiments 1 to 25 comprising a composition for determining the PSI value, the expression of small nuclear RNAs and the expression protein-coding genes.

40. The use of at least one of PSI values based on the occurrence of at least one mutually exclusive exon usage event, the expression of small nuclear RNAs, the expression of predictive protein-coding genes, combination thereof as a marker of the prognosis for a subject suffering from cancer.

41. The use of the cancer subtype identifier as defined in claims 1 to 25 as a marker of the prognosis for a subject suffering from cancer.

The invention will be illustrated further with reference to the examples that follow, without restricting the scope to the specific embodiments described. The invention includes all combinations of described and especially of preferred features that do not exclude each other.

### EXAMPLES

Utilizing total RNA-Seq data, in a first cohort of 429 CRC from Indivumed GmbH specimens that have definitive CMS subtypes, percent spliced-in (PSI) values for mutually exclusive exon usage events were calculated where only one of the adjacent exons is included in the transcript, but not both. These PSI values are estimated by using computational tools such as rMATS-turbo (github.com/Xinglab/rmats-turbo) or MISO (miso.readthedocs.io/en/fastmiso/). Non-negative matrix factorization of the PSI values revealed two latent stable groups that prognostically stratified CRC patients. The groups (MXE1 and MXE2 subtypes) were independent of CMS and tumor staging and can be combined with conventional clinical classifications to refine them. Based on PSI values from MXE events differentially spliced between the groups (FDR≤0.1 and |PSI|≥0.1), snRNAs and gene expression, an identifier was developed utilizing supervised machine learning methods for predicting these novel prognostic groups.

The classification system generates new prognostic groups that are independent from traditional staging and more predictive of patient outcomes. Furthermore, the underlying biology between these two groups are closely associated with changes in splicing and expression of spliceosomal small nuclear RNAs.

The identifier based on MXE events and snRNAs, were tested and confirmed on an independent validation cohort of 246 CRC samples, which had not been part of the process and could not be conclusively assigned to a single CMS group using expression-based classifiers. Publicly available datasets, such as The Cancer Genome Atlas (TCGA, second cohort), utilize a different RNA-Seq library preparation protocol than our (first) cohort. Specifically, TCGA employs an unstranded, polyA-selected library preparation, which can obscure the subtle changes in the splicing signal and cannot detect non-polyadenylated snRNAs. To address this, a surrogate model was developed based on the expression of protein-coding genes, capable of predicting these novel prognostic subtypes. This model was validated on both the internal validation dataset (cohort 1) and the TCGA dataset (cohort 2). For both validation cohorts, the progression free survival associated with the predicted groups were evaluated. The models effectively identified patients with poor prognosis and when combined with tumor staging, the novel groups stratified patients with stage Ill tumors into groups with unique prognostic outcomes.

Spatial transcriptomic profiling of FFPE samples (MXE1 n=3, MXE2 n=4) was performed to identify cell type-specific gene expression patterns. Spatially-aware clustering revealed distinct clusters with subtype-specific expression patterns. MXE2-upregulated genes from individual spatial clusters were used to generate MCP-counter enrichment scores in bulk RNA-Seq samples. Stratification by median enrichment score (enriched vs depleted) identified patient groups unique prognostic outcomes.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified. Methods of nucleotide amplification, cell transfection, and protein expression and purification are similarly within the level of skill in the art.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

We claim:

1. A method for classifying tumors using a cancer subtype identifier based on
percent spliced-in (PSI) values based on the occurrence of at least one mutually exclusive exon usage event, or
expression of small nuclear RNAs or
expression of predictive protein-coding genes, or
a combination thereof,
wherein the tumor is classified as MXE1 subtype and MXE2 subtype.

2. The method according to claim 1, for classifying tumors using a cancer subtype identifier based on PSI (percent spliced-in) values based on the occurrence of at least one mutually exclusive exon usage event,
expression of small nuclear RNAs,
expression of predictive protein-coding genes,
a combination thereof,
wherein the tumor is classified as MXE1 subtype and MXE2 subtype, wherein
the MXE1 subtype is characterized by
i) a distinct combination of PSI values from mutually exclusive exon usage events,
ii) a distinct combination of expressions of small nuclear RNAs
iii) a distinct combination of protein coding genes,
a combination of i) and ii), i) and iii), ii) and iii) and i), ii) and iii),
wherein MXE1 subtype is associated with a good prognosis compared to the MXE2 subtype.
and/or
the MXE2 subtype is characterized by
iv) a distinct combination of PSI values from mutually exclusive exon usage events,
v) a distinct combination of expressions of small nuclear RNAs
vi) a distinct combination of protein coding genes,
a combination of iv) and v), iv) and vi), v) and vi) and iv), v) and vi),
wherein the MXE2 subtype is associated with a poor prognosis compared to the MXE1 subtype,
wherein i) is different from iv), ii) is different from v), iii) is different from vi).

3. A method for predicting the prognosis for a subject suffering from cancer the method comprising using a cancer subtype identifier based on PSI values based on an occurrence of at least one mutually exclusive exon usage event, and/or expression of small nuclear RNAs and/or expression of protein-coding genes comprising the steps:
a) collecting a sample from a tumor and/or body fluid and/or body excretion of a subject;
b) determining at least one of
the PSI values based on the occurrence of at least one mutually exclusive exon usage event,
deregulation of the expression of small nuclear RNAs,
expression of subtype predictive protein-coding genes, and
combinations thereof;
c) converting of values of step b) into a rating scale to indicate the disease prognosis.

4. A method for administering an anti tumor treatment to a subject suffering from cancer the method comprising using a cancer subtype identifier based on PSI values based on an occurrence of at least one mutually exclusive exon event, and/or the deregulation of expression of small nuclear RNAs and/or deregulation of expression of subtype predictive protein-coding genes comprising the steps:
a′) collecting a sample from a tumor and/or body fluid and/or body excretion of a subject;
b′) determining at least one of
the PSI values based on the occurrence of at least one mutually exclusive exon usage event,
deregulation of the expression of small nuclear RNAs,
expression of subtype predictive protein-coding genes,
combinations thereof;
c′) conversion of the values of step b) into a rating scale to indicate the disease prognosis by classifying the tumors as MXE1 subtype or MXE2 subtype;
d′) subjecting the subject to an anti tumor treatment if the rating scale indicates that the subject is likely to experience an increased progression of the disease.

5. The method according to claim 1, wherein the cancer is selected from the group consisting of lung cancer, breast cancer, gastric cancer, esophageal cancer, skin cancer, ovarian cancer, cervical cancer, liver cancer, bladder cancer, kidney cancer, pancreatic cancer, prostate cancer, thyroid cancer, head cancer, neck cancer, oral cancer, laryngeal cancer, rectal cancer and colon cancer, in particular colorectal cancer and non-small lung cancer (lung adenocarcinoma).

6. The method according claim 1, wherein for identification of MXE1 subtype at least one primary mutually exclusive exon usage event is present, characterized by differential splicing of exons that are incorporated into different transcripts, as exon 1 and exon 2, wherein the mutually exclusive splicing events are optionally selected from exon1 (with genomic coordinates chr21: 45480466-45480520) of gene collagen type XVIII alpha 1 chain (COL18A1) (ENSG00000182871.14), compared to exon2 (with genomic coordinates chr21: 45480699-45480858); exon1 (with genomic coordinates chr19: 33012646-33012724) of gene rhophilin Rho GTPase binding protein 2 (RHPN2) (ENSG00000131941.7), compared to exon2 (with genomic coordinates chr19: 33021570-33021646); exon1 (with genomic coordinates chr19: 10986188-10986593) of gene SWI/SNF related, matrix associated, actin dependent regulator of chromatin, subfamily a, member 4 (SMARCA4) (ENSG00000127616.18), compared to exon2 (with genomic coordinates chr19: 10986904-10987003); exon1 (with genomic coordinates chr17: 69304668-69304810) of gene ATP binding cassette subfamily A member 5 (ABCA5) (ENSG00000154265.15), compared to exon2 (with genomic coordinates chr17: 69306724-69306954); exon1 (with genomic coordinates chr11: 66564087-66564146) of gene cathepsin F (CTSF) (ENSG00000174080.10), compared to exon2 (with genomic coordinates chr11: 66564557-66564648); exon1 (with genomic coordinates chr1: 200844781-200844869) of gene calmodulin regulated spectrin associated protein family member 2 (CAMSAP2) (ENSG00000118200.14), compared to exon2 (with genomic coordinates chr1: 200848031-200850234); exon1 (with genomic coordinates chr2: 188988080-188988134) of gene collagen type III alpha 1 chain (COL3A1) (ENSG00000168542.14), compared to exon2 (with genomic coordinates chr2: 188989395-188989449); exon1 (with genomic coordinates chr1: 54205294-54205410) of gene mitochondrial ribosomal protein L37 (MRPL37) (ENSG00000116221.15), compared to exon2 (with genomic coordinates chr1: 54209945-54210131); exon1 (with genomic coordinates chr6: 149836084-149836297) of gene LDL receptor related protein 11 (LRP11) (ENSG00000120256.9), compared to exon2 (with genomic coordinates chr6: 149837337-149837463); exon1 (with genomic coordinates chrX: 41140655-41140771) of gene ubiquitin specific peptidase 9 X-linked (USP9X) (ENSG00000124486.12), compared to exon2 (with genomic coordinates chrX: 41140965-41141217); exon1 (with genomic coordinates chr6: 31642828-31643115) of gene BAG cochaperone 6 (BAG6) (ENSG00000204463.12), compared to exon2 (with genomic coordinates chr6: 31644081-31644194); exon1 (with genomic coordinates chr6: 33303954-33303989) of gene TAP binding protein (TAPBP) (ENSG00000231925.11), compared to exon2 (with genomic coordinates chr6: 33304127-33304217); exon1 (with genomic coordinates chr7: 34966893-34966971) of gene dpy-19 like C-mannosyltransferase 1 (DPY19L1) (ENSG00000173852.14), compared to exon2 (with genomic coordinates chr7: 34969432-34969532); exon1 (with genomic coordinates chr2: 24299908-24300171) of gene intersectin 2 (ITSN2) (ENSG00000198399.14), compared to exon2 (with genomic coordinates chr2: 24301153-24301239); exon1 (with genomic coordinates chr10: 112438335-112438389) of gene zinc finger DHHC-type palmitoyltransferase 6 (ZDHHC6) (ENSG00000023041.11), compared to exon2 (with genomic coordinates chr10: 112440533-112440695); exon1 (with genomic coordinates chr10: 112438335-112438389) of gene zinc finger DHHC-type palmitoyltransferase 6 (ZDHHC6) (ENSG00000023041.11), compared to exon2 (with genomic coordinates chr10: 112442191-112442351).

7. The method according claim 1, wherein for the identification of MXE2 subtype at least one primary mutually exclusive exon usage event is present, characterized by differential splicing of exons that are incorporated into different transcripts, as exon1 and exon2, wherein the mutually exclusive splicing events are optionally selected from exon1 (with genomic coordinates chr20: 48988490-48988662) of gene ADP ribosylation factor guanine nucleotide exchange factor 2 (ARFGEF2) (ENSG00000124198.8), compared to exon2 (with genomic coordinates chr20: 48989284-48989436); exon1 (with genomic coordinates chr18: 62266700-62266749) of gene RAB11 Binding and LisH domain (KIAA1468) (ENSG00000134444.14), compared to exon2 (with genomic coordinates chr18: 62268868-62268948); exon1 (with genomic coordinates chr16: 30081258-30081310) of gene protein phosphatase 4 catalytic subunit (PPP4C) (ENSG00000149923.13), compared to exon2 (with genomic coordinates chr16: 30082483-30082534); exon1 (with genomic coordinates chr15: 34255261-34255392) of gene solute carrier family 12 member 6 (SLC12A6) (ENSG00000140199.11), compared to exon2 (with genomic coordinates chr15: 34258812-34258944); exon1 (with genomic coordinates chr14: 22905605-22905659) of gene RNA binding motif protein 23 (RBM23) (ENSG00000100461.17), compared to exon2 (with genomic coordinates chr14: 22908332-22908380); exon1 (with genomic coordinates chr12: 7142085-7142139) of gene calsyntenin 3 (CLSTN3) (ENSG00000139182.13), compared to exon2 (with genomic coordinates chr12: 7142868-7143026); exon1 (with genomic coordinates chr20: 13534021-13534141) of gene taspase 1 (TASP1) (ENSG00000089123.15), compared to exon2 (with genomic coordinates chr20: 13559007-13559114); exon1 (with genomic coordinates chr8: 52630469-52630528) of gene RB1 inducible coiled-coil 1 (RB1CC1) (ENSG00000023287.12), compared to exon2 (with genomic coordinates chr8: 52634920-52634968); exon1 (with genomic coordinates chr1: 150156065-150156206) of gene pleckstrin homology domain containing 01 (PLEKHO1) (ENSG00000023902.13), compared to exon2 (with genomic coordinates chr1: 150157384-150157486); exon1 (with genomic coordinates chr2: 32607454-32607643) of gene baculoviral IAP repeat containing 6 (BIRC6) (ENSG00000115760.13), compared to exon2 (with genomic coordinates chr2: 32611447-32611582); exon1 (with genomic coordinates chr11: 9430874-9431003) of gene importin 7 (IPO7) (ENSG00000205339.9), compared to exon2 (with genomic coordinates chr11: 9433569-9433636); exon1 (with genomic coordinates chr17: 82081163-82081352) of gene fatty acid synthase (FASN) (ENSG00000169710.8), compared to exon2 (with genomic coordinates chr17: 82081600-82081843); exon1 (with genomic coordinates chr6: 157873101-157873176) of gene sorting nexin 9 (SNX9) (ENSG00000130340.15), compared to exon2 (with genomic coordinates chr6: 157875050-157875176); exon1 (with genomic coordinates chr7: 5390484-5390628) of gene trinucleotide repeat containing 18 (TNRC18) (ENSG00000182095.14), compared to exon2 (with genomic coordinates chr7: 5394439-5394595); exon1 (with genomic coordinates chr17: 50195922-50195976) of gene collagen type I alpha 1 chain (COL1A1) (ENSG00000108821.13), compared to exon2 (with genomic coordinates chr17: 50196154-50196199).

8. The method according claim 1, wherein for the identification of the MXE1 subtype primary small nuclear RNA expression shows up-regulation as compared to the MXE2 subtype exhibiting the highest predictive importance for MXE subtype classification selected from
RNU4-2 (ENSG00000202538.1) SEQ ID NO:1:
AGCTTTGCGCAGTGGCAGTATCGTAGCCAATGAGGTTTATCCGAGGCGCGATTATTGCTAAT
TGAAAACTTTTCCCAATACCCCGCCATGACGACTTGAAATATAGTCGGCATTGGCAATTTTTG
ACAGTCTCTACGGAGA
RNVU1-7 (ENSG00000206585.1) SEQ ID NO: 2:
ATACTTACCTGGCAGGGGAGATACCATGATCACGAAGGTGGTTTTCCCAGGGCGAGGCTTA
TCCATTGCACTCCGGATGTGCTGACCCCTGCGATTTCCCCAAATGTGGGAAACTCGACTGCA
TAATTTGTGGTAGTGGGGGACTGCGTCCGCGCTTTCCCCTG
RNVU1-6 (ENSG00000201558.1) SEQ ID NO: 3:
ATACTTACCTGGCAGGAGAGATACCCTGGTCACGAAGGTGGTTTTTCCAGAGCGAGTCTTAT
CCATTGCACTCCGGATCTGCTGACCCCTGCAGTTTCCCCAAATGTGGGAAACTTGACTGTAT
AATTTGTGGCAGTGGTAGACTGCATTCGCGCTTTCCCCTG.

9. The method according to claim 1, wherein for the identification of the MXE2 subtype the primary small nuclear RNA expression shows up-regulation as compared to the MXE1 subtype exhibiting the highest predictive importance for MXE subtype classification selected from
RNU5B-1 (ENSG00000200156.1) SEQ ID NO: 32:
ATACTCTGGTTTCTCTTCAGATCGTATAAATCTTTCGCCTTTTACTAAAGATTTCCGTGGAGAG
GAACAACTCTGAGTCTTAAGCTAATTTTTTGAGGCCTTGTTCCGACAAGGCT
RNU12 (ENSG00000276027.1) SEQ ID NO: 33:
ATGCCTTAAACTTATGAGTAAGGAAAATAACGATTCGGGGTGACGCCCGAATCCTCACTGCT
AATGTGAGACGAATTTTTGAGCGGGTAAAGGTCGCCCTCAAGGTGACCCGCCTACTTTGCG
GGATGCCTGGGAGTTGCGATCTGCCCG
RNU11 (ENSG00000274978.1) SEQ ID NO: 34:
AAAAAGGGCTTCTGTCGTGAGTGGCACACGTAGGGCAACTCGATTGCTCTGCGTGCGGAAT
CGACATCAAGAGATTTCGGAAGCATAATTTTTTGGTATTTGGGCAGCTGGTGATCGTTGGTC
CCGGCGCCCTT
RNU5E-1 (ENSG00000199347.1) SEQ ID NO: 35:
ATACTCTGGTTTCTCTTCAAATCGTATAAATCTTTCGCCTTTTACTAAAGATTTCCGTGGAGAG
AAACGAGTGTGAGTCTGAAACCAATTTTTTGAGGCCTTGCGTTTCTTAGCAGGGCT
RNU6-9(ENSG00000207507.1) SEQ ID NO: 36:
GTGCTCGCTTCGGCAGCACATATACTAAAATTGGAACGATACAGAGAAGATTAGCATGGCCC
CTGCGCAAGGATGACACGCAAATTCGTGAAGCGTTCCATATTTTT
RNU5D-1 (ENSG00000200169.1) SEQ ID NO: 37:
ATGCTCTGGTTTCTCTTCAAATCGTATAAATCTTTCGCCTTTTACTAAAGATTTCCGTGGAGAG
AAACCGTTTTGAGTTTCAAGCAAATTTTTTGAAGCCCTATGTTGGTGGGGTT
RNU5A-1 (ENSG00000199568.1) SEQ ID NO: 38:
ATACTCTGGTTTCTCTTCAGATCGCATAAATCTTTCGCCTTTTACTAAAGATTTCCGTGGAGA
GGAACAACTCTGAGTCTTAACCCAATTTTTTGAGGCCTTGCTTTGGCAAGGCT
RNVU1-15 (ENSG00000207205.1) SEQ ID NO: 39:
ATACTTACTTGGCGGGGGAGATACCATGATCACGAAGGTGGTTTTCTCAGGGCGAGGCTTAT
CCGTTATGTTCCGGGTGTACTGACCCCTGCCATTTTCCCCCAATGTGAGGAACTCGACTGCA
TAACTTGTGATAGTAGGGGACTGCGTTCGCGCTTTCCCCTG.

10. The method according claim 1, wherein for the identification of the MXE1 subtype the expression of the predictive protein-coding genes is characterized by up-regulation in the MXE1 subtype as compared to a MXE2 subtype of primary group, selected from kelch like family member 21 (KLHL21) (ENSG00000162413.16), mitotic arrest deficient 2 like 2 (MAD2L2) (ENSG00000116670.14), angiotensin II receptor associated protein (AGTRAP) (ENSG00000177674.15), NBL1, DAN family BMP antagonist (NBL1) (ENSG00000158747.14), zinc finger DHHC-type palmitoyltransferase 18 (ZDHHC18) (ENSG00000204160.11), solute carrier family 9 member A1 (SLC9A1) (ENSG00000090020.10), transmembrane protein 222 (TMEM222) (ENSG00000186501.14), platelet activating factor receptor (PTAFR) (ENSG00000169403.11), penta-EF-hand domain containing 1 (PEF1) (ENSG00000162517.12), NHS like 3 (KIAA1522) (ENSG00000162522.10), chromosome 1 open reading frame 122 (C1orf122) (ENSG00000197982.13), bone morphogenetic protein 8a (BMP8A) (ENSG00000183682.7), importin 13 (IPO13) (ENSG00000117408.10), diphthamide biosynthesis 2 (DPH2) (ENSG00000132768.13), ATPase H+ transporting V0 subunit b (ATP6V0B) (ENSG00000117410.13), aldo-keto reductase family 1 member A1 (AKR1A1) (ENSG00000117448.13), leucine rich repeat containing 41 (LRRC41) (ENSG00000132128.16), PDZK1 interacting protein 1 (PDZK1IP1) (ENSG00000162366.7), 24-dehydrocholesterol reductase (DHCR24) (ENSG00000116133.11), CD53 molecule (CD53) (ENSG00000143119.12), WD repeat domain 77 (WDR77) (ENSG00000116455.13), peroxisomal biogenesis factor 11 beta (PEX11B) (ENSG00000131779.10), aph-1 homolog A, gamma-secretase subunit (APH1A) (ENSG00000117362.12), proteasome 20S subunit beta 4 (PSMB4) (ENSG00000159377.10), S100 calcium binding protein A2 (S100A2) (ENSG00000196754.10), jumping translocation breakpoint (JTB) (ENSG00000143543.14), RUN and SH3 domain containing 1 (RUSC1) (ENSG00000160753.15), methyltransferase like 25B (RRNAD1) (ENSG00000143303.11), SLAM family member 8 (SLAMF8) (ENSG00000158714.10), protoporphyrinogen oxidase (PPOX) (ENSG00000143224.17), uridine-cytidine kinase 2 (UCK2) (ENSG00000143179.15), SFT2 domain containing 2 (SFT2D2) (ENSG00000213064.9), transmembrane protein 9 (TMEM9) (ENSG00000116857.16), NUAK family kinase 2 (NUAK2) (ENSG00000163545.8), left-right determination factor 1 (LEFTY1) (ENSG00000243709.1), jumonji domain containing 4 (JMJD4) (ENSG00000081692.12), solute carrier family 35 member F6 (SLC35F6) (ENSG00000213699.8), solute carrier family 5 member 6 (SLC5A6) (ENSG00000138074.14), sorting nexin 17 (SNX17) (ENSG00000115234.10), testis expressed 261 (TEX261) (ENSG00000144043.11), SMYD family member 5 (SMYD5) (ENSG00000135632.11), coiled-coil domain containing 142 (CCDC142) (ENSG00000135637.13), ssemaphorin 4F (SEMA4F) (ENSG00000135622.12), trans-golgi network protein 2 (TGOLN2) (ENSG00000152291.13), transmembrane protein 127 (TMEM127) (ENSG00000135956.8), mitotic spindle organizing protein 2B (MZT2B) (ENSG00000152082.13), Rho guanine nucleotide exchange factor 4 (ARHGEF4) (ENSG00000136002.18), COP9 signalosome subunit 7B (COPS7B) (ENSG00000144524.17), hes family bHLH transcription factor 6 (HES6) (ENSG00000144485.10), G protein-coupled receptor 35 (GPR35) (ENSG00000178623.11), actin related protein 2/3 complex subunit 4 (ARPC4) (ENSG00000241553.12), jagunal homolog 1 (JAGN1) (ENSG00000171135.13), interleukin 17 receptor C (IL17RC) (ENSG00000163702.18), transmembrane protein with metallophosphoesterase domain (TMPPE) (ENSG00000188167.8), ribosomal protein SA (RPSA) (ENSG00000168028.13), ubiquinol-cytochrome c reductase core protein 1 (UQCRC1) (ENSG00000010256.10), DALR anticodon binding domain containing 3 (DALRD3) (ENSG00000178149.16), cytochrome b561 family member D2 (CYB561D2) (ENSG00000114395.10), POC1 centriolar protein A (POC1A) (ENSG00000164087.7), solute carrier family 41 member 3 (SLC41A3) (ENSG00000114544.16), transmembrane protein adipocyte associated 1 (TPRA1) (ENSG00000163870.14), ALG3 alpha-1,3-mannosyltransferase (ALG3) (ENSG00000214160.9), EEF1A lysine methyltransferase 4 (EEF1AKMT4) (ENSG00000284753.1), major facilitator superfamily domain containing 10 (MFSD10) (ENSG00000109736.14), interferon regulatory factor 2 (IRF2) (ENSG00000168310.10), solute carrier family 35 member A4 (SLC35A4) (ENSG00000176087.14), NADH:ubiquinone oxidoreductase subunit A2 (NDUFA2) (ENSG00000131495.8), solute carrier family 36 member 1 (SLC36A1) (ENSG00000123643.12), antioxidant 1 copper chaperone (ATOX1) (ENSG00000177556.11), ADP ribosylation factor like GTPase 10 (ARL10) (ENSG00000175414.6), beta-1,4-galactosyltransferase 7 (B4GALT7) (ENSG00000027847.13), 5-phosphohydroxy-L-lysine phospho-lyase (PHYKPL) (ENSG00000175309.14), H3 clustered histone 2 (HIST1H3B) (ENSG00000274267.1), H1.2 linker histone, cluster member (HIST1H1C) (ENSG00000187837.3), H3 clustered histone 10 (HIST1H3H) (ENSG00000278828.1), olfactory receptor family 2 subfamily I member 1 pseudogene (OR2I1P) (ENSG00000237988.5), RNA polymerase I subunit H (ZNRD1) (ENSG00000066379.14), splicing factor 3b subunit 5 (SF3B5) (ENSG00000169976.6), aquaporin 1 (Colton blood group) (AQP1) (ENSG00000240583.11), receptor activity modifying protein 3 (RAMP3) (ENSG00000122679.8), NOP2/Sun RNA methyltransferase 5 (NSUN5) (ENSG00000130305.16), elastin (ELN) (ENSG00000049540.16), zona pellucida glycoprotein 3 (ZP3) (ENSG00000188372.14), cytochrome P450 family 51 subfamily A member 1 (CYP51A1) (ENSG00000001630.15), solute carrier family 12 member 9 (SLC12A9) (ENSG00000146828.17), monoacylglycerol O-acyltransferase 3 (MOGAT3) (ENSG00000106384.10), RNA polymerase II subunit J2 (POLR2J2) (ENSG00000228049.7), smoothened, frizzled class receptor (SMO) (ENSG00000128602.9), cell cycle regulator of NHEJ (C7orf49) (ENSG00000122783.16), MT-RNR2 like 6 (pseudogene) (MTRNR2L6) (ENSG00000270672.1), Fas activated serine/threonine kinase (FASTK) (ENSG00000164896.19), heparan-alpha-glucosaminide N-acetyltransferase (HGSNAT) (ENSG00000165102.14), grainyhead like transcription factor 2 (GRHL2) (ENSG00000083307.11), F-box and leucine rich repeat protein 6 (FBXL6) (ENSG00000182325.10), ribonuclease P/MRP subunit p25 like (RPP25L) (ENSG00000164967.9), phosphatidylinositol glycan anchor biosynthesis class O (PIGO) (ENSG00000165282.13), haloacid dehalogenase like hydrolase domain containing 3 (HDHD3) (ENSG00000119431.9), WD repeat domain 38 (WDR38) (ENSG00000136918.7), solute carrier family 2 member 8 (SLC2A8) (ENSG00000136856.17), torsin family 2 member A (TOR2A) (ENSG00000160404.17), ST6 N-acetylgalactosaminide alpha-2,6-sialyltransferase 6 (ST6GALNAC6) (ENSG00000160408.14), zinc finger DHHC-type palmitoyltransferase 12 (ZDHHC12) (ENSG00000160446.18), TBC1 domain family member 13 (TBC1D13) (ENSG00000107021.15), protein phosphatase 2 phosphatase activator (PPP2R4) (ENSG00000119383.19), calcium channel flower domain containing 1 (CACFD1) (ENSG00000160325.14), transmembrane protein 141 (TMEM141) (ENSG00000244187.7), transmembrane protein 203 (TMEM203) (ENSG00000187713.6), trypsin like peroxisomal matrix peptidase 1 (TYSND1) (ENSG00000156521.13), leucine rich repeat containing 20 (LRRC20) (ENSG00000172731.13), V-set immunoregulatory receptor (VSIR) (ENSG00000107738.19), SEC24 homolog C, COPII coat complex component (SEC24C) (ENSG00000176986.15), tectonic family member 3 (TCTN3) (ENSG00000119977.20), phosphatidylinositol 4-kinase type 2 alpha (PI4K2A) (ENSG00000155252.13), F-box and WD repeat domain containing 4 (FBXW4) (ENSG00000107829.13), glutathione S-transferase omega 1 (GSTO1) (ENSG00000148834.12), Bet1 golgi vesicular membrane trafficking protein like (BET1L) (ENSG00000177951.17), tetraspanin 4 (TSPAN4) (ENSG00000214063.10), chitinase domain containing 1 (CHID1) (ENSG00000177830.17), tumor suppressing subtransferable candidate 4 (TSSC4) (ENSG00000184281.14), FHF complex subunit HOOK interacting protein 1B (FAM160A2) (ENSG00000051009.10), peroxisomal biogenesis factor 16 (PEX16) (ENSG00000121680.15), solute carrier family 39 member 13 (SLC39A13) (ENSG00000165915.13), cytochrome b561 family member A3 (CYB561A3) (ENSG00000162144.9), transmembrane protein 258 (TMEM258) (ENSG00000134825.15), bestrophin 1 (BEST1) (ENSG00000167995.15), beta-1,3-glucuronyltransferase 3 (B3GAT3) (ENSG00000149541.9), integrator complex subunit 5 (INTS5) (ENSG00000185085.2), cytochrome c oxidase subunit 8A (COX8A) (ENSG00000176340.3), sorting nexin 32 (SNX32) (ENSG00000172803.17), frizzled class receptor 4 (FZD4) (ENSG00000174804.3), solute carrier family 37 member 4 (SLC37A4) (ENSG00000137700.17), NLR family member X1 (NLRX1) (ENSG00000160703.15), F-box and leucine rich repeat protein 14 (FBXL14) (ENSG00000171823.6), non-SMC condensin I complex subunit D2 (NCAPD2) (ENSG00000010292.12), myeloid leukemia factor 2 (MLF2) (ENSG00000089693.10), complement C3a receptor 1 (C3AR1) (ENSG00000171860.4), G protein-coupled receptor class C group 5 member A (GPRC5A) (ENSG00000013588.7), transmembrane protein 106C (TMEM106C) (ENSG00000134291.11), tubulin alpha 1b (TUBA1B) (ENSG00000123416.15), major facilitator superfamily domain containing 5 (MFSD5) (ENSG00000182544.8), ATP synthase membrane subunit C locus 2 (ATP5G2) (ENSG00000135390.17), retinol dehydrogenase 5 (RDH5) (ENSG00000135437.9), premelanosome protein (PMEL) (ENSG00000185664.14), cyclin dependent kinase 4 (CDK4) (ENSG00000135446.16), CTD small phosphatase 2 (CTDSP2) (ENSG00000175215.10), polypeptide N-acetylgalactosaminyltransferase 4 (GALNT4) (ENSG00000257594.3), phospholipase B domain containing 2 (PLBD2) (ENSG00000151176.7), paxillin (PXN) (ENSG00000089159.16), refilin A (FAM101A) (ENSG00000178882.14), transmembrane protein 253 (TMEM253) (ENSG00000232070.8), abhydrolase domain containing 4, N-acyl phospholipase B (ABHD4) (ENSG00000100439.10), proteasome 20S subunit beta 5 (PSMB5) (ENSG00000100804.18), phosphoenolpyruvate carboxykinase 2, mitochondrial (PCK2) (ENSG00000100889.11), ER membrane protein complex subunit 9 (EMC9) (ENSG00000100908.13), ribosomal protein S29 (RPS29) (ENSG00000213741.10), sushi domain containing 6 (SUSD6) (ENSG00000100647.7), acyl-CoA thioesterase 1 (ACOT1) (ENSG00000184227.7), acyl-CoA thioesterase 4 (ACOT4) (ENSG00000177465.4), ATP binding cassette subfamily D member 4 (ABCD4) (ENSG00000119688.20), NPC intracellular cholesterol transporter 2 (NPC2) (ENSG00000119655.10), angel homolog 1 (ANGEL1) (ENSG00000013523.9), CLOCK interacting pacemaker (CIPC) (ENSG00000198894.7), G protein-coupled receptor 68 (GPR68) (ENSG00000119714.10), solute carrier family 25 member 29 (SLC25A29) (ENSG00000197119.12), SIVA1 apoptosis inducing factor (SIVA1) (ENSG00000184990.12), G protein-coupled receptor 176 (GPR176) (ENSG00000166073.10), sorting nexin 22 (SNX22) (ENSG00000157734.13), pleckstrin homology domain containing O2 (PLEKHO2) (ENSG00000241839.9), cartilage intermediate layer protein (CILP) (ENSG00000138615.5), stomatin like 1 (STOML1) (ENSG00000067221.13), COMM domain containing 4 (COMMD4) (ENSG00000140365.15), phosphatidylinositol glycan anchor biosynthesis class Q (PIGQ) (ENSG00000007541.16), transmembrane protein 204 (TMEM204) (ENSG00000131634.13), NME/NM23 nucleoside diphosphate kinase 3 (NME3) (ENSG00000103024.7), NUBP iron-sulfur cluster assembly factor 2, cytosolic (NUBP2) (ENSG00000095906.16), presequence translocase associated motor 16 (PAM16) (ENSG00000217930.7), coronin 7 (CORO7) (ENSG00000262246.5), PAXIP1 associated glutamate rich protein 1 (PAGR1) (ENSG00000280789.1), CDP-diacylglycerol-inositol 3-phosphatidyltransferase (CDIPT) (ENSG00000103502.13), phosphorylase kinase catalytic subunit gamma 2 (PHKG2) (ENSG00000156873.15), ORAI calcium release-activated calcium modulator 3 (ORAI3) (ENSG00000175938.6), serine protease 8 (PRSS8) (ENSG00000052344.15), adhesion G protein-coupled receptor G1 (ADGRG1) (ENSG00000205336.11), chemokine like factor (CKLF) (ENSG00000217555.12), CKLF like MARVEL transmembrane domain containing 3 (CMTM3) (ENSG00000140931.19), transmembrane protein 208 (TMEM208) (ENSG00000168701.18), THAP domain containing 11 (THAP11) (ENSG00000168286.2), proteasome 20S subunit beta 10 (PSMB10) (ENSG00000205220.11), Tax1 binding protein 3 (TAX1BP3) (ENSG00000213977.7), GABA type A receptor-associated protein (GABARAP) (ENSG00000170296.9), transient receptor potential cation channel subfamily V member 2 (TRPV2) (ENSG00000187688.14), N-acetyltransferase domain containing 1 (NATD1) (ENSG00000274180.1), DNA polymerase delta interacting protein 2 (POLDIP2) (ENSG00000004142.11), notchless homolog 1 (NLE1) (ENSG00000073536.17), NFKB inhibitor interacting Ras like 2 (NKIRAS2) (ENSG00000168256.17), N-acetyl-alpha-glucosaminidase (NAGLU) (ENSG00000108784.9), reticulophagy regulator family member 3 (FAM134C) (ENSG00000141699.10), glucose-6-phosphatase catalytic subunit 3 (G6PC3) (ENSG00000141349.8), intercellular adhesion molecule 2 (ICAM2) (ENSG00000108622.10), G protein-coupled receptor class C group 5 member C (GPRC5C) (ENSG00000170412.16), thymidine kinase 1 (TK1) (ENSG00000167900.11), actin gamma 1 (ACTG1) (ENSG00000184009.10), FA core complex associated protein 100 (FAAP100) (ENSG00000185504.16), solute carrier family 25 member 10 (SLC25A10) (ENSG00000183048.11), phospholipid phosphatase 2 (PLPP2) (ENSG00000141934.9), secretory carrier membrane protein 4 (SCAMP4) (ENSG00000227500.9), MOB kinase activator 3A (MOB3A) (ENSG00000172081.13), fucosyltransferase 6 (FUT6) (ENSG00000156413.13), NADH:ubiquinone oxidoreductase subunit A11 (NDUFA11) (ENSG00000174886.12), calcyphosine (CAPS) (ENSG00000105519.15), PET100 cytochrome c oxidase chaperone (C19orf79) (ENSG00000229833.9), CD320 molecule (CD320) (ENSG00000167775.10), sphingosine-1-phosphate receptor 2 (S1PR2) (ENSG00000267534.3), autophagy related 4D cysteine peptidase (ATG4D) (ENSG00000130734.9), phospholipid phosphatase related 2 (PLPPR2) (ENSG00000105520.10), WD repeat domain 83 opposite strand (WDR83OS) (ENSG00000105583.9), notch receptor 3 (NOTCH3) (ENSG00000074181.8), adaptor related protein complex 1 subunit mu 1 (AP1M1) (ENSG00000072958.8), ubiquitin A-52 residue ribosomal protein fusion product 1 (UBA52) (ENSG00000221983.7), kelch like family member 26 (KLHL26) (ENSG00000167487.11), armadillo repeat containing 6 (ARMC6) (ENSG00000105676.13), transmembrane protein 161A (TMEM161A) (ENSG00000064545.14), nuclear receptor 2C2 associated protein (NR2C2AP) (ENSG00000184162.14), MAU2 sister chromatid cohesion factor (MAU2) (ENSG00000129933.20), transmembrane protein 147 (TMEM147) (ENSG00000105677.11), presenilin enhancer, gamma-secretase subunit (PSENEN) (ENSG00000205155.7), cytochrome c oxidase subunit 7A1 (COX7A1) (ENSG00000161281.10), Charcot-Leyden crystal galectin (CLC) (ENSG00000105205.6), zinc finger protein 574 (ZNF574) (ENSG00000105732.12), ETHE1 persulfide dioxygenase (ETHE1) (ENSG00000105755.7), adaptor related protein complex 2 subunit sigma 1 (AP2S1) (ENSG00000042753.11), transient receptor potential cation channel subfamily M member 4 (TRPM4) (ENSG00000130529.15), BCL2 like 12 (BCL2L12) (ENSG00000126453.9), sialic acid binding Ig like lectin 14 (SIGLEC14) (ENSG00000254415.3), formyl peptide receptor 1 (FPR1) (ENSG00000171051.8), protein phosphatase 2 scaffold subunit Aalpha (PPP2R1A) (ENSG00000105568.17), zinc finger protein 320 (ZNF320) (ENSG00000182986.13), ribosomal protein S5 (RPS5) (ENSG00000083845.8), small nuclear ribonucleoprotein polypeptides B and B1 (SNRPB) (ENSG00000125835.18), U-box domain containing 5 (UBOX5) (ENSG00000185019.16), acyl-CoA synthetase short chain family member 1 (ACSS1) (ENSG00000154930.14), glycogen phosphorylase B (PYGB) (ENSG00000100994.11), AAR2 splicing factor (AAR2) (ENSG00000131043.11), acyl-CoA thioesterase 8 (ACOT8) (ENSG00000101473.16), phosphorylated CTD interacting factor 1 (PCIF1) (ENSG00000100982.11), gamma-glutamyltransferase 5 (GGT5) (ENSG00000099998.17), rhomboid domain containing 3 (RHBDD3) (ENSG00000100263.13), galectin 1 (LGALS1) (ENSG00000100097.11), platelet derived growth factor subunit B (PDGFB) (ENSG00000100311.16), centromere protein M (CENPM) (ENSG00000100162.14), translocator protein (TSPO) (ENSG00000100300.17), gem nuclear organelle associated protein 8 (GEMIN8) (ENSG00000046647.13), NADH:ubiquinone oxidoreductase subunit B11 (NDUFB11) (ENSG00000147123.10), ribosomal protein L10 (RPL10) (ENSG00000147403.16), L antigen family member 3 (LAGE3) (ENSG00000196976.7), AC004556.1 (ENSG00000276345.1), peptidyl arginine deiminase 2 (PADI2) (ENSG00000117115.12), phospholipase A2 group IIA (PLA2G2A) (ENSG00000188257.10), collagen type IX alpha 2 chain (COL9A2) (ENSG00000049089.14), cytochrome P450 family 4 subfamily X member 1 (CYP4X1) (ENSG00000186377.7), enoyl-CoA hydratase domain containing 2 (ECHDC2) (ENSG00000121310.16), maestro heat like repeat family member 7 (MROH7) (ENSG00000184313.19), protein kinase CAMP-activated catalytic subunit beta (PRKACB) (ENSG00000142875.19), sterile alpha motif domain containing 13 (SAMD13) (ENSG00000203943.8), coagulation factor III, tissue factor (F3) (ENSG00000117525.13), 3-hydroxy-3-methylglutaryl-CoA synthase 2 (HMGCS2) (ENSG00000134240.11), selenium binding protein 1 (SELENBP1) (ENSG00000143416.20), regulator of G protein signaling 5 (RGS5) (ENSG00000143248.12), cathepsin E (CTSE) (ENSG00000196188.10), immunoglobulin kappa constant (IGKC) (ENSG00000211592.8), chromosome 2 open reading frame 88 (C2orf88) (ENSG00000187699.10), integral membrane protein 2C (ITM2C) (ENSG00000135916.15), FAM3 metabolism regulating signaling molecule D (FAM3D) (ENSG00000198643.6), ABI family member 3 binding protein (ABI3BP) (ENSG00000154175.16), resistin like beta (RETNLB) (ENSG00000163515.6), solute carrier organic anion transporter family member 2A1 (SLCO2A1) (ENSG00000174640.12), mucin 4, cell surface associated (MUC4) (ENSG00000145113.21), chromosome 4 open reading frame 19 (C4orf19) (ENSG00000154274.14), UDP glucuronosyltransferase family 2 member B17 (UGT2B17) (ENSG00000197888.2), joining chain of multimeric IgA and IgM (JCHAIN) (ENSG00000132465.10), N-acylethanolamine acid amidase (NAAA) (ENSG00000138744.14), nuclear receptor subfamily 3 group C member 2 (NR3C2) (ENSG00000151623.14), C-C motif chemokine ligand 28 (CCL28) (ENSG00000151882.11), marginal zone B and B1 cell specific protein (MZB1) (ENSG00000170476.15), solute carrier family 26 member 2 (SLC26A2) (ENSG00000155850.7), CD74 molecule (CD74) (ENSG00000019582.14), chloride intracellular channel 5 (CLIC5) (ENSG00000112782.16), 5′-nucleotidase ecto (NT5E) (ENSG00000135318.11), sphingomyelin phosphodiesterase acid like 3A (SMPDL3A) (ENSG00000172594.12), scinderin (SCIN) (ENSG00000006747.14), histone deacetylase 9 (HDAC9) (ENSG00000048052.21), transient receptor potential cation channel subfamily A member 1 (TRPA1) (ENSG00000104321.10), stathmin 2 (STMN2) (ENSG00000104435.13), glutamic-pyruvic transaminase (GPT) (ENSG00000167701.13), prostaglandin D2 synthase (PTGDS) (ENSG00000107317.12), Rho related BTB domain containing 1 (RHOBTB1) (ENSG00000072422.16), cadherin related family member 1 (CDHR1) (ENSG00000148600.14), 3′-phosphoadenosine 5′-phosphosulfate synthase 2 (PAPSS2) (ENSG00000198682.12), actin filament associated protein 1 like 2 (AFAP1L2) (ENSG00000169129.14), X-ray radiation resistance associated 1 (XRRA1) (ENSG00000166435.15), calpain 5 (CAPN5) (ENSG00000149260.15), coiled-coil domain containing 89 (CCDC89) (ENSG00000179071.4), synaptotagmin like 2 (SYTL2) (ENSG00000137501.17), matrix metallopeptidase 7 (MMP7) (ENSG00000137673.8), neurexophilin and PC-esterase domain family member 1 (NXPE1) (ENSG00000095110.8), neurexophilin and PC-esterase domain family member 4 (NXPE4) (ENSG00000137634.9), tripartite motif containing 29 (TRIM29) (ENSG00000137699.16), V-set and immunoglobulin domain containing 2 (VSIG2) (ENSG00000019102.11), solute carrier family 37 member 2 (SLC37A2) (ENSG00000134955.11), ST3 beta-galactoside alpha-2,3-sialyltransferase 4 (ST3GAL4) (ENSG00000110080.18), BARX homeobox 2 (BARX2) (ENSG00000043039.6), lysophosphatidic acid receptor 6 (LPAR6) (ENSG00000139679.15), heat shock protein family A (Hsp70) member 2 (HSPA2) (ENSG00000126803.9), bradykinin receptor B2 (BDKRB2) (ENSG00000168398.6), creatine kinase B (CKB) (ENSG00000166165.12), calpain 3 (CAPN3) (ENSG00000092529.23), beta-2-microglobulin (B2M) (ENSG00000166710.17), RNA binding fox-1 homolog 1 (RBFOX1) (ENSG00000078328.20), class II major histocompatibility complex transactivator (CIITA) (ENSG00000179583.19), endoplasmic reticulum to nucleus signaling 2 (ERN2) (ENSG00000134398.14), lactate dehydrogenase D (LDHD) (ENSG00000166816.13), carbohydrate sulfotransferase 5 (CHST5) (ENSG00000135702.14), carbonic anhydrase 4 (CA4) (ENSG00000167434.9), axin 2 (AXIN2) (ENSG00000168646.12), secreted and transmembrane 1 (SECTM1) (ENSG00000141574.7), solute carrier family 14 member 1 (Kidd blood group) (SLC14A1) (ENSG00000141469.17), ring finger protein 152 (RNF152) (ENSG00000176641.10), plasmalemma vesicle associated protein (PLVAP) (ENSG00000130300.8), cation channel sperm associated auxiliary subunit gamma (CATSPERG) (ENSG00000099338.22), Fc gamma binding protein (FCGBP) (ENSG00000275395.5), kallikrein 1 (KLK1) (ENSG00000167748.10), glycine receptor alpha 2 (GLRA2) (ENSG00000101958.13), claudin 2 (CLDN2) (ENSG00000165376.10), MT-ND3 (ENSG00000198840.2).

11. The cancer subtype identifier according claim 1, wherein for the identification of MXE2 subtype the expression of the predictive protein-coding genes is characterized by their up-regulation in the MXE2 subtype as compared to the MXE1 subtype of primary group, selected from of TAR DNA binding protein (TARDBP) (ENSG00000120948.16), heterogeneous nuclear ribonucleoprotein C like 4 (HNRNPCL4) (ENSG00000179412.10), capping actin protein of muscle Z-line subunit beta (CAPZB) (ENSG00000077549.17), heterogeneous nuclear ribonucleoprotein R (HNRNPR) (ENSG00000125944.19), single stranded DNA binding protein 3 (SSBP3) (ENSG00000157216.15), nexilin F-actin binding protein (NEXN) (ENSG00000162614.18), far upstream element binding protein 1 (FUBP1) (ENSG00000162613.16), cellular communication network factor 1 (CYR61) (ENSG00000142871.16), SET like protein (SETSIP) (ENSG00000230667.5), major facilitator superfamily domain containing 14A (MFSD14A) (ENSG00000156875.13), SAS-6 centriolar assembly protein (SASS6) (ENSG00000156876.9), pre-mRNA processing factor 38B (PRPF38B) (ENSG00000134186.11), TATA-box binding protein associated factor 13 (TAF13) (ENSG00000197780.9), RAP1A, member of RAS oncogene family (RAP1A) (ENSG00000116473.14), H2A clustered histone 19 (HIST2H2AA4) (ENSG00000272196.2), H3 clustered histone 15 (HIST2H3A) (ENSG00000203852.3), H4 clustered histone 15 (HIST2H4B) (ENSG00000270276.2), acidic nuclear phosphoprotein 32 family member E (ANP32E) (ENSG00000143401.14), S100 calcium binding protein A10 (S100A10) (ENSG00000197747.8), regulator of G protein signaling 13 (RGS13) (ENSG00000127074.14), ribosomal protein S27a pseudogene 5 (RP11-92K2.2) (ENSG00000231767.4), ubiquitin C-terminal hydrolase L5 (UCHL5) (ENSG00000116750.13), LIM homeobox 9 (LHX9) (ENSG00000143355.15), zinc finger BED-type containing 6 (ZBED6) (ENSG00000257315.2), zinc finger CCCH-type containing 11B (ZC3H11B) (ENSG00000215817.5), ras homolog family member U (RHOU) (ENSG00000116574.5), translin associated factor X (TSNAX) (ENSG00000116918.13), myelin transcription factor 1 like (MYT1L) (ENSG00000186487.18), protein phosphatase 1 catalytic subunit beta (PPP1CB) (ENSG00000213639.9), protein phosphatase, Mg2+/Mn2+ dependent 1B (PPM1B) (ENSG00000138032.20), C1D nuclear receptor corepressor (C1D) (ENSG00000197223.11), poly(rC) binding protein 1 (PCBP1) (ENSG00000169564.6), RANBP2 like and GRIP domain containing 1 (RGPD1) (ENSG00000187627.15), RANBP2 like and GRIP domain containing 2 (RGPD2) (ENSG00000185304.14), RANBP2 like and GRIP domain containing 3 (RGPD3) (ENSG00000153165.18), RANBP2 like and GRIP domain containing 4 (RGPD4) (ENSG00000196862.9), GRIP and coiled-coil domain containing 2 (GCC2) (ENSG00000135968.20), RANBP2 like and GRIP domain containing 5 (RGPD5) (ENSG00000015568.12), RANBP2 like and GRIP domain containing 6 (RGPD6) (ENSG00000183054.11), diazepam binding inhibitor, acyl-CoA binding protein (DBI) (ENSG00000155368.16), RAB6C, member RAS oncogene family (RAB6C) (ENSG00000222014.5), POTE ankyrin domain family member I (POTEI) (ENSG00000196834.11), POTE ankyrin domain family member J (POTEJ) (ENSG00000222038.3), UBX domain protein 4 (UBXN4) (ENSG00000144224.16), ADP ribosylation factor like GTPase 5A (ARL5A) (ENSG00000162980.16), membrane associated ring-CH-type finger 7 (MARCH7) (ENSG00000136536.14), peptidylprolyl isomerase G (PPIG) (ENSG00000138398.15), small RNA binding exonuclease protection factor La (SSB) (ENSG00000138385.15), tousled like kinase 1 (TLK1) (ENSG00000198586.13), corepressor interacting with RBPJ, CIR1 (CIR1) (ENSG00000138433.15), heterogeneous nuclear ribonucleoprotein A3 (HNRNPA3) (ENSG00000170144.20), MOB family member 4, phocein (MOB4) (ENSG00000115540.14), tubulin alpha 4b (TUBA4B) (ENSG00000243910.7), prothymosin alpha (PTMA) (ENSG00000187514.16), myeloma overexpressed 2 pseudogene (MYEOV2) (ENSG00000172428.10), synapsin II (SYN2) (ENSG00000157152.16), LSM3 homolog, U6 small nuclear RNA and mRNA degradation associated (LSM3) (ENSG00000170860.3), protein kinase C delta (PRKCD) (ENSG00000163932.13), MT-RNR2 like 12 (pseudogene) (MTRNR2L12) (ENSG00000269028.3), filamin A interacting protein 1 like (FILIP1L) (ENSG00000168386.18), PEST proteolytic signal containing nuclear protein (PCNP) (ENSG00000081154.11), RAB7A, member RAS oncogene family (RAB7A) (ENSG00000075785.12), WW domain containing transcription regulator 1 (WWTR1) (ENSG00000018408.14), structural maintenance of chromosomes 4 (SMC4) (ENSG00000113810.15), SKI like proto-oncogene (SKIL) (ENSG00000136603.13), cytoplasmic polyadenylation element binding protein 2 (CPEB2) (ENSG00000137449.15), MOB kinase activator 1B (MOB1B) (ENSG00000173542.8), Ras association domain family member 6 (RASSF6) (ENSG00000169435.13), heterogeneous nuclear ribonucleoprotein D like (HNRNPDL) (ENSG00000152795.17), DnaJ heat shock protein family (Hsp40) member B14 (DNAJB14) (ENSG00000164031.16), family with sequence similarity 241 member A (C4orf32) (ENSG00000174749.5), La ribonucleoprotein 7, transcriptional regulator (LARP7) (ENSG00000174720.15), progesterone receptor membrane component 2 (PGRMC2) (ENSG00000164040.16), tryptophan 2,3-dioxygenase (TDO2) (ENSG00000151790.8), palladin, cytoskeletal associated protein (PALLD) (ENSG00000129116.17), 15-hydroxyprostaglandin dehydrogenase (HPGD) (ENSG00000164120.13), inhibitor of growth family member 2 (ING2) (ENSG00000168556.6), cilia and flagella associated protein 97 (CFAP97) (ENSG00000164323.12), NADH:ubiquinone oxidoreductase subunit S6 (NDUFS6) (ENSG00000145494.11), SUB1 regulator of transcription (SUB1) (ENSG00000113387.11), NADH:ubiquinone oxidoreductase complex assembly factor 2 (NDUFAF2) (ENSG00000164182.10), CWC27 spliceosome associated cyclophilin (CWC27) (ENSG00000153015.15), protein geranylgeranyltransferase type I subunit beta (PGGT1B) (ENSG00000164219.9), coiled-coil domain containing 112 (CCDC112) (ENSG00000164221.12), lysyl oxidase (LOX) (ENSG00000113083.13), ubiquitin conjugating enzyme E2 B (UBE2B) (ENSG00000119048.7), RNA binding motif protein 27 (RBM27) (ENSG00000091009.7), H4 clustered histone 11 (HIST1H4J) (ENSG00000197238.4), H4 clustered histone 12 (HIST1H4K) (ENSG00000273542.1), CD2 associated protein (CD2AP) (ENSG00000198087.7), KIAA1586 (KIAA1586) (ENSG00000168116.13), FKBP prolyl isomerase family member 1C (FKBP1C) (ENSG00000198225.5), high mobility group nucleosomal binding domain 3 (HMGN3) (ENSG00000118418.14), SEC63 homolog, protein translocation regulator (SEC63) (ENSG00000025796.13), myristoylated alanine rich protein kinase C substrate (MARCKS) (ENSG00000277443.2), RWD domain containing 1 (RWDD1) (ENSG00000111832.12), NUS1 dehydrodolichyl diphosphate synthase subunit (NUS1) (ENSG00000153989.7), cellular communication network factor 2 (CTGF) (ENSG00000118523.5), BCL2 associated transcription factor 1 (BCLAF1) (ENSG00000029363.16), LTV1 ribosome biogenesis factor (LTV1) (ENSG00000135521.8), TGF-beta activated kinase 1 (MAP3K7) binding protein 2 (TAB2) (ENSG00000055208.18), peptidylprolyl isomerase like 4 (PPIL4) (ENSG00000131013.3), RB associated KRAB zinc finger (RBAK) (ENSG00000146587.17), transmembrane protein 106B (TMEM106B) (ENSG00000106460.18), aryl hydrocarbon receptor (AHR) (ENSG00000106546.13), transformer 2 alpha homolog (TRA2A) (ENSG00000164548.10), sperm microtubule inner protein 4 (C7orf31) (ENSG00000153790.11), heterogeneous nuclear ribonucleoprotein A2/B1 (HNRNPA2B1) (ENSG00000122566.20), zinc and ring finger 2 (ZNRF2) (ENSG00000180233.10), ITPR interacting domain containing 1 (CCDC129) (ENSG00000180347.13), POU class 6 homeobox 2 (POU6F2) (ENSG00000106536.19), RAS like proto-oncogene A (RALA) (ENSG00000006451.7), inhibin subunit beta A (INHBA) (ENSG00000122641.10), zinc finger protein 680 (ZNF680) (ENSG00000173041.11), fibrinogen like 2 (FGL2) (ENSG00000127951.6), A-kinase anchoring protein 9 (AKAP9) (ENSG00000127914.16), family with sequence similarity 133 member B (FAM133B) (ENSG00000234545.7), DnaJ heat shock protein family (Hsp40) member C2 (DNAJC2) (ENSG00000105821.14), NADH:ubiquinone oxidoreductase subunit A5 (NDUFA5) (ENSG00000128609.14), chromosome 7 open reading frame 73 (C7orf73) (ENSG00000243317.7), protein kinase AMP-activated non-catalytic subunit gamma 2 (PRKAG2) (ENSG00000106617.13), neurofilament medium chain (NEFM) (ENSG00000104722.13), dynactin subunit 6 (DCTN6) (ENSG00000104671.7), CCAAT enhancer binding protein delta (CEBPD) (ENSG00000221869.4), syntrophin gamma 1 (SNTG1) (ENSG00000147481.15), RAB2A, member RAS oncogene family (RAB2A) (ENSG00000104388.14), translocation associated membrane protein 1 (TRAM1) (ENSG00000067167.7), zinc finger C2HC-type containing 1A (ZC2HC1A) (ENSG00000104427.11), tumor protein D52 (TPD52) (ENSG00000076554.15), ribosomal biogenesis factor (C8orf59) (ENSG00000176731.11), ubiquinol-cytochrome c reductase binding protein (UQCRB) (ENSG00000156467.9), RNA polymerase II, I and III subunit K (POLR2K) (ENSG00000147669.10), poly(A) binding protein cytoplasmic 1 (PABPC1) (ENSG00000070756.15), glycine decarboxylase (GLDC) (ENSG00000178445.9), zinc finger DHHC-type palmitoyltransferase 21 (ZDHHC21) (ENSG00000175893.11), PC4 and SRSF1 interacting protein 1 (PSIP1) (ENSG00000164985.14), caspase activity and apoptosis inhibitor 1 (CAAP1) (ENSG00000120159.11), TATA-box binding protein associated factor 1 like (TAF1L) (ENSG00000122728.6), carnosine N-methyltransferase 1 (CARNMT1) (ENSG00000156017.12), riboflavin kinase (RFK) (ENSG00000135002.11), RecQ mediated genome instability 1 (RMI1) (ENSG00000178966.16), acidic nuclear phosphoprotein 32 family member B (ANP32B) (ENSG00000136938.8), actin binding transcription modulator (FAM206A) (ENSG00000119328.11), TATA-box binding protein associated factor 3 (TAF3) (ENSG00000165632.7), chondroitin sulfate N-acetylgalactosaminyltransferase 2 (CSGALNACT2) (ENSG00000169826.7), ubiquitin conjugating enzyme E2 D1 (UBE2D1) (ENSG00000072401.14), VPS26 retromer complex component A (VPS26A) (ENSG00000122958.14), eukaryotic translation initiation factor 5A like 1 (EIF5AL1) (ENSG00000253626.3), kinesin family member 20B (KIF20B) (ENSG00000138182.14), FRA10A associated CGG repeat 1 (FRA10AC1) (ENSG00000148690.11), survival motor neuron domain containing 1 (SMNDC1) (ENSG00000119953.12), coiled-coil domain containing 186 (CCDC186) (ENSG00000165813.18), pleckstrin homology like domain family A member 2 (PHLDA2) (ENSG00000181649.6), MT-RNR2 like 8 (pseudogene) (MTRNR2L8) (ENSG00000255823.4), lin-7 homolog C, crumbs cell polarity complex component (LIN7C) (ENSG00000148943.11), glycine-N-acyltransferase like 1 (GLYATL1) (ENSG00000166840.13), membrane spanning 4-domains A6E (MS4A6E) (ENSG00000166926.8), heterogeneous nuclear ribonucleoprotein U like 2 (HNRNPUL2) (ENSG00000214753.2), FKBP prolyl isomerase 2 (FKBP2) (ENSG00000173486.12), spliceosome associated factor 1, recruiter of U4/U6.U5 tri-snRNP (SART1) (ENSG00000175467.14), remodeling and spacing factor 1 (RSF1) (ENSG00000048649.13), cysteine and histidine rich domain containing 1 (CHORDC1) (ENSG00000110172.11), ankyrin repeat domain 49 (ANKRD49) (ENSG00000168876.8), family with sequence similarity 76 member B (FAM76B) (ENSG00000077458.12), coiled-coil domain containing 82 (CCDC82) (ENSG00000149231.12), transmembrane protein 123 (TMEM123) (ENSG00000152558.14), defective in cullin neddylation 1 domain containing 5 (DCUN1D5) (ENSG00000137692.11), RNA binding motif protein 7 (RBM7) (ENSG00000076053.10), H2A.X variant histone (H2AFX) (ENSG00000188486.3), parathymosin (PTMS) (ENSG00000159335.15), basic helix-loop-helix family member e41 (BHLHE41) (ENSG00000123095.5), ERGIC and golgi 2 (ERGIC2) (ENSG00000087502.17), glucoside xylosyltransferase 1 (GXYLT1) (ENSG00000151233.10), zinc finger CCHC-type and RNA binding motif containing 1 (ZCRB1) (ENSG00000139168.7), twinfilin actin binding protein 1 (TWF1) (ENSG00000151239.13), activating transcription factor 1 (ATF1) (ENSG00000123268.8), oxysterol binding protein like 8 (OSBPL8) (ENSG00000091039.16), RNA 5′-phosphate and 3′-OH ligase 1 (C12orf29) (ENSG00000133641.17), centrosomal protein 290 (CEP290) (ENSG00000198707.14), early endosome antigen 1 (EEA1) (ENSG00000102189.16), small nuclear ribonucleoprotein polypeptide F (SNRPF) (ENSG00000139343.10), nuclear transcription factor Y subunit beta (NFYB) (ENSG00000120837.7), mitochondrial calcium uptake 2 (MICU2) (ENSG00000165487.13), poly(A) binding protein cytoplasmic 3 (PABPC3) (ENSG00000151846.8), casein kinase 1 alpha 1 like (CSNK1A1L) (ENSG00000180138.7), laccase domain containing 1 (LACC1) (ENSG00000179630.10), GPALPP motifs containing 1 (GPALPP1) (ENSG00000133114.17), tumor protein, translationally-controlled 1 (TPT1) (ENSG00000133112.16), zinc finger CCCH-type containing 13 (ZC3H13) (ENSG00000123200.16), integral membrane protein 2B (ITM2B) (ENSG00000136156.12), cytoskeleton associated protein 2 (CKAP2) (ENSG00000136108.14), mitotic spindle organizing protein 1 (MZT1) (ENSG00000204899.5), KLF transcription factor 5 (KLF5) (ENSG00000102554.13), potassium channel tetramerization domain containing 12 (KCTD12) (ENSG00000178695.5), coiled-coil domain containing 168 (CCDC168) (ENSG00000175820.3), arginine and glutamate rich 1 (ARGLU1) (ENSG00000134884.13), serine palmitoyltransferase small subunit A (SPTSSA) (ENSG00000165389.6), ras homolog family member J (RHOJ) (ENSG00000126785.12), golgin A6 family like 6 (GOLGA6L6) (ENSG00000277322.1), golgin A6 family like 2 (GOLGA6L2) (ENSG00000174450.11), ER membrane protein complex subunit 7 (EMC7) (ENSG00000134153.9), eukaryotic translation initiation factor 3 subunit J (EIF3J) (ENSG00000104131.12), COP9 signalosome subunit 2 (COPS2) (ENSG00000166200.14), RAB27A, member RAS oncogene family (RAB27A) (ENSG00000069974.15), SAFB like transcription modulator (SLTM) (ENSG00000137776.16), reticulocalbin 2 (RCN2) (ENSG00000117906.13), methenyltetrahydrofolate synthetase (MTHFS) (ENSG00000136371.10), zinc finger AN1-type containing 6 (ZFAND6) (ENSG00000086666.18), nuclear receptor subfamily 2 group F member 2 (NR2F2) (ENSG00000185551.14), centrosomal protein 20 (FOPNL) (ENSG00000133393.12), centriolar coiled-coil protein 110 (CCP110) (ENSG00000103540.16), nuclear pore complex interacting protein family member B5 (NPIPB5) (ENSG00000243716.10), eukaryotic translation initiation factor 3 subunit C like (EIF3CL) (ENSG00000205609.12), terminal nucleotidyltransferase 4B (PAPD5) (ENSG00000121274.12), tubulin polymerization promoting protein family member 3 (TPPP3) (ENSG00000159713.10), NIN1 (RPN12) binding protein 1 homolog (NOB1) (ENSG00000141101.12), contactin associated protein family member 4 (CNTNAP4) (ENSG00000152910.18), MAF bZIP transcription factor (MAF) (ENSG00000178573.6), CBFA2/RUNX1 partner transcriptional co-repressor 3 (CBFA2T3) (ENSG00000129993.14), ankyrin repeat domain containing 11 (ANKRD11) (ENSG00000167522.15), ER membrane protein complex subunit 6 (TMEM93) (ENSG00000127774.6), lysine demethylase 6B (KDM6B) (ENSG00000132510.10), ribosomal protein L26 (RPL26) (ENSG00000161970.13), SUZ12 polycomb repressive complex 2 subunit (SUZ12) (ENSG00000178691.10), insulin like growth factor binding protein 4 (IGFBP4) (ENSG00000141753.6), keratin 10 (KRT10) (ENSG00000186395.7), transducer of ERBB2, 1 (TOB1) (ENSG00000141232.4), nascent polypeptide associated complex subunit alpha 2 (NACA2) (ENSG00000253506.2), SRY-box transcription factor 9 (SOX9) (ENSG00000125398.6), ring finger protein 138 (RNF138) (ENSG00000134758.13), immediate early response 3 interacting protein 1 (IER3IP1) (ENSG00000134049.5), methyl-CpG binding domain protein 2 (MBD2) (ENSG00000134046.11), SEC11 homolog C, signal peptidase complex subunit (SEC11C) (ENSG00000166562.8), coiled-coil domain containing 102B (CCDC102B) (ENSG00000150636.17), zinc finger and BTB domain containing 7A (ZBTB7A) (ENSG00000178951.8), scaffold attachment factor B2 (SAFB2) (ENSG00000130254.11), scaffold attachment factor B (SAFB) (ENSG00000160633.12), tubulin beta 4A class IVa (TUBB4A) (ENSG00000104833.11), RAB11B, member RAS oncogene family (RAB11B) (ENSG00000185236.11), solute carrier family 1 member 6 (SLC1A6) (ENSG00000105143.12), KLF transcription factor 2 (KLF2) (ENSG00000127528.5), zinc finger protein 708 (ZNF708) (ENSG00000182141.10), PAF1 homolog, Paf1/RNA polymerase II complex component (PAF1) (ENSG00000006712.14), transforming growth factor beta 1 (TGFB1) (ENSG00000105329.9), novel protein, readthrough between CEACAM5-CEACAM6 (AC011513.3) (ENSG00000267881.1), CD177 molecule (CD177) (ENSG00000204936.9), potassium voltage-gated channel subfamily A member 7 (KCNA7) (ENSG00000104848.1), ribosomal protein L28 (RPL28) (ENSG00000108107.14), zinc finger protein 580 (ZNF580) (ENSG00000213015.8), ESF1 nucleolar pre-rRNA processing protein homolog (ESF1) (ENSG00000089048.14), charged multivesicular body protein 4B (CHMP4B) (ENSG00000101421.3), DNA topoisomerase I (TOP1) (ENSG00000198900.5), prefoldin subunit 4 (PFDN4) (ENSG00000101132.9), PRELI domain containing 3B (PRELID3B) (ENSG00000101166.15), synaptonemal complex protein 2 (SYCP2) (ENSG00000196074.12), TATA-box binding protein associated factor 4 (TAF4) (ENSG00000130699.18), cholinergic receptor nicotinic alpha 4 subunit (CHRNA4) (ENSG00000101204.16), SRY-box transcription factor 18 (SOX18) (ENSG00000203883.6), histone H2B type F-S-like (CH507-42P11.1) (ENSG00000274559.3), ATP synthase peripheral stalk subunit F6 (ATP5J) (ENSG00000154723.12), GA binding protein transcription factor subunit alpha (GABPA) (ENSG00000154727.10), SR-related CTD associated factor 4 (SCAF4) (ENSG00000156304.14), H2B clustered histone 12 like (H2BFS) (ENSG00000234289.5), BCLAF1 And THRAP3 family member 3 (CXorf23) (ENSG00000173681.16), proprotein convertase subtilisin/kexin type 1 inhibitor (PCSKIN) (ENSG00000102109.8), MT-RNR2 like 10 (pseudogene) (MTRNR2L10) (ENSG00000256045.2), ATRX chromatin remodeler (ATRX) (ENSG00000085224.21), transcription elongation factor A like 5 (TCEAL5) (ENSG00000204065.2), glutamate dehydrogenase 2 (GLUD2) (ENSG00000182890.4), hypoxanthine phosphoribosyltransferase 1 (HPRT1) (ENSG00000165704.14), oxidoreductase core subunit 6 (MT-ND6) (ENSG00000198695.2), enolase 1 (ENO1) (ENSG00000074800.14), solute carrier family 2 member 1 (SLC2A1) (ENSG00000117394.21), adenylate kinase 4 (AK4) (ENSG00000162433.14), tuftelin 1 (TUFT1) (ENSG00000143367.15), activating transcription factor 3 (ATF3) (ENSG00000162772.16), regenerating family member 1 alpha (REG1A) (ENSG00000115386.5), dual specificity phosphatase 2 (DUSP2) (ENSG00000158050.4), pyruvate dehydrogenase kinase 1 (PDK1) (ENSG00000152256.13), basic helix-loop-helix family member e40 (BHLHE40) (ENSG00000134107.4), C-X-C motif chemokine ligand 8 (CXCL8) (ENSG00000169429.10), secreted phosphoprotein 1 (SPP1) (ENSG00000118785.13), secreted frizzled related protein 2 (SFRP2) (ENSG00000145423.4), natriuretic peptide receptor 3 (NPR3) (ENSG00000113389.15), stanniocalcin 2 (STC2) (ENSG00000113739.10), immediate early response 3 (IER3) (ENSG00000137331.11), heat shock protein family A (Hsp70) member 1B (HSPA1B) (ENSG00000204388.6), alpha-2-glycoprotein 1, zinc-binding (AZGP1) (ENSG00000160862.12), potassium voltage-gated channel subfamily H member 2 (KCNH2) (ENSG00000055118.14), BCL2 interacting protein 3 like (BNIP3L) (ENSG00000104765.15), PLAG1 zinc finger (PLAG1) (ENSG00000181690.7), N-myc downstream regulated 1 (NDRG1) (ENSG00000104419.14), carbonic anhydrase 9 (CA9) (ENSG00000107159.12), UDP-N-acetylglucosamine pyrophosphorylase 1 like 1 (UAP1L1) (ENSG00000197355.10), prolyl 4-hydroxylase subunit alpha 1 (P4HA1) (ENSG00000122884.12), interferon induced transmembrane protein 1 (IFITM1) (ENSG00000185885.15), interferon induced transmembrane protein 3 (IFITM3) (ENSG00000142089.15), cleavage stimulation factor subunit 3 (CSTF3) (ENSG00000176102.11), protein kinase C and casein kinase substrate in neurons 3 (PACSIN3) (ENSG00000165912.15), fatty acid desaturase 2 (FADS2) (ENSG00000134824.13), fatty acid desaturase 1 (FADS1) (ENSG00000149485.18), enolase 2 (ENO2) (ENSG00000111674.8), COPI coat complex subunit zeta 1 (COPZ1) (ENSG00000111481.9), syntaxin 2 (STX2) (ENSG00000111450.13), endoplasmic reticulum oxidoreductase 1 alpha (ERO1A) (ENSG00000197930.12), dipeptidase 1 (DPEP1) (ENSG00000015413.9), lectin, mannose binding 1 (LMAN1) (ENSG00000074695.5), kallikrein related peptidase 6 (KLK6) (ENSG00000167755.14), troponin T1, slow skeletal type (TNNT1) (ENSG00000105048.16), ribophorin II (RPN2) (ENSG00000118705.16), WAP four-disulfide core domain 3 (WFDC3) (ENSG00000124116.18), deoxynucleotidyltransferase terminal interacting protein 1 (DNTTIP1) (ENSG00000101457.12), ubiquitin conjugating enzyme E2 C (UBE2C) (ENSG00000175063.16), dolichyl-phosphate mannosyltransferase subunit 1, catalytic (DPM1) (ENSG00000000419.12).

12. The method according to claim 10, wherein for distinction of MXE1 from MXE2, the PSI value of exon1 is higher than exon2 in MXE1 subtype indicating that exon1 preferentially is retained over exon2 for the exons as defined in claim 11.

13. The method according to claim 10, wherein for distinction of MXE1 from MXE2, the PSI value exon1 is higher than exon2 in MXE2 subtype indicating that exon1 is preferentially retained over exon 2 for the exons as defined in claim 11.

14. The method according to claim 5, wherein in MXE1 subtype, primarily exon1 (with genomic coordinates chr21: 45480466-45480520) of gene collagen type XVIII alpha 1 chain (COL18A1) (ENSG00000182871.14), is 18.3%+/−0.9 (SE) higher compared to exon2 (with genomic coordinates chr21: 45480699-45480858); exon1 (with genomic coordinates chr19: 33012646-33012724) of gene rhophilin Rho GTPase binding protein 2 (RHPN2) (ENSG00000131941.7), is 12.2%+/−0.6 (SE) higher compared to exon2 (with genomic coordinates chr19: 33021570-33021646); exon1 (with genomic coordinates chr19: 10986188-10986593) of gene SWI/SNF related, matrix associated, actin dependent regulator of chromatin, subfamily a, member 4 (SMARCA4) (ENSG00000127616.18), is 16.8%+/−0.9 (SE) higher compared to exon2 (with genomic coordinates chr19: 10986904-10987003); exon1 (with genomic coordinates chr17: 69304668-69304810) of gene ATP binding cassette subfamily A member 5 (ABCA5) (ENSG00000154265.15), is 23%+/−1.7 (SE) higher compared to exon2 (with genomic coordinates chr17: 69306724-69306954); exon1 (with genomic coordinates chr11: 66564087-66564146) of gene cathepsin F (CTSF) (ENSG00000174080.10), is 20.7%+/−1.1 (SE) higher compared to exon2 (with genomic coordinates chr11: 66564557-66564648); exon1 (with genomic coordinates chr1: 200844781-200844869) of gene calmodulin regulated spectrin associated protein family member 2 (CAMSAP2) (ENSG00000118200.14), is 16.7%+/−1.2 (SE) higher compared to exon2 (with genomic coordinates chr1: 200848031-200850234); exon1 (with genomic coordinates chr2: 188988080-188988134) of gene collagen type III alpha 1 chain (COL3A1) (ENSG00000168542.14), is 15.8%+/−0.6 (SE) higher compared to exon2 (with genomic coordinates chr2: 188989395-188989449); exon1 (with genomic coordinates chr1: 54205294-54205410) of gene mitochondrial ribosomal protein L37 (MRPL37) (ENSG00000116221.15), is 13.7%+/−0.5 (SE) higher compared to exon2 (with genomic coordinates chr1: 54209945-54210131); exon1 (with genomic coordinates chr6: 149836084-149836297) of gene LDL receptor related protein 11 (LRP11) (ENSG00000120256.9), is 13.6%+/−0.6 (SE) higher compared to exon2 (with genomic coordinates chr6: 149837337-149837463); exon1 (with genomic coordinates chrX: 41140655-41140771) of gene ubiquitin specific peptidase 9 X-linked (USP9X) (ENSG00000124486.12), is 16.4%+/−0.7 (SE) higher compared to exon2 (with genomic coordinates chrX: 41140965-41141217); exon1 (with genomic coordinates chr6: 31642828-31643115) of gene BAG cochaperone 6 (BAG6) (ENSG00000204463.12), is 14.8%+/−0.9 (SE) higher compared to exon2 (with genomic coordinates chr6: 31644081-31644194); exon1 (with genomic coordinates chr6: 33303954-33303989) of gene TAP binding protein (TAPBP) (ENSG00000231925.11), is 13.4%+/−0.6 (SE) higher compared to exon2 (with genomic coordinates chr6: 33304127-33304217); exon1 (with genomic coordinates chr7: 34966893-34966971) of gene dpy-19 like C-mannosyltransferase 1 (DPY19L1) (ENSG00000173852.14), is 18.3%+/−1.3 (SE) higher compared to exon2 (with genomic coordinates chr7: 34969432-34969532); exon1 (with genomic coordinates chr2: 24299908-24300171) of gene intersectin 2 (ITSN2) (ENSG00000198399.14), is 14.9%+/−0.7 (SE) higher compared to exon2 (with genomic coordinates chr2: 24301153-24301239); exon1 (with genomic coordinates chr10: 112438335-112438389) of gene zinc finger DHHC-type palmitoyltransferase 6 (ZDHHC6) (ENSG00000023041.11), is 16.4%+/−0.8 (SE) higher compared to exon2 (with genomic coordinates chr10: 112440533-112440695); exon1 (with genomic coordinates chr10: 112438335-112438389) of gene zinc finger DHHC-type palmitoyltransferase 6 (ZDHHC6) (ENSG00000023041.11), is 15.9%+/−0.8 (SE) higher compared to exon2 (with genomic coordinates chr10: 112442191-112442351).

15. The method according to claim 5, wherein in MXE2 subtype primarily, exon1 (with genomic coordinates chr20: 48988490-48988662) of gene ADP ribosylation factor guanine nucleotide exchange factor 2 (ARFGEF2) (ENSG00000124198.8), is 15%+/−0.6 (SE) higher compared to exon2 (with genomic coordinates chr20: 48989284-48989436); exon1 (with genomic coordinates chr18: 62266700-62266749) of gene RAB11 binding and LisH domain (KIAA1468) (ENSG00000134444.14), is 15.8%+/−1 (SE) higher compared to exon2 (with genomic coordinates chr18: 62268868-62268948); exon1 (with genomic coordinates chr16: 30081258-30081310) of gene protein phosphatase 4 catalytic subunit (PPP4C) (ENSG00000149923.13), is 16.1%+/−0.8 (SE) higher compared to exon2 (with genomic coordinates chr16: 30082483-30082534); exon1 (with genomic coordinates chr15: 34255261-34255392) of gene solute carrier family 12 member 6 (SLC12A6) (ENSG00000140199.11), is 14.1%+/−1 (SE) higher compared to exon2 (with genomic coordinates chr15: 34258812-34258944); exon1 (with genomic coordinates chr14: 22905605-22905659) of gene RNA binding motif protein 23 (RBM23) (ENSG00000100461.17), is 19.2%+/−1.2 (SE) higher compared to exon2 (with genomic coordinates chr14: 22908332-22908380); exon1 (with genomic coordinates chr12: 7142085-7142139) of gene calsyntenin 3 (CLSTN3) (ENSG00000139182.13), is 17.2%+/−1 (SE) higher compared to exon2 (with genomic coordinates chr12: 7142868-7143026); exon1 (with genomic coordinates chr20: 13534021-13534141) of gene taspase 1 (TASP1) (ENSG00000089123.15), is 14.1%+/−0.8 (SE) higher compared to exon2 (with genomic coordinates chr20: 13559007-13559114); exon1 (with genomic coordinates chr8: 52630469-52630528) of gene RB1 inducible coiled-coil 1 (RB1CC1) (ENSG00000023287.12), is 14.1%+/−0.8 (SE) higher compared to exon2 (with genomic coordinates chr8: 52634920-52634968); exon1 (with genomic coordinates chr1: 150156065-150156206) of gene pleckstrin homology domain containing 01 (PLEKHO1) (ENSG00000023902.13), is 12.8%+/−0.7 (SE) higher compared to exon2 (with genomic coordinates chr1: 150157384-150157486); exon1 (with genomic coordinates chr2: 32607454-32607643) of gene baculoviral IAP repeat containing 6 (BIRC6) (ENSG00000115760.13), is 13.4%+/−0.6 (SE) higher compared to exon2 (with genomic coordinates chr2: 32611447-32611582); exon1 (with genomic coordinates chr11: 9430874-9431003) of gene importin 7 (IPO7) (ENSG00000205339.9), is 14.4%+/−0.6 (SE) higher compared to exon2 (with genomic coordinates chr11: 9433569-9433636); exon1 (with genomic coordinates chr17: 82081163-82081352) of gene fatty acid synthase (FASN) (ENSG00000169710.8), is 13.5%+/−0.7 (SE) higher compared to exon2 (with genomic coordinates chr17: 82081600-82081843); exon1 (with genomic coordinates chr6: 157873101-157873176) of gene sorting nexin 9 (SNX9) (ENSG00000130340.15), is 15.5%+/−0.6 (SE) higher compared to exon2 (with genomic coordinates chr6: 157875050-157875176); exon1 (with genomic coordinates chr7: 5390484-5390628) of gene trinucleotide repeat containing 18 (TNRC18) (ENSG00000182095.14), is 21.6%+/−1 (SE) higher compared to exon2 (with genomic coordinates chr7: 5394439-5394595);
exon1 (with genomic coordinates chr17: 50195922-50195976) of gene collagen type I alpha 1 chain (COL1A1) (ENSG00000108821.13), is 13%+/−0.4 (SE) higher compared to exon2 (with genomic coordinates chr17: 50196154-50196199).

16. The method of claim 3, wherein in step c) the conversion of values of step b) to indicate the disease prognosis classifies the tumors as MXE1 subtype or MXE2 subtype.

17. The method of claim 3, wherein in step c) the rating determine whether the subject is subjected to a treatment by anti-tumor therapy and/or surveillance of progress of the disease.

18. The method of claim 3, wherein in step c) the conversion of the values of step b) to indicate the disease prognosis is combined with tumor classification subtypes other than MXE1 subtype and MXE2 subtype, preferably is combined with at least one tumor classification subtypes selected from CMS and immune infiltrate.
